(19) United States
(12) Patent Application Publication
(10) Pub. No.: US 2005/0080260 A1
(43) Pub. Date: Apr. 14, 2005

(54) Preparation of prodrugs for selective drug delivery

(76) Inventors: Randell L. Mills, Cranbury, NJ (US); Guo-Zhang Wu, Belle Mead, NJ (US)

Correspondence Address: MANELLI DENISON & SELTER, 2000 M STREET NW SUITE 700, WASHINGTON, DC, 20036-3307, US

(21) Appl. No.: 10/828,558

(22) Filed: Apr. 21, 2004

Related U.S. Application Data
(60) Provisional application No. 60/464,354, filed on Apr. 22, 2003.

(51) Int. Cl. C07D 237/30; C07C 211/27

(52) U.S. Cl. 544/237; 564/338

(57) ABSTRACT

Synthesis of a chemical compound having the formula A-B-C that may serve for applications such as drug delivery where A is a chemiluminescent, moiety, B is a photochromic moiety, and C is a biologically active moiety where A-B-C may serve as a prodrug. Novel synthetic methods of the present invention to form the prodrug comprised the steps of (1) forming a benzophenone, (2) forming a diaryl ethylene, (3) attaching a phthalimide moiety to at least one of the aryl groups of the ethylene to form a phthalimide-ethylene conjugate, (4) condensing two ethylene-phthalimide conjugates to form a phthalimide-pentadiene conjugate, (5) converting the phthalimide to the phthalhydrazide by reaction with hydrazine to form a carrier compound according to the present invention, and (6) reacting the carrier compound with an nucleophilic moiety of the drug to form the corresponding prodrug. Alternatively the carrier can be prepared by using the halo-substituted diaryl ethylene to make the corresponding cationic leuco dye-like compound with known methods. The cationic compound then is protected by reacting with a nucleophile and coupled with the aminophathalimide by palladium-catalyzed amination to form the protected phthalimide-pentadiene conjugate. The latter is refluxed with hydrazine to convert its phthalimide to the phthalhydrazide and acidified to give the carrier. An additional aspect of the present invention relates to the use of these compounds as antiviral agents for the treatment of viral infections such as HIV and as anticancer agents for the treatment of cancers such as bowel, lung, and breast cancer.

This application claims priority to U.S. Provisional Application No. 60/464,354, filed on Apr. 22, 2003, the complete disclosure of which is incorporated herein by reference.

### FIELD OF THE INVENTION

This invention relates generally to the field of organic chemistry, and discloses novel methods of synthesis of prodrugs disclosed in Mills prior US patents, U.S. Pat. No. 5,773,592, Randell L. Mills, Jun. 30, 1998, entitled, “Prodrugs for Selective Drug Delivery” and U.S. Pat. No. 5,428,163, Randell L. Mills, Jun. 27, 1995 entitled “Prodrugs for Selective Drug Delivery” which are herein incorporated in their entirety by reference and herein after referred to as “Mills Prior patents”. The present invention relates to the synthesis of therapeutic pharmaceutical agents which may be activated intracellularly by reaction with cellular electron carriers or free radicals to cause release of a free and active drug molecule. An additional aspect of the present invention relates to the use of these compounds as antiviral agents for treatment of infection of at least one of the group of Human Immunodeficiency Virus (HIV), herpes viruses such as Herpes Simplex Virus, (HSV), Epstein-Barr Virus (EBV), Varicella Zoster (VZV), Cytomegalovirus (CMV), HSV-6, and HSV-8 (Kaposi's sarcoma), Human Papilloma Virus (HPV), rhinoviruses, and hepatitis-linked viruses. Another aspect of the present invention relates to the use of these compounds as anticancer agents for the treatment of cancers of least one not limited to the cancers from the group of colon, breast, lung, renal, retinal, and skin.

### BACKGROUND OF THE INVENTION

Many compounds are known which have receptor or nonreceptor mediated in vitro activity as appears in Handbook of Enzyme Inhibitors, Mahendra Kumor Jain, 1982, Wiley Interscience, New York, hereby incorporated by reference. However, only a small percentage produce the desired functional change in vivo or have a high therapeutic ratio because they are toxic in their free form; they are rapidly inactivated or excreted; or, they cannot obtain access to their target receptor or site of action because they are impermeant to cells or biological barriers such as the blood brain barrier due to unfavorable energetics due, for example, to the possession of polar or charge groups; or, they are toxic as a consequence of being nonselective with regards to their access to and action with receptors in one biological environment or compartment relative to another. In these cases, compounds which demonstrate in vitro efficacy are ineffective therapeutics.

Past attempts to increase the bioavailability of drugs include bulk delivery strategies, including the use of liposomes, and drug delivery strategies involving the formation of derivatives of drugs such as ester derivatives. The major limitations in the case of liposomes are the inability to direct the bulk release to a specific tissue for the most part, the lack of a mechanism to increase the permeability of the drug, and the clearing of the liposomes by the reticuloendothelial system (liver). The major weakness of the esterified drugs strategy is that the mechanism of free drug release depends on the existence of an enzyme of the organism to cleave the bond between the ester and the drug. Such enzymes are typically not present or have little activity in the target cells or biological compartment on the prodrug.

Many potent antiHIV drugs comprise nucleoside or nucleotide analogs which are effective reverse transcriptase or polymerase inhibitors, but have poor bioavailability due to low lipophilicity with poor diffusion capability across cell membranes. In our prodrug studies, the drug comprised a reverse transcriptase inhibitor, either phosphonoformate (Foscarnet) or dideoxycytidine (ddc). Foscarnet showed great promise as an antiHIV drug as indicated by in vivo screening assays and in clinical trials. In the later case, it was found that significant HIV suppression over two weeks at mean serum concentrations of. However, the treatment was interrupted due to renal function impairment [1]. In an attempt to improve the bioavailability of this very lipophilic drug and to improve the therapeutic ratio, many prodrug schemes have developed such as Foscarnet encapsulated in liposomes [2-3] or linked to alkylalcohols [4-5], covalent lipid conjugates [6-7], steroid derivatives [8], and glycerolphospholipid derivatives [9]. In addition, more lipophilic analogs such as those containing sulfur [10], 2-hydroxy-1,4,2-dioxaphosphorinane-2,3-dioxide derivatives [11], glucosyl esters [12], and ester derivatives [13] have been synthesized. Limitations of these strategies are the requirement of a means to cause selective intracellular cleavage of the modified group to recover the free drug and a reduction of the potency of the modified compounds compared to that of Foscarnet alone. Furthermore, antiHIV prodrugs of nucleotides such as carbonates of zidovudine (AZT) have been synthesized and evaluated wherein cyclic intramolecular rearrangement recovers the original drug [14].

The present invention relates to the synthesis of prodrugs with increased bioavailability. In one embodiment, a prodrug comprises a three-part molecule, A-B-C, where each part is a functionality with a defined purpose. One embodiment relates to cellular permeant prodrugs where intracellular drug release may occur when the prodrug reacts with cellular free radicals via a mechanism involving chemiluminescence, photochromism, and intramolecular energy transfer.

### SUMMARY OF THE INVENTION

Methods of synthesis of a broad class of pharmaceutical agents is disclosed herein as the Luminide class of pharmaceuticals. Luminide agents are three part or four part molecules where each part is a functionality with a defined purpose. Exemplary Luminides are A-B-C, D-A-B-C, A-D-B-C, and 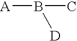
where A represents a functionality which is activatable by the environment and capable of transferring energy from its own excited state to the B functionality which is an energy acceptor. Upon receiving energy from A, B achieves an excited state which relaxes through the heterolytic cleavage of the covalent bond of B with C where C is a drug moiety which is released into the intracellular compartment where activation of A occurred. Released C can act locally or at a distant site. D serves as an electron transfer functionality which gains (loses) electrons from (to) the environment and donates (accepts) electrons to (from) A to activate it so that the energy of excited A is transferred to B with release of C as occurs for the three functionality case.

In both cases, free C is a drug molecule. The released drug molecule effects a therapeutic functional change by a mechanism which comprises receptor mediated mechanisms including reversible or irreversible competitive agonism or antagonism including a suicide substrate or transition state analogue mechanism or a noncompetitive or uncompetitive agonism or antagonism or the action is by a nonreceptor mediated mechanism including a “counterfeit incorporation mechanism”.

The chemical and physical properties of the Luminide agents such as permeance and reactivity to different oxidoreductase enzymes, electron carriers, or different free radicals including those of oxygen are exploited to control the environment into which C is released. Permeance of the Luminide agent to the blood brain barrier or cell membranes, or affinity of the Luminide agent to plasma proteins which results in a decreased excretion rate relative to free C, or lack of reactivity of extracellular enzymes with the Luminide agent relative to free C are exemplary mechanism where by Luminides provide for the release of active free C in the proper biological compartment or in the presence of the target receptor so that the desired therapeutic-change is achieved. Thus, Luminides serve as therapeutic drugs. And, the present invention, Luminides, a broad class of pharmaceutical agents comprises antilipidemic drugs, anticholesterol drugs, contraceptive agents, anticoagulants, anti-inflamatory agents, immuno-suppressive drugs, antiarrhythmic agents, antineoplastic drugs, antihypertensive drugs, epinephrine blocking agents, cardiac inotropic drugs, antidepressant drugs, diuretics, antifungal agents, antibacterial drugs, anxiolytic agents, sedatives, muscle relaxants, anticonvulsants, agents for the treatment of ulcer disease, agents for the treatment of asthma and hypersensitivity reactions, antithroboembolic agents, agents for the treatment of muscular dystrophy, agents to effect a therapeutic abortion, agents for the treatment of anemia, agents to improve allograft survival, agents for the treatment of disorders of purine metabolism, agents for the treatment of ischemic heart disease, agents for the treatment of opiate withdrawal, agents which activate the effects of secondary messenger inositol triphosphate, agents to block spinal reflexes, and antiviral agents including a drug for the treatment of AIDS.

The novel synthetic methods of the present invention to form a Luminide prodrug, which are illustrated in the General Scheme below, comprise the steps of 1.) forming a benzophenone, G1, 2.) forming a diaryl ethylene, G2, 3.) attaching a phthalimide moiety to at least one of the aryl groups of the ethylene to form a phthalimide-ethylene conjugate, G3, 4.) condensing two ethylene-phthalimide conjugates to form a phthalimide-pentadiene conjugate, G4, 5.) converting the phthalimide to the phthalhydrazide by reaction with hydrazine to form a carrier compound according to the present invention, G5, and 6.) reacting the carrier compound with a nucleophilic moiety of the drug to form the corresponding prodrug such as G6. Alternatively the carrier G5 can be prepared by starting with halo-substituted diaryl ethylene, G2, to make the corresponding dye G7 with known methods. The cationic dye G7 then is protected by reacting with an nucleophile to form G8 and coupled with the aminophathalimide to form the protected phthalimide-pentadiene conjugate G9. G9 is refluxed with hydrazine to convert its phthalimide to the phthalhydrazide and acidified to give the carrier G5.

An additional aspect of the present invention relates to the use of these compounds as antiviral agents for the treatment of viral infections such as HIV.

These and other features, aspects, and advantages of this invention will become better understood with regard to the following detailed description and appended claims.

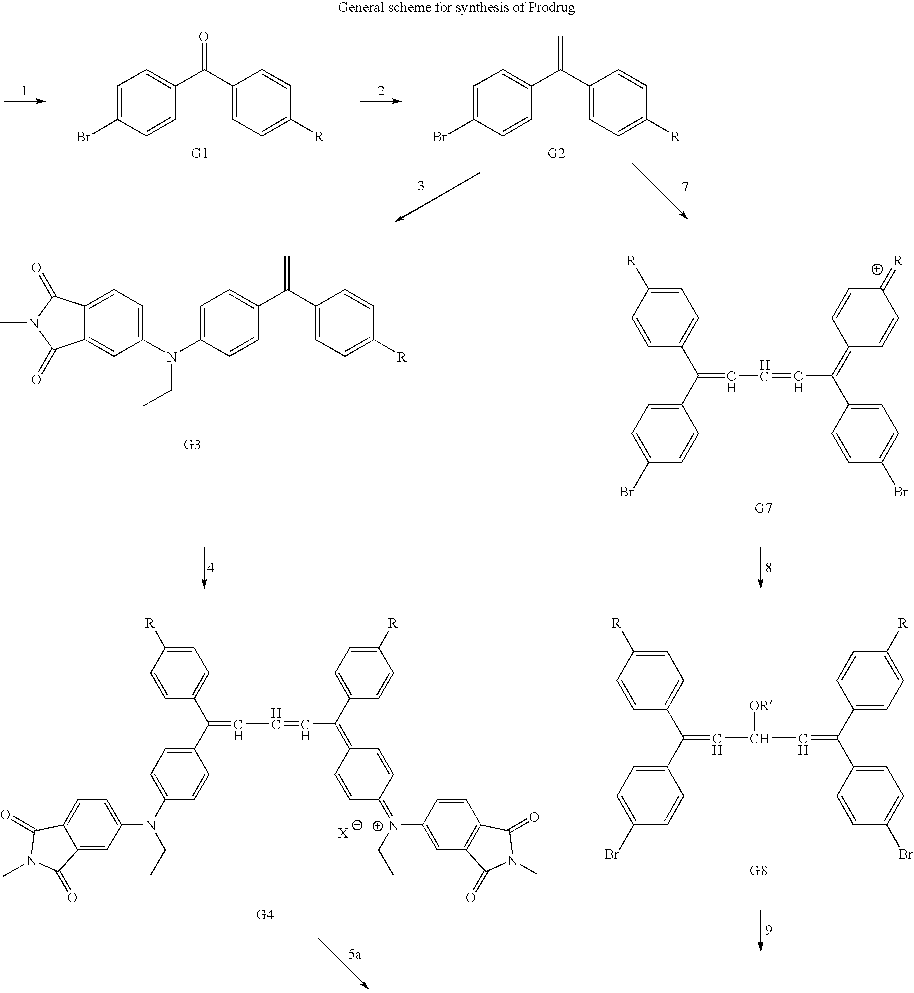

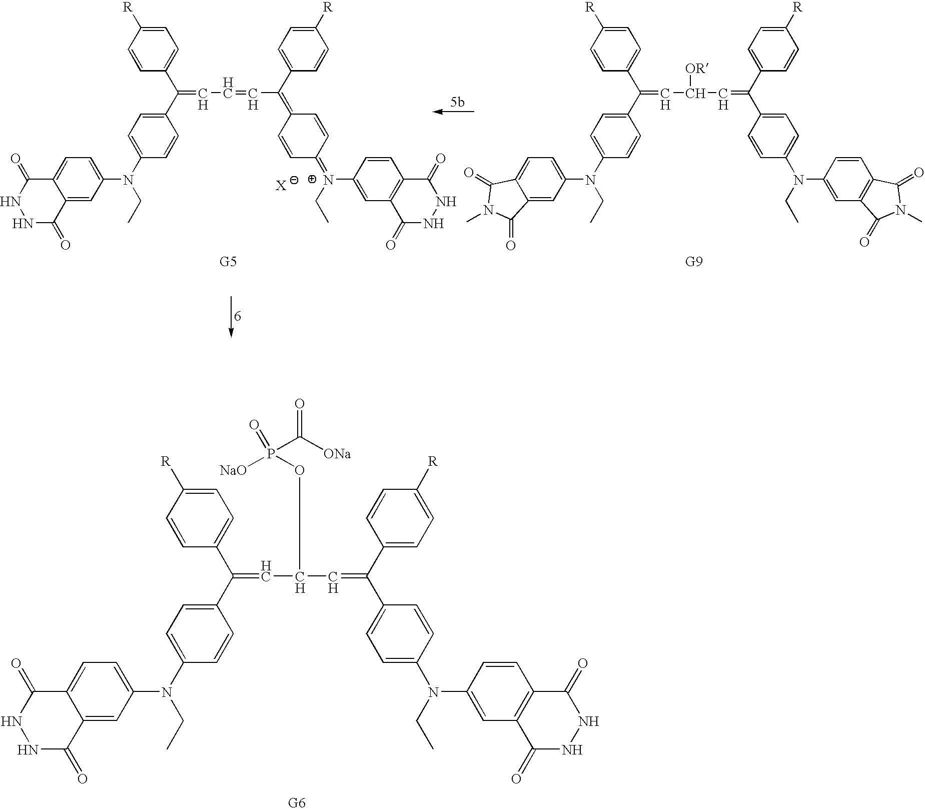

The invention comprises a method of synthesis of a chemical compound having the formula A-B-C
where the A is a chemiluminescent moiety,
B is an energy acceptor moiety, and
C is a biologically active moiety
   comprising the steps of
forming a benzophenone,
forming a diaryl ethylene, and performing at least one of
(a) attaching a precursor to generate a phthalhydrazide such as phthalimide, aminophthalic acid diester, aminophthalic acid dihydrazide, aminophthalic anhydride., and phthalhydrazide protected by a hydrolyzable group to form the precursor-ethylene conjugate, and condensing two ethylene-precursor conjugates to form a precursor-pentadiene conjugate, and
(b) condensing two diaryl ethylene to form a pentadiene, and attaching a precursor to generate a phthalhydrazide such as phthalimide, aminophthalic acid diester, aminophthalic acid dihydrazide, aminophthalic anhydride and phthalhydrazide protected by a hydrolyzable group, to form the precursor-pentadiene conjugate, and
converting the precursor to the phthalhydrazide by at least one of the corresponding reactions
phthalimide with hydrazine,
aminophthalic acid diester with hydrazine,
aminophthalic anhydride with hydrazine, and
hydrolysis of phthalhydrazide protected by a hydrolyzable group to form a carier compound, and
reacting the carrier compound with the biologically active moiety to form a corresponding conjugate.

The compound serves to delivery the C moiety to a desired biological compartment wherein the compound is a prodrug.

The compound may serve as a prodrug for at least one of antiviral agents for the treatment of viral infections and anticancer agents for the treatment of cancers.

The compound may serve as a prodrug for the treatment of at least one of the group of viruses comprising Human Immunodeficiency Virus (HIV), herpes viruses such as Herpes Simplex Virus, (HSV), Epstein-Barr Virus (EBV), Varicella Zoster (VZV), Cytomegalovirus (CMV), HSV-6, and HSV-8 (Kaposi's sarcoma), Human Papilloma Virus (HPV), rhinoviruses, and hepatitis-linked viruses.

The compound may serve as a prodrug for the treatment of at least one of the group of cancers comprising colon, breast, lung, renal, retinal, and skin.

The prodrugs have increased bioavailability.

In an embodiment, A-B-C is a cellular permeant prodrug.

Intracellular drug release may occur when the prodrug reacts with cellular free radicals via a mechanism involving chemiluminescence, photochromism, and intramolecular energy transfer.

In an embodiment, the C moiety is a pharmaceutical agent or drug.

The pharmaceutical agent may be at least one of the group of antilipidemic drugs, anticholesterol drugs, contraceptive agents, anticoagulants, anti-inflamatory agents, immunosuppressive drugs, antiarrhythmic agents, antineoplastic drugs, antihypertensive drugs, epinephrine blocking agents, cardiac inotropic drugs, antidepressant drugs, diuretics, antifungal agents, antibacterial drugs, anxiolytic agents, sedatives, muscle relaxants, anticonvulsants, agents for the treatment of ulcer disease, agents for the treatment of asthma and hypersensitivity reactions, antithroboembolic agents, agents for the treatment of muscular dystrophy, agents to effect a therapeutic abortion, agents for the treatment of anemia, agents to improve allograft survival, agents for the treatment of disorders of purine metabolism, agents for the treatment of ischemic heart disease, agents for the treatment of opiate withdrawal, agents which activate the effects of secondary messenger inositol triphosphate, agents to block spinal reflexes, and antiviral agents including a drug for the treatment of AIDS.

The C moiety may be released by an oxidation reduction reaction with the target cell's electron carriers or by reaction with free radicals produced as a consequence of electron transport.

The C moiety may be released into a desired compartment in active form.

The released C moiety may have a greater therapeutic effect or therapeutic ratio relative to the free C agent alone.

The released C moiety may have a greater therapeutic effect or therapeutic ratio relative to the free C agent alone as a consequence of at least one of altered pharmacokinetics or pharmacodynamics such as a desirable kinetics of release, a resistance to inactivation or excretion, greater solubility, enhanced absorption, a diminished toxicity, or greater access to the cellular or biological compartment which is the site of action of C.

In an embodiment, A represents a functionality which undergoes at least one of
an oxidation reduction reaction where electrons are transferred directly between A and the target cell's electron carriers, and
a reaction with free radicals of oxygen which are produced as a consequence of electron transport
such that an excited state is produced in A as a consequence of its participation in one of these reactions.

In an embodiment, A undergoes intramolecular energy transfer from its own excited state to the B functionality which is an energy acceptor. Upon receiving energy from A, B achieves an excited state which relaxes through heterolytic cleavage of the covalent bond of B with C where C is a drug moiety which is released into the environment.

In an embodiment, the released drug molecule effects a therapeutic functional change by a mechanism which comprises receptor mediated mechanisms including reversible and irreversible competitive agonism or antagonism including a molecule known as a suicide substrate or a transition state analogue mechanism or a noncompetitive or uncompetitive agonism or antagonism or the action is by a nonreceptor mediated mechanism including a “counterfeit incorporation-mechanism”.

The chemiluminescent molecule comprises at least one of the group of
molecules undergoing reaction involving peroxides and oxygen free radicals,
molecules undergoing reaction involving oxidation or reduction, and
molecules undergoing both reaction with peroxides and oxygen free radicals followed by an oxidation or reduction reaction.

The chemiluminescent molecule may comprise at least one of the group of luminol and its derivatives, lucigenin and its derivatives, Lophine and its derivatives, acridinium esters and acridans, tetraphenylpyrrole, phthalhydrazides, acyloins, biacridinium salts, vinylcarbonyls, vinylnitriles, tetrakis (dimethylamino) ethylene, acylperoxides, indoles, tetracarbazoles and active oxalates.

The chemiluminescent molecule may comprise at least one of the group of ruthenium chelates 2,6-diaminopyrene, or cation radicals and molecules which follow a Chemically Initiated Electron Exchange Luminescence mechanism such as certain dioxetans and dioxetanones.

The chemiluminescent molecule may comprise at least one of the group of dioxene derivatives and other compounds that form a dioxetan by reaction with superoxide and then produce efficient chemiluminescence by a CIEEL mechanism.

The chemiluminescent molecule may comprise at least one of the group of compounds given in Table 1.

The B moiety my be a photochromic compound.

The photochromic compound may comprise one which demonstrate photochromic behavior with electromagnetic radiation and bleaching agents.

In an embodiment, the A functionality is chemiluminescent, and the B functionality is such that the photodissociative drug release spectrum of B overlaps the chemiluminescence spectrum of A.

The photochromic compound may comprise a cationic dye.

In an embodiment, the cationic dye comprises at least one of a di and triarylmethane dyes, triarylmethane lactones and cyclic ether dyes, cationic indoles, pyronines, phthaleins, oxazines, thiazines, acridines, phenazines, and anthocyanidins, and cationic polymethine dyes and azo and diazopolymethines, styryls, cyanines, hemicyanines, dialkylaminopolyenes, and other related dyes.

In another embodiment, the cationic dye comprises at least one of the compounds given in Table 2.

The C moiety may be any molecule which exhibits bleaching behavior with the B moiety and has an increased therapeutic effect or therapeutic ratio as a consequence of its delivery as part of a prodrug.

The C moiety may have a nucleophilic group that bonds to the B moiety.

The C moiety may be derivatized to have a nucleophilic group that bonds to the B moiety.

The C moiety may be derivatized by at least one of the nucleophilic groups comprising cinnamate, sulfite, phosphate, carboxylate, thiol, amide, alkoxide, or amine.

In an embodiment, the C moiety is at least one of the group of compounds given in Table 3.

In an embodiment, the C moiety is at least one or a derivative or analog of one of the group of
prostaglandins
prostaglandin A.sub.1 A.sub.2 B.sub.1 E.sub.1, E.sub.2 or an analog which possesses a vasodilatory effect on coronary arteries and other human vascular beds
prostaglandin E, F, A or an analog which possesses a positive cardiac inotropic effect
prostaglandin A, E, or an analogue prostaglandin which possesses natriuretic and diuretic activity
prostaglandin A, G, E.sub.1, E.sub.2 or an analogue such as 15(S)-15-methyl PGE 2 methylester, 16,16-dimethyl PGE.sub.2, . . . AY-22,093, AY . . . 22,469, AY-22,443, or 15(R)-15-methyl PGE.sub.2 which inhibits gastric acid secretion
prostaglandin D.sub.2, E.sub.1 or an analogue which inhibits platelet aggregation
prostaglandin E.sub.1, E.sub.2 or an analogue which causes bronchial dilatation
prostaglandin F2 or an analogue which causes abortion by luteolysis
prostaglandin A.sub.2, E.sub.1, E.sub.2, or an analogue which induces erythropoiesis
prostaglandin E or an analogue which modulates T lymphocytes to decrease their ability to reject an allogenic graft
2′-isopropyl-4′-(trimethylammonium chloride)-5′-methylphenyl piperidine-1-carboxylate (Amo 1618) or an analog which inhibits the cyclization of trans-geranyl-geranyl-PP to copalyl-PP during Kaurene synthesis
adenosine cyclic 3′,5′-monophosphate or an analogue which inhibits the release and formation of phlogistic mediators such as histamine and kinins
4′-sulfamylphenyl
2-azo-7-acetamid-1-hydroxynaphthalene-3,6-disulfonate (Neoprontosil), 4′-sulfamyl-2, 4-diaminoazobenzene (Prontosil), or 5-(p-sulfamylphenylazo) salicylic acid (Lutazol) or analog which possess potent carbonic acid anhydrase inhibition
analogue of S-adenosyl homocysteine or sinefungin
phosphoglycolohydroxamate which inhibits Class II aldolases present in bacterial and fungi and is noninhibitory of Class I aldolases present in animals,
inosine analogue such as formycin B which inhibits nucleotide phosphorylase during nucleotide metabolism
phosphonoformate (Foscarnet) or an analog which inhibits the HIV reverse transcriptase enzyme
gamma.-amino-butyric acid (GABA) or an analog which is the major inhibitory neurotransmitter in the mannalian central nervous system
gabaculine, N-(5′-phosphopyridoxyl)-4-aminobutyric acid, ethanolamine-o-sulfate, .gamma.-vinyl GABA, or .gamma.-acetylenic GABA or an analog that is an inhibitor of the GABA-degrading enzyme, GABA: 2-oxoglutarate aminotransferase
Baclofen or a compound that inhibits GABA release
an oligonucleotide which binds to RNA or DNA and blocks transcription or translation of HIV or P-glycoprotein gene products adenosine which binds to brain purinergic receptors to suppress opiate withdrawal
adensoine whihc causes coronary vasodilatation
3-hydroxy-3-methylglutarate, 3-hydroxybutyrate, 3-hydroxy-3-methylpentanoate, 4-bromocrotonyl-CoA, but-3-ynoyl-CoA, pent-3-ynoyl-CoA, dec-3-ynoyl-CoA, ML-236A, ML-236B (compactin), ML-236C, mevinolin, mevinolinic acid, or a mevalonic acid analogue which is an inhibitor of 3-hydroxy-3-methylglutaryl-CoA reductase which catalyzes the rate-limiting and irreversible step of cholesterol synthesis where inhibition at this step does not lead to the accumulation of nonmetabolizable precursors
thioinosinate which suppresses T lymphocytes
Suramin, which is a powerful inhibitor of energy driven calcium uptake by the sarcoplasmic reticulum and is an intracellular inhibitor of Na.sup.+-K.sup.+ATPase where both activities increase intracellular calcium concentrations with a concomitant inotropic effect
norepinephrine N-methyltransferase inhibitor such as 2,3-dichloro-.alpha.-methylbenzylamine, 2,3-dichlorobenzylamine, 2,3-dichlorobenzamidine, or 3,4-dichlorophenylacetamidine
adenosine cyclic 3′,5′-monophosphate or a cAMP analogue which blocks the synthesis of fatty acids and cholesterol in the liver is an antilipidemic agent,
an inhibitor of dihydroxyphenylalanine decarboxylase during the synthesis of epinephrine and norepinephrine such as psitectorigenin, genistein, 3′, 4′,5,7-tetrahydroxy-8-methylisoflavone, orbol, 8-hydroxygenistein, 3′,5,7-trihydroxy-4′,6-dimethylisoflavone, 3′,5,7-trihydroxy-4′,8-dimethoxyisoflavone, D,L-B-(5-hydroxy-3-indolyl)-.alpha.-hydrazinopropionic acid, D,L-.alpha.-hydrazino-.alpha.-methyldopa, D,L-B-(3-indolyl), -.alpha.-hydrazinopropionic acid, a derivative of phenylalanine such as N-methyl-3,4-dopa, .alpha.-acetamido-3,4-dimethyoxycinnamic acid, DL-.alpha.-methyl-3,4-dopa, alpha.-methyl-B-(3-hydroxy-4-methoxyphenyl)alanine, alpha.-methyl-3,4-dimethoxyphenylalanine, or d-catechin; D,L-B-(3-indolyl)-.alpha.-methyl-.alpha.-hydrazinopropionic acid (R)-3õ3,4-dihydroxyphenyl!-1-fluoropropylamine, (S)-.alpha.-fluoromethyldopa, (S)-.alpha.-fluoromethyltyrosine, 5-(3,4-dihydroxycinnamoyl) salicylic acid, 3-hydroxycinnamic acid, caffeic acid, 3-mercaptocinnamic acid, .alpha.-methyl-3-hydroxycinnamic acid, .alpha.-ethyl-3-hydroxycinnamic acid, 3-hydroxy-w-nitrostyrene, 3,4-dihydroxyhydrocinnamic acid, 3-hydroxybenzalacetone, 3-hydroxychalone, 3-hydroxybenzal furanyl ketone, 3-hydroxybenzal thiophenyl ketone, 3′,4′-dihydroxyflavone, 8-O-glucoseflavone, flavone, 3-hydroxyphenyl pyruvic acid, 3,4-dihydroxyphenylpyruvic acid phenylthiopyruvic acid, 4-hydroxyphenylpyruvic acid, dithiosalicyclic acid, 1-hydroxy-2-naphthoic acid, 3-hydroxy-7-sulfo-2-naphtholic acid, 3,5-dihydroxy-2-naphtholic acid, 4-chlorocinnamic acid, 2-chlorocinnamic acid, 2,4-dichlorocinnamic acid, 3-nitrocinnamic acid, 3,5-dibromo-2-hydroxycinnamic acid, 2,4,6-triiodo-3-hydroxycinnamic acid, 2-hydroxy-4′-cyanochalone, 4-(4-hydroxycinnamoyl) benzylnitrile, 2-(4-hydroxycinnamoyl)-1,4-dihydroxybenzene, quercetin-6′-sulfonic acid, 5-(2-hydroxy-3,5-dibromocinnamoyl) salicylic acid or 5-(3-hydroxycinnamoyl) salicylic acid
an inhibitor of acrosin, a proteolytic enzyme located in the acrosome of sperm, such as tosyl lysine chloromethyl ketone, N-.alpha.-tosyl-L-arginine chloromethyl ketone, or ethyl p-guanidinobenzoate,
adenosine cyclic 3′,5′-monophosphate (cAMP), N.sup.6, O.sup.2-dibutyryladenosine cyclic 3′,5′-monophosphate or an analogue which produces an inotropic response,
adenosine kinase enzyme inhibitor such as 6,6′-dithiobis (9-B-D-ribofuranosylpurine),
inhibitor of monoamine oxidase such as phenylhydrazine, phenylethylidenehydrazine, isopropylhydrazine, or iproniazid,
an inhibitor of catechol-o-methyltrasferase such as 3,5-diiodo-4-hydroxybenzoic acid, S-3′-deoxyadenosylL-homocysteine, pyrogallol, R04-4602, gallic acid, 3,5-dihydroxy-4-methylbenzoic acid, 1,3-dihydroxy-2-methoxybenzene, 1-hydroxy-2,3-dimethoxybenzene, 2-hydroxy-1,3-dimethoxybenzene, 1,3-dihydroxy-4-methoxybenzene, catechol, 3,4-dihydroxybenzoic acid, caffeic acid, 5,6-dihydroxyindole, noradnamine, dopacetamide, H 22/54, quercetin, nordihydroguaiaretic acid, U-0521, arterenone, methylspinazarin, MK 486, dopa, papaveroline, isoprenaline, 7,8-dihydroxy-chlorpromazine, 3-hydroxy-4-pyridone, tetrahydroisoquinoline pyridoxal 5′-phosphate, iodoacetic acid, 3-mercaptotyramine, dehydrodicaffeic acid dilactone, methylspinazorin, 3′,5,7-trihydroxy-4′,6-dimeth-oxyisoflavone, 3′,5,7-trihydroxy-4′,8-dimeth-oxyisoflavone, 6,7-dihydromethylspinazarin, S-adenosylhomocysteine, S-tubercidinylhomocysteine, 3′,8-dihydroxy-4′,6,7-trimethoxyisoflavone, 7-O-methylspi nochrome B, 6-(3-hydroxybutyl)-7-O-methylspinachrome B, 3,5-diiodosalicyclic acid, or pyridoxal-5′-phosphate,
an inhibitor of adenosine deaminase which blocks the metabolism of adenosine such as coformycin, arabinosyl-6-thiopurine, 6-methylthioinosine, 6-thioinosine, 6-thioguanosine, N.sup.1-methyladenosine, N.sup.6-methyladenosine, 2-fluorodeoxyadenosine, 2-fluoroadenosine, inosine, 2′-deoxyinosine, deoxycoformycin, 1,6-dihydro-6-hydroxymethyl purine ribonucleoside, erythro-9-(2-hydroxy-3-nonyl)adenine, or 9-B-D-arabinofuranosyl-6-hydroxylaminopurine,
an inhibitor of adenylate kinase, 5′-nucleotidase, and adenosine translocase such as p.sup.1 p.sup.5-diadenosine pentaphosphate, .alpha.,.beta.-methylene adenosine diphosphate, and nitrobenzyl-6-thioinosine, respectively,
an inhibitor of .GAMMA.-aminobutyric acid uptake such as D,L-2,4-diaminobutyric acid, D,L-B-hydroxy GABA, (−)-nipecotic acid, trans-4-aminocrotonic acid, cis-3-aminocyclopentane-1-carboxylic acid, trans-3-aminocyclopentane-1-carboxylic acid, B-guanidinopropionic acid, homohypotaurine, 4-aminopentanoic acid, homotaurine, B-alanine, imidazoleacetic acid, 6-aminohexanoic acid, D,L-carnitine, D,L-2,6-diaminopimetic acid, D,L-2-fluoro GABA, guanidino acetic acid, 2-hydrazinopropionic acid, taurine, D,L-ornithine, or sulphanilamine which potentiates the inhibitory action of GABA,
inositol 1,4,5-triphosphate,
guanosine 5′ cyclic monophosphate or 8-bromo guanosine 5′ cyclic monophosphate which relaxes smooth muscle,
an inhibitor of the uptake system for glycine, the inhibitory synaptic transmitter of the spinal cord, such as hydrazinoacetic acid,
isoquinoline-sulfonamide inhibitor of protein kinase C, cAMP-dependant protein kinase, or cGMP-dependent protein kinase such as N-(2-aminoethyl)-5-isoquino-linesulfonamide,
Ribavirin which is active against HSV-1 and 2, hepatitis, and influenza viruses, or phosphonoacetic acid which is a highly specific inhibitor of Herpes Simplex virus induced polymerase and is active against HSV-1 and HSV-2, or adenine arabinoside (ara-A), cytosine arabinoside (Ara-C), ara-A 5′-monophosphate (ara-AMP), or hypoxanthine arabinoside (ara-Hx) which is active against HSV or phagicin which is active against vaccinia and HSV, or 4-fluoroimidazole, 4-fluoroimidazole-5-carboxylic acid, 4-fluoroimidazole-5-carboxamide, 5-fluoro-1-B-D-ribofurano-sylimidazole-4-carboxamide, 5-amino-1-B-D-ribofuranosyl-imidazole-4-carboxamide, poly (I).multidot.poly (C), sinefungin, iododeoxyuridine, 9-(2-hydroxy-ethoxymethyl) guanine, gliotoxin, distamycin A, netropsin, congocidine, cordycepin, 1-B-D-arabinofuranosylthymine, 5,6-di-hydroxy-5-azathymidine, pyrazofurin, toyocamycin, or tunicamycin,
an inhibitor of fungal chitin synthetase such as polyoxin D, nikko-mycin Z, or nikkomycin X,
an impermeant antifungal agent such as ezomycin A.sub.1, A.sub.2, B.sub.1, B.sub.2, C.sub.1, C.sub.2, D.sub.1, or D.sub.2 or platenocidin, septacidin, sinefungin, A9145A, A9145C, or thraustomycin,
an inhibitor of central nervous system carbonic anhydrase such as methazolamide, or 2-benzoylimino-3-methyl-.DELTA.sup.4-1,3,4-thiadiazoline-5-sulfonamide substituted at the benzolyl group with 3,4,5-trimethoxy, 2,4,6-trimethoxy, 2,4,5-trimethoxy, 4-chloro, 4-bromo, 4-iodo, or hydrogen,
an inhibitor of dopamine-B-hydroxylase during the synthesis of norepinephrine and epinephrine such as fuscaric acid, 5-(3′,4′-dibromobutyl)picolinic acid, 5-(3′-bromobutyl) picolinic acid, 5-(3′,4′-dichlorobutylpicolinic acid, YP-279, benxyloxyamine, p-hydroxybenzyloxyamine, U-21,179, U-7231, U-6324, U-0228, U-5227, U-10,631, U-10,157, U-1238, U-19,963, U-19,461, U-6628, U-20,757, U-19,440, U-15,957, U-7130, U-14,624, U-22,996, U-15,030, U-19,571, U-18,305, U-17,086, U-7726, dimethyldithiocarbamate, diethyldithiocarbamate, ethyldithiocarbamate, 2-mercaptoethylguanidine, thiophenol, 2-mercaptoethylamine, 3-mercaptopropylguanidine, 3-mercap-toprbpyl-N-methylguanidine, 2-mercaptoethanol, 2-mercaptoethyl-N-methylguanidine, 2-mercaptoethyl-N,N′-dimethylguanidine, 4,4,6-trimethyl-3,4-dihydropyrimidine-2-thiol, N-phenyl-N′-3-(4H-1,2,4-trizolyl)thiourea, methylspinazarin, 6,7-dimethylspinazarin, 7-O-methy-spinochrome B, 6-(3-hydroxybutyl)-7-O-methylspinachrome B, aquayamycin, chrothiomycin, frenoclicin, N-n-butyl-N′-3-(4H-1,2,4-trazolyl) thiourea, propylthiouracil, mimosine, mimosinamine, or mimosinic acid,
an inhibitor of histidine decarboxylation during the synthesis of histamine such as .sup.2-hydroxy-5-carbomethoxybenzyloxyamine, 4-toluene-sulfonic acid hydrazide, 3-hydroxy benzyloxyamine, hydroxylamine, aminooxyacetic acid, 4bromo-3-hydroxybenzyloxyamine (NSD-1055), rhodanine substituted in the 3 position with p-chlorophenethyl, p-chlorobenzyl, p-methylthiobenzyl, p-methylbenzyl, p-fluorobenzyl, amino, 3,4-dichlorobenzyl, p-bromobenzyl, p-methoxybenzyl, p-bromoanilino, p-iodoanilino, p-chloroanilino, p-toluidino, anilino, 2,5-dichloroanilino, dimethylamino, or p-methoxyphenyl; 2-mercaptobenzimidazole-1,3-dimethylol, 4-bromo-3-hydroxy-benzoic acid, 4-bromo-3-hydroxybenzyl alcohol, 4-bromo-3-hydroxyhippuric acid, (R,S)-.alpha.-fluoromethyl-histidine, (S)-.alpha.-fluoromethylester, L-histidine ethyl ester, L-histidinamide, D,L-3-amino-4-(4-imidazolyl)-2-butanone, 2-bromo-3-hydroxybenzyloxyamine, 5-bromo-3-hydroxybenzyloxyamine, 4,6-dibromo-3-hydroxybenzyloxyamine, aminooxypropionic acid, benzyloxyamine, 4-bromo-3-benzenesulfonyloxybenzyloxyamine, 3′,5,7-trihydroxy-4′,6-dimethoxyisoflavone, lecanoric acid, N-(2,4-dihydroxybenzoyl)-4-aminosalicylic acid, or 3′,5,7-trihydroxy-4′,8-dimethoxyisoflavone,
an pharamaceutical aget of drug that appear in Physicians Desk Reference, Edward R. Barnhart, 41th ed., 1987, Medical Economics Company Inc., N.J.; USAN and the Dictionary of Drug Names, ed. by Mary C. Griffiths, The United States Pharmacopedial Convention, (1986); and The Pharmacological Basis of Therapeutics, ed. by A. G. Gilman, L. Goodman, A. Gilman, 7th ed., (1985), MacMillan Publishing Co., N.Y., N.Y.,
a centrally acting converting enzyme inhibitor such as captopril,
an antibacterial agent such as penicillin, cephalosporin, or cephamycin, with B-lactamase resistance,
an agent which blocks bacterial synthesis of tetrahydrofolate such as a sulfonamide (an analogue of p-aminobenzoic acid) including sulfanilamide, sulfadiazine, sulfamethoxazole, sulfisoxazole, or sulfacetamide
an inhibitor of dihydrofolate reductace including pyrimethamine, cycloguanil, trimethoprin, isoaminopterin, 9-oxofolic acid, or isofolic acid,
a bactericidal agent such as nalidixic acid or oxolinic acid,
an inhibitor of bacterial protein synthesis such as vancomycin, an aminogylcoside, erythromycin, tetracyclin, or chloramphenicol,
an inhibitor of viral DNA polymerase such as vidarabine, tuberculostatic or tuberculocidal agent such as isoniazid or aminosalicyclic acid,
an anthelmintic agent such as oxamniquine, piperazine, metronidazole, diethylcarbamazine, paromomycin, niclosamide, bithionol, metrifonate, hycanthone, dichlorophen, or niclosamide,
an H.sub.2-blocking agent such as cimetidine or ranitidine,
an agent which blocks release of norepinephrine such as sotalol, guanethidine, pindolol, pronethalol, KO 592, practolol, oxprenolol, or pronethalol,
a xanthine oxidase inhibitor such as allopurinol, thioinosinate, 5,7-dihydroxypyrazolo õ1,5-a! pyrimidine substituted at the 3 position with hydrogen, nitro, bromo, chloro, phenyl, 3-pyridyl, p-bromophenyl, p-chlorophenyl, p-acetylanilino, p-tolulyl, m-tolulyl, naphthyl, or. 3,4-methylenedioxyphenyl; 8-(m-bromoacetamidobenzylthio)hypoxanthine, 8-(m-bromoacetamidobenzylthio)hypoxanthine, guanine substituted at the 9 position with phenyl, 4-chlorophenyl, 3-chlorophenyl, 3,4-dichlorophenyl, 4-methoxyphenyl, 3,4-dimethoxyphenyl, 4-dimethylaminophenyl, 4-aminophenyl, 3-aminophenyl, 3-trifluormethylphenyl, 4-benzamido, 4-carboxylphenyl, 4-methylpheyl, 4-ethylphenyl, 3-methylphenyl, B-naphthyl, or 4-ethoxyphenyl; 4,6-dihydroxypyrazolo o3,4-d! pyrimidine, 4-trifluoromethylimidazoles substituted at the 2 position with phenyl, p-chlorophenyl, p-methoxyphenyl, p-acetylanilino, p-nitrophenyl, p-dimethylaminophenyl, p-cyanophenyl, p-fluorophenyl, p-carboxyphenyl, m-chlorophenyl, 3,4-dichlorophenyl, 4-pyridyl, 3-pyridyl, 2-quinolyl, 6-quinolyl, 4-quinolyl, 7-quinolyl, 2-pyrazinyl, or 1-(2-pyridyl-4-trifluoromethyl-5-bromoimidazolyl; 5-(4-pyridyl)-1,2,4-triazoles substituted at the 5 position with 4-pyridyl, 3-pyridyl, 2-pyridyl, phenyl, p-chlorophenyl, m-chlorophenyl, p-sulfonamidophenyl, 3,5-dichlorophenyl, 3,5-dicarboxyphenyl, 6-quinolyl, 2-fuiryl, 4-pyridazinyl, 2-thienyl, 2-pyrimidinyl, 4-pyrimidinyl, or 4-pyrazinyl; difimisal, 4(or 5)-(2-aminoethylthio-azo)imidazole-5(or 4)-carboxamide, 4 (or 5)-diazoimidazole-5(or 4)-carboxamide, or S-õ5(or 4)-carbamoyl-4(or 5)-imidazolyl azo! cysteine,
an agent which inhibits DNA synthesis such as a bis-thiosemicarbazone, 3,5-diisopropylsalicyl-hydroxamic acid, 4-hydroxybenzoylhydroxamic acid, 3-methylsalicylhydroxamic acid 2,5-dihydroxybenzoylhydroxamic acid, or 2-hydroxy-3,4,5-trimethoxybenzoylhydroxamic acid; or which inhibits nucleotide synthesis such as N-(phosphoacetyl)-L-aspartate which inhibits asparatate transcarbamylase during pyrimidine synthesis, or azaserine or 6-diazo-5-oxo-L-norleucine which inhibits purine synthesis at the phosphoribosyl-formyl-glycineamidine synthetase step; or which is an antifolate such as methotrexate, 2,4-diamino-5-benxyl-6-(4-phenylbutyl) pyrimidine, 2,4-diamino-5-phenyl-6-(4-phenylbutyl) pyrimidine, 2,4-diamino-5-phenyl-6-(3-anilinopropyl) pyrimidine, 2-amino-4-hydroxy-5-phenyl-6-(3-p-aminobenzoylglutamic acid propyl) pyrimidine, N-(p-oo(2,4-diamino-6-quinazolinyl)methyl-methylamino-benzoyl-L-glutamic acid, N-õp-õ2,4-diamino-5-methylquinazolinyl)methylamino!benzoyl-L-aspartic acid, N-op-õõ(2-amino-4-hydroxy-6-quinazolinyl) methyl-!methylamino! benzoyl!-L-glutamic acid, 2,4-diaminoquinazolines: CCNSC 105952, CCNSC 112846, CCNSC 121346, CCNSC 122761, CCNSC 122870, CCNSC 529859, CCNSC 529860, or CCNSC 529861; 8-aza GMP, 7-deaza-8-aza GMP, 2′-dGMP, B-D-arabinosyl GMP, pentopyranine A-G, B-ribofuranosyl-1,3-oxazine-2,4-dione, pyrazofurin, 6-(p-chloroacetylanilinomethyl)-5-cetylvinylanilinomethyl)-5-(p-chlorophen yl)-2,4-diaminopyridine, 6-(p-chloroacetyl-ethylanilino-methyl)-5-(p-chlorophenyl)-2,4-diamino pyridine, 6-(p-chlorophenylbutylanilinomethyl)-5-(p-chlorophenyl)-2,4-diamino pyridine, p-(2,6-diamino-1,2-dihydro-2,2-dimethyl-S-triazin-1-yl) phenylpropionyl sulfanilylfluoride or variants of the propionamide bridge of acrylamido, N-ethylsulfonamido, N-ethylcaboxamido, oxyacetamido, or oxythyloxy; or which inhibits purine or pyrimidine synthesis such as xylosyladenine, 6-azauridine, 5-aminouridine, 5-azaorotic acid; or which inhibits nucleotide interconversion such as hadacidin, 6-mercaptopurine, azathioprine, nitro-dUMP, psicofuranine, decoyinines 5-fluorouracil, 5-fluorodeoxyuridine, shadowmycin; or which inhibits nucleotide utilization such as cytosine arabinoside, arabinosyladenine; or which becomes incorporated into polynucleotides such as 8-azaguanine, tubercidine, toyocamycin, sangivamycin, formycin, 7-deazainosine, 8-azainosine, or 7-thia-7,9-dideazainosine; or which is a glyoxalase inhibitor such as Glyo-I, or Glyo-II,
an agent which blocks synthesis of prostaglandin A.sub.2 which effects platelett aggregation such as salicylic acid, pyrogallol, 5,8,11,14-eicosatetraynoic acid, .alpha.-naphthol, guaiacol, propylgallate, nordihydroguiaretic acid, N-0164, benzydamine, 9,11-azoprosta-5, 13-dienoic acid, 2-isopropyl-3-nicotinylindole,
an agent which blocks prostaglandin synthetase such as indomethacin, sulindac, tolmetin, mefenamic acid, ibuprofen, naprozen, fenoprofen, fluribiprofen, ketoprofen, meclofenamic acid, flufenamic acid, niflumic acid, benzydamine, oxyphenbutazone, asprin, acetaminophen, salicylamide, O-carboxydiphenylamine, tolectin, diclofenac, 2,7-dihydroxynaphthalene, 5-(4-chlorobenzoyl)-1-methylpyrrole-2-acetic acid, 5-(4-methylbenzoyl)-1,4-dimethylpyrrole-2-acetic acid, 5-(4-chlorobenzoyl)-1,4-dimethylpyrrole-2-acetic acid, 5-(4-fluorobenzoyl)-1,4-dimethylpyrrole-2-acetic acid, 5-(4-chlorobenzoyl)-1,4-dimethylpyrrole-2-(2-propionic acid), 5,6-dehydroarachidonate, 11,12-dehydroarachidonate, or 5,8,11,14-eicosatetraynoate; or of an agent which blocks lipoxygenase or blocks leukotriene action such as BW755C, FPL 55712, or U-60,257
an antiarrhythmic agent such as procainamide or quinidine,
an inhibitor of hepatic synthesis of Vitamin K dependent clotti-ng factors such as warfarin sodium, dicumarol, 4-hydroxycoumarin, phenprocoumon, or acenocoumarol,
an agent which relaxes vascular smooth muscle such as hydralazine, minoxidil, or isoxsuprine,
a Na.sup.+-K.sup.+-ATPase inhibitor such as digtoxigenin, digoxigenin, cymarol, periplogenin, or strophanthidiol, or ouabain glycosides, cardenolides, or basic esters, or ICI-63,632, ICI-63,605, ICI-62-655, ICI-62,838, ICI-69,654, ICI-58,622, ICI-61,374, ICI-57,267, ICI-61,424, ICI-61,411, ICI-65,199, ICI-70,898, ICI-70,899, ICI-70,900, ICI-70,901, ICI-62,966, ICI-65,210, ICI-63,116, ICI-62,936, ICI-65,551, ICI-63,978, ICI-62,276, ICI-63,056, ICI-67,135, ICI-67,167, ICI-67,134, ICI-67,875, ICI-67,880, or ICI-61,558,
a calcium channel blocker such as prenylamine, verapamil, fendiline, gallopamil, cinnarizine, tiapamil, diltiazem, bencyclan, or nifedipine; or an agent which stabalizes calcium binding to cellular calcium stores and thereby inhibits the release of this calcium by contractile stimuli such as 8-(N,N-diethylamino)-octyl 3,4,5-trimethoxybenzoate (TMB-8),
a monoamine oxidase inhibitor such as tranylcypromine, phenylethylamine, trans-cinnamic acid, phenelzine, or isocarboxazid,
a benzodiazepine compound such as clorazepate,
valproic acid,
an agent which causes repression of the synthesis of HMG-COA reductase such as 20-.alpha.-hydroxycholesterol, 22-ketocholesterol, 22-.alpha.-hydroxycholesterol, 25-hydroxycholesterol, 22-B-hydroxycholesterol, 7-.alpha.-hydroxycholesterol, 7-B-hydroxycholesterol, 7-ketocholesterol, or kryptogenin; or of an agent which inhibits HMG-COA reductase such as, lorelco; or of an agent which inhibits lipolysis such as 5-methylpyrazole-3-carboxylic acid (U-19425), nicotinic acid, uridine, inosine, 3,5-dimethylisoxazole (U-21221), 3,5-dimethypyrazole, prostaglandin E.sub.2, eritadenine, or eritadenine isoamyl ester; or of an agent which inhibits lipogenesis such as ascofuranone, (−)-hydroxycitrate, or tetrolyl-CoA; or of an agent which is hypocholesterolemic such as lentysine; or of an agent which lowers triglycerides such as lopid; or of an agent which is an inhibitor of acetyl-CoA carboxylase during lipogenesis such as 2-methyl-2-op-(1,2,3,4-tetrahydro-1-naphthyl)-phenoxy!-propionat e (SU13437), sup.2-(p-chlorophenoxy)-2-methylpropionate, kynurenate, xanthurenate, kynurenine, 3-hydroxyanthranilate, or 2-methyl-2-op-(p-chlorophenyl)phenoxy! propionate; or of an agent which is an inhibitor of hepatic B-lipoprotein production such as orotic acid,
a vasodilater such as WS-1228A, or WS-1228B; or of an anti-inflammatory agent such as amicomacin A,
a protease inhibitor such as leupeptin; or which is an inhibitor of pepsin such as a pepstatin, a pepstanone, or a hydroxypepstatin,
an inhibitor of cell surface enzymes such as bestatin, amastatin, forphenicine, ebelactone, or forphenicin,
a phosphodiesterase inhibitor such as theophyllineacetic acid, theophylline, dyphylline, disodium cromoglycate, 6-n-butyl-2,8-dicarboxy-4,10-dioxo-1,4,7,10-tetrahydro-1,7-phenanthrolin, 2-chloroadenosine, dipyridamole, EG 626, AY-17,605, AY-17,611, AY-22,252, AY-22,241, cis-hinokiresinol, oxy-cis-hinokiresinol, tetrahydro-cis-hinokiresinol, trans-hinokiresinol, dehydrodicaffeic acid, 2,6,4′-trihydroxy-4-methoxybenzophenone, p-hydroxyphenyl crotonic acid, papaverine, 3-(5-tetrazolyl)-thioxanthone-10,10-dioxide, 3-carboxythioxanthone-10,10-dioxide, W-7, HA-558, MY-5445, OPC-3689, OPC-13135, or OPC-13013, reticulol, PDE-I, or PDE-II,
an inhibitor of tyrosine hydroxylase, the enzyme catalyzing the rate-limiting reaction in the biosynthesis of norepinephrine, such as azadopamine, isopropylazadopamine, dimethylazadopamine; triphenolic compounds such as n-propylgallate; diphenolic benzoic acid derivatives such as 3,4-dihydroxybenzoic acid; phenylcarbonyl derivatives such as 3,4-dihydroxybenzaldehyde, arterenone, or adrenalone H 22/54, 3-iodo-L-tyrosine, D,L-.alpha.-methyl-p-tyrosine, L-3-iodo-.alpha.-methyltyrosine, 3-bromo-.alpha.-methyltyrosine, gentistic acid, 3-chloro-.alpha.-methyltyrosine, phenylalanine derivatives, 3,5-diiodo-L-tyrosine, 3,5-dibromo-L-tyrosine, 3-bromo-.alpha.-methyl-L-tyrosine, 3-fluro-.alpha.-methyl-L-tyrosine, catechol analogues, 3,4-dihydroxyphenylethylacetamide, 3,4-dihydroxyphenylisoproplyacetamide, 3,4-dihydroxyphenylbutylacetamide, 3,4-di-hydroxyphenylisobutylacetamide, D,L-.alpha.-methylphenylalanine, D,L-3-iodophenylalanine, D,L-4-iodophenylalanine, D,L-.alpha.-methyl-3-iodophenylalanine, D,L-a-methyl-3-bromophenylalanine, D,L-.alpha.-methyl-3-chlorophenylalanine, D,L-.alpha.-methyl-3-fluorophenylalanine, mimosine, mimosinamine, mimosinic acid, 7-O-methylspinochrome B, 6-(3-hydroxybutyl)-7-O-methylspinachrome B, aquayamycin, chrothiomycin, frenolicin, fuscaric acid, pentylpicolinic acid, dopstatin, methylspinazarin, 6,7-dihydroxymethylspinazarin, 3-ethyl-.alpha.-methyltyrosine, 3-methyl-.alpha.-methyltyrosine, 3-isopropyl-x-methyltyrosine, 3-allyl-.alpha.-methyltyrosine, 3-õ4-hydroxy-3-(2-methylallyl)-phenyl!-2-methylalanine, 3-õ3-(2,3-epoxypropyl)-4-hydroxyphenyl!-2-methylalanine, 3-isobutyl-.alpha.-methyltyrosine, 3-methylvinyl-.alpha.-methyltyrosine, 5-methyl-6,7-diphenyltetrahydropterin, 3-(2,3-dihydro-2,2-dimethyl-5-benzofuranyl!-2-methylalanine, 3-õ2,3-dihydro-2,2-dimethyl-5-benzofuranyl!-2-methylalan ine, alpha.-methyldopa, or ethyl-3-amino-4H-pyrrolo õ3,4c! isoxazole carboxylate, and
proteins including enzymes and hormones such as insulin, erythropoietin, interleuken 2, interferon, growth hormone, atrial natriuretic factor, tissue plasminogen activator.

The C moiety may comprise at least one of the group of herbicides, fungicides, miticides, nematocides, fumigants, growth regulators, repellants, defoliants, rodenticides, molluscicides, algicides, desicants, antehelmintics, and bactericides.

The C moiety may be one from the those given in Chemical Week Pesticides Register, Robert P. Ovellette and John A. King, 1977, McGraw-Hill Book Company.

The invention further comprises a method of synthesis of a chemical compound having the formula (A-B-C)ₓ-P-E_{y}
where the A is a chemiluminescent moiety,
B is an energy acceptor moiety, and
C is a biologically active moiety, and
P is a substrate
E is an enzyme and x and y are integers
   comprising the steps of
forming a benzophenone,
forming a diaryl ethylene,
attaching a phthalimide moiety to at least one of the aryl groups of the ethylene to form a phthalimide-ethylene conjugate,
condensing two ethylene-phthalimide conjugates to form a phthalimide-pentadiene conjugate,
converting the phthalimide to the phthalhydrazide by reaction with hydrazine to form a carrier compound, and
reacting the carrier compound with a biologically active moiety to form a corresponding conjugate,
reacting A-B-C with a polymer to form (A-B-C)ₓ-P, and
reacting E with (A-B-C)ₓ-P to form (A-B-C)ₓ-P-E_{y}.

This compound may provide controlled extra cellular release of the C moiety.

The C moiety may comprise at least one of drugs and proteins including enzymes and hormones.

The C moiety may comprise at least one insulin, erythropoietin, interleuken 2, interferon, growth hormone, atrial natriuretic factor, tissue plasminogen activator, an anti-inflammatory drug, an antihypertensive drug, an inotropic drug, and a contraceptive drug.

Extraacellular drug release may occur when the prodrug reacts with cellular free radicals via a mechanism involving chemiluminescence, photochromism, and intramolecular energy transfer.

The pharmaceutical agent may be at least one of the group of antilipidemic drugs, anticholesterol drugs, contraceptive agents, anticoagulants, anti-inflamatory agents, immunosuppressive drugs, antiarrhythmic agents, antineoplastic drugs, antihypertensive drugs, epinephrine blocking agents, cardiac inotropic drugs, antidepressant drugs, diuretics, antifungal agents, antibacterial drugs, anxiolytic agents, sedatives, muscle relaxants, anticonvulsants, agents for the treatment of ulcer disease, agents for the treatment of asthma and hypersensitivity reactions, antithroboembolic agents, agents for the treatment of muscular dystrophy, agents to effect a therapeutic abortion, agents for the treatment of anemia, agents to improve allograft survival, agents for the treatment of disorders of purine metabolism, agents for the treatment of ischemic heart disease, agents for the treatment of opiate withdrawal, agents which activate the effects of secondary messenger inositol triphosphate, agents to block spinal reflexes, and antiviral agents including a drug for the treatment of AIDS.

The C moiety may be released by an oxidation reduction reaction with the target cell's electron carriers or by reaction with free radicals produced as a consequence of electron transport.

A may represent a functionality which undergoes at least one of
an oxidation reduction reaction where electrons are transferred directly between A and the target cell's electron carriers, and
a reaction with free radicals of oxygen which are produced as a consequence of electron transport
such that an excited state is produced in A as a consequence of its participation in one of these reactions.

A may undergo intramolecular energy transfer from its own excited state to the B functionality which is an energy acceptor.

Upon receiving energy from A, B may achieve an excited state which relaxes through heterolytic cleavage of the covalent bond of B with C where C is a drug moiety which is released into the environment.

The chemiluminescent molecule may comprise at least one of the group of
molecules undergoing reaction involving peroxides and oxygen free radicals,
molecules undergoing reaction involving oxidation or reduction, and
molecules undergoing both reaction with peroxides and oxygen free radicals followed by an oxidation or reduction reaction.

The chemiluminescent molecule may comprise at least one of the group of lumifiol and its derivatives, lucigenin and its derivatives, Lophine and its derivatives, acridinium esters and acridans, tetraphenylpyrrole, phthalhydrazides, acyloins, biacridinium salts, vinylcarbonyls, vinylnitriles, tetrakis (dimethylamino) ethylene, acylperoxides, indoles, tetracarbazoles and active oxalates.

The chemiluminescent molecule may comprise at least one of the group of ruthenium chelates 2,6-diaminopyrene, or cation radicals and molecules which follow a Chemically Initiated Electron Exchange Luminescence mechanism such as certain dioxetans and dioxetanones.

The chemiluminescent molecule may comprise at least one of the group of dioxene derivatives and other compounds that form a dioxetan by reaction with superoxide and then produce efficient chemiluminescence by a CEEEL mechanism.

The chemiluminescent molecule may comprise at least one of the group of componds given in Table 1.

In an embodiment, the B moiety is a photochromic compound.

The photochromic compound may comprise one which demonstrate photochromic behavior with electromagnetic radiation and bleaching agents.

In an embodiment, the A functionality is chemiluminescent, and the B functionality is such that the photodissociative drug release spectrum of B overlaps the chemiluminescence spectrum of A.

In an embodiment, the photochromic compound comprises a cationic dye.

The cationic dye may comprise at least one of a di and triarylmethane dyes, triarylmethane lactones and cyclic ether dyes, cationic indoles, pyronines, phthaleins, oxazines, thiazines, acridines, phenazines, and anthocyanidins, and cationic polymethine dyes and azo and diazopolymethines, styryls, cyanines, hemicyanines, dialkylaminopolyenes, and other related dyes.

The cationic dye may comprise at least one of the compounds given in Table 2.

The C moiety may be any molecule which exhibits bleaching behavior with the B moiety and has an increased therapeutic effect or therapeutic ratio as a consequence of its delivery as part of a prodrug.

The C moiety may have a nucleophilic group that bonds to the B moiety.

The C moiety may be derivatized to have a nucleophilic group that bonds to the B moiety.

The C moiety may be derivatized by at least one of the nucleophilic groups comprising cinnamate, sulfite, phosphate, carboxylate, thiol, amide, alkoxide, or amine.

The C moiety may be at least one of the group of compounds given in Table 3.

The A-B-C moieties may be attached to P by a bond between P and at least one of A and B.

The E moieties may be attached to (A-B-C)ₓ-P by a bond between E and at least one of A, B, and P.

The E moieties may be enzymes that react with a desired substrate and form substances that cause the release of C from A-B-C.

The E moieties may be enzymes that react with a desired substrate and form peroxide or free radicals that cause the release of C from A-B-C.

The E moiety, substrate, and C moiety may be at least one of the group of
glucose oxidase, glucose, and insulin, and
xanthine oxidase, xanthine, and tissue plasminogen activator (TPA).

The invention further comprises a method of synthesis of a chemical compound having the formula (A-B-C)ₓ-P
where the A is a chemiluminescent moiety,
B is an energy acceptor moiety, and
C is a biologically active moiety, and
P is a substrate and x is an integer
   comprising the steps of
forming a benzophenone,
forming a diaryl ethylene,
attaching a phthalimide moiety to at least one of the aryl groups of the ethylene to form a phthalimide-ethylene conjugate,
condensing two ethylene-phthalimide conjugates to form a phthalimide-pentadiene conjugate,
converting the phthalimide to the phthalhydrazide by reaction with hydrazine to form a carrier compound, and
reacting the carrier compound with a strong base such as an alkali hydride and the biologically active moiety to form a corresponding conjugate,
reacting A-B-C with a polymer to form (A-B-C)ₓ-P.

One or more of the moieties can be modified to further candidate components by addition of functional groups.

The groups may comprise at least on of alkyl, cycloalkyl, alkoxycarbonyl, cyano, carbamoyl, heterocyclic rings containing C, O, N, S, sulfo, sulfamoyl, alkoxysulfonyl, phosphono, hydroxyl, halogen, alkoxy, alkylthiol, acyloxy, aryl, alkenyl, aliphatic, acyl, carboxyl, amino, cyanoalkoxy, diazonium, carboxyalkylcarboxamido, alkenylthio, cyanoalkoxycarbonyl, carbamoylalkoxycarbonyl, alkoxy carbonylamino, cyanoalkylamino, alkoxycarbonylalkylamino, sulfoalkylamino, alkylsulfamoylaklylamino, oxido, hydroxy alkyl, carboxy alkylcarbonyloxy, cyanoalkyl, carboxyalkylthio, arylamino, heteroarylamino, alkoxycarbonyl, alkylcarbonyloxy, cyanoalkoxy, alkoxycarbonylalkoxy, carbamoylalkoxy, carbamoylalkyl carbonyloxy, sulfoalkoxy, nitro, alkoxyaryl, halogenaryl, amino aryl, alkylaminoaryl, tolyl, alkenylaryl, allylaryl, alkenyloxyaryl, allyloxyaryl, cyanoaryl, carbamoylaryl, carboxyaryl, alkoxycarbonylaryl, alkylcarbonyoxyaryl, sulfoaryl, alkoxysulfoaryl, sulfamoylaryl, and nitroaryl.

In an embodiment, the invention comprises a method to synthesize a compound having the structure of general formula 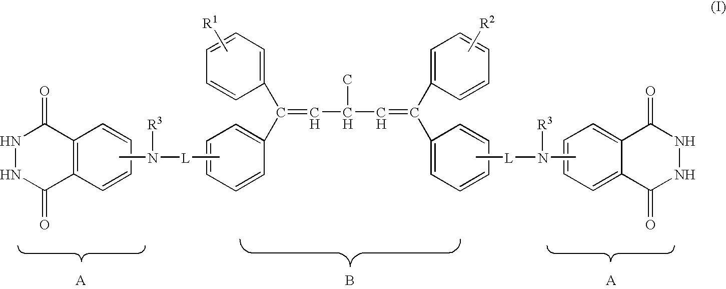
wherein the functionality A may be at least one of aminophthalhydrazide derivatives, sulfonyloxamides and active oxalates,
the functionality B may be at least one of 1,1,5,5-tetrakisarylpentadiene and 1,1,5-trisarylpentadiene derivatives,
the functionality C may be a drug molecule such as Foscarnate, or ddc; and
R is a functional group, and
L is a linker such as an aliphatic chain between A and B.

The L functionality may be between one 20 carbon atoms.

B may be a 1,1,5-trisarylpentadiene derivative and the compound has the formula

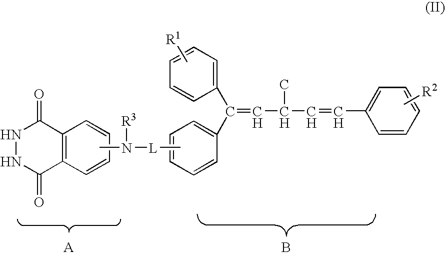

A may be a sulfonyloxamide or active oxalate and the compound has the formula

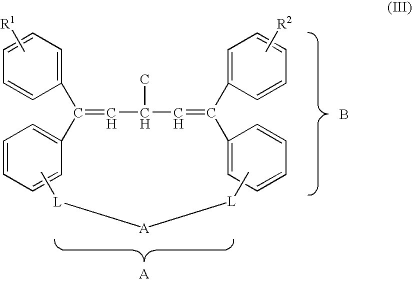

A luminol derivative may be directly attached through one or more amino groups to the aryl groups of a photochromic dye.

C may comprise the formula of at least one of

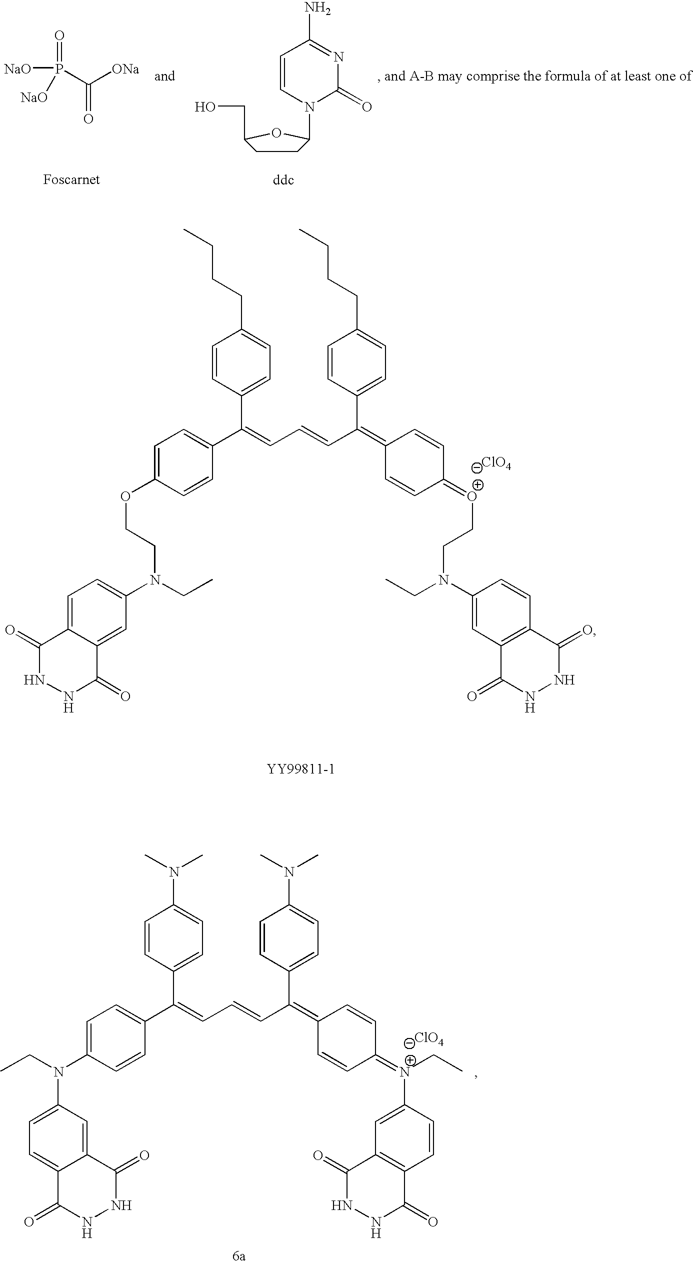

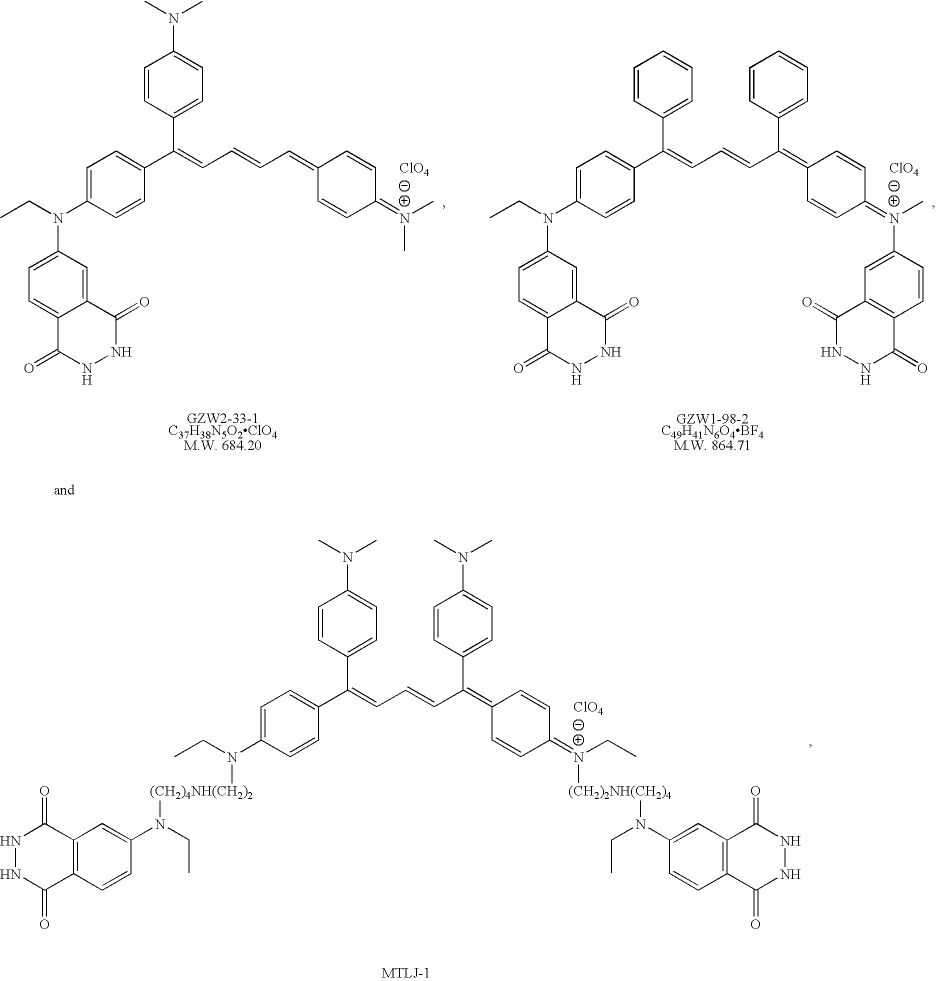

The compound A-B-C may comprise the formula 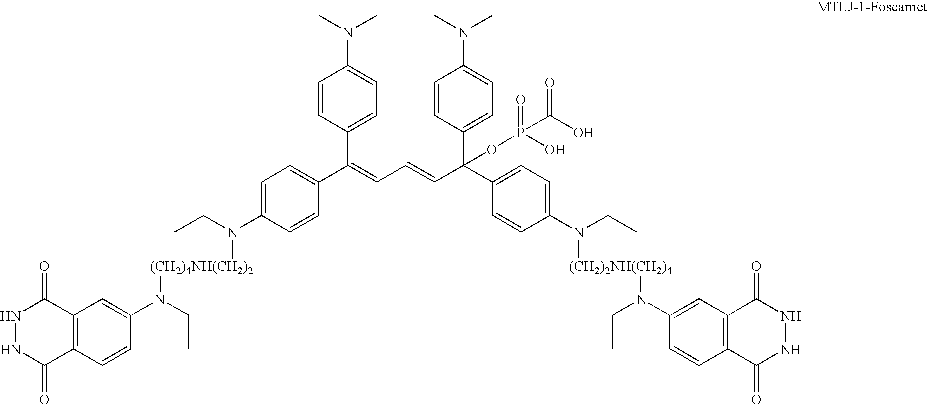
The hydrolyzable group that protects phthalhydrazide may be at least one of acetyl and t-butyloxycarbonyl.

The aminophthalimide-substituted precursors for the dye may be prepared through amination of an aryl halide such as palladium-catalyzed amination of aryl halides.

Halo-substituted aryl groups of a starting B moiety or an intermediate may be coupled with the aminophthalimide by methods such as the aryl amination under palladium catalysis to form the aminophthalimide-substituted precursors for the dye.

Halo-substituted aryl groups of a starting phthalimide or an intermediate may be coupled with the amino-substituted dye by methods such as the aryl amination under palladium catalysis to form the aminophthalimide-substituted precursors for the dye.

Amino-substituted aryl groups may be obtained by the amination of the halo-substituted compounds with an imine such as benzophenoneimine.

The aminophthalimide-attached dye may be formed by the condensation of two aminophthalimide-attached ethylene molecules by reaction with triethyl orthoformate and a strong acid such as perchloric acid in acetic anhydride or acetic acid.

During the step of converting the phthalimide moiety to the aminophthalhydrazide to obtain A-B, the B moiety may be protected from reaction with hydrazine by reacting with base such as sodium hydroxide, sodium methoxide and amines.

The phthalimide-B conjugate with a protected B moiety may be refluxed with hydrazine in a suitable solvent such as an alcoholic solvent in inert atmosphere and then treated with acid such as perchloric acid, tetrafluroboric acid to regenerate a corresponding unaltered B moiety of the A-B conjugate.

A-B my be reacted with one nucleophilic species of C to form A-B-C.

A-B may be formed by starting with B comprising halo-substituted dyes, such as 1,5-bis(p-bromophenyl)-1,5-bis(p-dimethylaminophenyl)-pentadienium perchlorate.

Cationic dyes may be protected by reacting with base such as alkoxide and then coupled with the aminophthalimide by amination of aryl halide such as the palladium-catalyzed amination of aryl halide to obtain the alkoxide-protecting aminophthalimide-substituted dyes.

The aminophthalimide-B conjugate with a protected B moiety may be refluxed with hydrazine in a suitable solvent such as an alcoholic solvent to convert the amino-phthalimide moiety to the aminophthalhydrazide moiety and then treated with acid to generate A-B.

B may comprise a tetraarylpolymethine, the aminophthalhydrazide precursor may be an aminophthalic acid diester and the conjugate to form A-B my be amino-phthalimideluminol-tetraaryl-polymethine.

Halo-substituted diarylketone may be formed by at least one of direct acylation of arene with halo-substituted benzoyl halide under ferric chloride catalysis according to the following representative scheme 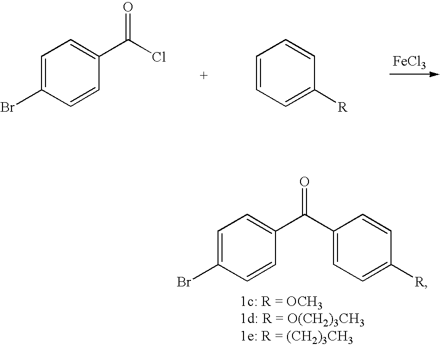
and or indirect acylation according to the following representative scheme

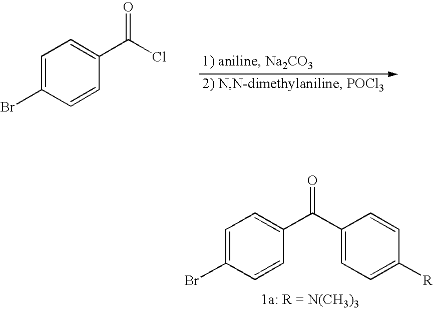

The halo-substituted diarylketone may converted to the corresponding halo-substituted diarylketene such as halo-substituted 1,1-diarylethene.

The halo-substituted diarylketene may be coupled with a precursor of aminophthalhydrazide such as aminophthalimide, aminophthalic acid diester, by aryl amination such as the palladium-catalyzed amination of aryl halides to form the aminophthalimide-substituted 1,1-diarylethene.

The ethene may be condensed with an orthoester such as triethylorthoformate in a nonaqueous solvent such as acetic anhdydide, containing an acid catalyst such as perchloric acid, tetrafluoroboric acid, to form the aminophthalimide-substituted tetraarylpolymethine dye.

The aminophthalimide moiety may be converted to the aminophthalhydrazide to obtain A-B.

In an embodiment, the B moiety is a cationic dye that is first protected by reacting with an anion such as hydroxide, methoxide and amine and the phthalimide-B conjugate with a protected B moiety is refluxed with hydrazine in a suitable solvent such as an alcoholic solvent in inert atmosphere and then treated with acid such as perchloric acid, tetrafluroboric acid to regenerate a corresponding unaltered B moiety of the A-B conjugate.

A-B may be reacted with one nucleophilic species of a C such as a drug 2′,3′-dideoxycytidine, Foscarnet, acycloguanosine to form A-B-C comprising a prodrug.

In an embodiment, two halo-substituted diarylketene precursor compounds are condensed with an orthoester such as triethylorthoformate in a nonaqueous solvent such as acetic anhydride containing acid catalyst such as perchloric acid, tetrafluoroboric acid to form the halo-substituted tetraarylpolymethine dyes such as 1,5-bis(p-bromophenyl)-1,5-bis(p-dimethylaminophenyl)pentadienium perchlorate.

In an embodiment, the B moiety is a cationic dye that is protected by reacting with an anion such as alkoxide and then coupled with the aminophthalimide by amination of aryl halide such as the palladium-catalyzed amination of aryl halide to obtain the alkoxide-protected aminophthalimide-substituted tetraarylpolymethine dye.

In an embodiment, the alkoxide-protected aminophthalimide-substituted tetraarylpolymethine dye is refluxed with hydrazine in a suitable solvent such as an alcoholic solvent to convert the amino-phthalimide moiety to the aminophthalhydrazide moiety and then treated with acid to generate A-B comprising a luminol-tetraarylpolymethine compound.

In an embodiment, the method of synthesis comprisies the general steps given by following representative formula

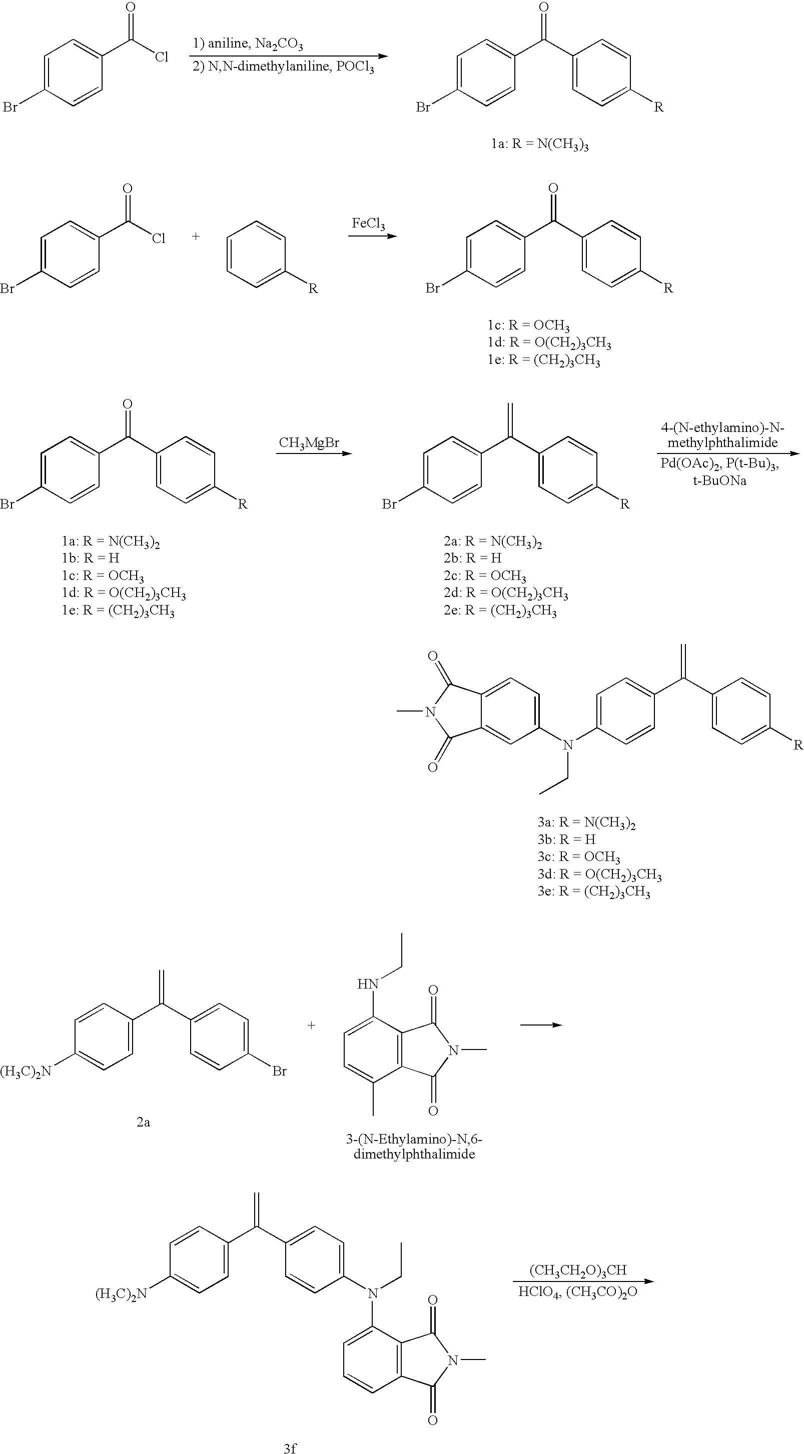

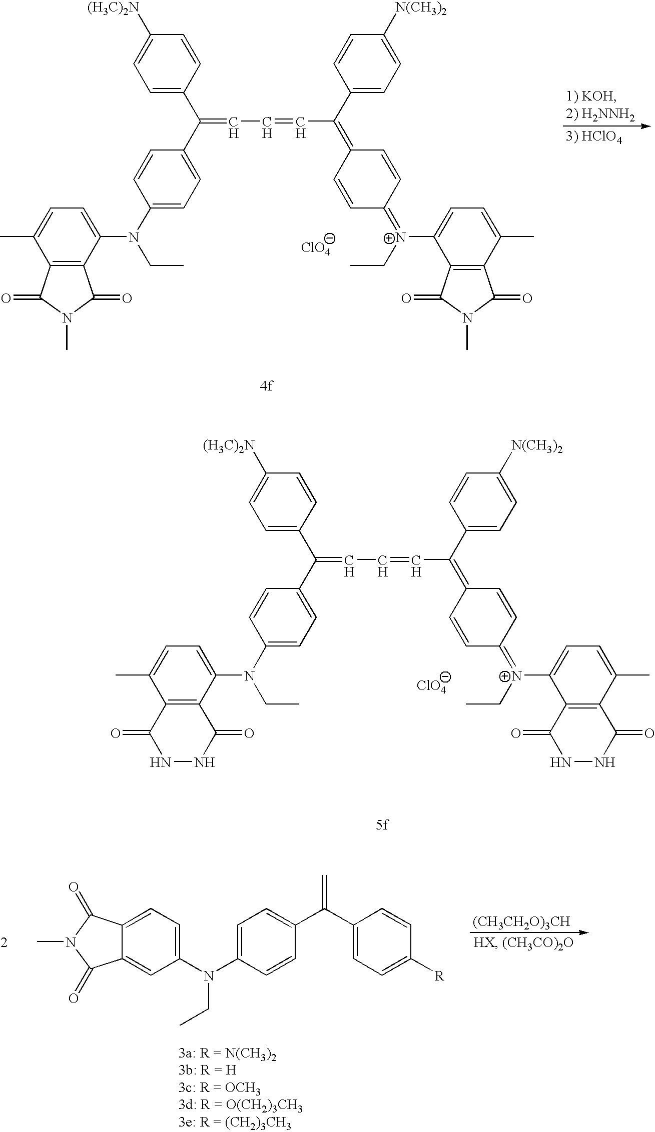

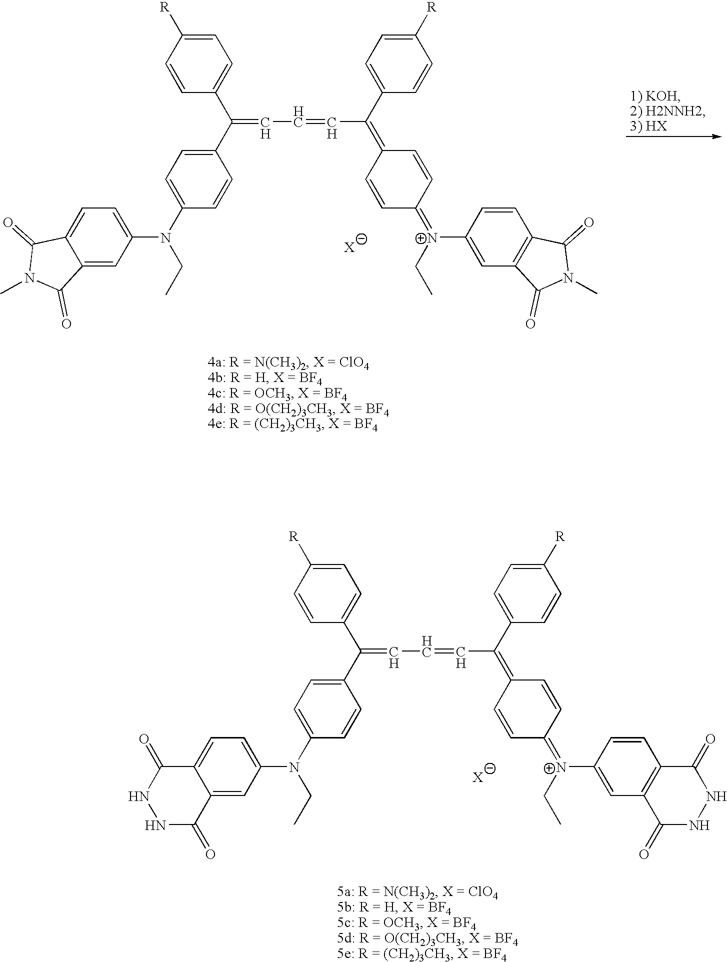

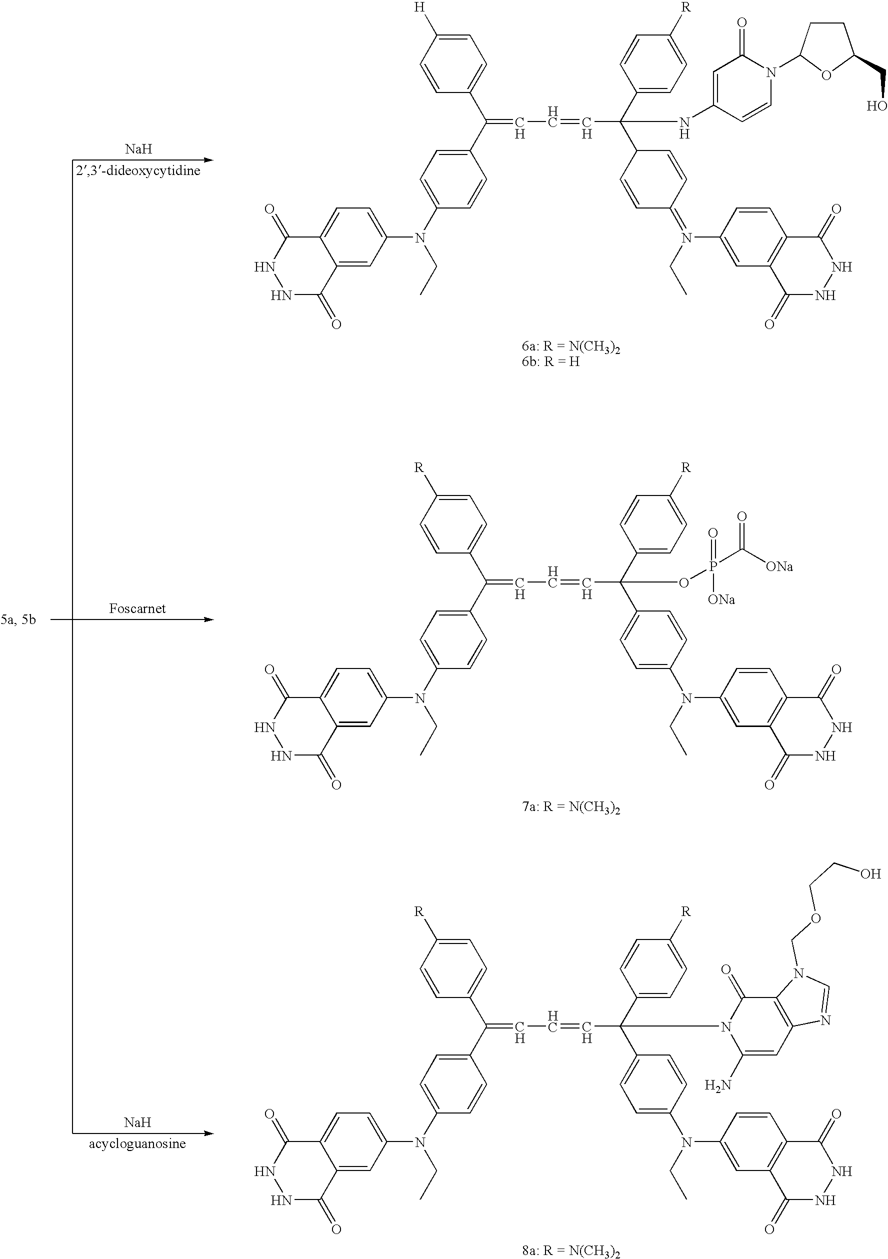

In an embodiment, the A functionality of A-B-C comprises a phthalhydrazide such as a luminol derivative and the B functionality comprises a photochromic dye wherein A is attached to aryl groups of B comprising the steps of
forming a diaryl ketone,
forming a diaryl ketene from the diaryl ketone,
forming a protected aminophthalhydrazide such as aminophthalimide or aminophthalic acid diester,
adding a hydrocarbon linker to the protected aminophthalhydrazide, and
attaching the protected aminophthalhydrazide through the molecular linker to the aryl groups of diarylketene to form the precursor aminophthalimide-linked diarylketene, and reacting according to at least one of
   (a) forming the A functionality from the precursor, and condensing two molecules of B precursor linked to A to form A-B, and
   (b) condensing two precursor aminophthalimide-linked diarylketene molecules to form A precursor linked to B, and
forming the A functionality from the A precursor to form A-B.

The diaryl ketone may be formed by a classical Friedel-Crafts acylation between a benzoyl halide and aryl compound with a hydrocarbon linker having a leaving group.

In an embodiment, the aryl compound with a hydrocarbon linker having a leaving group comprises at least one of a halogenated-alkyl-aryl ether and a halogenated-aklyl-aryl amine wherein the halogen is the leaving group.

In an embodiment, the halogenated-alkyl-aryl ether comprises 2-bromoethoxybenzene to give an aryl ketone such as 4-(2-bromoethoxy)benzophenone.

In an embodiment, the halogenated-aklyl-aryl amine comprises 2-bromoethyl aminobenzene to give an aryl ketone such as 4-(2-bromoethyl amino)benzophenone.

In an embodiment, the diaryl ketone is converted to the corresponding diarylketene by reacting with a methylating reagent such as a methyl Grignard reagent, methyl lithium reagent, lithium dimethylcopper reagent and then dehydration with acid.

The diaryl ketone may be converted to the corresponding diarylketene by reacting with methylmagnesium bromide and then dehydration with acid.

The diaryl ketone may be converted to the corresponding diarylketene by a Wittig reaction.

A linker may be attached to the protected aminophthalhydrazide by a reaction of a nucleophilic group of the linker or protected aminophthalhydrazide with a leaving group of the linker or protected aminophthalhydrazide.

A linker may be attached to the protected aminophthalhydrazide by reaction to form a bond between at least one of a nitrogen, oxygen, or carbon atom of the linker and at least one of a nitrogen, oxygen, or carbon atom of group of the protected aminophthalhydrazide by an addition or a substitution reaction of a leaving group.

A linker may be attached to the protected aminophthalhydrazide by a substitution reaction of at least one of a halogen, tosylate group, ester group with a nitrogen, oxygen, or carbon atom.

In an embodiment, attaching the protected aminophthalhydrazide through the molecular linker to one of the aryl groups of diarylketene to form the precursor aminophthalimide-linked diarylketene is by a reaction of a nucleophilic group of the linker or aryl group of diarylketene with a leaving group of the linker or aryl group of diarylketene.

A linker may be attached to the aryl group of diarylketene by reaction to form a bond between at least one of a nitrogen, oxygen, or carbon atom of the linker and at least one of a nitrogen, oxygen, or carbon atom of group of the protected aminophthalhydrazide by an addition or a substitution reaction of a leaving group.

A linker may be attached to the aryl group of diarylketene by a substitution reaction of at least one of a halogen, tosylate group, ester group with a nitrogen, oxygen, or carbon atom.

The precursor aminophthalimide-linked diarylketene may further reacted by condensation of two aminophthalimide-linked diarylketenes with an orthoester to form B linked to the A precursor wherein the condensing reagent may be triethylorthoformate.

The precursor aminophthalimide-linked diarylketene may comprise at least one of the formula 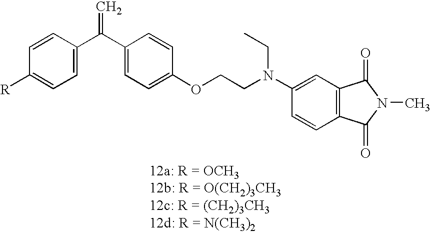
and the precursor of A-B may comprise at least one of the formula

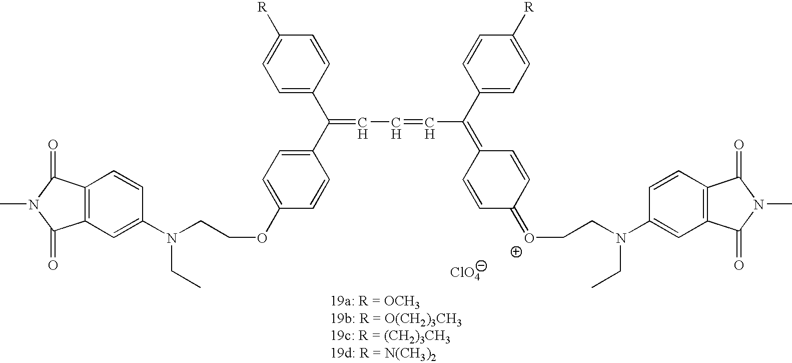

The phthalimide moiety of the A precursor may be converted to the phthalhydrazide A functionality by treating with hydrazine, forming A-B.

The B functionality may be protected by reacting with an anion such as hydroxide, methoxide and amine, the A-B precursor is refluxed with hydrazine in a suitable solvent such as an alcoholic solvent in inert atmosphere and then treated with acid such as perchloric acid, tetrafluroboric acid to form A-B.

The phthalimide moiety of the A precursor of the precursor aminophthalimide-linked diarylketene may be converted to the phthalhydrazide A functionality by treating with hydrazine, forming A attached to a B precursor.

The A-linked diarylketene may be further reacted by condensation of two A-linked diarylketenes with an orthoester to form A-B wherein condensing reagent may be triethylorthoformate.

The A-linked diarylketene may comprise at least one of the formula 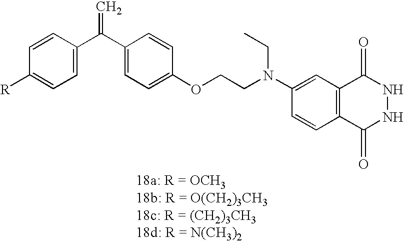
and A-B may comprise at least one of the formula

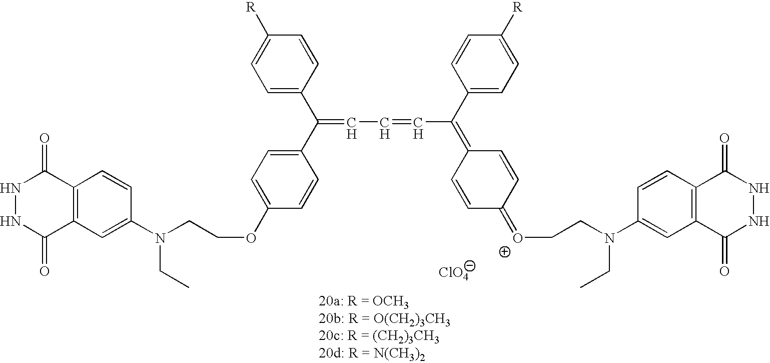

The method of synthesis if the present invention may further comprise the step of reacting the B functionality of A-B with one nucleophilic species of a C functionality such as Foscarnet to form A-B-C.

In an embodiment, the method of synthesis of the compound A-B-C comprises the general steps given by following representative formula

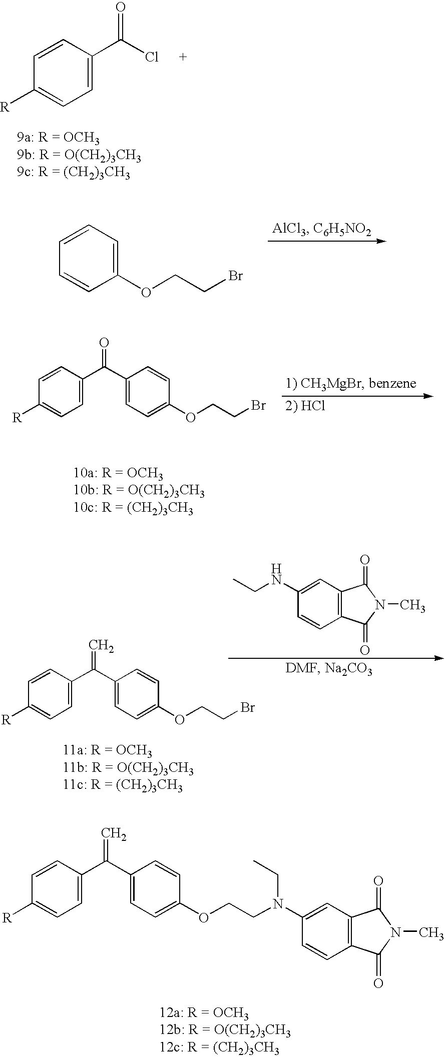

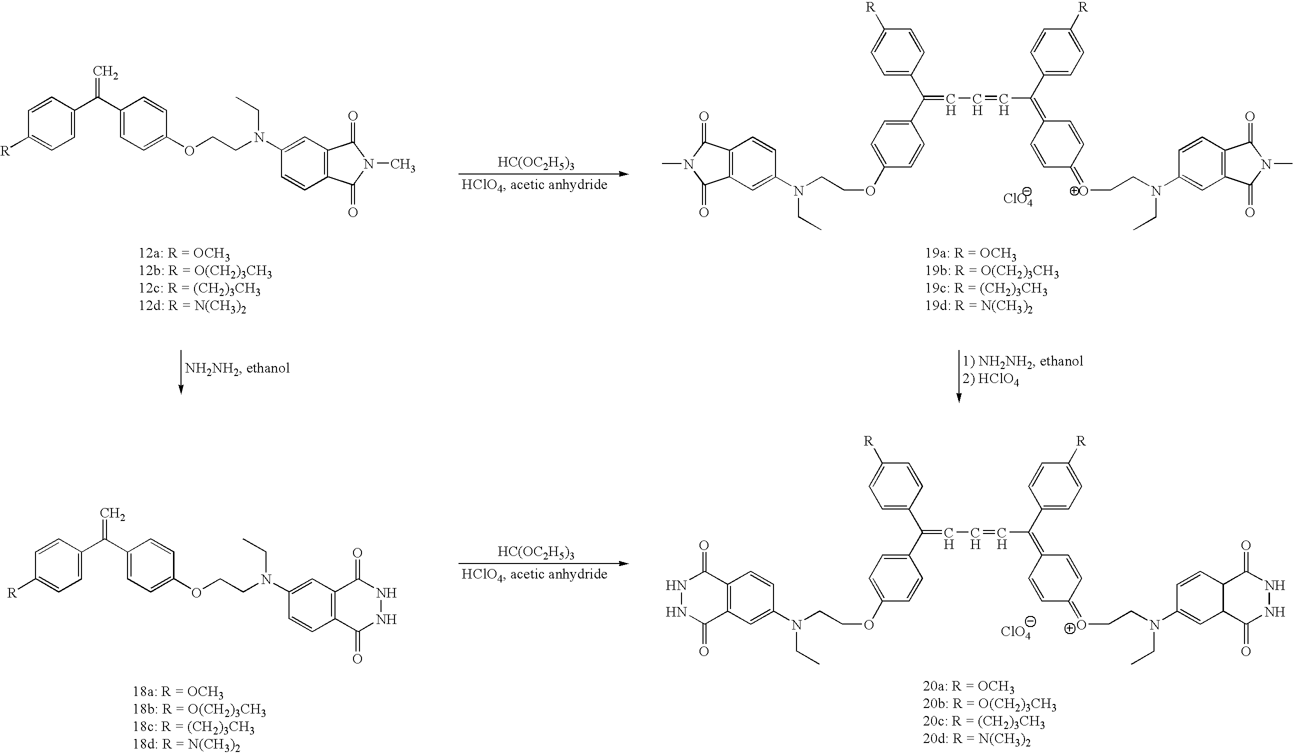

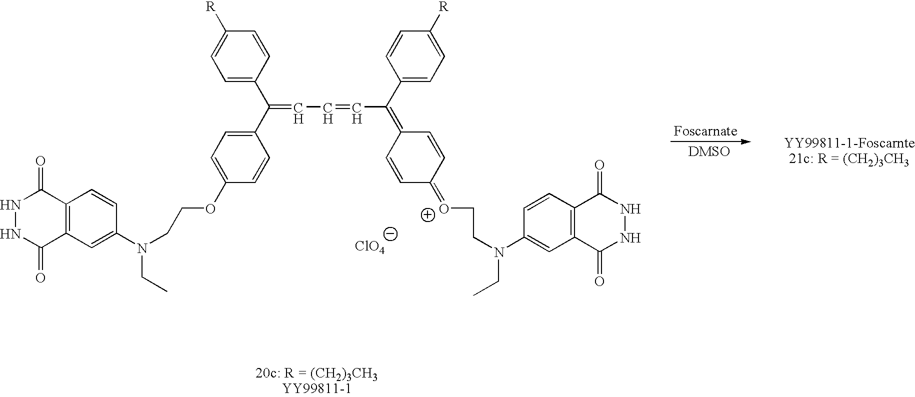

In an embodiment, the method of synthesis of the compound A-B-C comprises the general steps given by following representative formula

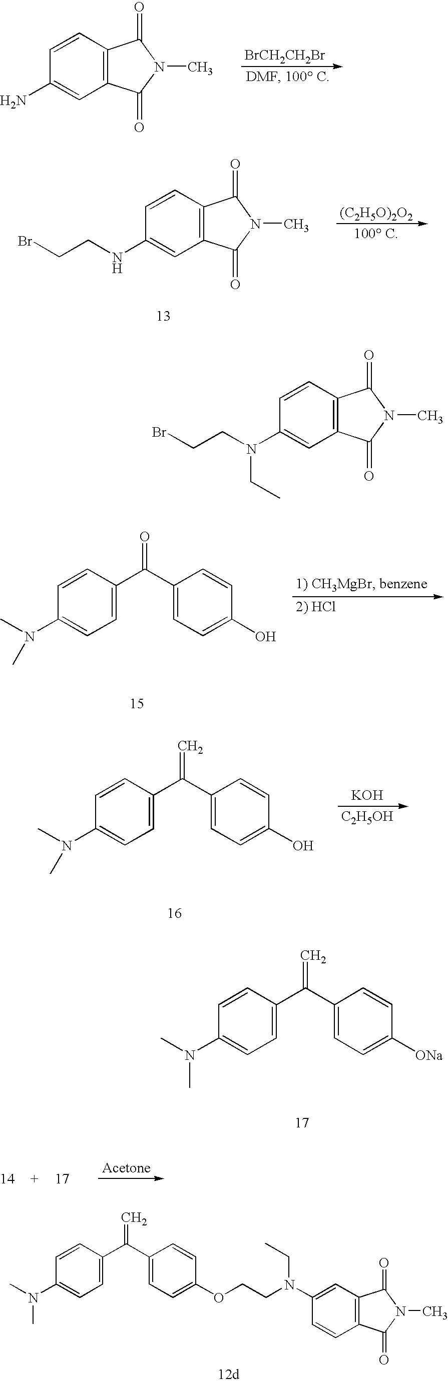

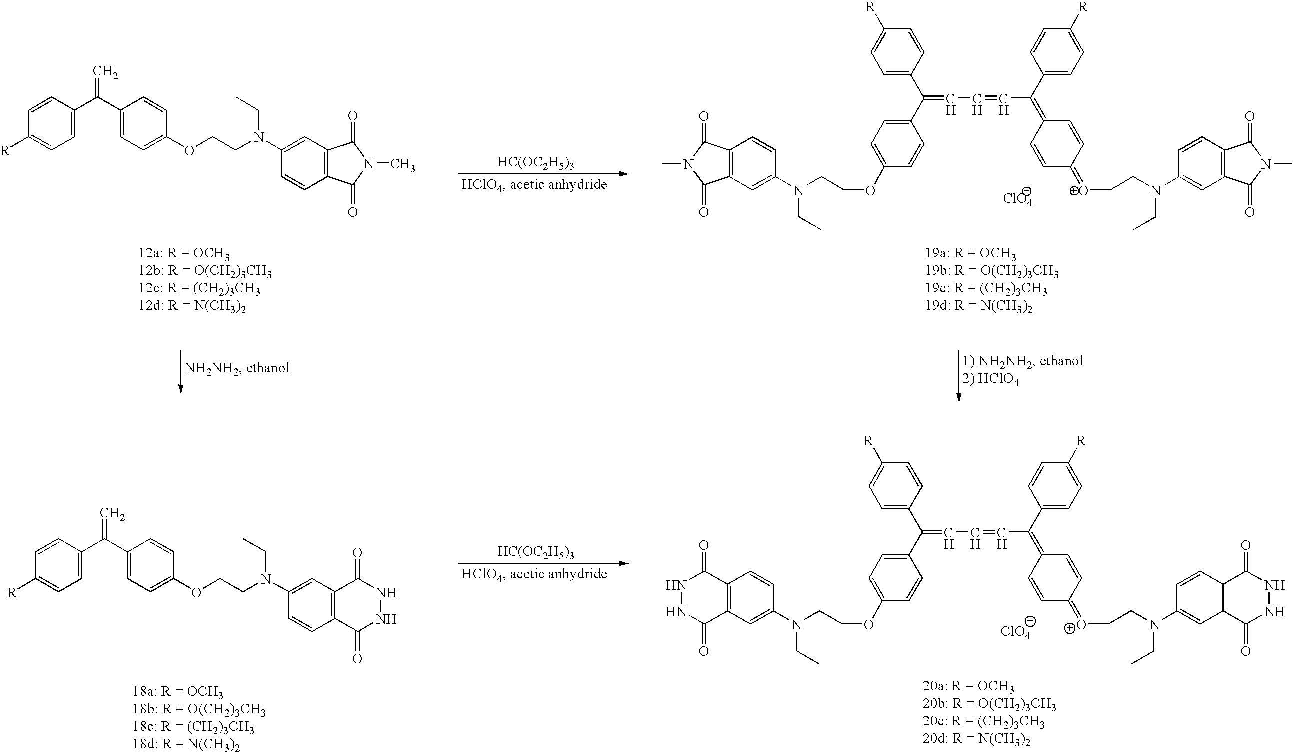

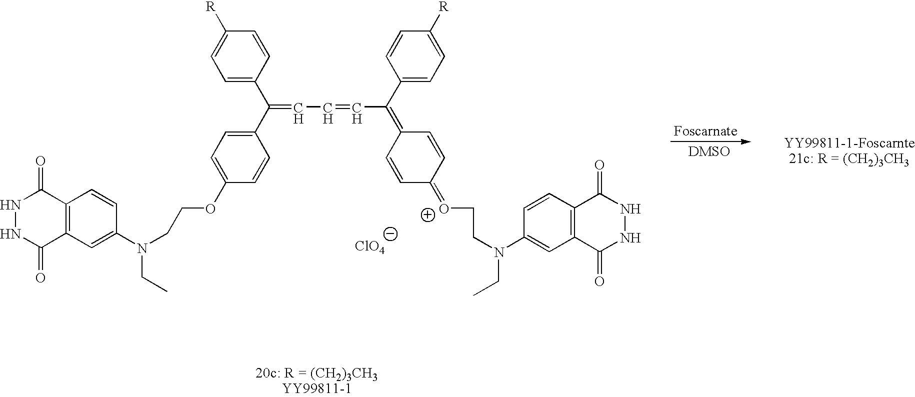

In an embodiment of the method of synthesis of the compound A-B, the A functionality comprises a phthalhydrazide such as a luminol derivative and the B functionality comprises a triarylpolymethine photochromic dye wherein A is attached to aryl groups of B; the method comprises the steps of
forming a diaryl ketone,
forming a diaryl ketene from the diaryl ketone,
forming a protected aminophthalhydrazide such as aminophthalimide or aminophthalic acid diester,
adding a hydrocarbon linker to the protected aminophthalhydrazide, and
attaching the protected aminophthalhydrazide through the molecular linker to the aryl groups of diarylketene to form the precursor aminophthalimide-linked diarylketene, and reacting according to at least one of
   (a) forming the A functionality from the precursor, and condensing the A-linked diarylketene with an aryl alkene aldehyde to form A-B, and
   (b) condensing the precursor aminophthalimide-linked diarylketene with an aryl alkene aldehyde to form A precursor linked to B, and
forming the A functionality from the A precursor to form A-B.

The diaryl ketone may be formed by a classical Friedel-Crafts acylation between a benzoyl halide and aryl compound with a hydrocarbon linker having a leaving group.

The aryl compound with a hydrocarbon linker having a leaving group may comprise at least one of a halogenated-alkyl-aryl ether and a halogenated-aklyl-aryl amine wherein the halogen is the leaving group.

The halogenated-alkyl-aryl ether may comprise 2-bromoethoxybenzene to give an aryl ketone such as 4-(2-bromoethoxy)benzophenone.

The halogenated-aklyl-aryl amine may comprise 2-bromoethyl aminobenzene to give an aryl ketone such as 4-(2-bromoethyl amino)benzophenone.

The diaryl ketone may be converted to the corresponding diarylketene by reacting with a methylating reagent such as a methyl Grignard reagent, methyl lithium reagent, lithium dimethylcopper reagent and then dehydration with acid.

The diaryl ketone may be converted to the corresponding diarylketene by reacting with methylmagnesium bromide and then dehydration with acid.

The diaryl ketone may be converted to the corresponding diarylketene by a Wittig reaction.

A linker may be attached to the protected aminophthalhydrazide by a reaction of a nucleophilic group of the linker or protected aminophthalhydrazide with a leaving group of the linker or protected aminophthalhydrazide.

A linker may be attached to the protected aminophthalhydrazide by reaction to form a bond between at least one of a nitrogen, oxygen, or carbon atom of the linker and at least one of a nitrogen, oxygen, or carbon atom of group of the protected aminophthalhydrazide by an addition or a substitution reaction of a leaving group.

A linker may be attached to the protected aminophthalhydrazide by a substitution reaction of at least one of a halogen, tosylate group, ester group with a nitrogen, oxygen, or carbon atom.

Attaching the protected aminophthalhydrazide through the molecular linker to one of the aryl groups of diarylketene to form the precursor aminophthalimide-linked diarylketene ma be by a reaction of a nucleophilic group of the linker or aryl group of diarylketene with a leaving group of the linker or aryl group of diarylketene.

A linker may be attached to the aryl group of diarylketene by reaction to form a bond between at least one of a nitrogen, oxygen, or carbon atom of the linker and at least one of a nitrogen, oxygen, or carbon atom of group of the protected aminophthalhydrazide by an addition or a substitution reaction of a leaving group.

A linker may be attached to the aryl group of diarylketene by a substitution reaction of at least one of a halogen, tosylate group, ester group with a nitrogen, oxygen, or carbon atom.

The precursor aminophthalimide-linked diarylketene may be further reacted by condensation with an aryl alkene aldehyde in a nonaqueous solvent, containing an acid catalyst to form B linked to the A precursor.

The precursor aminophthalimide-linked diarylketene may be an aminophthalimide-substituted 1,1-diarylethene,
the aryl alkene aldehyde is a p-aminophenyl alkene aldehyde such as p-(dimethylamino)cinnamaldehyde,
the nonaqueous solvent is acetic anhydride,
the acid catalyst is at least one of perchloric acid and tetrafluoroboric acid, and
the B linked to the A precursor comprises a aminophthalimide-substituted multiarylpolymethine dye.

The precursor aminophthalimide-linked diarylketene may comprise at least one of the formula 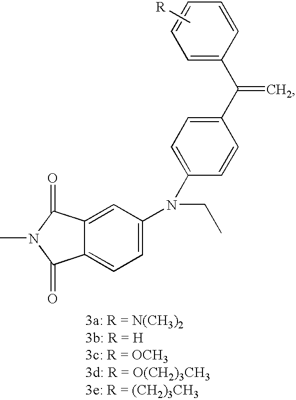
the aryl alkene aldehyde has the formula 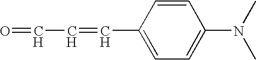
4-(Dimethylamino)cinnamaldehyde, and
the precursor of A-B comprises at least one of the formula

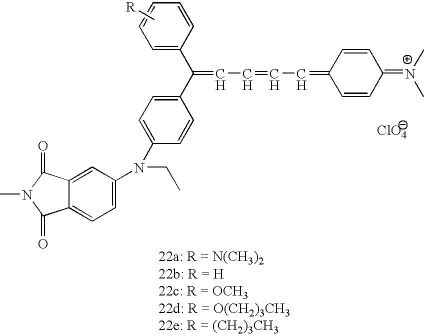

The phthalimide moiety of the A precursor may be converted to the phthalhydrazide A functionality by treating with hydrazine, forming A-B.

In an embodiment, the B functionality is protected by reacting with an anion such as hydroxide, methoxide and amine, the A-B precursor is refluxed with hydrazine in a suitable solvent such as an alcoholic solvent in inert atmosphere and then treated with acid such as perchloric acid, tetrafluroboric acid to form A-B.

The phthalimide moiety of the A precursor of the precursor aminophthalimide-linked diarylketene may be converted to the phthalhydrazide A functionality by treating with hydrazine, forming A attached to a B precursor.

The A-linked diarylketene may be further reacted by condensation with an aryl alkene aldehyde in a nonaqueous solvent, containing an acid catalyst to form A-B.

In an embodiment, the A-linked diarylketene is an aminophthalhydrazide-substituted 1,1-diarylethene,
the aryl alkene aldehyde is a p-aminophenyl alkene aldehyde such as p-(dimethylamino)cinnamaldehyde,
the nonaqueous solvent is acetic anhydride,
the acid catalyst is at least one of perchloric acid and tetrafluoroboric acid, and
A-B comprises a aminophthalhydrazide-substituted multiarylpolymethine dye.

In an embodiment, the A-linked diarylketene comprises at least one of the formula 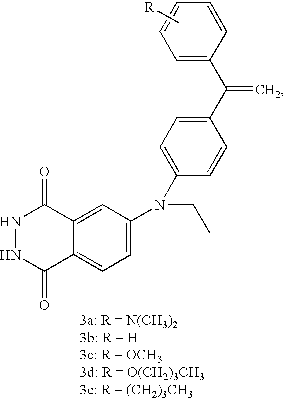
the aryl alkene aldehyde has the formula 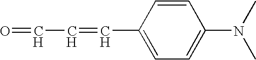
4-(Dimethylamino)cinnamaldehyde, and
A-B comprises at least one of the formula

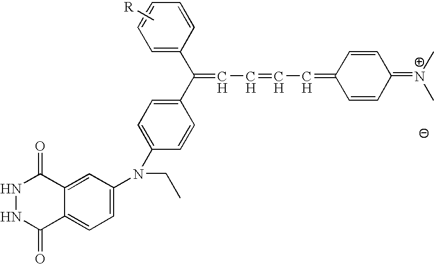

In an embodiment, the synthsis method further comprises the step of reacting the B functionality with one nucleophilic species of a C functionality such as Foscarnet to form A-B-C.

In an embodiment, the method of synthesis of the compound A-B-C comprises the general steps given by following representative formula

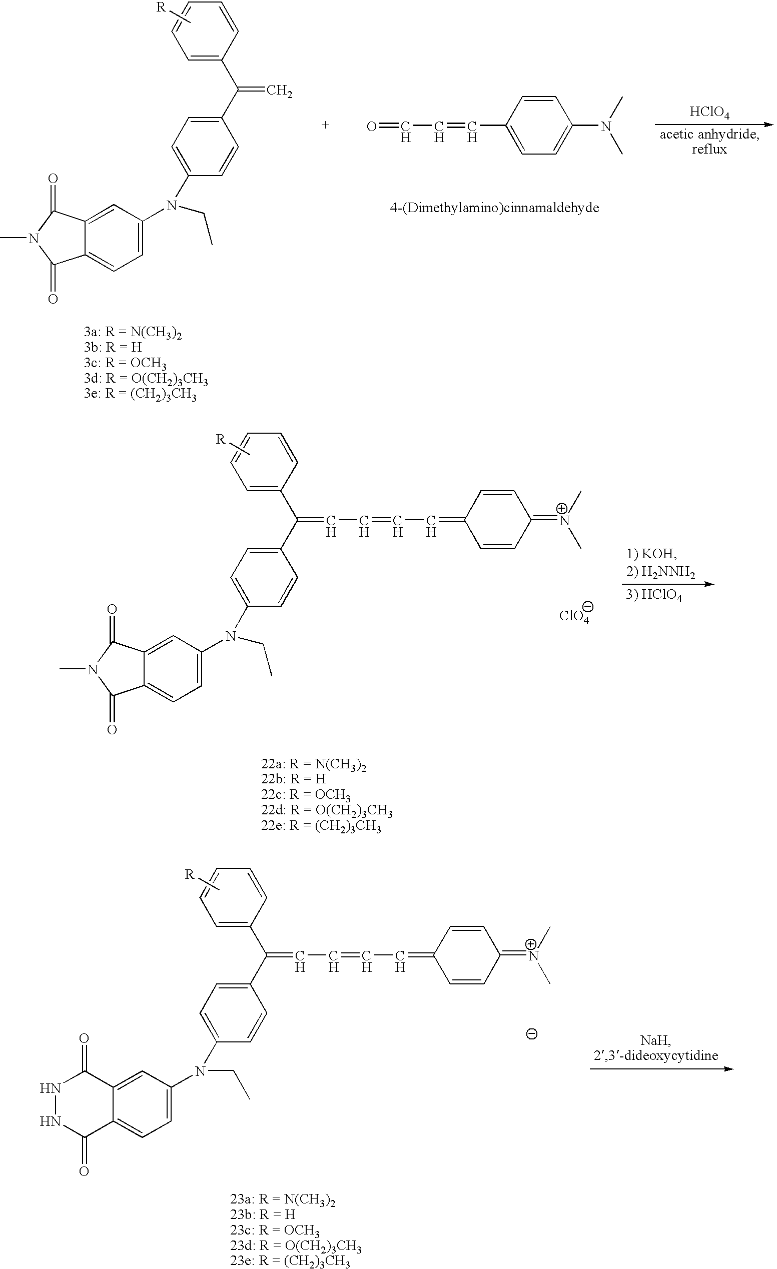

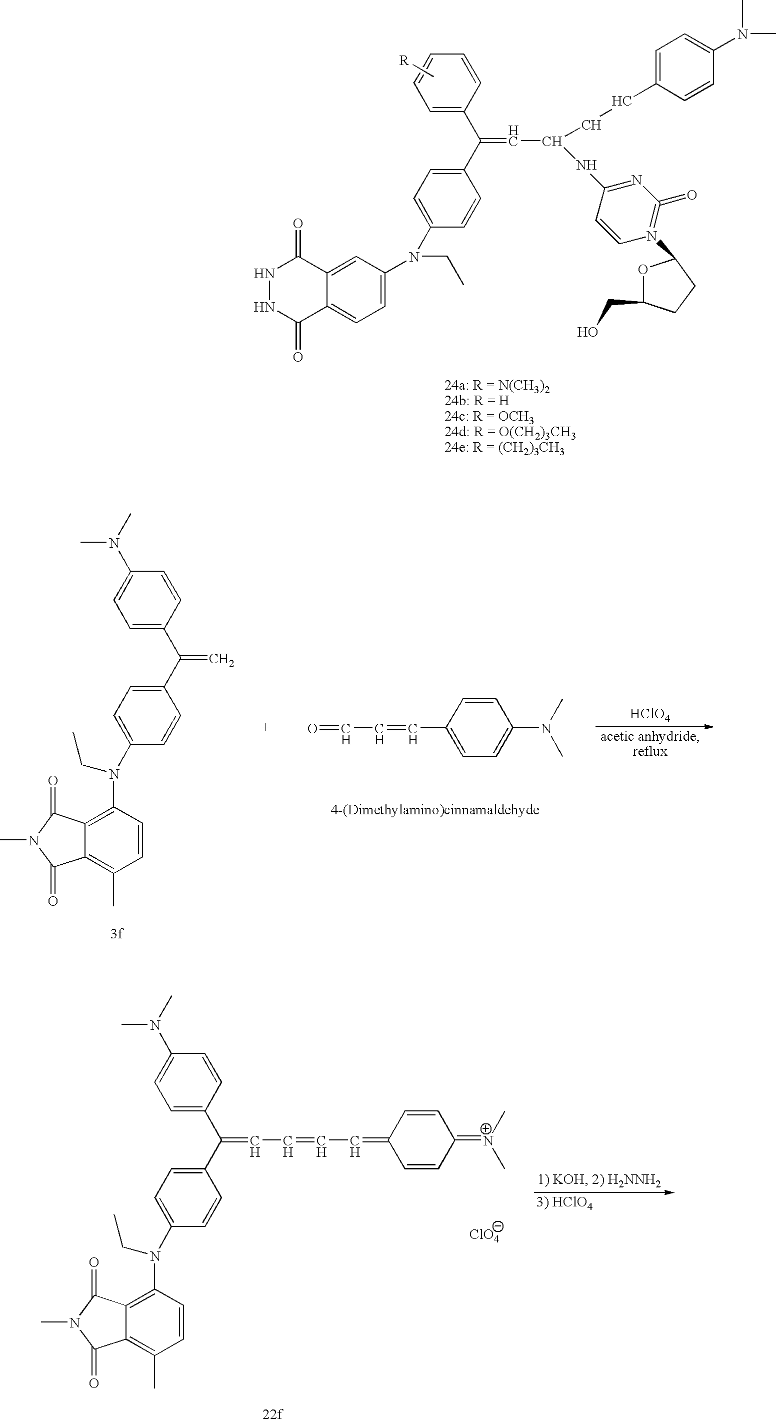

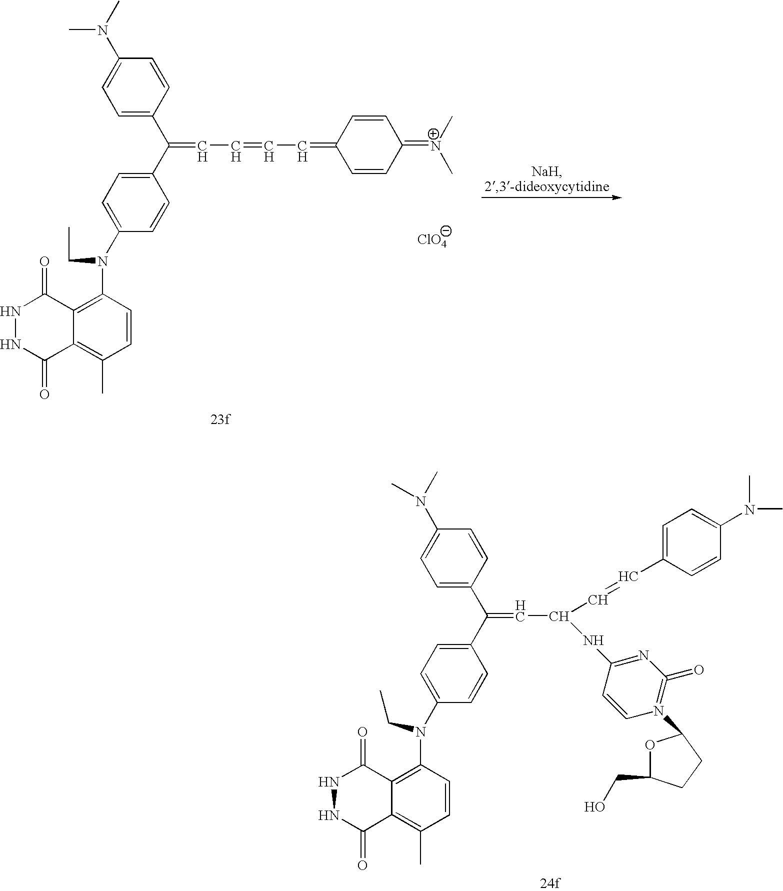

In an embodiment, the method of synthesis of the compound A-B-C comprises the general steps given by following representative formula

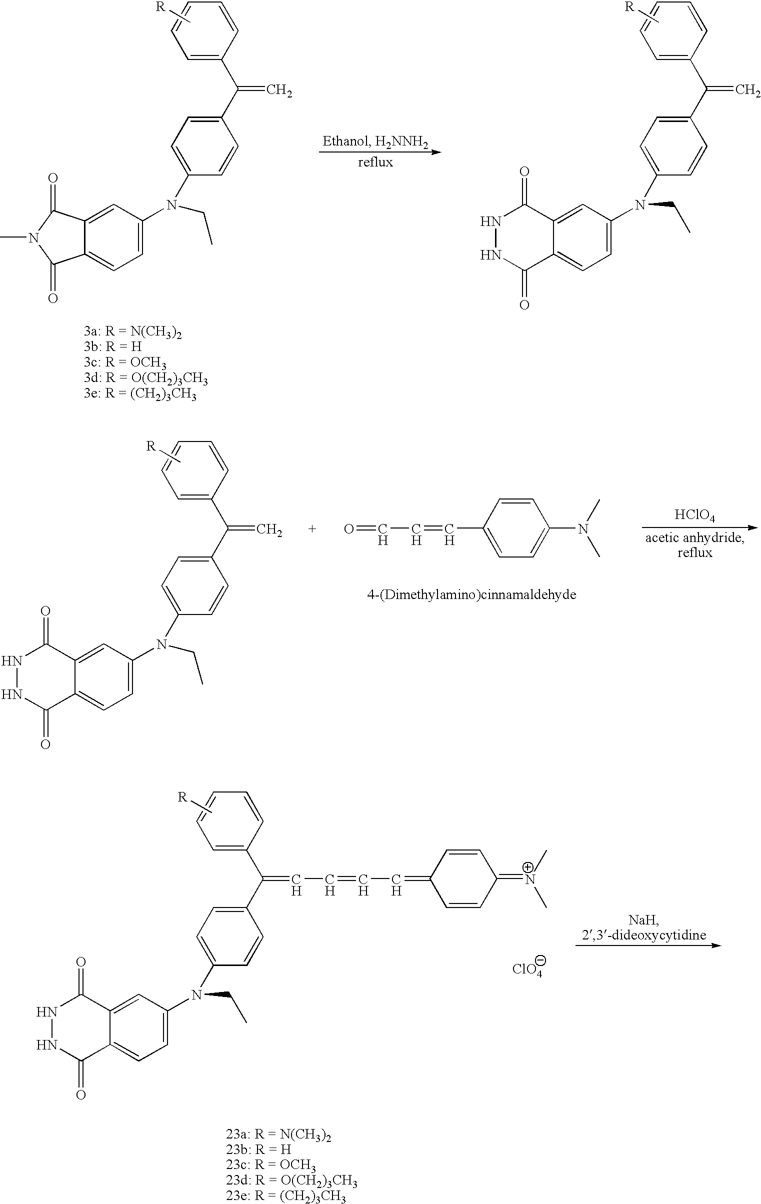

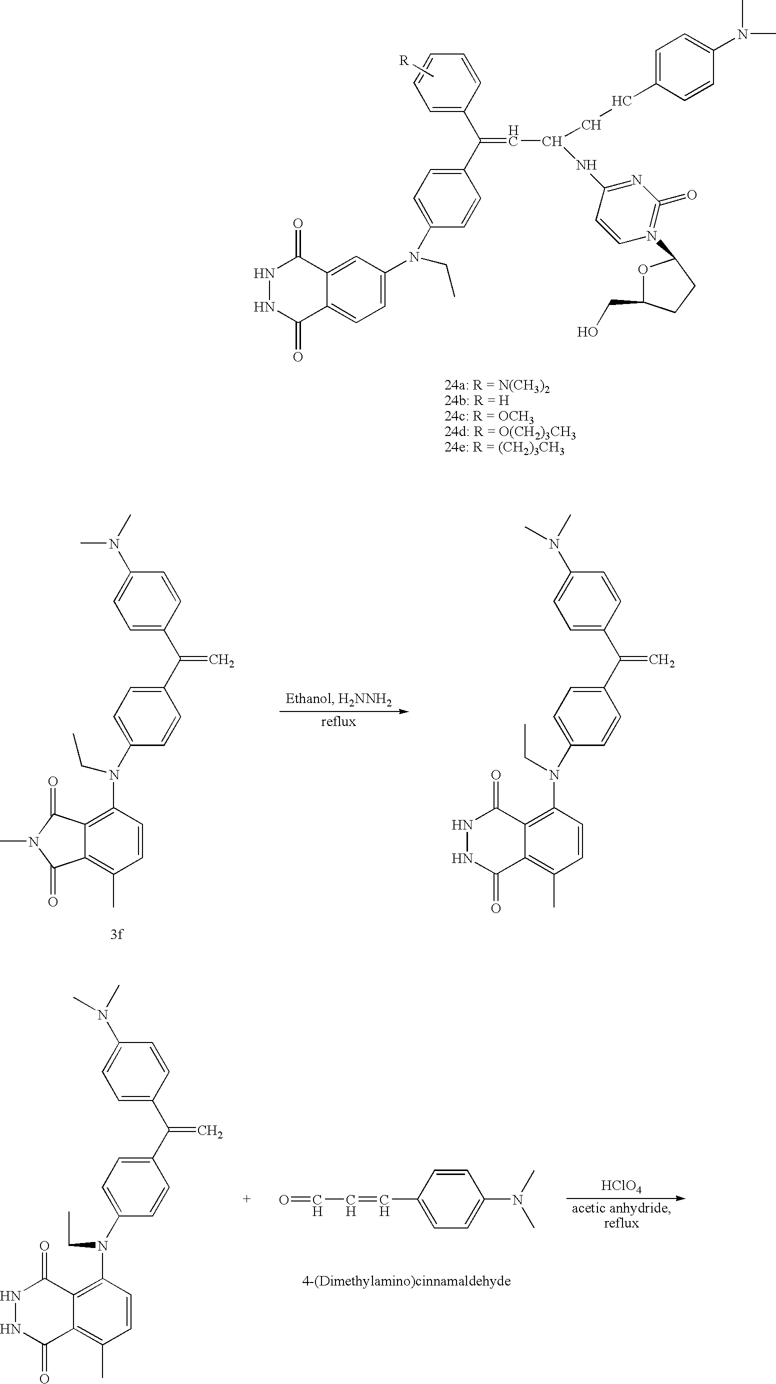

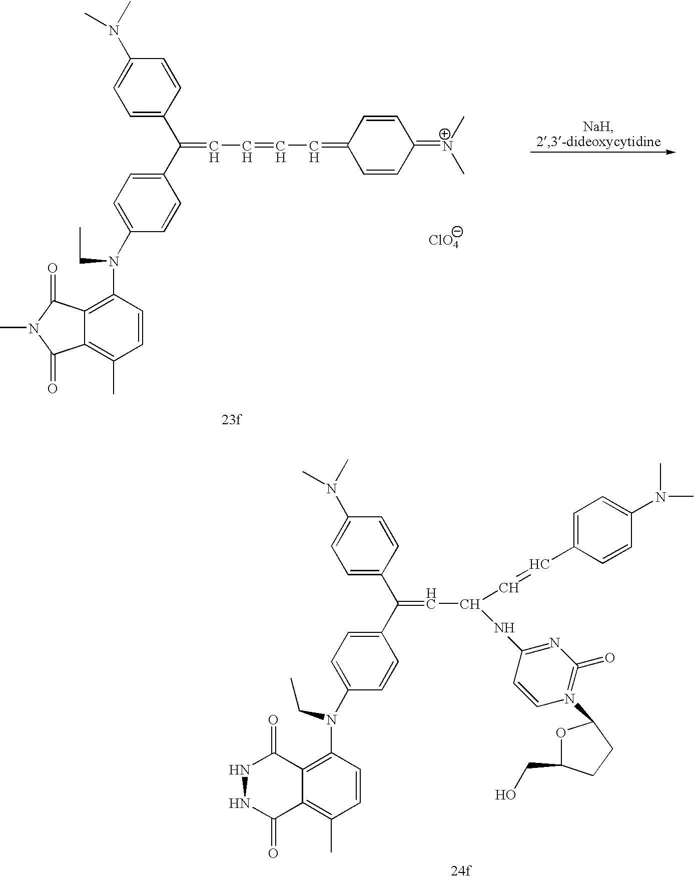

In an embodiment of the method of synthesis of the compound A-B of the present invention, the A functionality comprises a phthalhydrazide such as a luminol derivative and the B functionality comprises a triarylpolymethine photochromic dye wherein A is attached to aryl groups of B comprising the steps of
forming a diaryl ketone,
forming a diaryl ketene from the diaryl ketone,
condensing the diarylketene with an aryl alkene aldehyde to form B
forming a protected aminophthalhydrazide such as aminophthalimide or aminophthalic acid diester,
adding a hydrocarbon linker to the protected aminophthalhydrazide, and
attaching the protected aminophthalhydrazide through the molecular linker to the aryl groups of B to form the precursor aminophthalimide-linked B, and
forming the A functionality from the precursor to form A-B.

In an embodiment, at least one of the diaryl ketone and diarylketene is halo-substituted and the protected aminophthalhydrazide is attached through the linker by an amination reaction.

The halo-substituted diarylketene precursor compounds may comprise the formula of at least one of 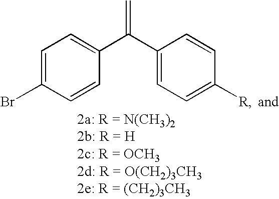
and
the halo-substituted multiarylpolymethine dyes, such as 1-(p-bromophenyl)-1,5-bis(p-dimethylaminophenyl)-pentadienium perchlorate, may be prepared by condensation with a p-aminophenyl alkene aldehyde such as p-(dimethylamino)cinnamaldehyde.

B may be protected by reacting with an anion such as alkoxide and then coupled with A by amination of aryl halide such as the palladium-catalyzed amination of aryl halide to obtain the alkoxide-protected aminophthalimide-substituted multiarylpolymethine dye.

In an embodiment, the protected aminophthalhydrazide-linked to B from the alkoxide-protected aminophthalimide-substituted multiarylpolymethine dye comprises at least one of the formula

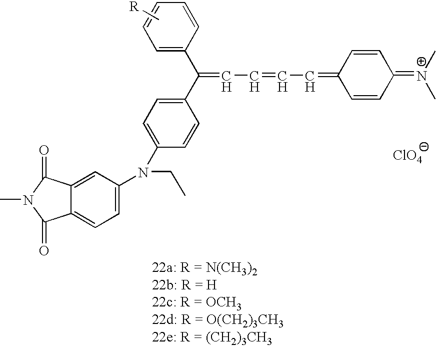

The alkoxide-protected aminophthalimide-substituted multiarylpolymethine dye may be refluxed with hydrazine in a suitable solvent such as an alcoholic solvent to convert the aminophthalimide moiety to the aminophthalhydrazide moiety and then treated with acid to generate A-B.

A-B may comprise at least one of the formula

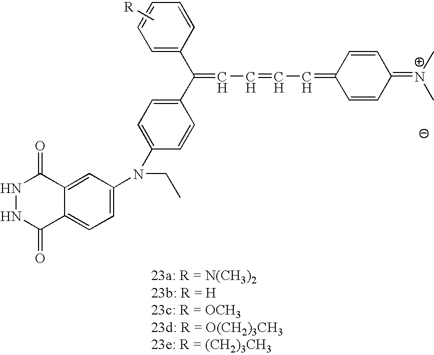

The method of synthesis of the compound A-B-C further comprises the step of reacting the B functionality with one nucleophilic species of a C functionality such as Foscarnet to form A-B-C.

In an embodiment, at least one of the diaryl ketone and diarylketene is halo-substituted and an aminophthalhydrazide is attached through the linker by an amination reaction.

In an embodiment of the method of synthesis of the compound A-B of the present invention, the A functionality comprises an active oxalate and the B functionality comprises a multiarylpolymethine photochromic dye wherein A is attached to aryl groups of B comprising the steps of
forming a halo-substituted diaryl ketone,
forming a halo-substituted diaryl ketene from the diaryl ketone,
amination of the halo-substituted diaryl ketene to give amino diarylketene,
substitution at the amino group of the ketene to forming the corresponding sulfonamide,
condensing the sulfonamide with a catalyst, and
react with oxalyl halide to form A-B.

In an embodiment of the method of synthesis of the compound A-B of the present invention, the A functionality comprises an cyclized active oxalate and the B functionality comprises a multiarylpolymethine photochromic dye wherein A is attached to aryl groups of B comprising the steps of
forming a halo-substituted diaryl ketone,
forming a halo-substituted diaryl ketene from the diaryl ketone,
amination of the halo-substituted diaryl ketene to give amino diarylketene,
substitution at the amino group of the ketene to forming the corresponding sulfonamide,
reacting 2 molar proportions of a N-substituted aminodiarylketene with 1 molar oxalyl halide to yield the N,N′-bisaryl oxamide,
condensing the oxamide with a catalyst to form A-B.

The halo-substituted diaryl ketene may be aminated using methods such as the palladium-catalyzed amination of aryl halide with benzophenoneimine to give the amino diarylketene.

The amino groups of the ketene may be substituted forming the corresponding sulfonamide by reacting with sulfonyl anhydride.

The oxamide may be condensed with an orthoester such as triethylorthofomate in a nonaqueous solvent such as acetic anhydride containing acid catalyst such as tetrafluoroboric acid, to form the cyclized oxamido-tetraarylpolymethine dye comprising A-B.

The method of synthesis of the compound A-B-C further comprises the step of reacting the B functionality with one nucleophilic species of a C functionality such as Foscarnet to form A-B-C.

In an embodiment, the method of synthesis of the compound A-B-C comprises the general steps given by following representative formula

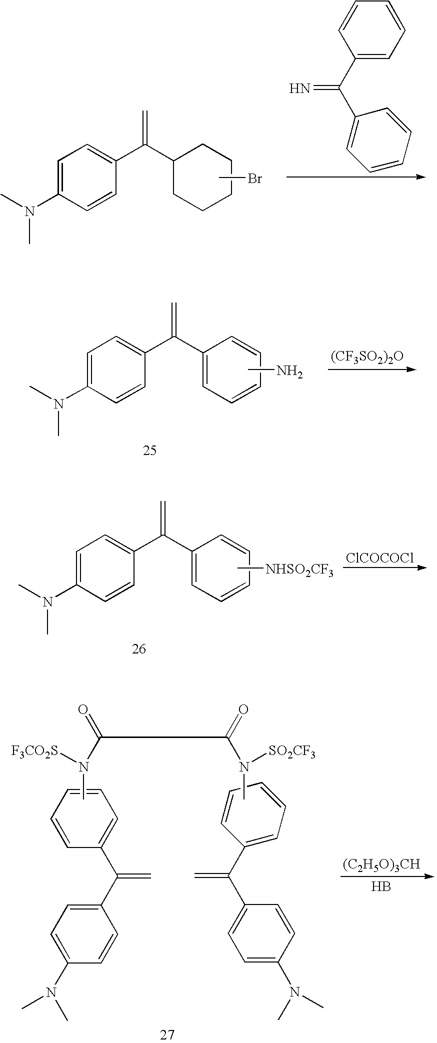

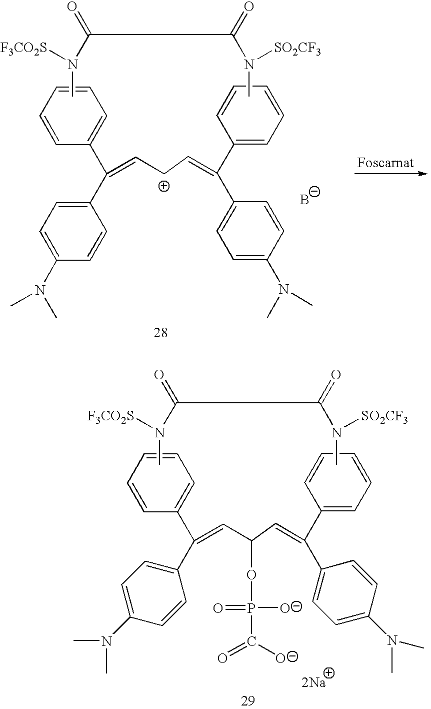

In an embodiment of the method of synthesis of the compound A-B of the present invention, the A functionality comprises an active oxalate and the B functionality comprises a multiarylpolymethine photochromic dye wherein A is attached to aryl groups of B through a molecular linker comprising the steps of
forming B comprising a functionalized tetraarylpolymethine dye,
reacting a substituted amine with a sulfonyl anhydride to form a substituted alkyl sulfonamide,
reacting the substituted alkyl sulfonamide with an oxalyl derivative to form a substituted oxamide,
reacting the substituted oxamide with the functionalized tetraarylpolymethine dye to form A-B comprising a cyclized oxamido-tetraarylpolymethine.

The substituted amine may be N-2-bromoethylsulfamide.

The oxalyl derivative may be oxalyl chloride.

The oxamide may be a N-2-bromoethyl-N-sulfonyloxamide derivative.

The oxalyl derivative may be oxalyl chloride.

The functionalized tetraarylpolymethine derivative may be a salt of a 1,5-bis(4-hydroxyphenyl)-1,5-diarylpentadiene derivative.

The cyclized oxamido-tetraarylpolymethine A-B compound may be a 1,5-(4,4′-(2,2′-N,N′-disulfonyloxamidodiethoxy)phenyl-1,5-diarylpentadiene cation derivative.

The method of synthesis of the compound A-B-C further comprises the step of reacting the B functionality with one nucleophilic species of a C functionality such as Foscarnet to form A-B-C.

In an embodiment, the method of synthesis of the compound A-B-C comprises the general steps given by following representative formula

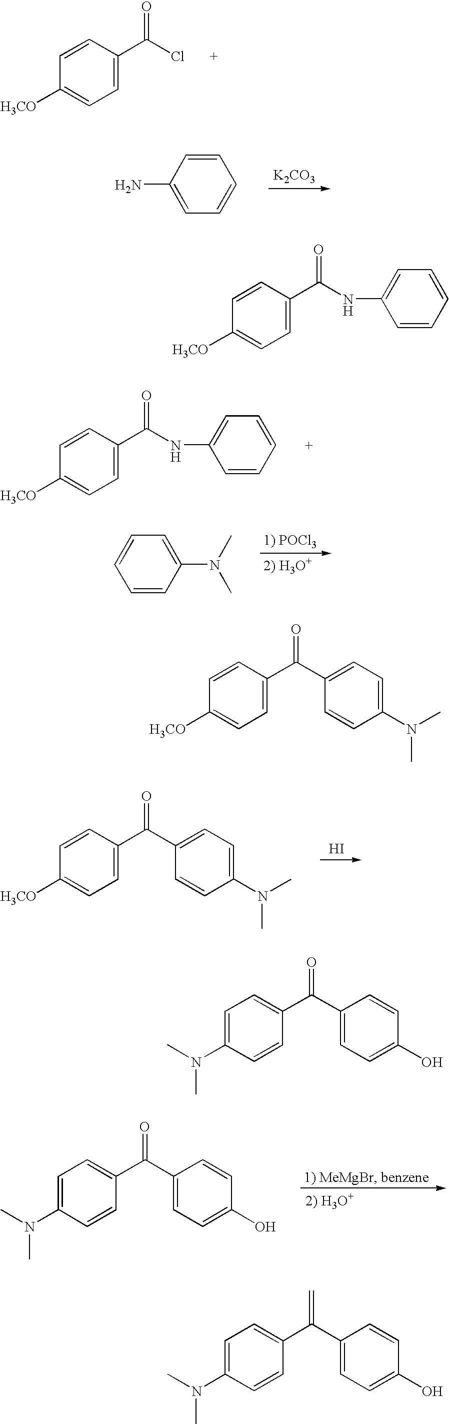

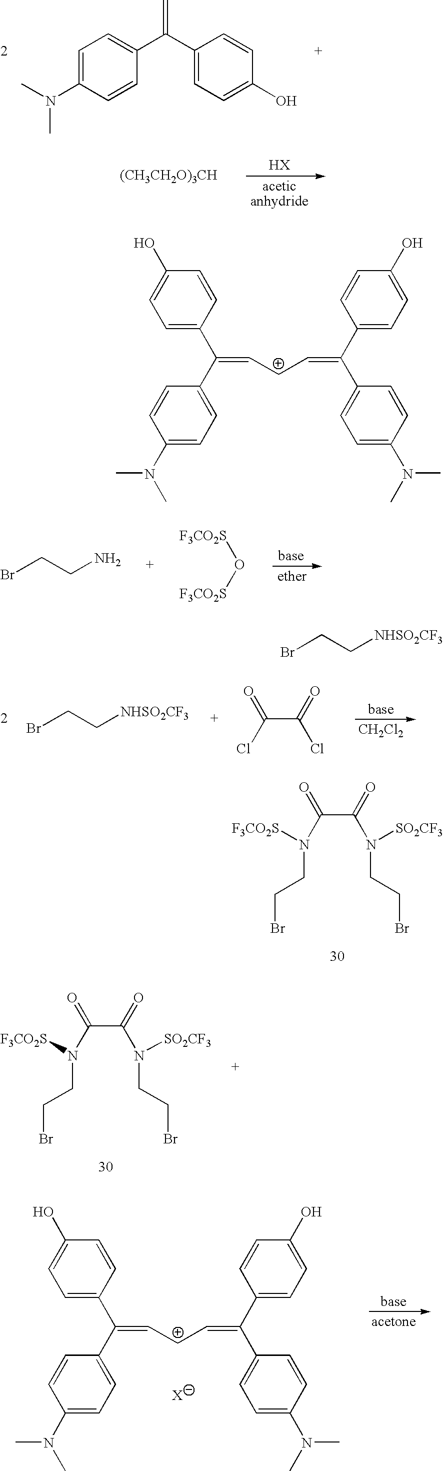

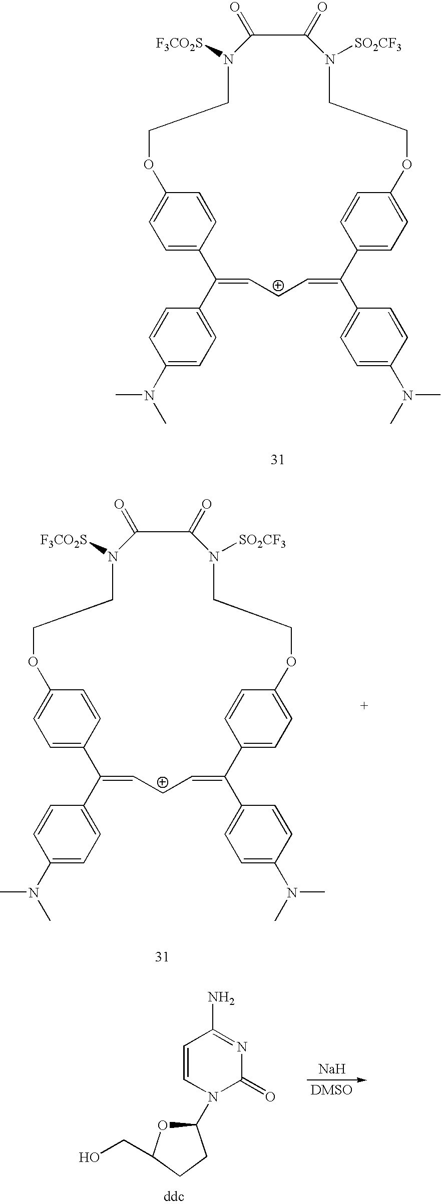

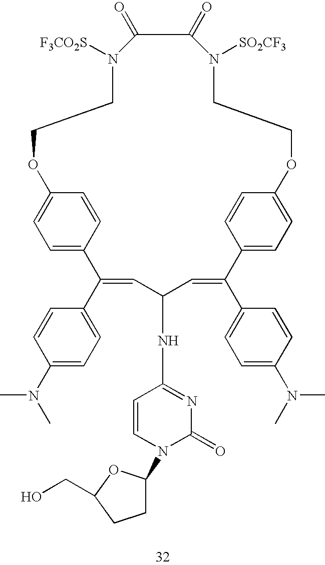

One or more of the moieties are modified to further candidate components by addition of functional groups.

In an embodiments, each of A, B, and C are modified to further candidate components by addition of functional groups one the group comprising alkyl, cycloalkyl, alkoxycarbonyl, cyano, carbamoyl, heterocyclic rings containing C, O, N, S, sulfo, sulfamoyl, alkoxysulfonyl, phosphono, hydroxyl, halogen, alkoxy, alkylthiol, acyloxy, aryl, alkenyl, aliphatic, acyl, carboxyl, amino, cyanoalkoxy, diazonium, carboxyalkylcarboxamido, alkenylthio, cyanoalkoxycarbonyl, carbamoylalkoxycarbonyl, alkoxy carbonylamino, cyanoalkylamino, alkoxycarbonylalkylamino, sulfoalkylamino, alkylsulfamoylaklylamino, oxido, hydroxy alkyl, carboxy alkylcarbonyloxy, cyanoalkyl, carboxyalkylthio, arylamino, heteroarylamino, alkoxycarbonyl, alkylcarbonyloxy, cyanoalkoxy, alkoxycarbonylalkoxy, carbamoylalkoxy, carbamoylalkyl carbonyloxy, sulfoalkoxy, nitro, alkoxyaryl, halogenaryl, amino aryl, alkylaminoaryl, tolyl, alkenylaryl, allylaryl, alkenyloxyaryl, allyloxyaryl, cyanoaryl, carbamoylaryl, carboxyaryl, alkoxycarbonylaryl, alkylcarbonyoxyaryl, sulfoaryl, alkoxysulfoaryl, sulfamoylaryl, and nitroaryl.

A further embodiment of the present invention comprises the compound comprising the formula A′-B-C
where the A′ is a chemiluminescent moiety precursor,
B is an energy acceptor moiety, and
C is a biologically active moiety.

A further embodiment of the present invention comprises the compound comprising the formula A′-B
where the A′ is a chemiluminescent moiety precursor, and
B is an energy acceptor moiety.

A further embodiment of the present invention comprises the compound comprising the formula A′-B′
where the A′ is a chemiluminescent moiety precursor, and
B′ is an energy acceptor moiety precursor.

A′ may be a precursor to generate a phthalhydrazide.

A′ may be at least one of phthalimide, aminophthalic acid diester, aminophthalic acid dihydrazide, and aminophthalic anhydride.

A′ may be a phthalhydrazide protected by a hydrolyzable group.

In an embodiment, the structure of A-B-C is given by at least one of the general formula 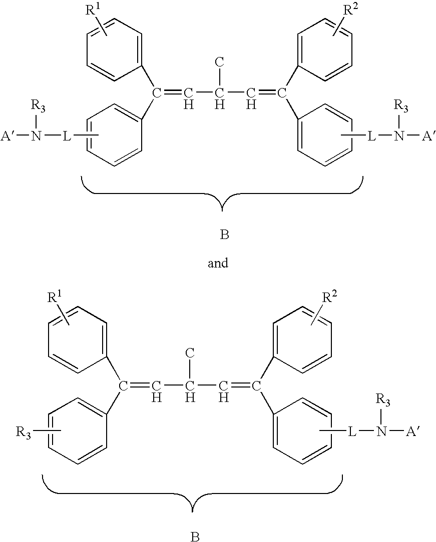
wherein the functionality A′ is at least one of precursor of an aminophthalhydrazide derivatives, sulfonyloxamides and active oxalates,
the functionality B is at least one of 1,1,5,5-tetrakisarylpentadiene derivative,
the functionality C is a drug molecule such as Foscarnate, or ddc, and
R is a functional group, and
L is a linker such as an aliphatic chain between A′ and B.

The L functionality may be between one 20 carbon atoms.

In an embodiment, the structure is given by at least one of the general formula 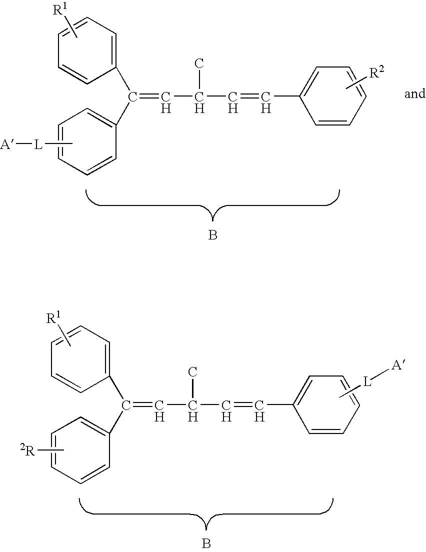
wherein the functionality A′ is at least one of precursor of an aminophthalhydrazide derivatives, sulfonyloxamides and active oxalates,
the functionality B is a 1,1,5-trisarylpentadiene derivatives,
the functionality C is a drug molecule such as Foscarnate, or ddc, and
R is a functional group, and
L is a linker such as an aliphatic chain between A′ and B.

The L functionality may between one 20 carbon atoms.

In an embodiment, the structure of A′ is given by at least one of the general formula 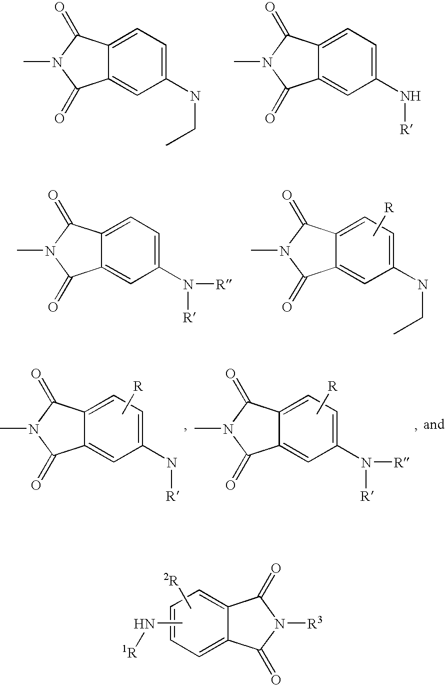
wherein R¹, R², R³, R⁴ are one of the following groups: alkyl, alkoxy, alkylamino or hydrogen, and R, R′, and R″ is at least one of alkyl, cycloalkyl, alkoxycarbonyl, cyano, carbamoyl, heterocyclic rings containing C, O, N, S, sulfo, sulfamoyl, alkoxysulfonyl, phosphono, hydroxyl, halogen, alkoxy, alkylthiol, acyloxy, aryl, alkenyl, aliphatic, acyl, carboxyl, amino, cyanoalkoxy, diazonium, carboxyalkylcarboxamido, alkenylthio, cyanoalkoxycarbonyl, carbamoylalkoxycarbonyl, alkoxy carbonylamino, cyanoalkylamino, alkoxycarbonylalkylamino, sulfoalkylamino, alkylsulfamoylaklylamino, oxido, hydroxy alkyl, carboxy alkylcarbonyloxy, cyanoalkyl, carboxyalkylthio, arylamino, heteroarylamino, alkoxycarbonyl, alkylcarbonyloxy, cyanoalkoxy, alkoxycarbonylalkoxy, carbamoylalkoxy, carbamoylalkyl carbonyloxy, sulfoalkoxy, nitro, alkoxyaryl, halogenaryl, amino aryl, alkylaminoaryl, tolyl, alkenylaryl, allylaryl, alkenyloxyaryl, allyloxyaryl, cyanoaryl, carbamoylaryl, carboxyaryl, alkoxycarbonylaryl, alkylcarbonyoxyaryl, sulfoaryl, alkoxysulfoaryl, sulfamoylaryl, and nitroaryl.

A′ may be a precursor to generate an active oxalate.

A′ may be a sulfonamide.

A′ may be a precursor to generate a cyclized active oxalate.

A′ may be a sulfonamide bound to at least one of a diaryl ketone, halo-substituted diaryl ketone, amino-substituted diaryl ketone, diaryl ketene, halo-substituted diaryl ketene, and amino-substituted diaryl ketene.

A′-B or A′-B′ may a precursor for a sulfonamide-cyclized oxamido derivative bound to a photochromic dye comprising A-B.

A′ may be attached to aryl groups of B through a molecular linker.

A′ may be at least one of substituted alkyl sulfonamide and a substituted oxamide.

The substituted amide may a N-2-bromoethylsulfamide derivative.

The oxamide may a N-2-bromoethyl-N-sulfonyloxamide derivative.

B may comprisie a functionalized tetraarylpolymethine derivative such as a salt of a 1,5-bis(4-hydroxyphenyl)-1,5-diarylpentadiene derivative.

The cyclized oxamido-tetraarylpolymethine A-B compound may be a 1,5-(4,4′-(2,2′-N,N′-disulfonyloxamidodiethoxy)phenyl-1,5-diarylpentadiene cation derivative.

The compound A-B may have the general formula

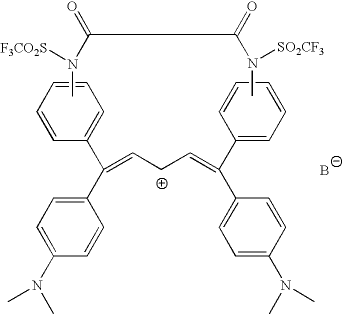

In an embodiment, the compound comprises A-B-C wherein the cyclized oxamido-tetraarylpolymethine A-B compound is a 1,5-(N,N′-disulfonyloxamido)phenyl-1,5-diarylpentadiene cation; or a 1,5-(4,4′-(2,2′-N,N′-disulfonyloxamidodiethoxy)phenyl-1,5-diarylpentadiene cation derivative and C is a biological active moiety such as the drug Foscarnet.

The compound A-B-C may be given by the general formula 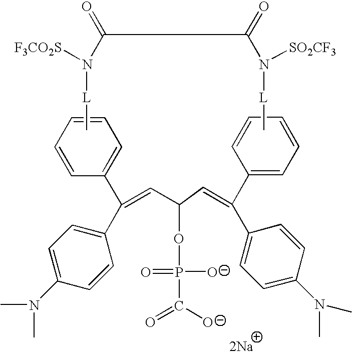
wherein L is a linker with 0 to 20 chain atoms.

The structure of A′ may be a oxalylate given by the general formula 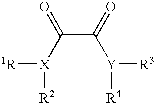
wherein X is O or N and y is O or N; R's are one the following groups: hydrogen, alkyl, cycloalkyl, alkoxycarbonyl, cyano, carbamoyl, heterocyclic rings containing C, O, N, S, sulfo, sulfamoyl, alkoxysulfonyl, phosphono, hydroxyl, halogen, alkoxy, alkylthiol, acyloxy, aryl, alkenyl, aliphatic, acyl, carboxyl, amino, cyanoalkoxy, diazonium, carboxyalkylcarboxamido, alkenylthio, cyanoalkoxycarbonyl, carbamoylalkoxycarbonyl, alkoxy carbonylamino, cyanoalkylamino, alkoxycarbonylalkylamino, sulfoalkylamino, alkylsulfamoylaklylamino, oxido, hydroxy alkyl, carboxy alkylcarbonyloxy, cyanoalkyl, carboxyalkylthio, arylamino, heteroarylamino, alkoxycarbonyl, alkylcarbonyloxy, cyanoalkoxy, alkoxycarbonylalkoxy, carbamoylalkoxy, carbamoylalkyl carbonyloxy, sulfoalkoxy, nitro, alkoxyaryl, halogenaryl, amino aryl, alkylaminoaryl, tolyl, alkenylaryl, allylaryl, alkenyloxyaryl, allyloxyaryl, cyanoaryl, carbamoylaryl, carboxyaryl, alkoxycarbonylaryl, alkylcarbonyoxyaryl, sulfoaryl, alkoxysulfoaryl, sulfamoylaryl, and nitroaryl.

A′ may be a sulfonyl oxamide given by the general formula 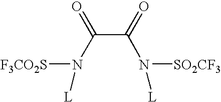
wherein L is aliphatic chain linker with 0 to 20 chain atoms.

The structure of A′ may be given by the general formula 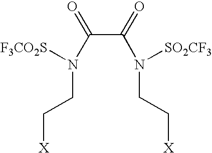
where X is a leaving group such as halide.

In an embodiment, the structure of A′-B is given by the general formula

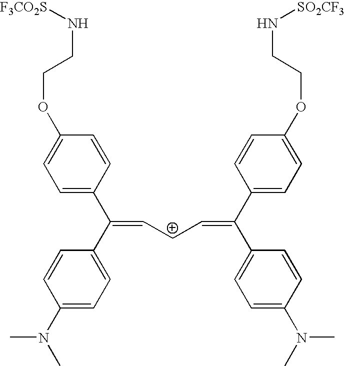

In an embodiment, the structure of A′-B′ is given by the general formula 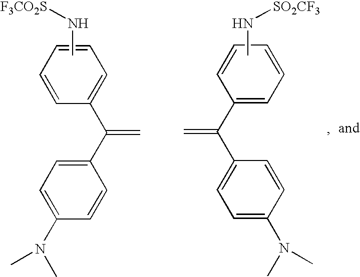
and
A-B′ is given by the general formula

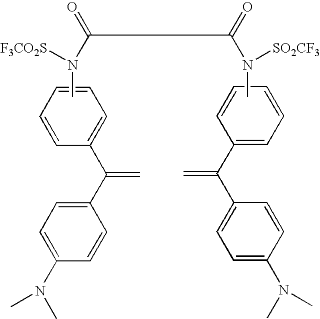

In an embodiment, the compound comprising the structure of A-B-C given by the general formula

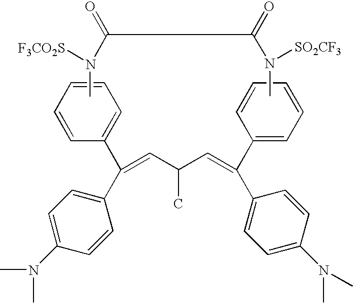

The B′ moiety may be a precursor photochromic compound.

The photochromic compound may comprise one which demonstrate photochromic behavior with electromagnetic radiation and bleaching agents.

The photochromic compound may comprise a cationic dye.

The cationic dye may comprise at least one of a di and triarylmethane dyes, triarylmethane lactones and cyclic ether dyes, cationic indoles, pyronines, phthaleins, oxazines, thiazines, acridines, phenazines, and anthocyanidins, and cationic polymethine dyes and azo and diazopolymethines, styryls, cyanines, hemicyanines, dialkylaminopolyenes, and other related dyes.

The cationic dye may comprises at least one of the compounds given in Table 2.

B′ may comprise a diaryl ketone.

B′ may comprise a diaryl ketene.

The diaryl ketone may comprise deivatives of the following representative formula

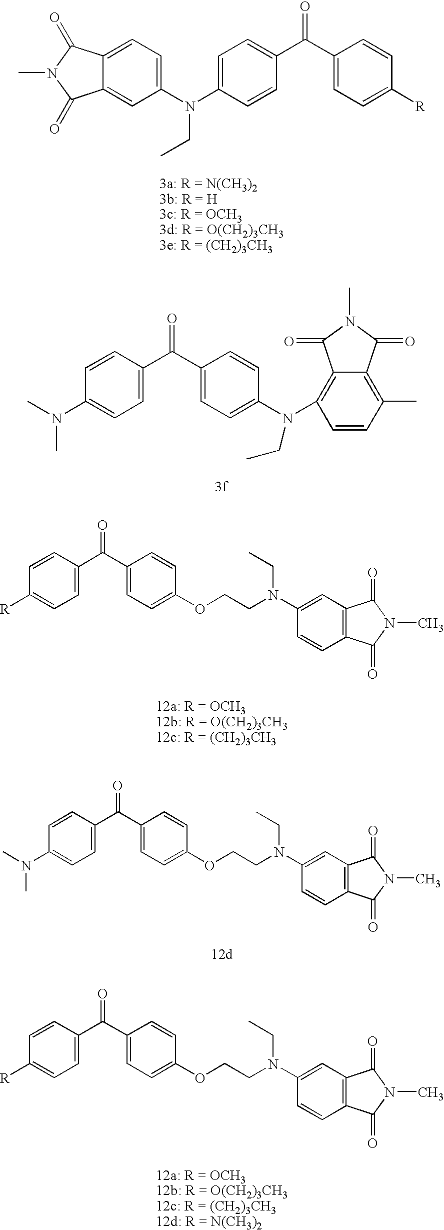

The diaryl ketene may comprise deivatives of the following representative formula

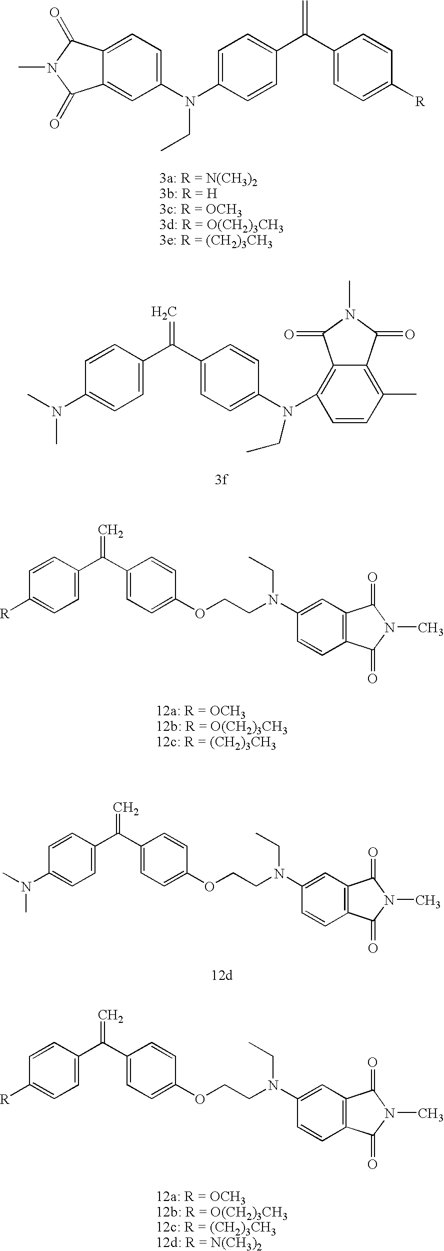

A′-B may comprise deivatives of the following representative formula

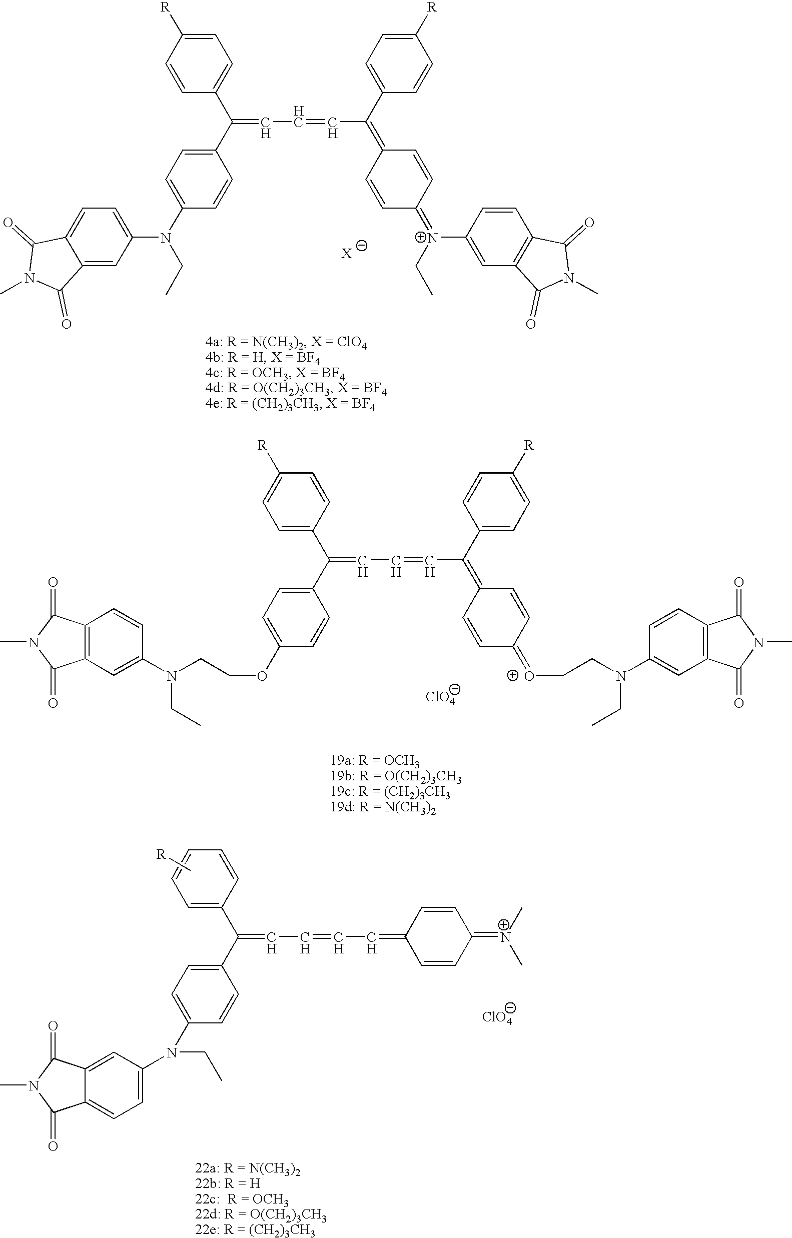

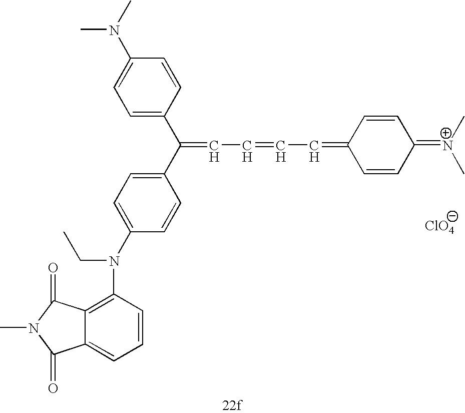

The C moiety may be at least one of the group of the compounds given in Table 3.

One or more of the moieties of at least A, B, C, A′, B′, and C′ wherein C′ is a modified biologically active compound or their precursors may be modified to further candidate components by addition of functional groups.

Each of at least A, B, C, A′, B′, and C′ wherein C′ is a modified biologically active compound or their precursors may be modified to further candidate components by addition of functional groups one the group comprising alkyl, cycloalkyl, alkoxycarbonyl, cyano, carbamoyl, heterocyclic rings containing C, O, N, S, sulfo, sulfamoyl, alkoxysulfonyl, phosphono, hydroxyl, halogen, alkoxy, alkylthiol, acyloxy, aryl, alkenyl, aliphatic, acyl, carboxyl, amino, cyanoalkoxy, diazonium, carboxyalkylcarboxamido, alkenylthio, cyanoalkoxycarbonyl, carbamoylalkoxycarbonyl, alkoxy carbonylamino, cyanoalkylamino, alkoxycarbonylalkylamino, sulfoalkylamino, alkylsulfamoylaklylamino, oxido, hydroxy alkyl, carboxy alkylcarbonyloxy, cyanoalkyl, carboxyalkylthio, arylamino, heteroarylamino, alkoxycarbonyl, alkylcarbonyloxy, cyanoalkoxy, alkoxycarbonylalkoxy, carbamoylalkoxy, carbamoylalkyl carbonyloxy, sulfoalkoxy, nitro, alkoxyaryl, halogenaryl, amino aryl, alkylaminoaryl, tolyl, alkenylaryl, allylaryl, alkenyloxyaryl, allyloxyaryl, cyanoaryl, carbamoylaryl, carboxyaryl, alkoxycarbonylaryl, alkylcarbonyoxyaryl, sulfoaryl, alkoxysulfoaryl, sulfamoylaryl, and nitroaryl.

### DETAILED DESCRIPTION OF THE INVENTION

Electron transferring and transporting elements are ubiquitous and are necessary for life. All eukaryotic and prokaryotic organisms depend on electron transferring and transporting elements which include metal containing hemes and nonmetal containing molecules such as flavins to convert the energy stored in the chemical bonds of foodstuffs into a form utilizable for the maintenance of the highly negative entropic state of life. The chemical energy conversion process generally involves a coupled series of electron carriers which is called an electron transport chain. Free radicals of oxygen are produced during aerobic respiration in mitochondria as electrons are carried by electron carriers of the electron transport chain to the ultimate electron acceptor, oxygen, and superoxide and peroxide, partial reduction products of oxygen, are continuously produced during cytosolic hydroxylation and oxygenation reactions as well as during other reactions which involve enzymatic reduction of oxygen. The cytosol as well as mitochondria of aerobic cells contain high concentrations of the enzyme superoxide dismutase which converts superoxide into hydrogen peroxide and molecular oxygen. Oxygen radicals which include hydrogen peroxide and superoxide are found in greater concentration in the mitochondria relative to the cytosol because reduction of oxygen occurs to a greater extent in the former compartment; however, appreciable concentration are found in both compartments.

Luminides are agents which are permeant to the desired biological compartment which undergo an oxidation reduction reaction with the target cell's electron carriers or react with free radicals produced as a consequence of electron transport and release a drug moiety into the desired compartment in active form to effect a greater therapeutic effect or therapeutic ratio relative to the free C agent as a consequence of altered pharmacokinetics or pharmacodynamics such as a desirable kinetics of release, a resistance to inactivation or excretion, greater solubility, enhanced absorption, a diminished toxicity, or greater access to the cellular or biological compartment which is the site of action of C.

Luminide agents are three or four part molecules where each part is a functionality with a defined purpose. Exemplary Luminides are A-B-C, D-A-B-C, A-D-B-C and 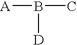
where A represents a functionality which undergoes an oxidation reduction reaction where electrons are transferred directly between A and the target cell's electron carriers or the electrons are transferred indirectly through an electron transfer functionality, D, which is described in more detail below. Alternatively, A represents a functionality which undergoes a reaction with free radicals of oxygen which are produced as a consequence of electron transport. An excited state is produced in A as a consequence of its participation in one of these reactions. Then A undergoes intramolecular energy transfer from its own excited state to the B functionality which is an energy acceptor. Upon receiving energy from A, B achieves an excited state which relaxes through heterolytic cleavage of the covalent bond of B with C where C is a drug moiety which is released into the environment. D serves as an electron transfer functionality which gains (loses) electrons from (to) the environment and donates (accepts) electrons to (from) A to activate it so that the energy of excited A is transferred to B with release of C as occurs for the three functionality case. In both cases, free C is a drug molecule. The released drug molecule effects a therapeutic functional change by a mechanism which comprises receptor mediated mechanisms including reversible and irrereversible competitive agonism or antagonism including a molecule known as a suicide substrate or a transition state analogue mechanism or a noncompetitive or uncompetitive agonism or antagonism or the action is by a nonreceptor mediated mechanism including a “counterfeit incorporation-mechanism”.

The energy donating functionality, A, is a molecule which reacts as previously described to form an excited state of high enough energy so that this subsequently transferred energy is of sufficient magnitude to break the covalent bond between the drug functionality, C, and the energy acceptor functionality, B. Chemiluminescent molecules can form highly excited states of the proper magnitude of energy, can undergo oxidation reduction reactions or react with free radicals, and possess a metastable excited state from which intramolecular energy transfer can occur; thus, they can serve as the A functionality. In general, chemiluminescent molecules relevant to this invention can be placed into three categories: 1) molecules undergoing reaction involving peroxides and oxygen free radicals; 2) molecules undergoing reaction involving oxidation or reduction and 3) molecules undergoing both reaction with peroxides and oxygen free radicals followed by an oxidation or reduction reaction. Molecules of the first category include Lophine and its derivatives, acridinium esters and acridans, tetraphenylpyrrole, phthalhydrazides, acyloins, biacridinium salts, vinylcarbonyls, vinylnitriles, tetrakis (dimethylamino) ethylene, acylperoxides, indoles, tetracarbazoles and active oxalates.

Molecules belonging to the second category include ruthenium chelates 2, 6-diaminopyrene, or cation radicals and molecules which follow a Chemically Initiated Electron Exchange Luminescence mechanism such as certain dioxetans and dioxetanones. Dioxene derivatives belong to the third category. They form a dioxetan by reation with superoxide and then produce efficient chemiluminescence by a CIEEL mechanism.

As an example from the first category, the chemiluminescent compound, luminol, has a chemiluminescent maximum in the region 390-400 nm in an aqueous solution. Chemiluminescence is produced by the reaction of luminol with oxygen free radicals where a large fraction of the product molecules are formed in their excited state. The nature of the excited state is electronic, and it has a mean lifetime of the order of seconds which is typically ten thousand times the period of a molecular vibration. Emission involves a quantum mechanically allowed singlet to singlet transition with energy of the order of 75 Kcal/mole. The quantum yield for forming the excited electronic state is 0.5. Because luminol undergoes a chemiluminescent reaction with oxygen radicals, this compound has been used as a molecular probe for these radicals by linkage to a molecule which directs the probe to a cellular compartment. For example, when luminol is attached to carnitine, the probe is transported into mitochondria and the intensity of chemiluminescence produced is proportional to the magnitude of electron transport activity which produces oxygen radicals. The chemiluminescent molecule, lucigenin, is also used as a probe for oxygen free radicals.

As for members of the second category, chemiluminescent molecules which undergo a redox reaction to produce an excited state react directly with electron carriers of the cell or undergo a redox reaction with the electron transfer functionality D.

As for the third category, a D functionality is optional. A chemiluminescent molecule of this category reacts with oxygen free radicals and forms an excited state, and chemiluminescence is produced but properties such as quantum yield or the relative ratio of singlet to triplet excited state can be altered by the transfer of electrons involving for example a D functionality. See TABLE 1 below for chemiluminescent molecules.

TABLE 1
Representative Chemiluminescent Molecules

| Name | Structure |
|---|---|
| 2,6-diaminopyrene | 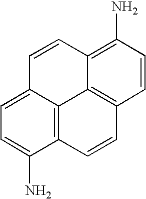 |
| Aminophthalhydrazide | 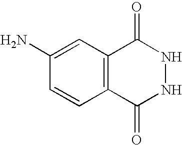 |
| Dioxene | 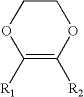 |
| Imidazole derivatives | 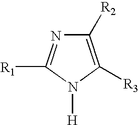 |
| Sulfonyloxamides | 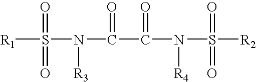 |
| Indole derivatives | 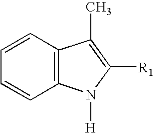 |
| Tetrakis(dialkylamino)-ethylene | 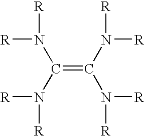 |
| 2,5,7,8-tetraoxabicyclo- [4.2.0.] octane | 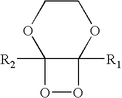 |
| Dioxetan | 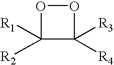 |
| Lucigenin | 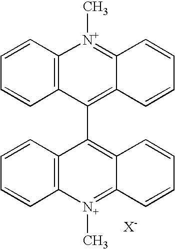 |
| Lophine | 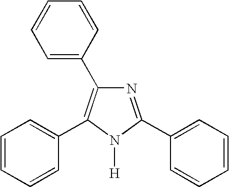 |
| Acridinium esters | 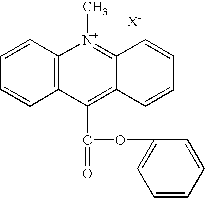 |
| Active oxalate | 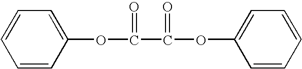 |
| Tris-2,2′- bipyridinedichlororuthenium (II) | 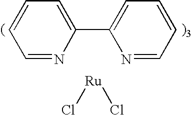 |
| Dioxetanone | 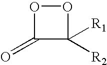 |
| Dipheyl peroxide | 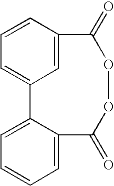 |

Exemplary energy acceptor molecules include those which demonstrate photochromic behavior with electromagnetic radiation and bleaching agents. If the A functionality is chemiluminescent, then the B functionality is such that the photodissociative drug release spectrum of B overlaps the chemiluminescence spectrum of A.

Triarylmethane dyes react with cyanide to form nitriles called leucocyanides which liberate cyanide ion with a quantum yield of approximately one when irradiated with UV light in the wavelength range of 250 to 320 nm. 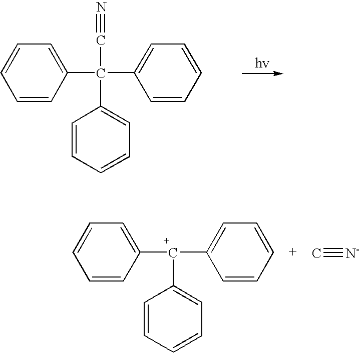
The spectrum of the photorelease reaction of cyanide ion can be extended to longer wavelengths in the case of triarylmethane dyes by substitutions of a naphthalene for an aryl group and also by using cationic polymethine dyes. The latter form nitriles, which are thermally stable, by the reaction of the carbonium ion of the dye with cyanide. The formation of the nitrile causes the colored dye to be bleached as is the case with triarylmethane dyes, and cyanide is released as the dye becomes colored upon absorption of 320-415 nm. Reversible bleaching by an agent and coloration by light is photochromic behavior.

Cationic dyes demonstrate this behavior and include di and triarylmethane dyes, triarylmethane lactones and cyclic ether dyes, cationic indoles, pyronines, phthaleins, oxazines, thiazines, acridines, phenazines, and anthocyanidins, and cationic polymethine dyes and azo and diazopolymethines, styryls, cyanines, hemicyanines, dialkylaminopolyenes, and other related dyes. See TABLE 2 below for structures for salt isomerism-type photochromic dyes. These photochromic molecules form covalent bonds with a number of agents called bleaching agents because they convert the compounds from colored to colorless form during bond formation. Bleaching agents are diverse and include hydroxide, cyanide, azide, bisulfide, and sulfite compounds, thiocyanate, ferrocyanide, chromate, tetraborate, acetate, nitrite, carbonate, citrate, aluminate, tungstate, molybdate, methoxide, 2-methoxyethoxide, cinnamate, and p-methoxycinnamate salts, and thiols and amines.

TABLE 2
Dye Name or Structure; CI Name and Number; Other Names

| | |
|---|---|
| Malachite Green | 42000 |
| Helvetia Green | 42020 |
| Basic Blue 1 | 42025 |
| Brilliant Blue | |
| Setoglaucine | |
| Basic Green 1 | 42040 |
| Brilliant Green | |
| Acid Blue 1 | 42045 |
| Xylene Blue VS | |
| Patent Blue V | |
| Alphazurine 2G | |
| Acid Blue 3 | 42051 |
| Brilliant Blue V | |
| Patent Blue V | |
| Food Green 3 | 42053 |
| FDC Green 3 | |
| Acid Green 6 | 42075 |
| Light Green SF Bluish | |
| Acid Blue 7 | 42080 |
| Xylene Blue AS | |
| Patent Blue A | |
| Acid Green 3 | 42085 |
| Acid Blue 9 | 42090 |
| Erioglaucine | |
| Acid Green 5 | 42095 |
| Light Green SF Yellowish | |
| Acid Green 9 | 42100 |
| Erioviridene B | |
| Acid Blue 147 | 42135 |
| Xylene Cyanol FF | |
| Basic Red 9 | 42500 |
| Pararosaniline | |
| Basic Violet 14 | 42510 |
| Fuchsin | |
| Magenta | |
| Basic Fuchsin | 42510B |
| Basic Violet 2 | 42520 |
| New Magenta | |
| Hoffman Violet | 42530 |
| Iodine Violet | |
| Basic Violet 1 | 42535 |
| Methyl Violet | |
| Basic Violet 13 | 42536 |
| Methyl Violet 6B | |
| Basic Violet 3 | 42555 |
| Crystal Violet | |
| Gentian Violet | |
| Iodine Green | 42556 |
| Basic Blue 8 | 42563 |
| Victoria Blue 4R | |
| Acid Blue 13 | 42571 |
| Fast Acid Violet 10B | |
| Acid Blue 75 | 42576 |
| Eriocyanine A | |
| Methyl Green | 42585 |
| Ethyl Green | 42590 |
| Basic Violet 4 | 42600 |
| Ethyl Violet | |
| Acid Violet 49 | 42640 |
| Wool Violet 5BN | |
| Acid Blue 15 | 42645 |
| Brilliant Milling Blue B | |
| Acid Violet 17 | 42650 |
| Acid Violet 6B | |
| Wood Violet 4BN | |
| Formyl Violet | |
| Acid Violet 5BS Conc. | |
| Acid Violet 19 | 42685 |
| Acid Fuchsin | |
| Red Violet 5R | 42690 |
| Acid Blue 22 | 42755 |
| Aniline Blue | |
| Soluble Blue | |
| Solvent Blue 3 | 42775 |
| Solvent Blue 3 | 42780 |
| Methyl Blue | |
| Aurin | 43800 |
| Mordant Blue 3 | 43820 |
| Eriochrome Cyanine R | |
| Acid Green 16 | 44025 |
| Naphthalene Green V | |
| Pontacyl Green NV Extra | |
| Basic Blue 11 | 44040 |
| Victoria Blue R | |
| Basic Blue 15 | 44085 |
| Night Blue | |
| Acid Green 50 | 44090 |
| Wool Green S | |
| Kiton Green S. Conc. | |
| Basic Green 3 | |
| Sevron Green B | |
| Brilliant Blue F & R Extra | |
| Brilliant Green Sulfonate | |
| Hexakis (hydroxyethyl) | |

Pararosaniline

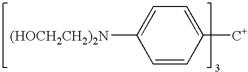

New Green

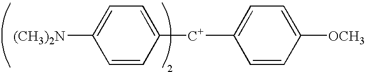

Phenolphthalein

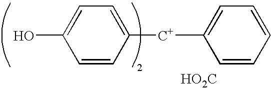

Malachite Green Ethiodide

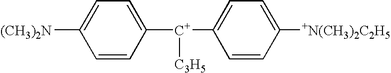

Hydroxyalkylated Pararosanilines

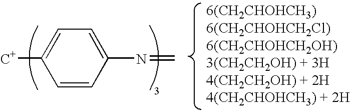

Hydroxyalkylated New Fuchsins

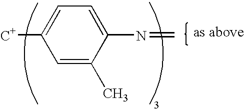

New Yellow

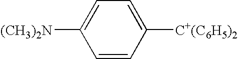

Docbner's Violet

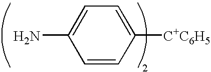

New Red

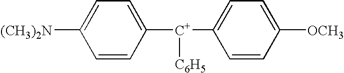

Bis(hydroxyethyl) Doebner's Violet

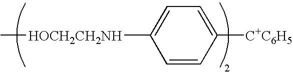

“New Magenta”

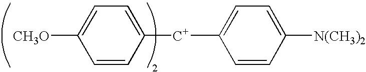

Tetrakis(hydroxyethyl) Doebner's Violet

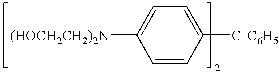

Trichloro Crystal Violet

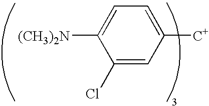

Slow Red

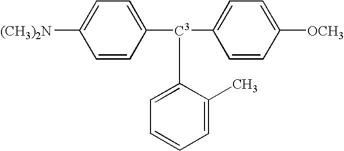

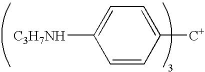

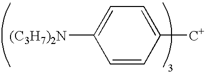

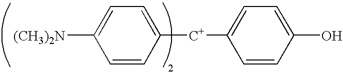

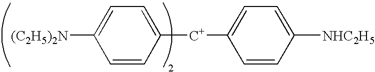

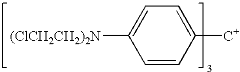

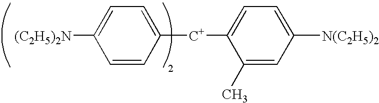

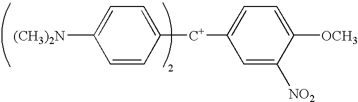

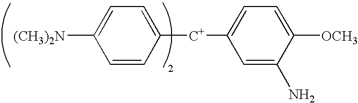

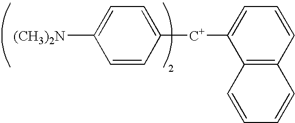

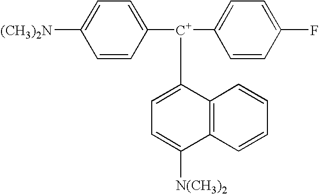

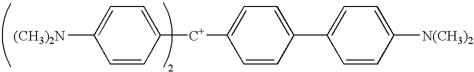

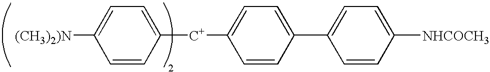

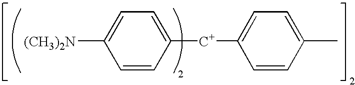

^{a}Only the cyanide, bisulfite, and hydroxide ions are considered, regardless of the other anions present
in the solution.
^{b}More detailed descriptions of the compositions of photochromic materials tested are given in
Macnair's review [255; tables 1A-4].
^{c}Ethanol.
^{d}Diethyl ether.
^{e}1,2-Dichloroethane.
^{f}1,1-Dichloroethane, cyclohexane-1,1-dichloroethane, or cyclohexane-1,2-dichloroethane mixtures.
^{g}Benzene.
^{h}Dimethylsulfoxide, neat and aqueous.
ⁱAcetone.
~^{j}ACETIC ACID.
^{k}Ethyl acetate.
^{l}Ethyl bromide.
^{m}2-Methoxyethanol
ⁿChloroform.
^{o}Ethanol with KCN.
^{p}Ethanol wiih KOH.
^{q}Carboxylic acids-acetic to stearic; hydrocinnamic acid; ethyl and butyl acid phthalates.
^{r}Octadecylnitrile, tributyl phosphate, aniline, 2-(p-tert-butylpheno xy)ethanol, tetraethyleneglycol
dimethyl ether, or poly(ethylene glycols).
^{s}Amides-formamide to stearamide; methylformamide or methylacetamide; dimethyl- or diethyl-
formamide or acetamide.
^{t}Three-to-one solutions of cellulose acetate with any of the following five-to-one plasticizer mixtures:
butyl stearate, Polyethylene Glycol 600-butyl acetoxystearate, butyl stearate, or Dowanol EP-butyl
acetoxystearate.
^{u}Water containing SO₂.
^{v}Water containing bisulfite and papain.
^{w}Poly(vinyl alcohol) with dimethylsulfoxide (5:1).
^{x}Films, containing residual solvent, cast from the following solutions: ethanol-acetone solutions of
vinyl acetate-vinyl alcohol copolymer; aqueous poly(vinyl alcohol); aqueous poly(vinyl pyrrolidone);
or aqueous methyl vinylether-maleic acid copolymer.
^{y}Methanol-dioxane with aqueous NH₄ HSO₃.
^{z}Paper impregnated with a toluene solution of poly(methyl methacrylate), stearic acid, and 2-(p-tert-
butylphenoxy)ethanol, then dried.
^{aa}Intramicellar impregnation of cellulose with the following swelling agents: n-propylamine, n-
butylamine, n-hexylamine, 2-aminoethanol, dimethylformamide, acetic acid, dimethylsulfoxide,
methylacetamide, dimethylacetamide, or formamide.
^{bb}Films cast from an approximately 4:3 mixture of a 20% solution and cellulose acetate butyrate in
toluene-ethyl acetate (1:1) and triallycyanurate in dioxane.
^{cc}Films cast from a 2:1 mixture of a 25% solution of cellulose acetate butyrate in toluene-ethyl acetate
(1:1) and the titanium esters of N,N,N′,N′-tetrakis(2-hydroxypropyl) ethylenediamine.
^{dd}Pure water.
^{ee}Films cast from aqueous gelatin or other hydrocolloids.
^{ff}Dimethylsulfoxide with methanolic KCN.
^{gg}2-Methoxyethanol with methanolic KCN.
^{hh}Water or aqueous methanol containing bisulfite.
ⁱⁱPaper impregnated with m-dimethoxybenzene, acetonitrile, acetic acid, or phenyl methyl carbinol.
^{jj}Ethanol-benzene.
^{kk}Aqueous ethanol, methanol, aqueous methanol, aqueous acetone, benzene-methanol, carbon
tetrachloride-methanol, cyclohexane-methanol, or chloroform-methanol.
^{ll}Films cast from 3:1 solutions of cellulose acetate and either Polyethylene Glycol 600 .RTM. or
ethylene glycol phenyl ether as plasticizer.
^{mm}Films, containing residual solvent, cast from solutions of either cellulose acetate in 2-
methoxyethanol or poly(vinyl alcohol) in aqueous ethanol.
ⁿⁿFilms, containing residual solvent, cast from solutions of either cellulose acetate butyrate in 2-
methoxyethanol or poly(vinyl acetate) in methanol.
^{oo}Ethanol containing ammonia.
^{pp}Aqueous methanol containing NH₄ HSO₃ and urease.
^{qq}Aqueous methanol containing NH₄ HSO₃, with or without sodium dithionite.
^{rr}Aqueous acid at pH 1.
^{ss}Aqueous ammonia containing KCN.
^{tt}Paper impregnated with aqueous solutions with or without hydrocolloids.
^{uu}2-Methoxyethanol containing HCl.
^{vv}Aqueous methanol containing NH₄ HSO₃, and glucose oxidase.
^{ww}9:1 Methanol-water.
^{xx}Aqueous NaOH.

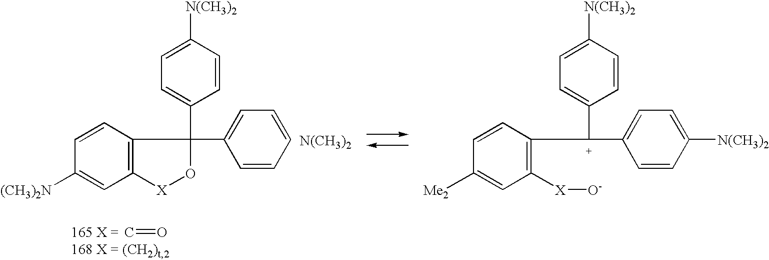

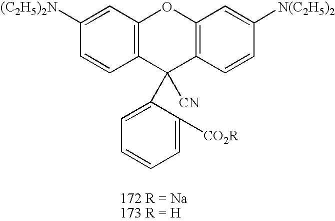

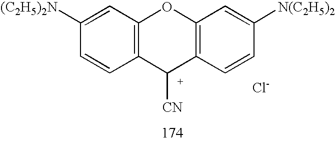

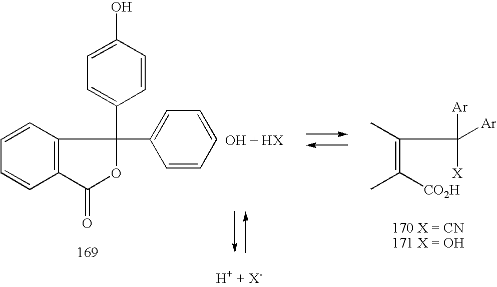

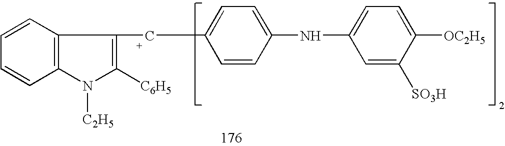

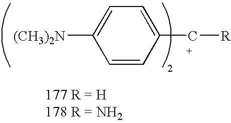

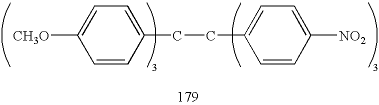

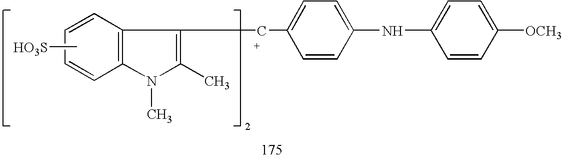

Photochromic Polymethine Dyes

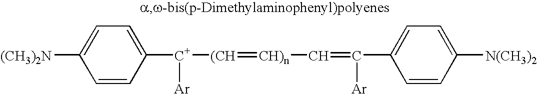

| Ar | n |
|---|---|
| C₆H₅ | 0, 1, 2 |
| 4-(CH₃)₂NC₆H₄ | 0, 1, 2 |
| 4-(CH₃)₂CHC₆H₄ | 0, 1, 2, 3, 4 |
| 4-CH₃OC₆H₄ | 0, 1, 2 |
| 4-C₄H₉OC₆H₄ | 0, 1, 2 |
| 3-CH₃C₆H₄ | 1, 2 |
| 4-t-C₄H₉C₆H₄ | 1, 2 |
| 4-C₂H₅OC₆H₄ | 1, 2 |
| 4-C₅H₁₁C₆H₄ | 1, 2 |
| 4-FC₆H₄ | 1 |
| 4-Fsub₃CC₆H₄ | 1 |
| 2-(C₆H₅)2NC6H4 | 1 |
| 3,4-H₂N(OCH₃)C₆H₃ | 1 |
| 2-Naphthyl | 1, 2 |
| 4-ClC₆H₄ | 2 |
| 2,4-Cl₂C₆H₃ | 2 |
| 1-Naphthyl | 2 |

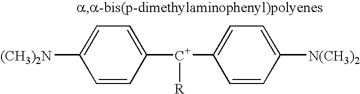

| R | R |
|---|---|
| 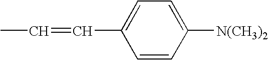 | 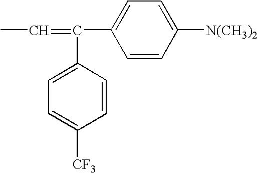 |
| 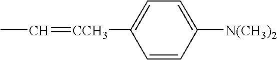 | |
| 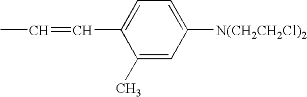 | 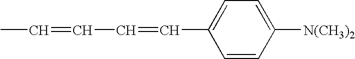 |
| 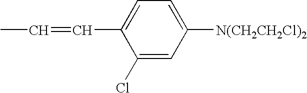 | 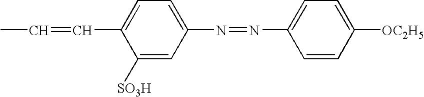 |
| 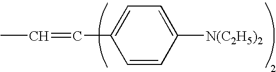 | —CH═N—N(C₆H₅)₂ |
| 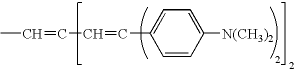 | 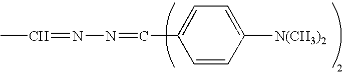 |
| | 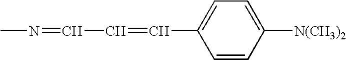 |
| 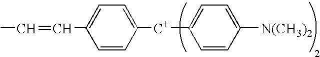 | 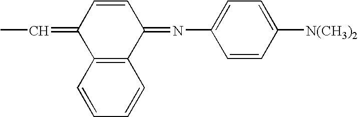 |
| 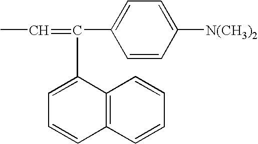 | 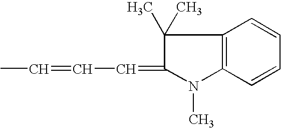 |

Miscellaneous polyenes

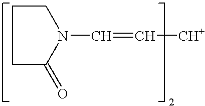

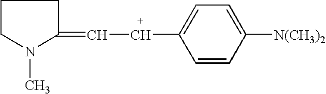

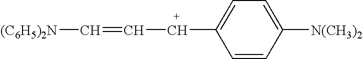

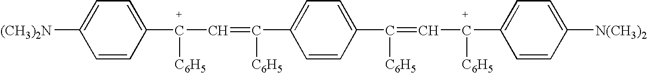

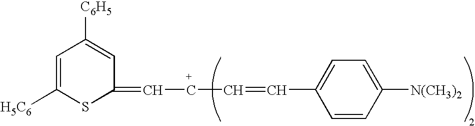

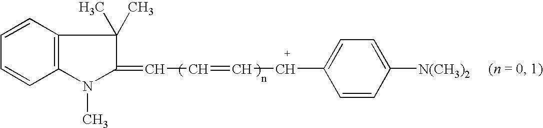

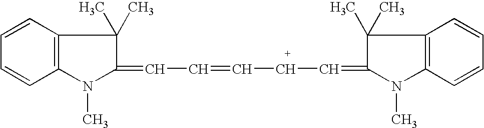

Basic Red 13

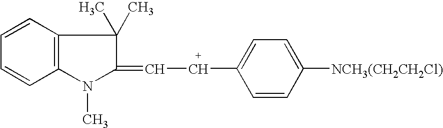

Basic Violet 7

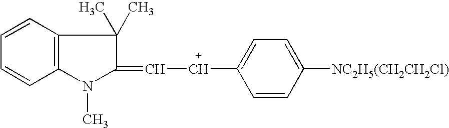

Basic Red 14
Basic Red 15
Basic Violet 15

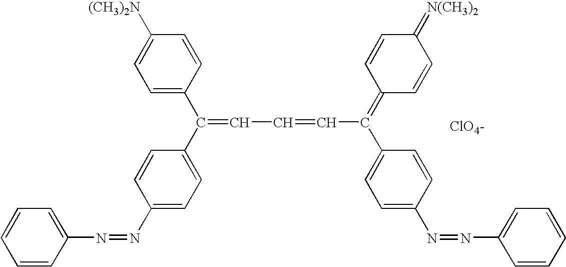

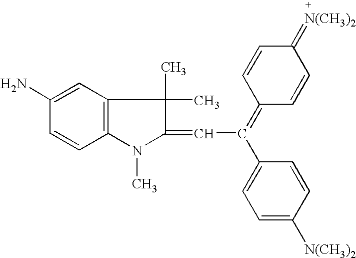

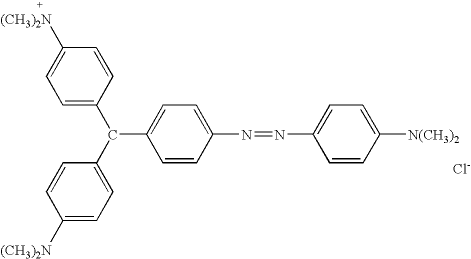

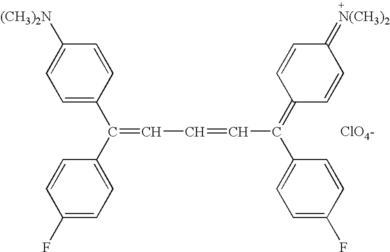

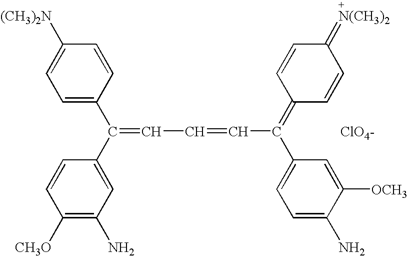

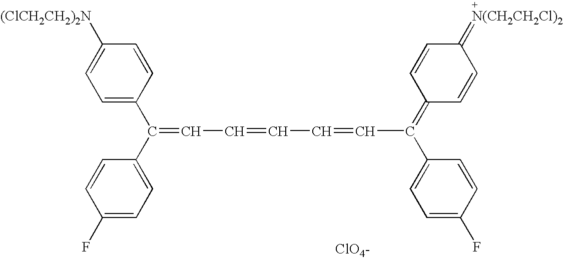

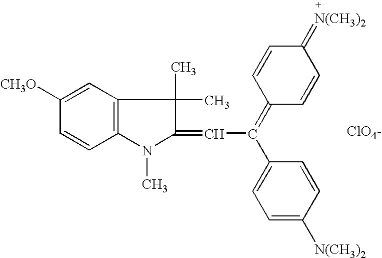

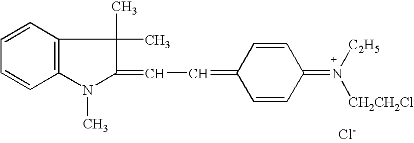

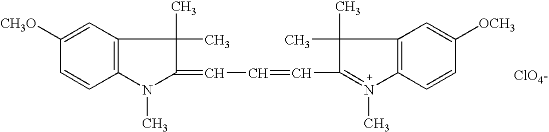

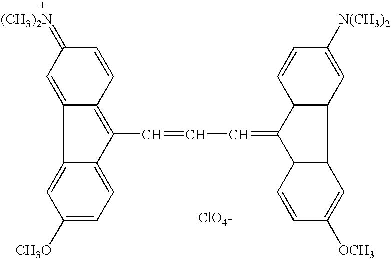

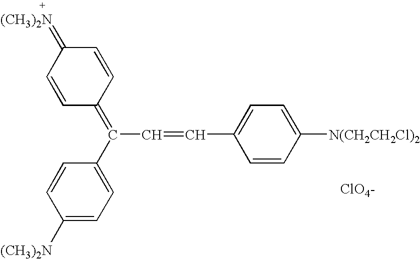

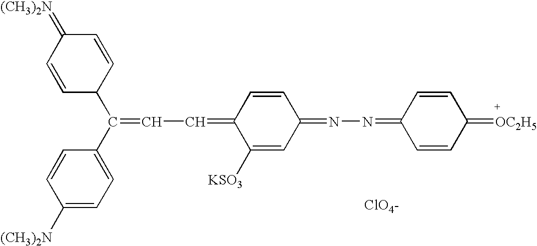

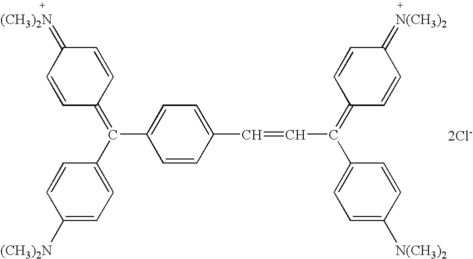

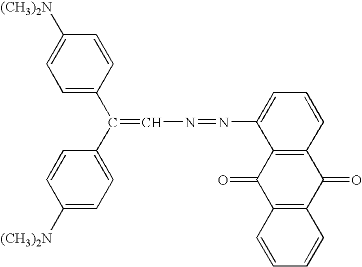

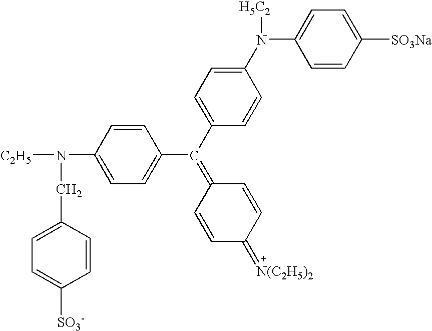

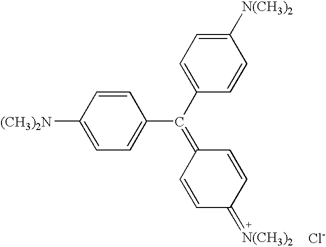

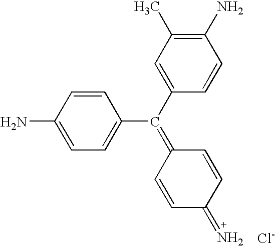

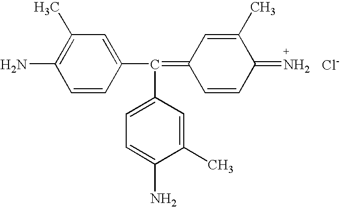

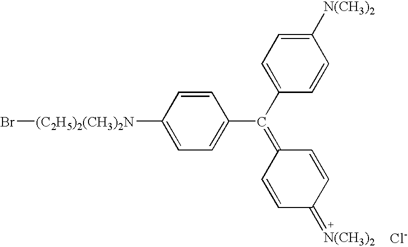

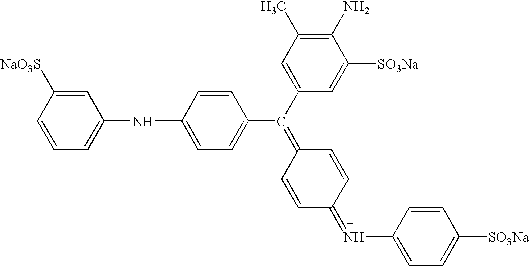

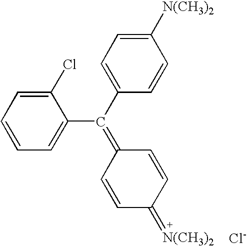

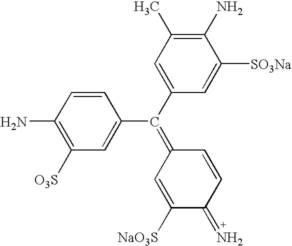

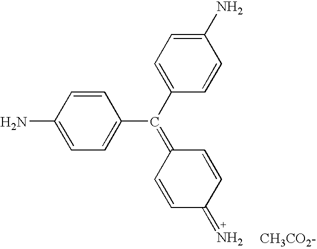

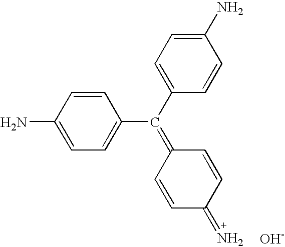

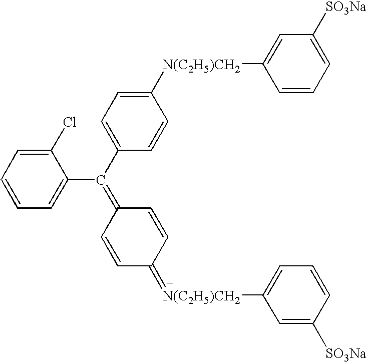

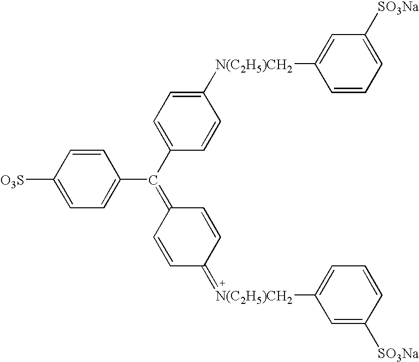

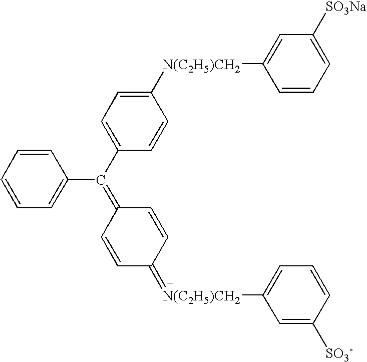

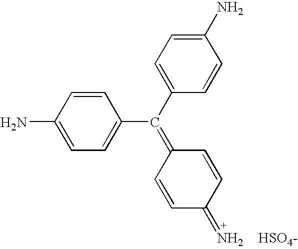

Salt-isomerism type phototropic dyes

| | |
|---|---|
| Night Blue | 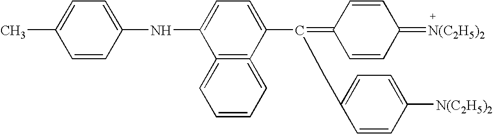 |
| Victoria Blue R | 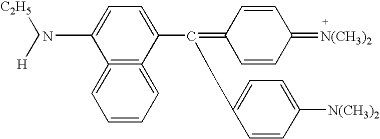 |
| Brilliant Milling Blue B Brilliant Blue F & R Ex. Eriocyanine A | 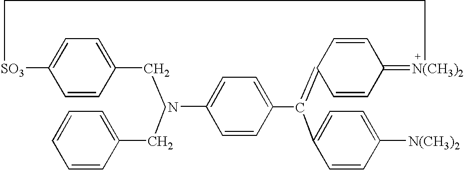 |
| Methyl Blue | 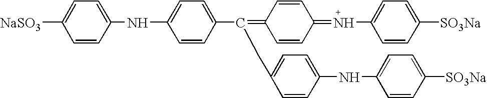 |
| Aniline Blue | 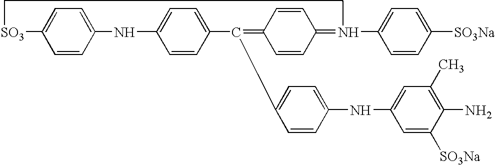 |
| Eriochrome Cyanine R | 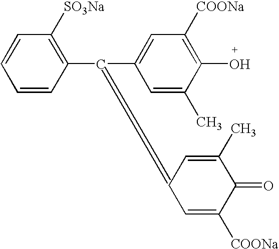 |
| Methyl Violet 6B | 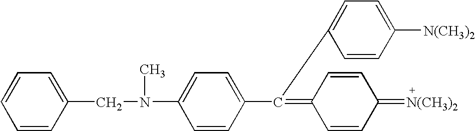 |
| Iodine Green | 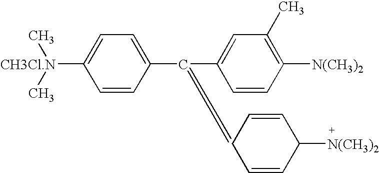 |
| Aniline Blue | 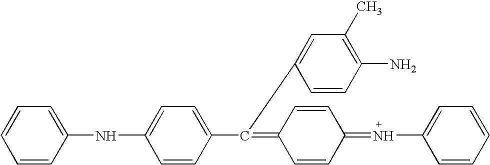 |
| Wool Violet 5 BN | 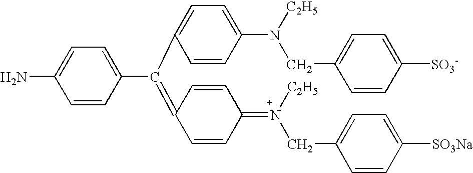 |
| Wool Violet 4 EM | 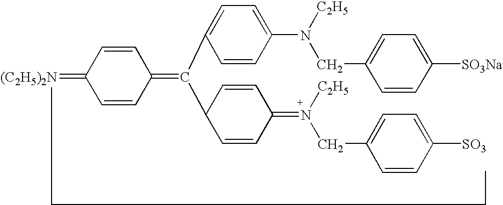 |
| Light Green SF Yellowish | 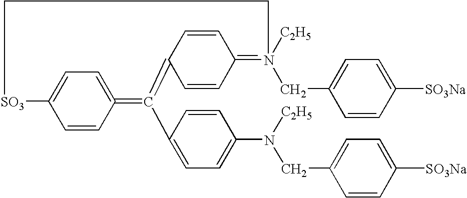 |
| Iodine Violet | 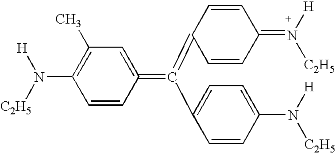 |
| Methyl Violet | 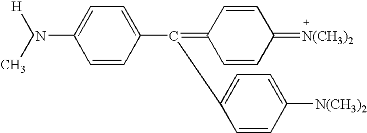 |
| Crystal Violet | 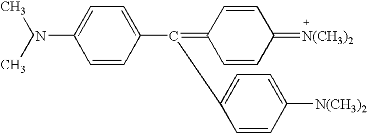 |
| Ethyl Violet | 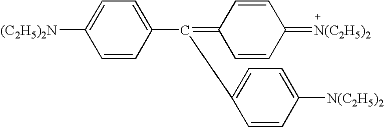 |
| Acid Green L Extra | 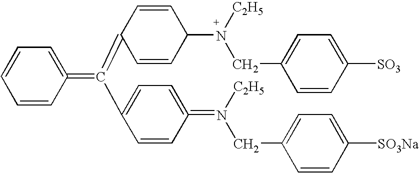 |
| Erioviridene B | 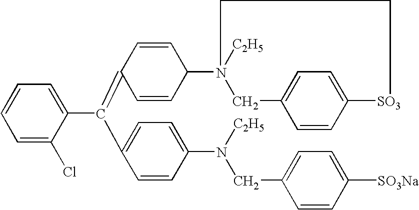 |
| Light Green SF | 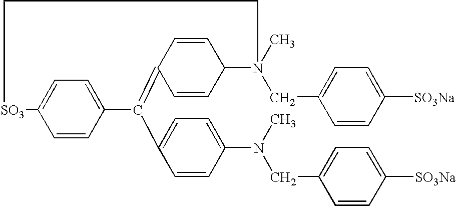 |
| Victoria Green (Malachite Green) | 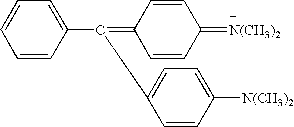 |
| Red-Violet SR | 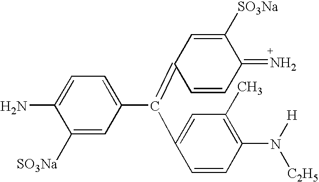 |
| Brilliant Green “B” | 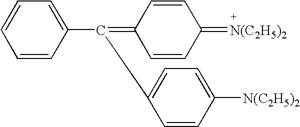 |
| Di-[4(N,N-diethylamime)phenyl]-[4- (N,N-diethyl-amine-2- methyl) phenyl] methyl carbonium | 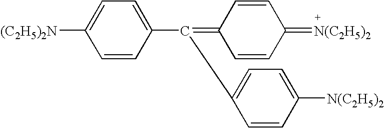 |
| Tri-[4(N,N-dipropylamino)phenyl]methyl carbonium | 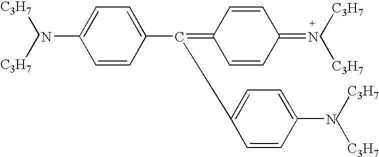 |
| Di-[4(N,N-diethylamino)phenyl]- [4(ethylamino)- phenyl] methyl carbonium | 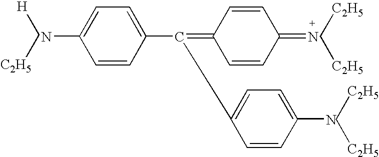 |
| Di-[4(N,N-diethylamino)phenyl]- [4(N,N-diethyl- amino)naphthyl]methyl carbonium | 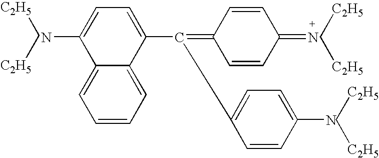 |
| Di-[4(N,N-dimethylamino)phenyl9 - [4(hydroxy)phenyl]methyl carbonium | 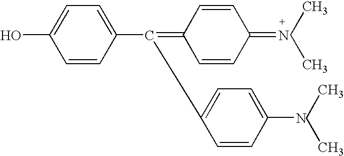 |
| Tri-[4(N-propylamina)phenyl]methyl carbonium | 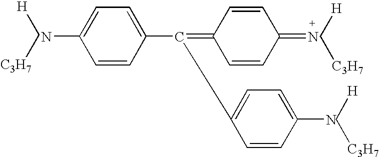 |
| Hectalene Blue DS-1398 Hectolene Blue DS-1823 Sevron Brilliant Red 4G Di-[4(N,N-dimethylamino)phenyl]- [4(hydroxy)phenyl]methyl carbonium | 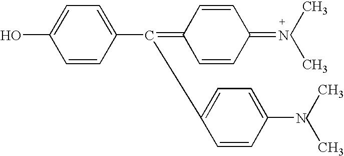 |
| Tri-[4(N-propylamino)phenyl]methyl carbonium | 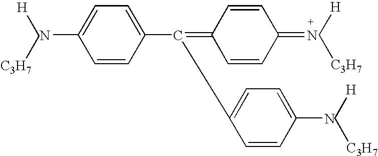 |
| Hectolene Blue DS-1398 Hectolene Blue DS-1823 Sevron Brilliant Red 4G Genacryl Red 6B Genacryl Pink G Sevrun Brilliant - Red B Sevron Brilliant - Red 3B 1,5-bis-[4(N,N-dimethylamiao)phenyl]- 1,5-bis-(phenyl)divinyl carbonium trifluoroacetate | 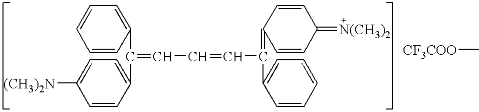 |
| 1,1,3,3-tetrakis[4(N,N- dimethylamino)phenyl]vinyl carbonium perchlorate | 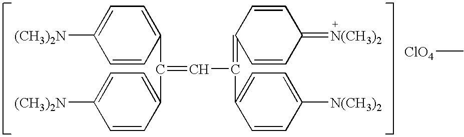 |
| 1,5-bis-[4(N,N-dimethylamino)phenyl]- 1,5-bis-(phenyl) divinyl carbonium p-toluenesulfonate | 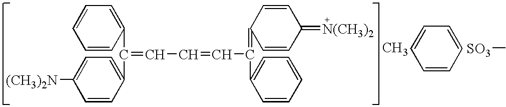 |
| 1,7-bis[4(N,N-dimethylamino)phenyl]- 1,7-bis-(2,4- dichlorophenyl) trivinyl carbonium perchlorate | 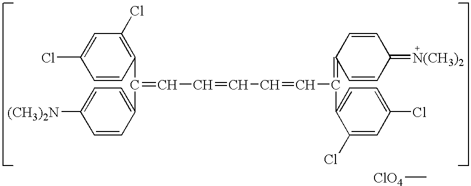 |
| Di-[4(N,N-dimethylamino)phenyl vinyl]-[2,4-di-phenyl-6- methane thiopyran]methyl carbonium perchlorate | 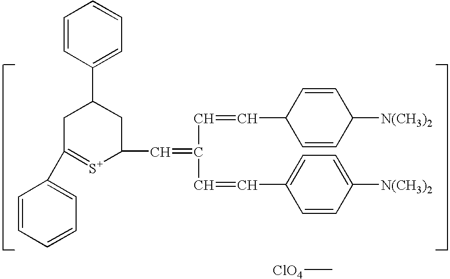 |
| 1,7-bis-[4(N,N-dimethylamino)phenyl]- 1,7-bis-(4-chlorophenyl) trivinyl carbonium trifluoroacetate | 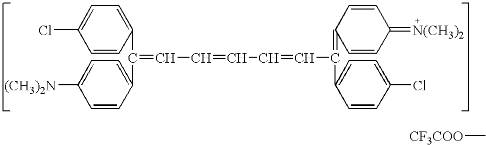 |
| 1,1,3-tris-[4-(N,N-dimethylamino) phenyl]]divinyl carbonium perchlorate | 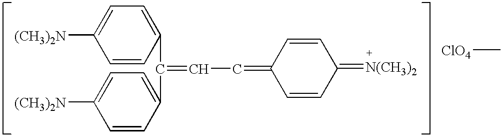 |
| 1,1,7,7-tetrakis-[4-(N,N- dimethylamino)phenyl]trivinyl carbonium perchlorate | 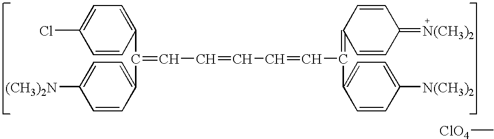 |
| 1,3-bis-[4-(N,N-dimethylamino) phenyl]-1,3-bis- (phenyl) vinyl carbonium perchlorate | 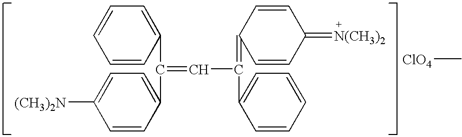 |
| 1,1,5,5-tetrakis-[4-(N,N- dimethylamino)phenyl]divinyl carbonium perchlorate | 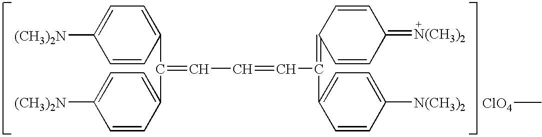 |
| 1,5-bis-[4-(N,N-dimethylamino) phenyl]-1,5-bis-(phenyl) divinyl carbonium perchlorate | 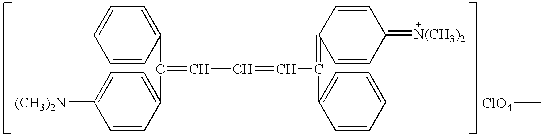 |
| 1,7-bis-[4-(N,N-dimethylamino) phenyl]-1,7-bis-(phenyl) trivinyl carbonium trifluoroacetate | 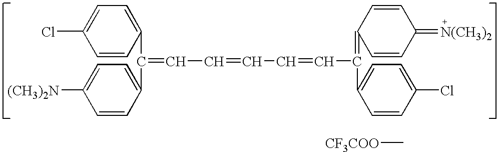 |
| 1(1,3,3-trimethyl indoline)-2-[4- (N,N-dimethyl-amino) phenyl] ethylene carbonium perchlorate | 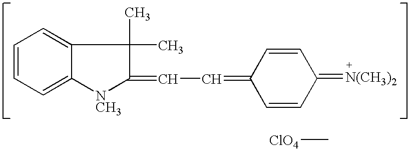 |
| 1(1,3,3-trimethyl indoline)-4-[4- (N,N-dimethyl-amino) phenyl] butylene carbonium perchlorate | 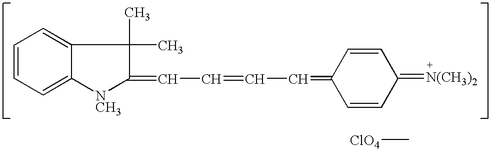 |
| 1,1,3,3-tetrakis-[4(N,N- diethylamino)phenyl]vinyl carbonium perchlorate | 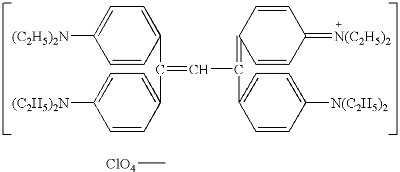 |
| 1,1-bis-[4-(N,N-diethylamino) phenyl]-3,3-bis- [4-(N,N-dimethylamino)phenyl]vinyl carbonium perchlorate | 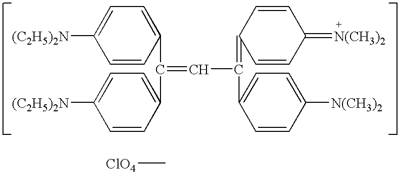 |
| 1,1,5,5-tetrakis-[4-(N,N- diethylamino)phenyl]divinyl carbomum perchlorate | 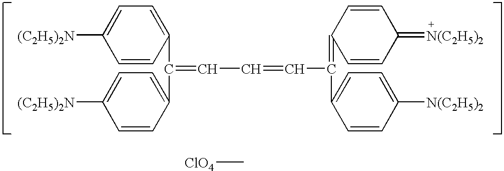 |
| 1,1-bis-[4-(N,N- dimethylamino)phenyl]-3- [4-(amino)phenyl]-3- methylvinyl carbonium perchlorate | 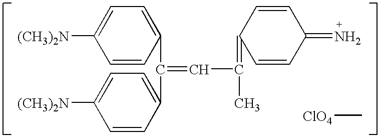 |
| Tris-[1,1-bis-[4(N,N- dimethylamino)phenyl]ethylene] methyl carbonium perchlorate | 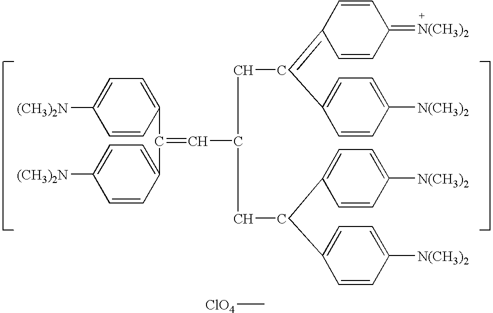 |
| Tris-[1,1-bis-[4-(N,N- diethylamino)phenyl]ethylene] methyl carbonium perchlorate | 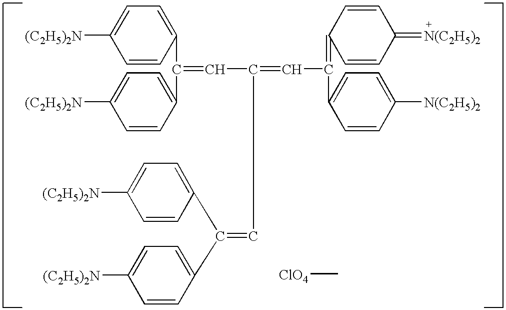 |
| 1,1,5-Tris-[4-(N,N- dimethylamino)phenyl]divinyl carbonium perchlorate | 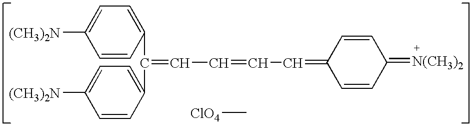 |
| N[4-(N,N-dimethylamino) cinnaniylidene] auramine | 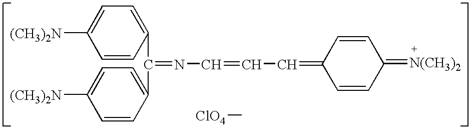 |
| 1,1-bis-[4-(N,N- dimethylamino)phenyl-3,4-bis- (phenyl)]-3,4-diazo butene carbanium | 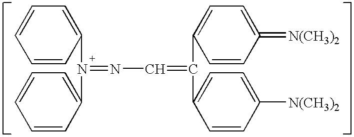 |
| 1,1,5,5-tetrakis-[4-(N,N- dimethylamino)phenyl]- 2,3-diazo pentene carbonium | 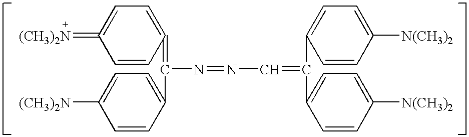 |
| N-(N′,N′-dimethylamino cinnamylidene)-N,N- diphenyl ammonium | 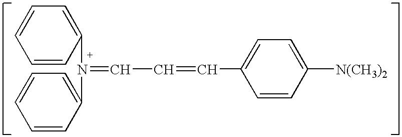 |
| Azo Polymethines Dyes of the general structural type | 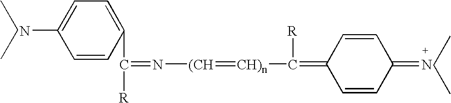 |

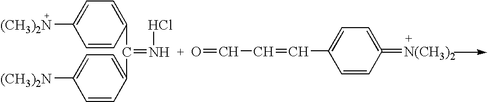

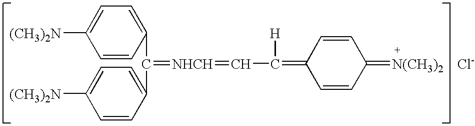

| | |
|---|---|
| Photochronic diazopolymethines | 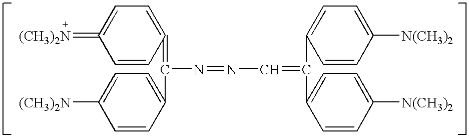 |
| 1,1,5,5-tetrakis-[4-(N,Np- dimethylamino)phenyl]- 2,3-diazo pentene carbonium | 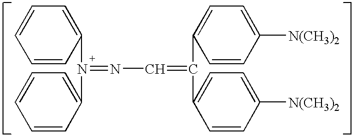 |
| 1,1-bis-[4-(N,N- | |
| dimethylamino)phenyl-3,4-bis- | |
| (phenyl)]-3,4-diazo | |
| butene carbonium | |

The drug functionality, C, includes any molecule which exhibits bleaching behavior with the B functionality and has an increased therapeutic effect or therapeutic ratio as a consequence of its delivery as part of a Luminide agent. For example, Foscarnet, a viral reverse transcriptase inhibitor possesses both a carboxylate and phosphate group which will bleach photochromic compounds; 4-bromocrotonyl-CoA, an acetoacetyl-CoA thiolase inhibitor, possesses a thiol group which will bleach photochromic compounds; L-3-iodo-α-methyltyrosine, a tyrosine hydroxylase inhibitor, possesses a carboxylate group which will bleach photochromic compounds, and Captopril, an antihypertensive pharmaceutical, possesses both a sulfide and carboxylate group which will bleach photochromic compounds. Furthermore, the pharmacokinetics and/or pharmacodynamics of these agents are altered via delivery to the site of action by way of a Luminide agent such that the therapeutic effect or therapeutic ratio is enhanced.

Other drugs which are not inherently photochromic bleaches in that they lack a nucleophilic group which will form a reversible covalent bond with the B functionality can be derivatized with a known bleaching nucleophilic group such as cinnamate, sulfite, phosphate, carboxylate, thiol, or amine group to transform them into bleaching agents of the B functionality such as a cationic dye. See TABLE 3 below for the structure of a exemplary drug molecules.

TABLE 3
Representative Drug Molecules

| Name | Structure |
|---|---|
| Captopril | 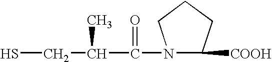 |
| Prostaglandin E₂ | 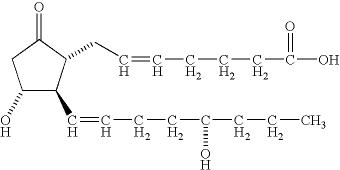 |
| 2,3-dichloro-α-methyl- benzylamine | 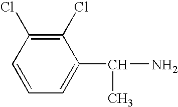 |
| 3′-deoxy-S-adenosyl-L- homocysteine | 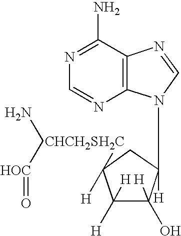 |
| Sinefungin | 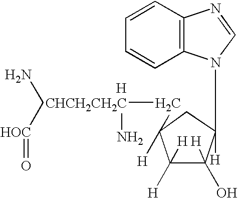 |
| 3,5-diiodo-4-hydroxy- benzoic acid | 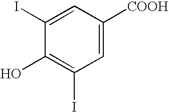 |
| 6,6′-dithiobis (9- B-D-ribofuranosylpurine) | 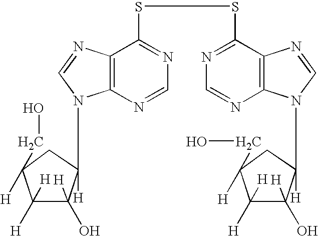 |
| γ-aminobutyric acid | H₂NCH₂CH₂CH₂COOH |
| Gabaculine | 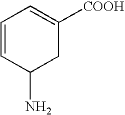 |
| N-(5′-phosphopyridoxy)- 4-aminobutyric acid | 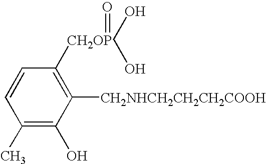 |
| 4-amino-hex-5-enoic acid | 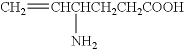 |
| Baclofen | 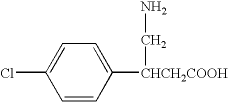 |
| Adenosine | 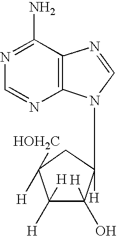 |
| 3-hydroxy-3-methyl- glutarate | 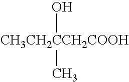 |
| Campactin | 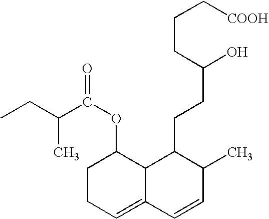 |
| But-3-ynoyl-CoA | 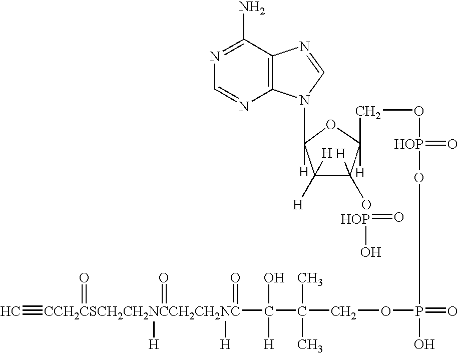 |
| Suramin | 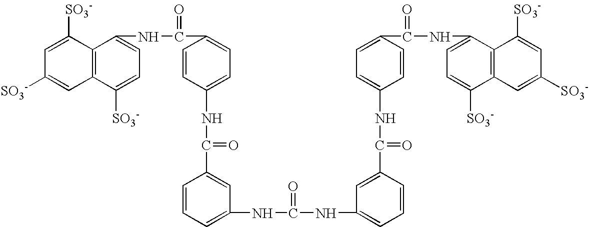 |
| L-3-iodotyrosine | 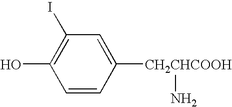 |
| L-3-iodo-α-methyltyrosine | 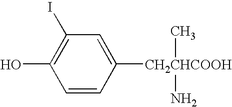 |
| Disodium cromoglycate | 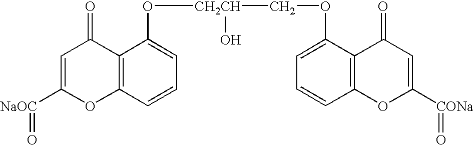 |
| Adenosine 3′,5′-cyclic monophosphate | 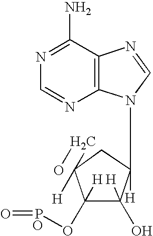 |
| D,L-B-(5-hydroxy-3- indolyl)-α-hydra- zinopropionic acid | 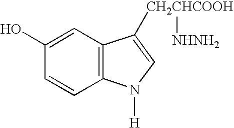 |
| D,L-α-hydrazino-α- methyldopa | 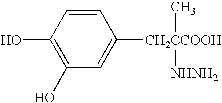 |
| α-methyldopa | 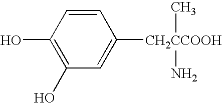 |
| 5-(3,4-dihydroxycinna- moyl)salicylic acid | 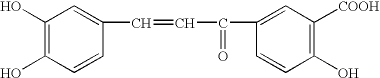 |
| N-(phosphonacetyl)- L-aspartate | 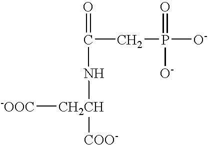 |
| P-glycolohydroxamate | 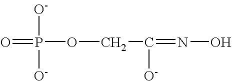 |
| 5-(p-sulfamylphenyl- azosalicylic acid | 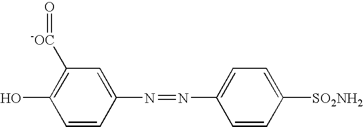 |
| Coformycin | 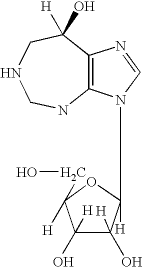 |
| Formycin B | 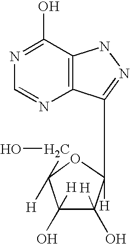 |
| Thioinosinate | 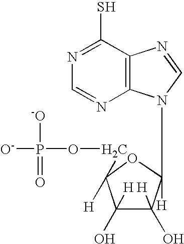 |
| Phosphonoformate | 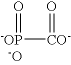 |
| Phosphonoacetate | 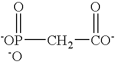 |
| Ridavirin | 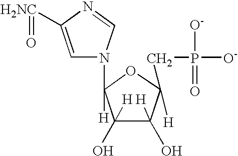 |
| Sotalol | 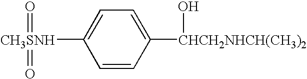 |
| Cimetidine | 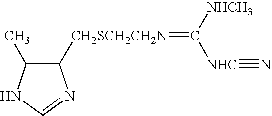 |
| Fuscaric acid | 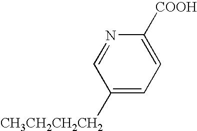 |
| 2-mercaptoethylamine | HSCH₂CH₂NH₃⁺ |
| Mimosine | 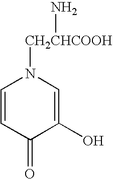 |
| U-7130 | 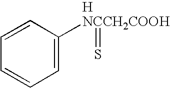 |
| Iproniazid | 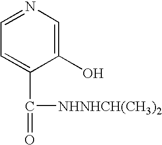 |
| Trans-4-aminoocrotonic | H₂NCH₂CH═CHCOOH |
| acid | |
| NSD 1055 | 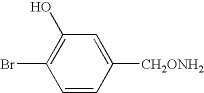 |
| Nicotinic acid | 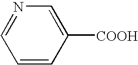 |
| Kynurenic acid | 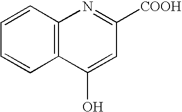 |
| Lentysine | 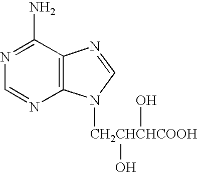 |
| Orotic acid | 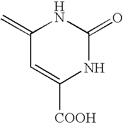 |
| Polyoxin D | 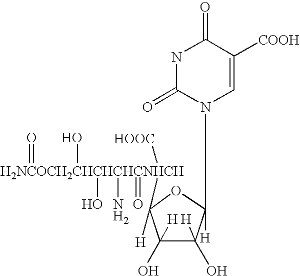 |
| Cephalosporin | 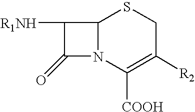 |
| Penicillin | 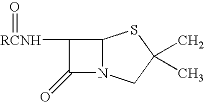 |

The electron transfer functionality, D, includes molecules which undergo a redox reaction which transfers electrons between the electron carriers and the A functionality where a redox reaction of A results in its activation to an excited energy state. The D functionality can be a natural electron carrier such as ubiquinone or a synthetic electron carrier such as methylene blue, phenazine methosulfate, or 2,6-dichlorophenolindophenol. Structures of electron transfer molecules appear below in TABLE 4.

TABLE 4
Representative Electron Transfer Molecules

| Name | Structure |
|---|---|
| Methylene Blue | 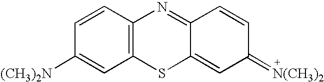 |
| Ubiquinone | 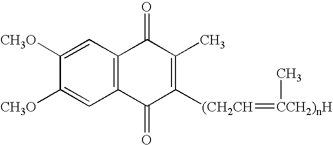 |
| 2,6-dichlorophenolindophenol | 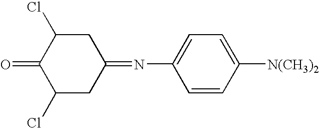 |
| Phenazine methosulfate | 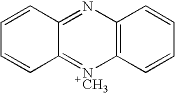 |
| Ferricyanide | 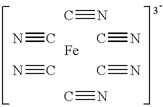 |

### A Representative Luminide

A representative Luminide is the product of the covalent linkage of the polymethine dye with a bleaching drug such as Foscarnet and with a chemiluminescent reactive molecule such as luminol. This conjugate represents a molecule which releases Foscarnet in the presence of oxygen free radicals. The energy of the reaction of luminol with oxygen radicals undergoes intramolecular electronic energy transfer by radiative and nonradiative mechanisms. The latter dominate and include coulombic interactions, dipole-dipole resonance, and exchange interaction. These processes increase the quantum yield for drug release above that which would be produced by luminescence transfer alone. For example, Forster, in a quantum mechanical treatment of resonance transfer, in the region of spectral overlap involving allowed transitions of two well separated molecules has only considered dipole-dipole interactions in deriving an experimentally verified formula which predicts a distance of 5-10 nm as the distance at which transfer and spontaneous decay of the excited donor are equally probable. The formula predicts the transfer probability is inversely proportional to the separation distance raised to the sixth power. However, the donor and acceptor functionalities of a Luminide are covalently linked; thus, since the separation distance is of the order of angstroms, the transfer probability is great. In fact, the efficiency of transfer has been studied in certain molecules which consist of two independent chromophores separated by one or more saturated bonds. In such cases, energy transfer over large distances has been observed to be in agreement with predictions from Forster's Theory.

### Exemplary Luminide Pharmaceuticals

Prostaglandins possess potent renal, cardiac, hemodynamic, and other physiological effects; however, the free agents are 95% inactivated during one passage through the pulmonary circulation and are essentially eliminated in 90 seconds from intravascular injection. A luminide which is resistant to intravascular inactivation comprising a C functionality of prostaglandin A₁, A₂, B₁, E₁, E₂ or an analogue which possesses a vasodilatory effect on coronary arteries and other human vascular beds is an agent for the treatment of ischemic heart disease and is a antihypertensive agent with a long halflife. A luminide which is resistant to intravascular inactivation comprising a C functionality of postaglandin E, F, A or an analogue which possesses a positive cardiac inotropic effect is an inotropic agent with a long halflife. A luminide which is resistant to intravascular inactivation comprising a C functionality of prostaglandin A, E, or an analogue prostaglandin which possesses natriuretic and diuretic activity is a diuretic agent with a long halflife. A luminide which is resistant to intravascular inactivation comprising a C functionality of prostaglandin A, G, E₁, E₂ or an analogue such as 15(S)-15-methyl PGE 2 methylester, 16,16-dimethyl PGE₂, AY-22,093, AY-22,469, AY-22,443, or 15(R)-15-methyl PGE₂ which inhibits gastric acid secretion is an agent for the treatment of peptic and duodenal ulcer disease with a long halflife. A luminide which is resistant to intravascular inactivation comprising a C functionality of prostaglandin D₂, E₁ or an analogue which inhibits platelet aggregation is an antithromboembolic agent with a long halflife. A luminide which is resistant to intravascular inactivation comprising a C functionality of prostaglandin E₁, E₂ or an analogue which causes bronchial dilatation is an agent for the treatment of asthma and allergic and hypersentivity reactions with a long halflife. A luminide which is resistant to intravascular inactivation comprising a C functionality of prostaglandin F2 or an analogue which causes abortion by luteolysis is an agent for therapeutic abortion with a long halflife. A luminide which is resistant to intravascular inactivation comprising a C functionality of prostaglandin A₂, E₁, E₂, or an analogue which induces erythropoiesis by stimulating the release of erythropoietin from the renal cortex is an agent for the treatment of anemia. A luminide which is resistant to intravascular inactivation comprising a C functionality of prostaglandin E or an analogue which modulates T lymphocytes to decrease their ability to reject an allogenic graft is an agent to prolong allograft survival.

A cellular permeant luminide comprising a C functionality of cellular impermeant 2′-isopropyl-4′-(trimethylammonium chloride)-5′-methylphenyl piperidine-1-carboxylate (Amo 1618) which inhibits the cyclization of trans-geranyl-geranyl-PP to copalyl-PP during Kaurene synthesis is a fungicidal agent.

A cellular permeant luminide comprising a C functionality of cellular impermeant adenosine cyclic 3′,5′-monophosphate or an analogue which inhibits the release and formation of phlogistic mediators such as histamine and kinins is an agent for treating asthma and hypersensitivity and anaphylactic reactions.

A cellular permeant luminide comprising a C functionality of cellular impermeant 4′-sulfamylphenyl-2-azo-7-acetamid-1-hydroxynaphthalene-3,6-disulfonate (Neoprontosil), 4′-sulfamyl-2,4-diaminoazobenzene (Prontosil), or 5-(p-sulfamylphenylazo) salicylic acid (Lutazol) which possess potent carbonic acid anhydrase inhibition is a diuretic agent.

A cellular permeant luminide comprising a C functionality of a cellular impermeant analogue of S-adenosyl homocysteine or sinefungin is an oncostatic agent.

A cellular permeant luminide comprising a C functionality of the cellular impermeant phosphoglycolohydroxamate which inhibits Class II aldolases present in bacterial and fungi and is noninhibitory of Class I aldolases present in animals is an antibacterial and antifungal agent.

A cellular permeant luminide comprising a C functionality of a cellular impermeant inosine analogue such as formycin B which inhibits nucleotide phosphorylase during nucleotide metabolism is an agent for disorders of purine metabolism such as gout, is an agent that alters the toxicity and/or antitumor behavior of other analogue-containing nucleosides such as 6-thioguanosine or 6-mercaptopurine ribonueleoside, and is an immunosuppressive agent by disruption of purine metabolism.

A cellular permeant luminide comprising a C functionality of cellular impermeant phosphonoformate (Foscarnet) which inhibits the HIV reverse transcriptase enzyme is an agent for the treatment of acquired immunodeficiency syndrome. The synthesis and the results of treatment of C3H mice infected with Raucher Spleen Focus Forming Virus with MTL J-1, a cellular permeant luminide comprising a C functionality of phosphonoformate, is given in Experimental Secions 1 and 3, respectively.

A cellular and blood-brain barrier permeant luminide comprising a C functionality of cellular and blood brain-barrier impermeant γ-amino-butyric acid (GABA) which is the major inhibitory neurotransmitter in the mannalian central nervous system or comprising a C functionality of a cellular and blood-brain barrier impermeant inhibitor of the GABA-degrading enzyme, GABA: 2-oxoglutarate aminotransferase such as gabaculine, N-(5′-phosphopyridoxyl)-4-aminobutyric acid, ethanolamine-o-sulfate, γ-vinyl GABA, or γ-acetylenic GABA; or comprising a C functionality of a cellular and blood-brain barrier impermeant compound which enhances GABA release such as Baclofen is an anti-convulsant, muscle relaxant, sedative, and anxiolytic agent.

A cellular permeant luminide comprising a C functionality of a cellular impermeant oligonucleotide which binds to RNA or DNA and blocks transcription or translation of HIV or P-glycoprotein gene products is an agent for the treatment of AIDs and chemotherapeutic drug, resistance, respectively.

A blood-brain barrier permeant luminide comprising a C functionality of blood-brain barrier impermeant adenosine which binds to brain purinergic receptors to suppress opiate withdrawal is an agent for the management of opiate withdrawal syndrome.

A slowly releasing peripherally acting luminide comprising a C functionality of adenosine which causes coronary vasodilatation is a long acting agent for the treatment of ischemic heart disease.

A cellular permeant luminide comprising a C functionality of cellular impermeant 3-hydroxy-3-methylglutarate, 3-hydroxybutyrate, 3-hydroxy-3-methylpentanoate, 4-bromocrotonyl-CoA, but-3-ynoyl-CoA, pent-3-ynoyl-CoA, dec-3-ynoyl-CoA, ML-236A, ML-236B (compactin), ML-236C, mevinolin, mevinolinic acid, or a mevalonic acid analogue which is an inhibitor of 3-hydroxy-3-methylglutaryl-CoA reductase which catalyzes the rate-limiting and irreversible step of cholesterol synthesis where inhibition at this step does not lead to the accumulation of nonmetabolizable precursors is an anticholesterol agent.

A cellular permeant luminide comprising a C functionality of cellular impermeant thioinosinate which suppresses T lymphocytes is an immunosuppressant agent.

A cellular permeant luminde comprising a C functionality of cellular impermeant Suramin, which is a powerful inhibitor of energy driven calcium uptake by the sarcoplasmic reticulum and is an intracellular inhibitor of Na⁺-K⁺ ATPase where both activities increase intracellular calcium concentrations with a concomitant inotropic effect is a cardiac inotropic agent.

A cellular permeant luminide comprising a C functionality of a cellular impermeant norepinephrine N-methyltransferase inhibitor such as 2,3-dichloro-α-methylbenzylamine, 2,3-dichlorobenzylamine, 2,3-dichlorobenzamidine, or 3,4-dichlorophenylacetamidine is a specific epinephrine action blocking agent.

A cellular permeant luminide comprising a C functionality of cellular impermeant adenosine cyclic 3′,5′-monophosphate or a cAMP analogue which blocks the synthesis of fatty acids and cholesterol in the liver is an antilipidemic agent.

A cellular permeant luminide comprising a C functionality of a cellular impermeant inhibitor of dihydroxyphenylalanine decarboxylase during the synthesis of epinephrine and norepinephrine such as psitectorigenin, genistein, 3′,4′,5,7-tetrahydroxy-8-methylisoflavone, orbol, 8-hydroxygenistein, 3′,5,7-trihydroxy-4′,6-dimethylisoflavone, 3′,5,7-trihydroxy-4′,8-dimethoxyisoflavone, D,L-β-(5-hydroxy-3-indolyl)-α-hydrazinopropionic acid, D,L-α-hydrazino-α-methyldopa, D,L-β-(3-indolyl)-α-hydrazinopropionic acid, a derivative of phenylalanine such as N-methyl-3,4-dopa, α-acetamido-3,4-dimethyoxycinnamic acid, D,L-α-methyl-3,4-dopa, α-methyl-β-(3-hydroxy-4-methoxyphenyl)alanine, α-methyl-3,4-dimethoxyphenylalanine, or d-catechin; D,L-β-(3-indolyl)-α-methyl-α-hydrazinopropionic acid (R)-3-[3,4-dihydroxyphenyl]-1-fluoropropylamine, (S)-α-fluoromethyldopa, (S)-α-fluoromethyltyrosine, 5-(3,4-dihydroxycinnamoyl)salicylic acid, 3-hydroxycinnamic acid, caffeic acid, 3-mercaptocinnamic acid, α-methyl-3-hydroxycinnamic acid, α-ethyl-3-hydroxycinnamic acid, 3-hydroxy-w-nitrostyrene, 3,4-dihydroxyhydrocinnamic acid, 3-hydroxybenzalacetone, 3-hydroxychalone, 3-hydroxybenzal furanyl ketone, 3-hydroxybenzal thiophenyl ketone, 3′,4′-dihydroxyflavone, 8-O-glucoseflavone, flavone, 3-hydroxyphenyl pyruvic acid, 3,4-dihydroxyphenylpyruvic acid phenylthiopyruvic acid, 4-hydroxyphenylpyruvic acid, dithiosalicyclic acid, 1-hydroxy-2-naphthoic acid, 3-hydroxy-7-sulfo-2-naphtholic acid, 3,5-dihydroxy-2-naphtholic acid, 4-chlorocinnamic acid, 2-chlorocinnamic acid, 2,4-dichlorocinnamic acid, 3-nitrocinnamic acid, 3,5-dibromo-2-hydroxycinnamic acid, 2,4,6-triiodo-3-hydroxycinnamic acid, 2-hydroxy-4′-cyanochalone, 4-(4-hydroxycinnamoyl)benzylnitrile, 2-(4-hydroxycinnamoyl)-1,4-dihydroxybenzene, quercetin-6′-sulfonic acid, 5-(2-hydroxy-3,5-dibromocinnamoyl) salicylic acid or 5-(3-hydroxycinnamoyl) salicylic acid is an antihypertensive agent.

A sperm permeant luminide comprising a C functionality of sperm impermeant, inhibitors of acrosin, a proteolytic enzyme located in the acrosome of sperm, such as tosyl lysine chloromethyl ketone, N-α-tosyl-L-arginine chloromethyl ketone, or ethyl p-guanidinobenzoate is a contraceptive agent.

A cellular permeant luminide comprising a C functionality of cellular impermeant adenosine cyclic 3′,5′-monophosphate (cAMP), N⁶,O²-dibutyryladenosine cyclic 3′,5′-monophosphate or an analogue which produces an inotropic response is a cardiac inotropic agent.

A cellular permeant luminide comprising a C functionality of a cellular impermeant adenosine kinase enzyme inhibitor such as 6,6′-dithiobis (9-β-D-ribofuranosylpurine) is a chemotherapeutic agent and an immunosuppressive agent.

A mitochondrial and blood-brain barrier permeant luminide comprising a C functionality of a mitochondrial and blood-brain barrier impermeant inhibitor of monoamine oxidase such as phenylhydrazine, phenylethylidenehydrazine, isopropylhydrazine, or iproniazid is an antidepressant.

A cellular and blood-brain barrier permeant luminide comprising a C functionality of a cellular and blood-brain barrier impermeant inhibitor of catechol-o-methyltrasferase such as 3,5-diiodo-4-hydroxybenzoic acid, S-3′-deoxyadenosylL-homocysteine, pyrogallol, R04-4602, gallic acid, 3,5-dihydroxy-4-methylbenzoic acid, 1,3-dihydroxy-2-methoxybenzene, 1-hydroxy-2,3-dimethoxybenzene, 2-hydroxy-1,3-dimethoxybenzene, 1,3-dihydroxy-4-methoxybenzene, catechol, 3,4-dihydroxybenzoic acid, caffeic acid, 5,6-dihydroxyindole, noradnamine, dopacetamide, H22/54, quercetin, nordihydroguaiaretic acid, U-0521, arterenone, methylspinazarin, MK 486, dopa, papaveroline, isoprenaline, 7,8-dihydroxy-chlorpromazine, 3-hydroxy-4-pyridone, tetrahydroi-soquinoline pyridoxal 5′-phosphate, iodoacetic acid, 3-mercaptotyramine, dehydrodicaffeic acid dilactone, methylspinazorin, 3′,5,7-trihydroxy-4′,6-dimeth-oxyisoflavone, 3′,5,7-trihydroxy-4′,8-dimeth-oxyisoflavone, 6,7-dihydromethylspinazarin, S-adenosylhomocysteine, S-tubercidinylhomocysteine, 3′,8-dihydroxy-4′,6,7-trimethoxy-isoflavone, 7-O-methylspi nochrome B, 6-(3-hydroxybutyl)-7-O-methylspinachrome B, 3,5-diiodosalicyclic acid, or pyridoxal-5′-phosphate is an antidepressant agent which increases brain levels of monoamines and is an agent to block the metabolism of L-dopa administered for the treatment of Parkinsonism.

A cellular permeant luminide comprising a C functionality of a cellular impermeant inhibitor of adenosine deaminase which blocks the metabolism of adenosine such as coformycin, arabinosyl-6-thiopurine, 6-methylthioinosine, 6-thioinosine, 6-thioguanosine, N¹-methyladenosine, N⁶-methyladenosine, 2-fluorodeoxyadenosine, 2-fluoroadenosine, inosine, 2′-deoxyinosine, deoxycoformycin, 1,6-dihydro-6-hydroxymethyl purine ribonucleoside, erythro-9-(2-hydroxy-3-nonyl)adenine, or 9-β-D-arabinofuranosyl-6-hydroxylaminopurine is a vasodilatory agent, an immunosuppressive agent, a chemotherapeutic potentiating agent, and an agent to enhance cardiac recovery following ischemia. The mechanism in the first case involves the accumulation of adenosine which is a vasodilatory agent; the mechanism in the second case involves disruption of purine metabolism; the mechanism in the third case involves the disruption of the degradation of purine analogue chemotherapeutic agents; the mechanism in the fourth case involves blocking the loss of catabolic products of adenosine triphosphate in the form of purine nucleotides and oxypurines during ischemia. Additional luminides effective in enhancing post ischemic cardiac recovery by the latter mechanism include those with C moietics of inhibitors of adenylate kinase, 5′-nucleotidase, and adenosine translocase such as p¹ p⁵-diadenosine pentaphosphate, α,β-methylene adeno sine diphosphate, and nitrobenzyl-6-thioino sine, respectively.

A blood-brain barrier permeant luminide comprising a C functionality of a blood-brain barrier impermeant inhibitor of γ-aminobutyric acid uptake such as D,L-2,4-diaminobutyric acid, D,L-α-hydroxy GABA, (−)-nipecotic acid, trans-4-aminocrotonic acid, cis-3-aminocyclopentane-1-carboxylic acid, trans-3-aminocyclopentane-1-carboxylic acid, β-guanidinopropionic acid, homohypotaurine, 4-aminopentanoic acid, homotaurine, β-alanine, imidazoleacetic acid, 6-aminohexanoic acid, D,L-carnitine, D,L-2,6-diaminopimetic acid, D,L-2-fluoro GABA, guanidino acetic acid, 2-hydrazinopropionic acid, taurine, D,L-omithine, or sulphanilamine potentiates the inhibitory action of GABA and is a muscle relaxant, anticonvulsant, sedative, and anxiolytic agent.

A cellular permeant luminide comprising a C functionality of cellular impermeant inositol 1,4,5-triphosphate which is a major second messenger for stimulating a whole range of cellular processes such as contraction, secretion, and metabolism is an agent for activating these processes including secretion of neural transmitters to function as an agent for the treatment of mental disorders or secretion of insulin to function as a hypoglycemic agent.

A cellular permeant luminide comprising a C functionality of cellular impermeant guanosine 5′ cyclic monophosphate or 8-bromo guanosine 5′ cyclic monophosphate which relaxes smooth muscle is an antihypertensive and bronchodilator agent.

A cellular and blood-brain barrier permeant luminide comprising a C functionality of a cellular and blood-brain barrier impermeant inhibitor of the uptake system for glycine, the inhibitory synaptic transmitter of the spinal cord, such as hydrazinoacetic acid is an agent for spinal reflex inhibition.

A cellular permeant luminide comprising a C functionality of a cellular impermeant isoquinoline-sulfonamide inhibitor of protein kinase C, cAMP-dependant protein kinase, or cGMP-dependent protein kinase such as N-(2-aminoethyl)-5-isoquino-linesulfonamide is an agent which blocks the secretion, contraction, and metabolic events regulated by these mediators of external phydsiologic stimuli.

A cellular permeant luminide comprising a C functionality of cellular impermeant Ribavirin which is active against HSV-1 and 2, hepatitis, and influenza viruses, or phosphonoacetic acid which is a highly specific inhibitor of Herpes Simplex virus induced polymerase and is active against HSV-1 and HSV-2, or adenine arabinoside (ara-A), cytosine arabinoside (Ara-C), ara-A 5′-monophosphate (ara-AMP), or hypoxanthine arabinoside (ara-Hx) which is active against HSV or phagicin which is active against vaccinia and HSV, or 4-fluoroimidazole, 4-fluoroimidazole-5-carboxylic acid, 4-fluoroimidazole-5-carboxamide, 5-fluoro-1-β-D-ribofurano-sylimidazole-4-carboxamide, 5-amino-1-β-D-ribofuranosyl-imidazole-4-carboxamide, poly (I).multidot.poly (C), sinefungin, iododeoxyuridine, 9-(2-hydroxy-ethoxymethyl)guanine, gliotoxin, distamycin A, netropsin, congocidine, cordycepin, 1-β-D-arabinofuranosylthymine, 5,6-di-hydroxy-5-azathymidine, pyrazofurin, toyocamycin, or tunicamycin is an antiviral agent.

A cellular permeant luminde which comprises a C functionality of a cellular impermeant inhibitor of fungal chitin synthetase such as polyoxin D, nikko-mycin Z, or nikkomycin X; or which comprises a C functionality of an impermeant antifungal agent such as ezomycin A₁, A₂, B₁, B₂, C₁, C₂, D₁, or D₂ or platenocidin, septacidin, sinefungin, A9145A, A9145C, or thraustomycin is an antifungal agent.

A blood-brain barrier permeant luminide comprising a c functionality of a blood-brain barrier impermeant inhibitor of central nervous system carbonic anhydrase such as methazolamide, or 2-benzoylimino-3-methyl-6⁴-1,3,4-thiadiazoline-5-sulfonamide subsgituted at the benzolyl group with 3,4,5-trimethoxy, 2,4,6-trimethoxy, 2,4,5-trimethoxy, 4-chloro, 4-bromo, 4-iodo, or hydrogen is an anticonvulsant agent.

A cellular and blood-brain barrier permeant luminide comprising a C functionality of a cellular and blood-brain barrier impermeant inhibitor of dopamine-B-hydroxylase during the synthesis of norepinephrine and epinephrine such as fuscaric acid, 5-(3′,4′-dibromobutyl)picolinic acid, 5-(3′-bromobutyl) picolinic acid, 5-(3′,4′-dichlorobutylpicolinic acid, YP-279, benxyloxyamine, p-hydroxybenzyloxyamine, U-21,179, U-7231, U-6324, U-0228, U-5227, U-10,631, U-10,157, U-1238, U-19,963, U-19,461, U-6628, U-20,757, U-19,440, U-15,957, U-7130, U-14,624, U-22,996, U-15,030, U-19,571, U-18,305, U-17,086, U-7726, dimethyldithiocarbamate, diethyldithiocarbamate, ethyldithiocarbamate, 2-mercaptoethylguanidine, thiophenol, 2-mercaptoethylamine, 3-mercaptopropylguanidine, 3-mercap-topropyl-N-methylguanidine, 2-mercaptoethanol, 2-mercaptoethyl-N-methylguanidine, 2-mercaptoethyl-N,N′-dimethylguanidine, 4,4,6-trimethyl-3,4-dihydropyrimidine-2-thiol, N-phenyl-N′-3-(4H-1,2,4-trizolyl)thiourea, methylspinazarin, 6,7-dimethylspinazarin, 7-O-methy-spinochrome B, 6-(3-hydroxybutyl)-7-O-methylspinachrome B, aquayamycin, chrothiomycin, frenoclicin, N-n-butyl-N′-3-(4H-1,2,4-trazolyl) thiourea, propylthiouracil, mimosine, mimosinamine, or mimosinic acid is an antihypertensive agent.

A cellular permeant luminide of a cellular impermeant inhibitor of histidine decarboxylation during the synthesis of histamine such as 2-hydroxy-5-carbomethoxybenzyloxyamine, 4-toluenesulfonic acid hydrazide, 3-hydroxybenzyloxyamine, hydroxylamine, aminooxyacetic acid, 4-bromo-3-hydroxybenzyloxyamine (NSD-1055), rhodanine substituted in the 3 position with p-chlorophenethyl, p-chlorobenzyl, p-methylthiobenzyl, p-methylbenzyl, p-fluorobenzyl, amino, 3,4-dichlorobenzyl, p-bromobenzyl, p-methoxybenzyl, p-bromoanilino, p-iodoanilino, p-chloroanilino, p-toluidino, anilino, 2,5-dichloroanilino, dimethylamino, or p-methoxyphenyl; 2-mercaptobenzimidazole-1,3-dimethylol, 4-bromo-3-hydroxy-benzoic acid, 4-bromo-3-hydroxybenzyl alcohol, 4-bromo-3-hydroxy-hippuric acid, (R,S)-aαfluoromethyl-histidine, (S)-α-fluoromethylester, L-histidine ethyl ester, L-histidinamide, D,L-3-amino-4-(4-imidazolyl)-2-butanone, 2-bromo-3-hydroxybenzyloxyamine, 5-bromo-3-hydroxybenzyloxyamine, 4,6-dibromo-3-hydroxybenzyloxyamine, aminooxypropionic acid, benzyloxyamine, 4-bromo-3-benzenesulfonyloxybenzyloxyamine, 3′,5,7-trihydroxy-4′,6-dimethoxyisoflavone, lecanoric acid, N-(2,4-dihydroxybenzoyl)-4-aminosalicylic acid, or 3′,5,7-trihydroxy-4′,8-dimethoxyisoflavone is an agent for the treatment of allergy, hypersensitivity, gastic ulcer, and inflamation.

Luminides also comprise C functionalities of pharmaceutical molecules as appear in Physicians Desk Reference, Edward R. Barnhart, 41th ed., 1987, Medical Economics Company Inc., N.J.; USAN and the Dictionary of Drug Names, ed. by Mary C. Griffiths, The United States Pharmacopedial Convention, (1986); and The Pharmacological Basis of Therapeutics, ed. by A. G. Gilman, L. Goodman, A. Gilman, 7th ed., (1985), MacMillan Publishing Co., N.Y., N.Y., (incorporated by reference) where the pharmacokinetics and/or the pharmacodynamics of these agents are altered via delivery to the site of action by way of a luminide agent such that the therapeutic effect or therapeutic ratio is enhanced. Some examples follow which are not meant to be exhaustive.

A luminide with high permeance to the blood-brain barrier comprising a C functionality of a centrally acting converting enzyme inhibitor such as captopril which possesses a lesser blood-barrier permeance is an agent with increased efficacy of the central nervous system antihypertensive effect of the centrally acting converting enzyme inhibition including captopril.

A luminide with an A moiety which reacts with free radicals and electron carriers in the cytosol of bacteria to effect release of the C moiety and which possesses greater permeance or β-lactamase resistance than its C moiety of a bacterial wall synthesis inhibitor such as a penicillin, cephalosporin, or cephamycin is a more efficacious and broad spectrum antibacterial agent than the free C moiety.

A luminide possessing more favorable pharmacokinetics or pharmacodynamics than its C moiety of an agent which blocks bacterial synthesis of tetrahydrofolate such as a sulfonamide (an analogue of p-aminobenzoic acid) including sulfanilamide, sulfadiazine, sulfamethoxazole, sulfisoxazole, or sulfacetamide or an inhibitor of dihydrofolate reductace including pyrimethamine, cycloguanil, trimethoprin, isoaminopterin, 9-oxofolic acid, or isofolic acid is a more efficacious antibacterial than the free C moiety.

A luminide possessing more favorable pharmacokinetics or pharmacodynamics than it C functionality of a bactericidal agent such as nalidixic acid or oxolinic acid is a more efficacious antibacterial than the free C moiety.

A luminide possessing more favorable pharmacokinetics or pharmacodynamics than its C moiety of an inhibitor of bacterial protein synthesis such as vancomycin, an aminogylcoside, erythromycin, tetracyclin, or chloramphenicol is a more efficacious antibacterial agent than the free C moiety.

A luminide prossessing more favorable pharmacokinetics or pharmacodynamics than its C moiety of an inhibitor of viral DNA polymerase such as vidarabine is a more efficacious antiviral agent than the free C moiety.

A luminide possessing more favorable pharmacokinetics or pharmacodynamics than its C moiety which is tuberculostatic or tuberculocidal such as isoniazid or aminosalicyclic acid is a more efficacious agent for the treatment of tuberculosis than the free C moiety.

A luminide possessing more favorable pharmacokinetics or pharmodynamics than its C moiety of an anthelmintic agent such as oxamniquine, piperazine, metronidazole, diethylcarbamazine, paromomycin, niclosamide, bithionol, metrifonate, hycanthone, dichlorophen, or niclosamide is a more efficacious anthelmintic agent than the free C moiety.

A luminide possessing more favorable pharmacokinetics or pharmacodynamics than its C moiety of an H₂-blocking agent such as cimetidine or ranitidine is a more efficacious anti-ulser agent than the free C moiety.

A luminide possessing more favorable pharmacokinetics or pharmacodynamics than its C moiety of an agent which blocks release of norepinephrine such as sotalol, guanethidine, pindolol, pronethalol, KO 592, practolol, oxprenolol, or pronethalol is an antiarrhythmic, antihypertensive and antipsychotic agent.

A luminide possessing more favorable pharmacokinetics or pharmacodynamics than its C moiety of a xanthine oxidase inhibitor such as allopurinol, thioinosinate, 5,7-dihydroxypyrazolo [1,5-a]pyrimidine substituted at the 3 position with hydrogen, nitro, bromo, chloro, phenyl, 3-pyridyl, p-bromophenyl, p-chlorophenyl, p-acetylanilino, p-tolulyl, m-tolulyl, naphthyl, or 3,4-methylenedioxyphenyl; 8-(m-bromoacetamidobenzylthio)hypoxanthine, 8-(m-bromoacetamidobenzylthio)hypoxanthine, guanine substituted at the 9 position with phenyl, 4-chlorophenyl, 3-chlorophenyl, 3,4-dichlorophenyl, 4-methoxyphenyl, 3,4-dimethoxyphenyl, 4-dimethylaminophenyl, 4-aminophenyl, 3-aminophenyl, 3-trifluormethylphenyl, 4-benzamido, 4-carboxylphenyl, 4-methylpheyl, 4-ethylphenyl, 3-methylphenyl, B-naphthyl, or 4-ethoxyphenyl; 4,6-dihydroxypyrazolo[3,4-d]pyrimidine, 4-trifluoromethylimidazoles substituted at the 2 position with phenyl, p-chlorophenyl, p-methoxyphenyl, p-acetylanilino, p-nitrophenyl, p-dimethylaminophenyl, p-cyanophenyl, p-fluorophenyl, p-carboxyphenyl, m-chlorophenyl, 3,4-dichlorophenyl, 4-pyridyl, 3-pyridyl, 2-quinolyl, 6-quinolyl, 4-quinolyl, 7-quinolyl, 2-pyrazinyl, or 1-(2-pyridyl-4-trifluoromethyl-5-bromoimidazolyl; 5-(4-pyridyl)-1,2,4-triazoles substituted at the 5 position with 4-pyridyl, 3-pyridyl, 2-pyridyl, phenyl, p-chlorophenyl, m-chlorophenyl, p-sulfonamidophenyl, 3,5-dichlorophenyl, 3,5-dicarboxyphenyl, 6-quinolyl, 2-furyl, 4-pyridazinyl, 2-thienyl, 2-pyrimidinyl, 4-pyrimidinyl, or 4-pyrazinyl; difinisal, 4(or 5)-(2-aminoethylthio-azo)imidazole-5(or 4)-carboxamide, 4 (or 5)-diazoimidazole-5(or 4)-carboxamide, or S-[5(or 4)carbamoyl-4(or 5)-imidazolylazo]cysteine is a more efficacious agent for the treatment of gout and hyperuricemic conditions than the free C moiety.

A luminide possessing more favorable pharmacokinetics or pharmacodynamics than its C moiety which inhibits DNA synthesis such as a bis-thiosemicarbazone, 3,5-diisopropylsalicyl-hydroxamic acid, 4-hydroxybenzoylhydroxamic acid, 3-methylsalicylhydroxamic acid 2,5-dihydroxybenzoylhydroxamic acid, or 2-hydroxy-3,4,5-trimethoxybenzoylhydroxamic acid; or which inhibits nucleotide synthesis such as N-(phosphoacetyl)-L-aspartate which inhibits asparatate transcarbamylase during pyrimidine synthesis, or azaserine or 6-diazo-5-oxo-L-norleucine which inhibits purine synthesis at the phosphoribosyl-formyl-glycineamidine synthetase step; or which is an antifolate such as methotrexate, 2,4-diamino-5-benxyl-6-(4-phenylbutyl) pyrimidine, 2,4-diamino-5-phenyl-6-(4-phenylbutyl) pyrimidine, 2,4-diamino-5-phenyl-6-(3-anilinopropyl) pyrimidine, 2-amino-4-hydroxy-5-phenyl-6-(3-p-aminobenzoylglutamic acid propyl) pyrimidine, N-(p-{[(2,4-diamino-6-quinazolinyl)methyl]methylamino}benzoyl)-L-glutamic acid, N-{p-[(2,4-diamino-5-methylquinazolinyl)methylamino]benzoyl}-L-aspartic acid, N-(p-{[(2-amino-4-hydroxy-6-quinazolinyl)methyl]methylamino}benzoyl)-L-glutamic acid, 2,4-diaminoquinazolines: CCNSC 105952, CCNSC 112846, CCNSC 121346, CCNSC 122761, CCNSC 122870, CCNSC 529859, CCNSC 529860, or CCNSC 529861; 8-aza GMP, 7-deaza-8-aza GMP, 2′-dGMP, β-D-arabinosyl GMP, pentopyranine A-G, β-ribofuranosyl-1,3-oxazine-2,4-dione, pyrazofurin, 6-(p-chloroacetylanilinomethyl)-5-cetylvinylanilinomethyl)-5-(p-chlorophen yl)-2,4-diaminopyridine, 6-(p-chloroacetyl-ethylanilino-methyl)-5-(p-chlorophenyl)-2,4-diamino pyridine, 6-(p-chlorophenylbutylanilinomethyl)-5-(p-chlorophenyl)-2,4-diamino pyridine, p-(2,6-diamino-1,2-dihydro-2,2-dimethyl-S-triazin-1-yl) phenylpropionyl sulfanilylfluoride or variants of the propionamide bridge of acrylamido, N-ethylsulfonamido, N-ethylcaboxamido, oxyacetamido, or oxythyloxy; or which inhibits purine or pyrimidine synthesis such as xylosyladenine, 6-azauridine, 5-aminouridine, 5-azaorotic acid; or which inhibits nucleotide interconversion such as hadacidin, 6-mercaptopurine, azathioprine, nitro-dUMP, psicofuiranine, decoyinine, 5-fluorouracil, 5-fluorodeoxyuridine, shadowmycin; or which inhibits nucleotide utilization such as cytosine arabinoside, arabinosyladenine; or which becomes incorporated into polynucleotides such as 8-azaguanine, tubercidine, toyocamycin, sangivamycin, formycin, 7-deazainosine, 8-azainosine, or 7-thia-7,9-dideazainosine; or which is a glyoxalase inhibitor such as Glyo-I, or Glyo-II, is a more efficacious antineoplastic agent than the free C moiety.

A luminide possessing more favorable pharmacokinetics or pharmacodynamics than its C moiety of an agent which blocks synthesis of prostaglandin A₂ which effects platelett aggregation such as salicylic acid, pyrogallol, 5,8,11,14-eicosatetraynoic acid, α-naphthol, guaiacol, propylgallate, nordihydroguiaretic acid, N-0164, benzydamine, 9,11-azoprosta-5,13-dienoic acid, 2-isopropyl-3-nicotinylindole, is a more efficacious antithrombotic agent than the free C moiety.

A luminide possessing more favorable pharmacokinetics or pharmacodynamics than its C moiety of an agent wnich blocks prostaglandin synthetase such as indomethacin, sulindac, tolmetin, mefenamic acid, ibuprofen, naprozen, fenoprofen, fluribiprofen, ketoprofen, meclofenamic acid, flufenamic acid, niflumic acid, benzydamine, oxyphenbutazone, asprin, acetaminophen, salicylamide, O-carboxydiphenylamine, tolectin, diclofenac, 2,7-dihydroxynaphthalene, 5-(4-chlorobenzoyl)-1-methylpyrrole-2-acetic acid, 5-(4-methylbenzoyl)-1,4-dimethylpyrrole-2-acetic acid, 5-(4-chlorobenzoyl)-1,4-dimethylpyrrole-2-acetic acid, 5-(4-fluorobenzoyl)-1,4-dimethylpyrrole-2-acetic acid, 5-(4-chlorobenzoyl)-1,4-dimethylpyrrole-2-(2-propionic acid), 5,6-dehydroarachidonate, 11,12-dehydroarachidonate, or 5,8,11,14-eicosatetraynoate; or of an agent which blocks lipoxygenase or blocks leukotriene action such as BW755C, FPL 55712, or U-60,257 is a more efficacious nonsteroidal anti-inflammatory agent than the free C moiety.

A luminide possessing more favorable pharmacokinetics or pharmacodynamics than its C moiety of an antiarrhythmic agent such as procainamide or quinidine is a more efficacious antiarrhythmic agent than the free C moiety.

A luminide possessing more favorable pharmacokinetics or pharmacodynamics than its C moiety of an inhibitor of hepatic synthesis of Vitamin K dependent clotti-ng factors such as warfarin sodium, dicumarol, 4-hydroxycoumarin, phenprocoumon, or acenocoumarol is a more efficacious anticoagulant than the free C moiety.

A luminide possessing more favorable pharmacokinetics or pharmacodynamics than its C moiety which directly relaxes vascular smooth muscle such as hydralazine, minoxidil, or isoxsuprine is a more efficacious antihypertensive agent than the free C moiety.

A luminide possessing more favorable pharmacokinetics or pharmacodynamics than its C moiety of a Na⁺-K⁺-ATPase inhibitor such as digtoxigenin, digoxigenin, cymarol, periplogenin, or strophanthidiol, or ouabain glycosides, cardenolides, or basic esters, or ICI-63,632, ICI-63,605, ICI-62-655, ICI-62,838, ICI-69,654, ICI-58,622, ICI-61,374, ICI-57,267, ICI-61,424, ICI-61,411, ICI-65,199, ICI-70,898, ICI-70,899, ICI-70,900, ICI-70,901, ICI-62,966, ICI-65,210, ICI-63,116, ICI-62,936, ICI-65,551, ICI-63,978, ICI-62,276, ICI-63,056, ICI-67,135, ICI-67,167, ICI-67,134, ICI-67,875, ICI-67,880, or ICI-61,558 is a more efficacious inotropic agent than the free C moiety.

A luminide possessing more favorable pharmacokinetics or pharmacodynamics than its C moiety which is a calcium channel blocker such as prenylamine, verapamil, fendiline, gallopamil, cinnarizine, tiapamil, diltiazem, bencyclan, or nifedipine; or an agent which stabalizes calcium binding to cellular calcium stores and thereby inhibits the release of this calcium by contractile stimuli such as 8-(N,N-diethylamino)-octyl 3,4,5-trimethoxybenzoate (TMB-8) is a more efficacious vasodilatory agent than its free moiety.

A luminide possessing more favorable pharmacokinetics or pharmacodynamics than its C moiety of a monoamine oxidase inhibitor such as tranylcypromine, phenylethylamine, trans-cinnamic acid, phenelzine, or isocarboxazid is a more efficacious antidepressant agent than the free C moiety.

A luminide possessing more favorable pharmacokinetics or pharmacodynamics than its C moiety of a benzodiazepine compound such as clorazepate is a more efficacious tranquillizer than the free C moiety.

A luminide possessing more favorable pharmacokinetics or pharmacodynamics than its C moiety of an antiseizure agent such as valproic acid is a more efficacious antiepileptic agent than the free C moiety. A luminide possessing more favorable pharmacokinetics or pharmacodynamics than its C moiety of an agent which causes repression of the synthesis of HMG-COA reductase such as 20-α-hydroxycholesterol, 22-ketocholesterol, 22-α-hydroxycholesterol, 25-hydroxycholesterol, 22-β-hydroxycholesterol, 7-α-hydroxycholesterol, 7-β-hydroxycholesterol, 7-ketocholesterol, or kryptogenin; or of an agent which inhibits HMG-COA reductase such as, lorelco; or of an agent which inhibits lipolysis such as 5-methylpyrazole-3-carboxylic acid (U-19425), nicotinic acid, uridine, inosine, 3,5-dimethylisoxazole (U-21221), 3,5-dimethypyrazole, prostaglandin E₂, eritadenine, or eritadenine isoamyl ester; or of an agent which inhibits lipogenesis such as ascofuranone, (−)-hydroxycitrate, or tetrolyl-CoA; or of an agent which is hypocholesterolemic such as lentysine; or of an agent which lowers triglycerides such as lopid; or of an agent which is an inhibitor of acetyl-CoA carboxylase during lipogenesis such as 2-methyl-2-op-(1,2,3,4-tetrahydro-1-naphthyl)-phenoxy!-propionate (SU13437), 2-(p-chlorophenoxy)-2-methylpropionate, kynurenate, xanthurenate, kynurenine, 3-hydroxyanthranilate, or 2-methyl-2-[p-(p-chlorophenyl)phenoxy]propionate; or of an agent which is an inhibitor of hepatic B-lipoprotein production such as orotic acid is a more efficacious hypolipidemic agent than its free C moiety.

A luminide possessing more favorable pharmacokinetics or pharmacodynamics than its C moiety of a vasodilater such as WS-1228A, or WS-1228B; or of an anti-inflammatory agent such as amicomacin A is a more efficacious vasodilator or anti-inflammatory agent, respectively, than the free C moiety.

A luminide with more favorable pharmacokinetics or pharmacodynamics than its C moiety which is a protease inhibitor such as leupeptin; or which is an inhibitor of pepsin such as a pepstatin, a pepstanone, or a hydroxypepstatin is a more efficacious agent for the treatment of muscular dystrophy or peptic ulcer disease, respectively, than its free C moiety.

A luminide with more favorable pharmacokinetics or pharmacodynamics than its C moiety of an inhibitor of cell surface enzymes such as bestatin, amastatin, forphenicine, ebelactone, or forphenicin is a more efficacious immunomodifier agent than its free C moiety.

A luminide with more favorable pharmacokinetics or pharmacodynamics such as enhanced permeability relative to its C moiety of a phosphodiesterase inhibitor such as theophyllineacetic acid, theophylline, dyphylline, disodium cromoglycate, 6-n-butyl-2,8-dicarboxy-4,10-dioxo-1,4,7,10-tetrahydro-1,7-phenanthrolin, 2-chloroadenosine, dipyridamole, EG 626, AY-17,605, AY-17,611, AY-22,252, AY-22,241, cis-hinokiresinol, oxy-cis-hinokiresinol, tetrahydro-cis-hinokiresinol, trans-hinokiresinol, dehydrodicaffeic acid, 2,6,4′-trihydroxy-4-methoxybenzophenone, p-hydroxyphenyl crotonic acid, papaverine, 3-(5-tetrazolyl)thioxanthone-10,10-dioxide, 3-carboxythioxanthone-10,10-dioxide, W-7, HA-558, MY-5445, OPC-3689, OPC-13135, or OPC-13013, reticulol, PDE-I, or PDE-II is a more efficacious cardiac stimulant, diuretic, vasadilator, platelett aggregation inhibitor, and an agent for the treatment of asthma and allergic reaction than its free C moiety. Such a luminide comprising a C moiety of ICI 74,917 is also a more efficacious agent for the treatment of asthma and allergic reactions.

A luminide possessing more favorable pharmacokinetics or pharmacodynamics such as enhanced cellular or blood-brain barrier permeability or resistance to inactivation by tissue dehalogenases and transaminases than its C functionality of an inhibitor of tyrosine hydroxylase, the enzyme catalyzing the rate-limiting reaction in the biosynthesis of norepinephrine, such as azadopamine, isopropylazadopamine, dimethylazadopamine; triphenolic compounds such as n-propylgallate; diphenolic benzoic acid derivatives such as 3,4-dihydroxybenzoic acid; phenylcarbonyl derivatives such as 3,4-dihydroxybenzaldehyde, arterenone, or adrenalone H 22/54, 3-iodo-L-tyrosine, D,L-α-methyl-p-tyrosine, L-3-iodo-α-methyltyrosine, 3-bromo-α-methyltyrosine, gentistic acid, 3-chloro-α-methyltyrosine, phenylalanine derivatives, 3,5-diiodo-L-tyrosine, 3,5-dibromo-L-tyrosine, 3-bromo-o-methyl-L-tyrosine, 3-fluro-α-methyl-L-tyrosine, catechol analogues, 3,4-dihydroxyphenylethylacetamide, 3,4-dihydroxyphenyliso-proplyacetamide, 3,4-dihydroxyphenylbutylacetamide, 3,4-di-hydroxyphenylisobutylacetamide, D,L-α-methylphenylalanine, D,L-3-iodophenylalanine, D,L-4-iodophenylalanine, D,L-α-methyl-3-iodophenylalanine, D,L-α-methyl-3-bromophenylalanine, D,L-α-methyl-3-chlorophenylalanine, D,L-α-methyl-3-fluorophenylalanine, mimosine, mimosinamine, mimosinic acid, 7-O-methylspinochrome B, 6-(3-hydroxybutyl)-7-O-methylspinachrome B, aquayamycin, chrothiomycin, frenolicin, fuscaric acid, pentylpicolinic acid, dopstatin, methylspinazarin, 6,7-dihydroxymethylspinazarin, 3-ethyl-α-methyltyrosine, 3-methyl-α-methyltyrosine, 3-isopropyl-x-methyltyrosine, 3-allyl-α-methyltyrosine, 3-[4-hydroxy-3-(2-methylallyl)-phenyl]-2-methylalanine, 3-[3-(2,3-epoxypropyl)-4-hydroxyphenyl]-2-methylalanine, 3-isobutyl-α-methyltyrosine, 3-methylvinyl-α-methyltyrosine, 5-methyl-6,7-diphenyltetrahydropterin, 3-(2,3-dihydro-2,2-dimethyl-5-benzofuranyl!-2-methylalanine, 3-[2,3-dihydro-2,2-dimethyl-5-benzofuranyl]-2-methylalan ine, α-methyldopa, or ethyl-3-amino-4H-pyrrolo[3,4c]isoxazole carboxylate is a more efficacious antihypertensive agent than the free C moiety.

In addition, luminides which provide controlled extracellular release of biologically active substances such as drugs and proteins including enzymes and hormones are herein disclosed as macromolecular luminides. Luminides, each comprising a C functionality of a drug or protein such as insulin, erythropoietin, interleuken 2, interferon, growth hormone, atrial natriuretic factor, tissue plasminogen activator, an anti-inflammatory drug, an antihypertensive drug, an inotropic drug, a contraceptive drug, etc., are attached to a polymeric material to which an enzyme is immobilized to form a macromolecular luminide. The enzyme molecules react with molecules in the ambient extracellular environment at a rate in proportion to their concentration to produce peroxide or free radicals which react with the A functionality molecules causing them to achieve a high energy electronic state which is followed by the release of the C molecules where the release of C is in proportion to the ambient concentration of, the substrate of the enzyme.

For example, a macromolecular luminide which provides a release of insulin in proportion to the ambient glucose concentration comprises luminide molecules, each comprising a C functionality of insulin, covalently bound to a biocompatible polymer to which the enzyme glucose oxidase is immobilized. The immobilized enzyme reacts with glucose at a rate proportional to the ambient glucose concentration to produce peroxide which reacts with the A functionality molecules of the attached luminide molecules to effect release of insulin. Because the insulin release is in proportion to the glucose concentration this macromolecular agent represents a very effective diabetic therapy.

As an additional example, cardiac ischemia results in the production and release of degradation products of purines such as xanthine. The enzyme xanthine oxidase oxidizes xanthine and directly reduces oxygen to hydrogen peroxide. Furthermore, tissue plasminogen activator (TPA) is an effective agent for the treatment of myocardial infarction because this agent effects the lysis of fibrin clots in coronary arteries to establish reperfusion. Cardiac recovery is enhanced by diminishing the delay between the occlusion event and the administration of TPA. Thus, a macromolecular luminide comprising luminide molecules, each comprising a C functionality of TPA, bound to a biocompatible polymer to which xanthine oxidase is immobilized is an agent which releases TPA in proportion to the products of cardiac ischemia. Thus, it is a highly effective agent to resolve myocardial infarctions.

In another embodiment, luminide molecules, each comprising an A functionality which achieves a high energy electronic state via a reduction reaction, are attached to a conducting polymer to which an enzyme is immobilized. The immobilized enzyme oxidizes molecules in the ambient environment and transfers electrons to the conducting polymer which reduces the A functionality molecules directly or indirectly via the optional D functionality molecules to effect release of the C molecules.

In the latter embodiment, the conducting polymer derivatized with an enzyme, can be replaced with an electrocatalytic polymer which is reduced directly by molecules in the ambient environment and transfers the electrons to the luminide molecules to effect release of the C molecules. For example, polyvinylferrocene and poly-N-(9,10-anthroquinone)-ethylenimine are conductive polymers and electrocatalytically oxidize glucose. Thus, a macromolecular luminide for the treatment of diabetes comprises a conducting polymer such as polyvinylferrocene to which glucose oxidase is optionally bound and to which luminide molecules are bound where the A functionality molecules of the polymer attached luminides achieve a high energy electronic state via a reduction reaction. The polymer is reduced when glucose oxidase accepts electrons from glucose and transfers them to the polymer. Or, the electrocatalytic polymer is reduced directly by glucose. The reduced polymer reduces the A functionality molecules directly or indirectly via the optional D functionality molecules to effect release of insulin molecules in proportion to the ambient glucose concentration.

Furthermore, macromolecular luminides can be directed to a specific extracellular target site such as an anatomical or biological compartment or organ by further attaching monoclonal antibody molecules to the polymer of the macromolecular luminide which bind to a molecule at the desired target site.

In addition to pharmaceutical agents, luminides also comprise pesticides including. herbicides, fungicides, miticides, nematocides, fuimigants, growth regulators, repellants, defoliants, rodenticides, molluscicides, algicides, desicants, antehelmintics, and bactericides. These luminides can be obtained by one skilled in the art by combining the functionalities, A, B, and optionally, D, of energy donor, energy acceptor, and electron transfer functionality, respectively, with a C moiety which possesses pesticidal activity. C moieties include those that appear in Chemical Week Pesticides Register, Robert P. Ovellette and John A. King, 1977, McGraw-Hill Book Company (incorporated by reference) and analogues of these agents. Enhanced pesticidal effectiveness is acheived via improved delivery of these agents to their target receptors by way of luminide molecules which possess desirable properties such as increased permeance to the cells of the organism relative the free C moieties.

### Representative Luminides with Outline of Synthetic Pathway

Luminides synthesis involves the chemical joining of three or four functionalities. A representative Luminide of three functionalities comprises an energy donor molecule such as a chemiluminescent molecule, an energy acceptor molecule such as a photochromic molecule, and a drug. A representative Luminide of four functionalities comprises the mentioned three functionalities and also an electron transfer functionality which can undergo an oxidation reduction reaction. The list of examples of reaction pathways is intended to be exemplary and other pathways can be devised by one skilled in the art. Furthermore, only a representative number of Luminides are shown and a vast number of other novel Luminides can be made by one skilled in the art following the guidelines herein disclosed.

The Luminides can be prepared by known reactions where necessary, appropriate derivatives of the subunits are formed before coupling. Representative examples of appropriate derivatization and coupling reactions are given in the following examples, illustrating the preparation of representative Luminides. The following examples involving representative structures shown in TABLE 5 are not to be taken as an exhaustive listing, but only illustrative of the possibilities according to the present invention.

TABLE 5
Structures of representative drugs, carriers and prodrugs which are illustrative of
representative synthesis methods and uses according to the present invention.

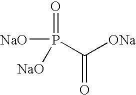

Foscarnet

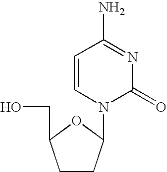

ddc

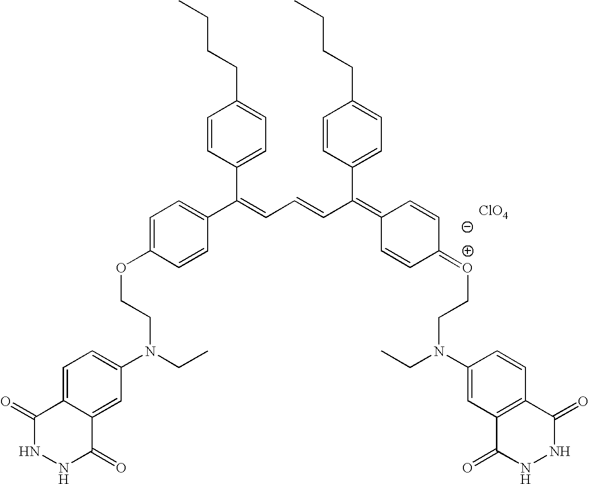

YY99811-1

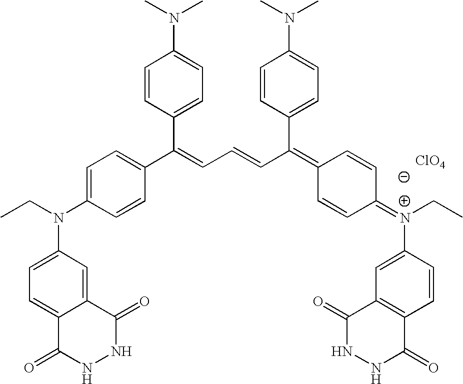

6a

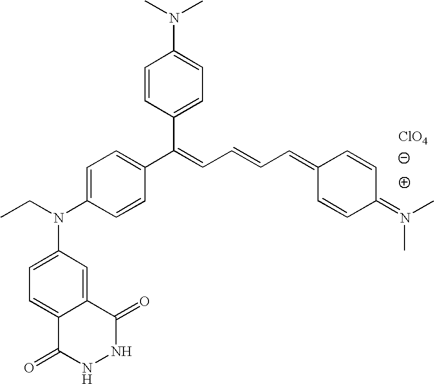

GZW2-33-1
C₃₇H₃₈N₅O₂ · ClO₄
M.W. 684.20

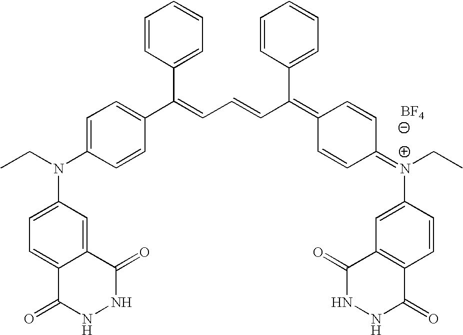

GZW1-98-2
C₄₉H₄₁N₆O₄ · BF₄
M.W. 864.71

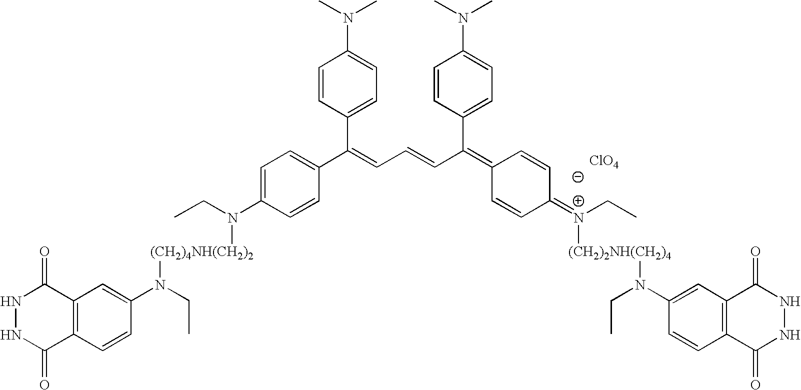

MTLJ-1

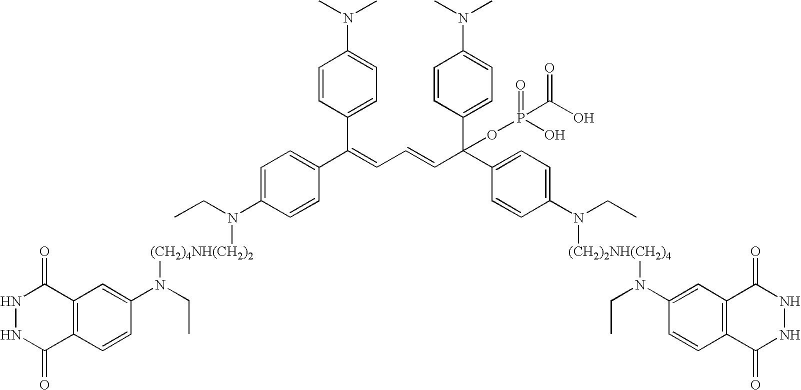

MTLJ-1-Foscarnet

And, the disclosed exemplary Luminides, and components: chemiluminescent molecules, photochromic molecules, energy transfer molecules, and drug molecules can be modified to further candidate components by addition of functional groups by one skilled in the art. Representative groups include alkyl, cycloalkyl, alkoxycarbonyl, cyano, carbamoyl, heterocyclic rings containing C, O. N. S, sulfo, sulfamoyl, alkoxysulfonyl, phosphono, hydroxyl, halogen, alkoxy, alkylthiol, acyloxy, aryl, alkenyl, aliphatic, acyl, carboxyl, amino, cyanoalkoxy, diazonium, carboxyalkylcarboxamido, alkenylthio, cyanoalkoxycarbonyl, carbamoylalkoxycarbonyl, alkoxy carbonylamino, cyanoalkylamino, alkoxycarbonylalkylamino, sulfoalkylamino, alkylsulfamoylaklylamino, oxido, hydroxy alkyl, carboxy alkylcarbonyloxy, cyanoalkyl, carboxyalkylthio, arylamino, heteroarylamino, alkoxycarbonyl, alkylcarbonyloxy, cyanoalkoxy, alkoxycarbonylalkoxy, carbamoylalkoxy, carbamoylalkyl carbonyloxy, sulfoalkoxy, nitro, alkoxyaryl, halogenaryl, amino aryl, alkylaminoaryl, tolyl, alkenylaryl, allylaryl, alkenyloxyaryl, allyloxyaryl, cyanoaryl, carbamoylaryl, carboxyaryl, alkoxycarbonylaryl, alkylcarbonyoxyaryl, sulfoaryl, alkoxysulfoaryl, sulfamoylaryl, and nitroaryl.

In the following examples, a three-functionality luminide has the structure of general formula (I):

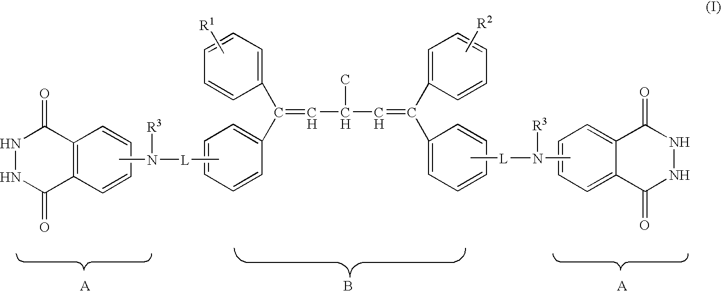

Where functionality A may be aminophthalhyrazide derivatives, sulfonyloxamides or active oxalates; functionality B represents 1,1,5,5-tetrakisarylpentadiene or 1,1,5-trisarylpentadiene derivatives; functionality C represents drug molecules such as Foscarnate, ddc; R represents substituents described as functional groups bellow; Linker L represents the aliphatic chain between A and B, which may be long as in MTLJ-1, short as in YY99811-1 or none as in 6a as shown in TABLE 5.

When B represents 1,1,5-trisarylpentadiene derivatives, the general formula (I) includes the following formula (II):

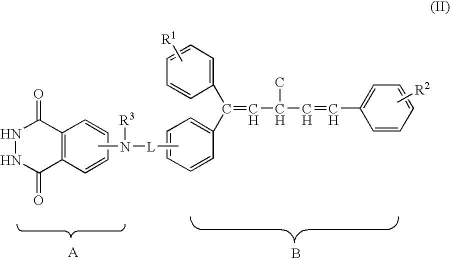

When A represents sulfonyloxamides or active oxalates, the general formula (I) includes formula (M).

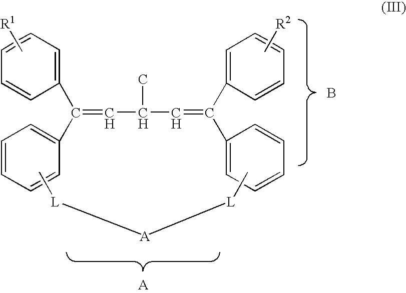

The intermediates to synthesize Luminides can be prepared by reactions known by those skilled in the art. The exemplary synthetic methods shown herein are not meant to be exhaustive. Similar methods and modified intermediates can be used to carry out the disclosed synthetic pathways by those skilled in the art.

General Instrumentation and Materials in the Synthesis of the Carriers and Prodrugs. A schematic for the synthesis of carriers and prodrugs is shown in TABLE 6. Unless otherwise specified, all organic and inorganic reagents and solvents were purchased from commercial suppliers and were used directly without further purification. Elemental Analyses (Anal.) were carried out at Atlantic Microlab, Inc., Norcross, Ga. Fast Atom Bombardment Mass Spectroscopy (FAB) was carried out on VG Analytical ZAB 2-SE high field mass spectrometer at M-Scan, Inc., West Chester, Pa. Melting points (m.p.) were obtained using IA9100 Electrothermal Digital Melting Point Apparatus. Majority of Proton Nuclear Magnetic Resonance spectra (¹H NMR) were recorded on Varian Unity Inova 400 MHz spectrometer at Spectral Data Services, Inc. at Champaign, Ill. Chemical shifts (δ) are reported in ppm relative to tetramethylsilane used as internal standard in deuterated dimethyl sulfoxide (DMSO-d₆). Thin layer Chromatography (Rf) was performed on Baker Si250F silica gel TLC plates.

In an embodiment of present invention, the three-functionality luminide having luminol derivative as the energy-donating moiety A can be synthesized as follows.

For the first type of luminides that the luminols are directly attached through their amino groups to the aryl groups of a photochromic dye listed in TABLE 2, forming the corresponding carriers such as 6a, gzw1-98-2, gzw2-33-1 in TABLE 5, the synthesis is comprised of the following steps (see TABLE 6). Other protecting forms of aminophthalhydrazide such as aminophthalic acid diester [Maeba, I.; Ishikawa, T.; Furukawa, H. Carbohydr. Res. 1985, 140(2), 332-335], aminophthalic acid dihydrazide [Asian J. Chem. 2001, 13(1), 111-118], aminophthalic anhydride [J. Institutional Chem. (India), 2000, 72(4), 146], can be used instead of the aminophthalimide in the following procedures.

1. Preparing the aminophthalimide-substituted precursors for the dye through amination of aryl halide such as palladium-catalyzed amination of aryl halides [Yamamoto, T.; Nishiyama, M.; Koie, Y. Tetrahedron Lett. 1998, 39, 2367-2370]. Following the general synthetic methods given [Mills, R. L. U.S. Pat. No. 5,773,592, Appendix I-VIII] for making the dyes in TABLE 2, the halo-substituted aryl groups of a starting material or an intermediate, such as 2a-e in TABLE 6, are coupled with the aminophthalimide by methods such as the aryl amination under palladium catalysis to form the aminophthalimide-substituted precursors for the dye, such as 3a-f in TABLE 6. Alternatively the precursors can be prepared under the similar conditions using the halo-phthalimides and amino-substituted aryl groups of the intermediates for the dye, which in turn can be obtained conveniently by the amination of the halo-substituted compounds with an imine such as benzophenoneimine.

2. Forming the aminophthalimide-attached dye, such as 4a-f in TABLE 6, by condensation according to the given methods [Mills, R. L. U.S. Pat. No. 5,773,592, Appendix I-VIII].

3. Converting the phthalimide moiety to the aminophthalhydrazide to obtain the carrier, such as 5a-f in TABLE 6. The cationic dyes such as 4a-f are first protected by reacting with base such as sodium hydroxide, sodium methoxide and amines, refluxed with hydrazine in a suitable solvent such as an alcoholic solvent in inert atmosphere and then treated with acid such as perchloric acid, tetrafluroboric acid to regenerate the cationic carriers, such as 5a-f in TABLE 6.

4. Reacting the carrier with one nucleophilic species of a drug to form the luminide prodrug, such as 6a, 7a and 8a.

Alternately the carriers, such as 5a-f in TABLE 6, can be synthesized as follows: By starting with halo-substituted precursor compounds proper halo-substituted dyes, such as 1,5-bis(p-bromophenyl)-1,5-bis(p-dimethylaminophenyl)-pentadienium perchlorate, can be prepared according to the given methods [Mills, R. L. U.S. Pat. No. 5,773,592, Appendix I-VIII]. The cationic dyes are protected by reacting with base such as alkoxide and then coupled with the aminophthalimide by amination of aryl halide such as the palladium-catalyzed amination of aryl halide to obtain the alkoxide-protecting aminophthalimide-substuted dyes, such as alkoxide-4a in TABLE 6. The protected dyes are refluxed with hydrazine in a suitable solvent such as an alcoholic solvent to convert the amino-phthalimide moiety to the aminophthlhydrazide moiety and then treated with acid to generate the carriers, such as 5a in TABLE 6.

A representative scheme of the above synthetic pathways wherein the dyes are the tetraarylpolymethines is given in TABLE 6, Scheme I. Other protecting forms of aminophthalhydrazide such as aminophthalic acid diester can be used instead of the amino-phthalimide in the following procedures. Following the general procedure for making tetraaryploymethine dyes in literature [Mills, R. L. U.S. Pat. No. 5,773,592, Appendix II, Method IIa], the carriers of the luminol-tetraaryl-polymethine luminides can be obtained as follows: (1) Making the halo-substituted diarylketone such as 1a-e by the known reactions such as the direct acylation of arene with halo-substituted benzoyl halide under ferric chloride catalysis or the indirect acylation as in the preparation of 1a. (2) Converting the halo-substituted diarylketone to the halo-substituted diarylketene (the halo-substituted 1,1-diarylethene) such as 2a-e. (3) Coupling the halo-substituted diarylketene with a precursor of aminophthalhydrazide such as aminophthalimide, aminophthalic acid diester, by aryl amination such as the palladium-catalyzed amination of aryl halides to form the aminophthalimide-substituted 1,1-diarylethene such as 3a-f. (4) Condensing the ethene with an orthoester such as triethylorthoformate in a nonaqueous solvent such as acetic anhdydide, containing an acid catalyst such as perchloric acid, tetrafluoroboric acid, to form the aminophthalimide-substituted tetraarylpolymethine dye such as 4a-f. (5) Converting the aminophthalimide moiety to the aminophthalhydrazide to obtain the carrier, such as 5a-f. The cationic dyes such as 4a-f are first protected by reacting with an anion such as hydroxide, methoxide and amine, refluxed with hydrazine in a suitable solvent such as an alcoholic solvent in inert atmosphere and then treated with acid such as perchloric acid, tetrafluroboric acid to regenerate the cationic carriers.

(6) Reacting the carrier with one nucleophilic species of a drug such as 2′,3′-dideoxycytidine, foscarnet, acycloguanosine to form the luminide prodrug, such as 6a, 7a, 8a.

Alternatively the aminophthalimide-substituted 1,1-diarylethene such as 3a-f can be prepared in the above amination conditions using the halo-phthalimides and amino-substituted 1,1-diarylethenes which in turn can be obtained conveniently by the amination of the corresponding halo-substitued diarylethenes with an imine such as benzophenoneimine.

Alternately the carriers of the luminol-tetraarylpolymethine luminides, such as 5a-f in Scheme I, can be synthesized as follows: By starting with halo-substituted diarylketene precursor compounds such as 2a-e, properly halo-substituted tetraarylpolymethine dyes, such as 1,5-bis(p-bromophenyl)-1,5-bis(p-dimethylaminophenyl)-pentadienium perchlorate, can be prepared by condensation with an orthoester such as triethylorthoformate in a nonaqueous solvent such as acetic anhydride containing acid catalyst such as perchloric acid, tetrafluoroboric acid, according to the given methods [Mills, R. L. U.S. Pat. No. 5,773,592, Appendix II, Method IHa]. The cationic dyes are protected by reacting with an anion such as alkoxide and then coupled with the aminophthalimide by amination of aryl halide such as the palladium-catalyzed amination of aryl halide to obtain the alkoxide-protecting aminophthalimide-substituted tetraarylpolymethine dyes, such as alkoxide-protecting 4a-f in Scheme I. The protected dyes are refluxed with hydrazine in a suitable solvent such as an alcoholic solvent to convert the amino-phthalimide moiety to the aminophthalhydrazide moiety and then treated with acid to generate the carriers, such as 5a-f in Scheme I.

TABLE 6
Representative Schematics for the Synthesis of Carriers and Prodrugs.

Scheme I

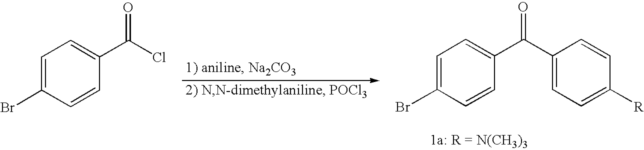

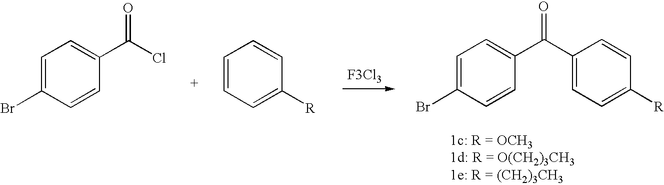

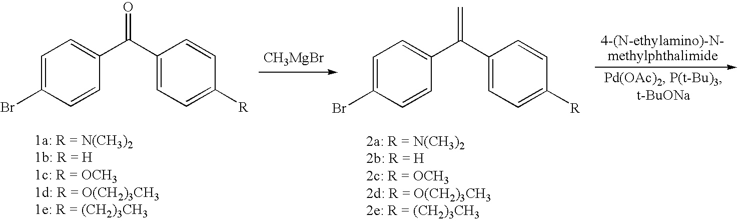

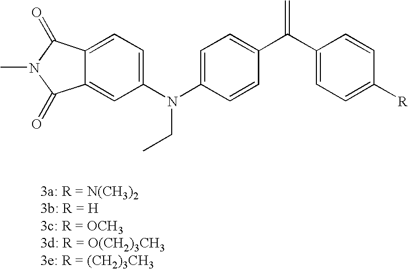

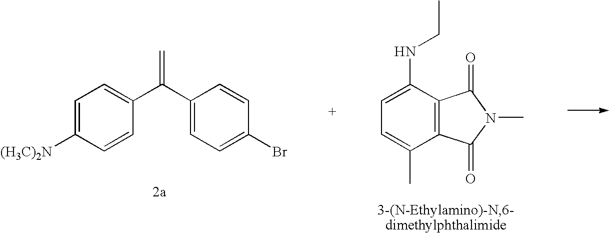

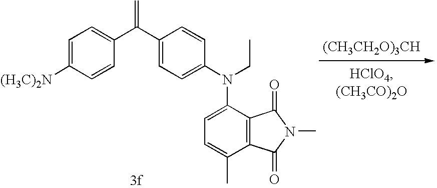

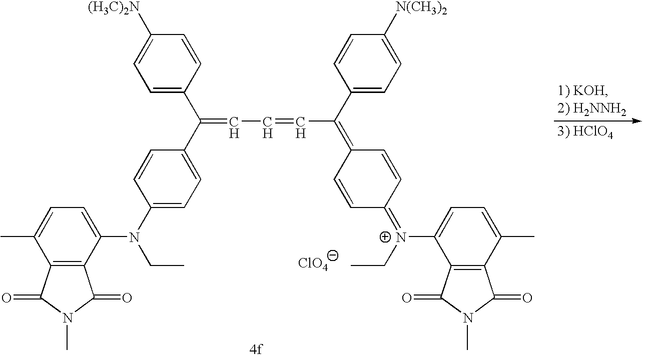

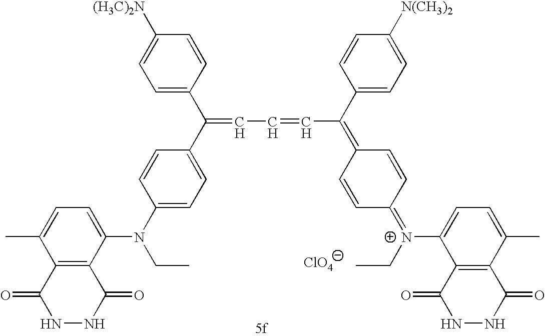

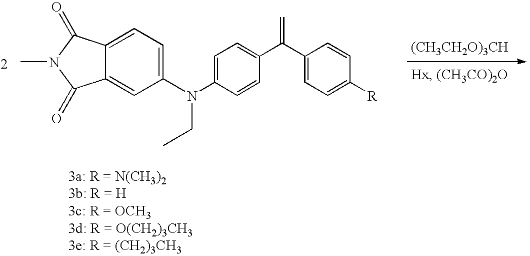

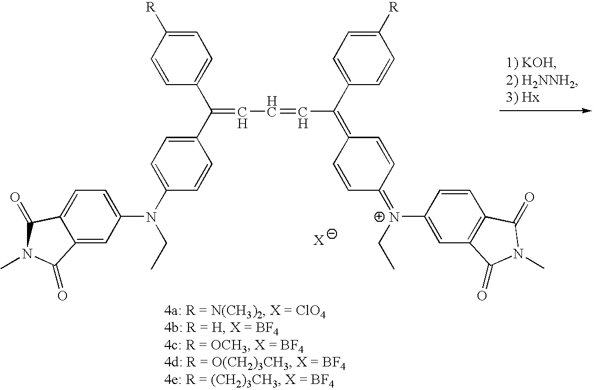

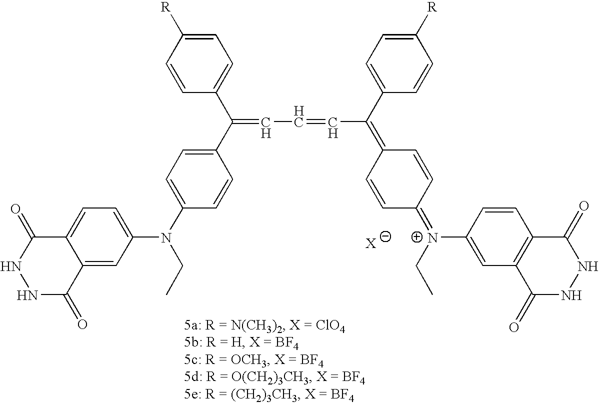

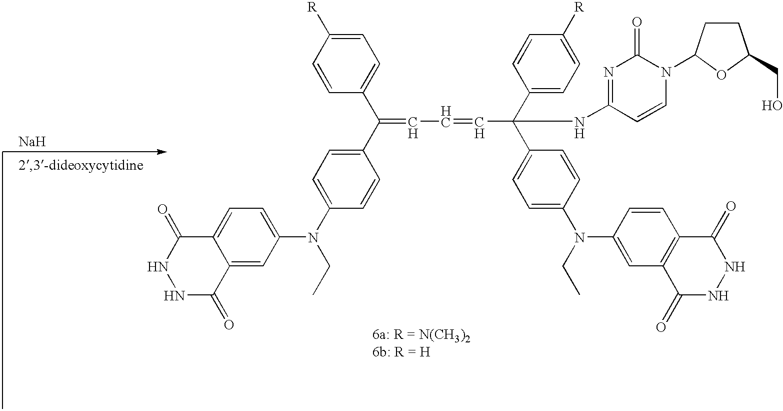

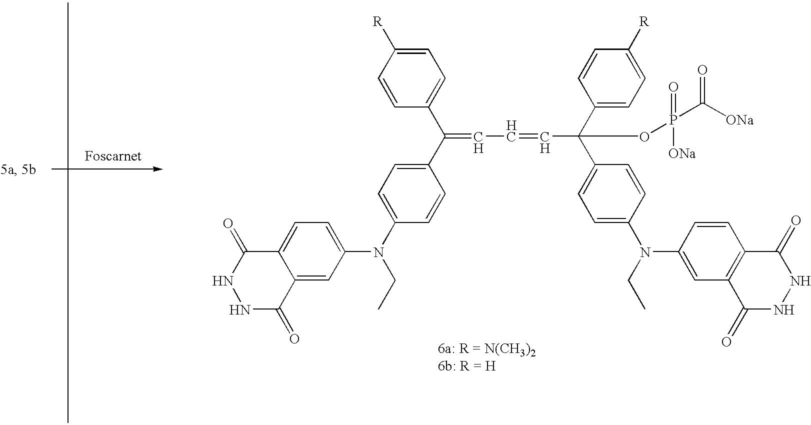

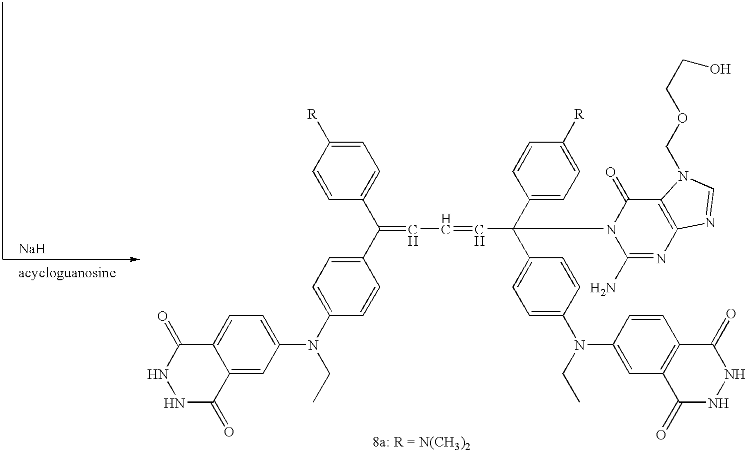

4-Bromo-4′-(N,N-dimethylamino)benzophenone (1a). A solution of aniline (19.1 g, 0.21 mol) and anhydrous sodium carbonate (42 g, 0.4 mol) in anhydrous tetrahydrofuran (250 mL) was brought to reflux under nitrogen. Upon stirring, 4-bromobenzoyl chloride (50 g, 0.23 mol) was added in portions over a period of one hour. The resulting mixture was refluxed for 4 hours and the solvent was then removed under reduced pressure using a rotary evaporator. The crude product deposited was stirred in cold water and collected by filtration. The washing procedure was repeated and the white powder (57.6 g, m.p. 202-203° C.) obtained was air-dried and used for the following reaction without further purification. A mixture of the white powder (30 g), N,N-dimethylaniline (43 g, 0.35 mol), and phosphorous oxychloride (25 g, 0.16 mol) was heated in an oil bath at 112° C. The exothermic displacement reaction occurred in the mixture was indicated by the color change from green to brown as well as by the rapid increase of the temperature to 140° C. The oil bath was removed and the mixture was cooled to 110° C. in an ice-water bath and then continuously stirred at 100-105° C. for 3 hours. The mixture was cooled to 60° C. and poured into an aqueous HCl solution (1.6 N, 220 mL) and then stirred at room temperature overnight. The crude product deposited was collected by filtration and washed with cold water 3 times and then air-dried to give 39 grams of green solids. The filtrate was diluted with 1 liter of water and the precipitate was collected by filtration and washed with cold water 3 times and then air-dried to give 3 grams of green solids. The green solids were combined and recrystallized from ethanol to give the desired product 1a (23.3 g, 0.076 mol, 69% yield) as green sandy crystal: m.p. 126.4-127.4° C.; MS (FAB, MH⁺, C₁₅H₁₄ONBr) calcd. 304, found 304; ¹H NMR (DMSO-d₆) δ 3.04 (s, 6H), 6.77 (d, J=9.0 Hz, 2H), 7.57 (d, J=8.2 Hz, 2H), 7.64 (d, J=8.9 Hz, 2H), 7.72 (d, J=8.2 Hz, 2H); Anal. C₁₅H₁₄ONBr, calcd. C, 59.23; H, 4.64; N, 4.61, Br 26.27, found C, 59.10; H, 4.67; N, 4.53, Br 26.09.

4-Bromo-4′-methoxybenzophenone (1c) [24]. A mixture of anisole (30 mL, 276 mmol), 4-bromobenzoyl chloride (12.4 g, 56.5 mmol), and ferric chloride (0.3 g, 1.85 mmol) was heated under argon atmosphere in an oil bath at 144° C. for 2 hours. The mixture was stirred at room temperature for 1 hour followed by refluxing the mixture in 50 mL of 10% KOH for 30 minutes. After cooling to room temperature, toluene (100 mL) was added to the mixture and the resulting solution was filtered. The organic layer was separated from the filtrate and washed with water (300 mL) twice. Toluene was removed from the solution under reduced pressure. The crude product deposited was recrystallized from toluene to give the product 1c (9.25 g, 31.77 mmol, 56% yield) as white sandy solids: m.p. 156.0-157.6° C.; ¹H NMR (300 MHz, CDCl₃) δ 3.89 (s, 3H), 6.97 (d, J=9.0 Hz, 2H), 7.62 (s, 4H), 7.79 (d, J=8.7 Hz, 2H); Anal. Cl₄H, O₂Br, calcd. C 57.76, H 3.81, found C 57.79, H 3.72.

4-Bromo-4′-n-butoxybenzophenone (1d) [24]. A mixture of n-butyl phenyl ether (30 g, 200 mmol), 4-bromobenzoyl chloride (11.4 g, 51.9 mmol), and ferric chloride (0.3 g, 1.85 mmol) was heated under argon atmosphere in an oil bath at 144° C. for 3 hours. The mixture was cooled to 60° C. followed by the addition of water (30 mL) and toluene (100 mL). The organic layer was separated from the resulting mixture and washed with 1 N HCl (30 mL×3) and water (30 mL×3). Toluene was removed from the resulting solution via rotary evaporator under reduced pressure. The crude product deposited was recrystallized from ethanol to give the product 1d (14.27 g, 42.8 mmol, 82% yield) as white sandy solids: m.p. 115.4-117.2° C.; ¹H NMR (300 MHz, CDCl₃) δ 0.99 (t, J=7.3 Hz, 3H), 1.51 (m, 2H), 1.80 (m, 2H), 4.04 (t, J=6.4, 2H), 6.95 (d, J=8.7 Hz, 2H), 7.62 (s, 4H), 7.79 (d, J=8.7 Hz, 2H); Anal. C₁₇H₁₇O₂Br, calcd. C 61.28, H 5.14, found C 61.35, H 5.20.

4-Bromo-4′-n-butylbenzophenone (1e) [24]. A mixture of n-butylbenzene (25 mL, 160 mmol), 4-bromobenzoyl chloride (12.3 g, 56 mmol), and ferric chloride (0.47 g, 2.8 mmol) was heated under argon atmosphere in an oil bath at 144° C. for 6 hours. After the mixture was cooled to 60° C., water was added (20 mL) and the resulting mixture was extracted by toluene (200 mL). Toluene was removed from the extract via rotary evaporator under reduced pressure. The crude product deposited was recrystallized from 90% ethanol solution to give 12.5 g of solids.

1-(4-Bromophenyl)-1-[4-(N,N-dimethylamino)phenyl]-ethene (2a). Upon stirring a benzene (35 mL) solution of 1a (3.3 g, 10.8 mmol) under a N₂ atmosphere, an ethereal solution of methylmagnesium bromide (3M, 6.5 mL, 20 mmol) was added dropwise. The resulting solution was refluxed under N₂ for 4 hours and then allowed to cool to room temperature. Upon stirring the resulting solution, a saturated aqueous solution of NH₄Cl (5 mL) was added and the final mixture was filtered through a filter paper. The precipitate collected was stirred in hot benzene (30 mL) and then filtered. The filtrates were combined and azeotropically removed the water by refluxing the solution in the presence of p-toluenesulfonic acid monohydrate (0.15 g, 0.8 mmol) for 1 hour. After cooling to room temperature, potassium bicarbonate (1 g, 10 mmol) was added and the mixture was stirred for 30 minutes and then filtered through a filter paper. Solvent was removed from the filtrate under reduced pressure using a rotary evaporator and the crude product deposited was recrystallized from ethanol to give the desired product 2a (2.4 g, 7.9 mmol, 74% yield) as white crystal: m.p. 126.0-126.8° C.; MS (FAB, MH⁺) 303; ¹H NMR (DMSO-d₆) δ 3.91 (s, 6H), 5.24 (s, 1H), 5.38 (s, 1H), 6.70 (d, J=8.9 Hz, 2H), 7.11 (d, J=8.7 Hz, 2H), 7.24 (d, J=8.5 Hz, 2H), 7.55 (d, J=8.5 Hz, 2H); Anal. C₁₆H₁₆NBr, calcd. C 63.59, H 5.34, N 4.64, Br 26.44, found C, 63.61; H, 5.35; N, 4.47, Br 26.63.

1-(4-Bromophenyl)-1-phenylethene (2b). The same procedure described for the synthesis of 2a was employed except that 4-bromobenzophenone (1b, 10 g, 38.3 mmol) and 1.2 equivalent of methylmagnesium bromide were used to generate the crude product 2b (10 g) as light yellow oil. The crude product was used for the preparation of 3b without further purification: ¹H NMR (DMSO-d₆) δ 5.52 (s, 1H), 5.54 (s, 1H), 7.25 (d J=8.3 Hz, 2H), 7.29 (m, 2H), 7.37 (m, 3H), 7.57 (d, J=8.3 Hz, 2H).

1-(4-Bromophenyl)-1-(4-methoxyphenyl)ethene (2c). The same procedure described for the synthesis of 2a was employed except that 4-Bromo-4′-methoxybenzophenone (1c, 7.0 g, 24.0 mmol) and 2.0 equivalent of methylmagnesium bromide were used to generate the crude product followed by recrystallization from ethanol to give 2c (4.88 g, 16.9 mmol, 70% yield): m.p. 91.2-92.6° C.; ¹H NMR (300 MHz, CDCl₃) δ □3.83 (s, 3H), 5.34 (s, 1H), 5.40 (s, 1H), 6.87 (d, J=9.0 Hz, 2H), 7.23 (m, 4H), 7.44 (d, J=8.7 Hz, 2H); Anal. C₁₅H₁₃OBr, calcd. C 62.30, H 5.34, N 4.53, found C 62.27, H 4.53.

1-(4-Bromophenyl)-1-(4-n-butoxyphenyl)ethene (2d). The same procedure described for the synthesis of 2a was employed except that 4-Bromo-4′-n-butoxybenzophenone (1d, 8.0 g, 24.0 mmol) and 1.5 equivalent of methylmagnesium bromide were used to generate the crude product 2d (7.85 g, 23.7 mmol, 98% yield) and it was used for the preparation of 3d. The crude product could be purified by recrystallization from ethanol to give white crystal: m.p. 71.8-73.0° C.; ¹H NMR (300 MHz, CDCl₃) δ 0.98 (t, J=7.2 Hz, 3H), 1.50 (m, 2H), 1.77 (m, 2H), 3.97 (t, J=6.6 Hz, 2H), 5.32 (s, 1H), 5.39 (s, 1H), 6.85 (d, J=8.7 Hz, 2H), 7.22 (m, 4H), 7.45 (d, J=8.4 Hz, 2H); Anal. C₁₈H₁₉OBr, calcd. C 65.27, H 5.78, found C 65.27, H 5.76.

1-(4-Bromophenyl)-1-(4-n-butylphenyl)ethene (2e). The same procedure described for the synthesis of 2a was employed except that 4-Bromo-4′-n-butylbenzophenone (1e, 9.0 g) and 18.9 mL of the 3M ethereal solution of methylmagnesium bromide were used to generate the crude product followed by recrystallization from ethanol to give 4.68 g, of solids.

1-(4-N,N-dimethylaminophenyl)-1-[4-N-ethyl-N-(N-methylphthalimid-4-yl)-aminophenyl]ethene (3a) [25]. An anhydrous toluene solution (5 mL) containing palladium acetate (18 mg, 0.08 mmol) and tri-tert-butylphosphine (66.6 mg, 0.30 mmol) was added to a suspension of 2a (1.0 g, 3.3 mmol), 4-(N-ethylamino)-N-methylphthalimide [26] (0.67 g, 3.3 mmol), and sodium tert-butoxide (0.40 g, 4.0 mmol) in anhydrous toluene (10 mL). The resulting red mixture was stirred at 100° C. under N₂ for 3 hours. After cooling to room temperature, water was added to the mixture and extracted with ethyl acetate (50 mL) 3 times. The organic extracts were combined and washed with saturated aqueous NaCl and then dried over MgSO₄ and filtered. The filtrate was concentrated to a syrup and then dissolved in a mixture of ethyl acetate and hexane (4:1, 100 mL). After allowing the mixture to stand at 4° C. overnight, the yellow crystals formed was collected by decanting the supernatant and dried under reduced pressure to give the desired product 3a (0.90 g, 2.1 mmol, 64% yield): m.p. 136.1-138.0° C.; MS (FAB, MH⁺) 425; ¹H NMR (DMSO-d₆) δ 1.19 (t, J=7.1 Hz, 3H), 2.92 (s, 6H), 2.97 (s, 3H), 3.88 (q, J=7.1 Hz, 2H), 5.31 (s, 1H), 5.37 (s, 1H), 6.73 (d, J=9.0 Hz, 2H), 6.97-7.00 (m, 2H), 7.18 (d, J=8.7 Hz, 2H), 7.26 (d, J=8.3 Hz, 2H), 7.42 (d, J=8.2 Hz, 2H), 7.62 (d, J=8.3 Hz, 1H); Anal. C₂₇H₂₇N₃O₂, calcd. C, 76.21; H, 6.40; N, 9.87, found C, 75.97; H, 6.50; N, 9.62.

1-[4-N-ethyl-N-(N-methylphthalimid-4-yl)-aminophenyl]-1-phenylethene (3b) [25]. Using a micro syringe, P(^{t}Bu)₃ (432 μL, 1.56 mmol) was added to a mixture of crude 2b (2.0 g), 4-(N-ethylamino)-N-methylphthalimide [26] (1.35 g, 6.61 mmol), Pd(OAc)₂ (88 mg, 0.39 mmol), and NaO'Bu (0.89 g, 9.26 mmol) in anhydrous toluene (20 mL). The red mixture was stirred at 100° C. under Ar for 4 hours. After cooling to room temperature, water (100 mL) was added to the mixture and extracted with ethyl acetate (100 mL) twice. The organic extracts were combined and washed with saturated aqueous NaCl and then dried over MgSO₄ and filtered. The filtrate was concentrated to a syrup and flash chromatographed on a silica gel (32-63 μm, 60 Å) column using 30% ethyl acetate in hexane as the eluent to give the desired product 3b as a yellow syrup (1.51 g): R_{f}=0.59; ¹H NMR (DMSO-d₆) δ 1.19 (t, J=7.1 Hz, 3H), 2.97 (s, 3H), 3.88 (q, J=7.0 Hz, 2H), 5.50 (s, 1H), 5.59 (s, 1H), 6.98-7.02 (m, 2H), 7.28 (d, J=8.6 Hz, 2H), 7.34-7.44 (m, 7H), 7.62 (d, J=8.3 Hz, 1H).

1-(4-Methoxyphenyl)-1-14-N-ethyl-N-(N-methylphthalimid-4-yl)aminophenylethene (3c) [25]. The same procedure described for the synthesis of 3a was employed except that 1-(4-bromophenyl)-1-(4-methoxyphenyl)ethene (2c, 1.0 g, 3.46 mmol), 4-(N-ethylamino)-N-methylphthalimide [26] (0.7 g, 3.47 mmol), Pd(OAc)₂ (40 mg, 0.18 mmol), tri-tert-butylphosphine (192 μL, 0.69 mmol), and NaO'Bu (0.4 g, 4.16 mmol) in anhydrous toluene (15 mL) were used to generate the crude product. The crude product was flash chromatographed on a silica gel (32-63 μm, 60 Å) column using 20% ethyl acetate in hexane as the eluent and the isolate obtained was recrystallized from a mixture of ethyl acetate and hexane to give the desired product 3c (279 mg, 0.67 mmol, 19% yield) as yellow sandy crystal: m.p. 122.1-122.7° C.; ¹H NMR (DMSO-d₆) δ 1.19 (t, J=7.0 Hz, 3H), 2.97 (s, 3H), 3.79 (s, 3H), 3.88 (q, J=7.0 Hz, 2H), 5.43 (s, 1H), 5.46 (s, 1H), 6.95-7.00 (m, 4H), 7.26-7.30 (m, 4H), 7.41 (d, J=8.6 Hz, 2H), 7.62 (d, J=8.3 Hz, 1H); Anal. C₂₆H₂₄N₂O₃, calcd. C, 75.71; H, 5.86; N, 6.79, found C, 75.97; H, 5.97; N, 6.84.

1-(4-n-Butoxyphenyl)-1-[4-N-ethyl-N-(N-methylphthalimid-4-yl)aminophenyl]ethene (3d) [25]. The same procedure described for the synthesis of 3a was employed except that 1-(4-bromophenyl)-1-(4-n-butoxyphenyl)ethene (2d, 1.0 g, 3.02 mmol), 4-(N-ethylamino)-N-methylphthalimide [26] (0.62 g, 3.02 mmol), Pd(OAc)₂ (34 mg, 0.15 mmol), tri-tert-butylphosphine (166 mL, 0.60 mmol), and NaO'Bu (0.35 g, 3.60 mmol) in anhydrous toluene (15 mL) were used to generate the crude product. The crude product was flash chromatographed on a silica gel (32-63 μm, 60 Å) column using 20% ethyl acetate in hexane as the eluent and the isolate obtained was recrystallized from a mixture of ethyl acetate and hexane to give the desired product 3d (603 mg, 1.33 mmol, 44% yield) as yellow crystal: m.p. 120.7-122.2° C.; ¹H NMR (DMSO-d₆) δ 0.94 (t, J=7.4 Hz, 3H), 1.19 (t, J=7.1 Hz, 3H), 1.44 (m, 2H), 1.72 (m, 2H), 2.97 (s, 3H), 3.88 (q, J=7.0 Hz, 2H), 3.99 (t, J=6.4 Hz, 2H), 5.42 (s, 1H), 5.45 (s, 1H), 6.93-7.01 (m, 4H), 7.24-7.29 (m, 4H), 7.41 (d, J=8.4 Hz, 2H), 7.62 (d, J=8.3 Hz, 1H); Anal. C₂₉H₃₀N₂O₃, calcd. C, 76.63; H, 6.65; N, 6.16, found C, 76.67; H, 6.74; N, 6.05.

1-(4-n-Butylphenyl)-1-[4-N-ethyl-N-(N-methylphthalimid-4-yl)-aminophenyl]ethene (3e) [25]. The same procedure described for the synthesis of 3a was employed except that 1-(4-bromophenyl)-1-(4-n-butylphenyl)ethene (2e, 1.0 g), 4-(N-ethylamino)-N-methylphthalimide [26] (0.66 g, 3.2 mmol), Pd(OAc)₂ (37 mg, 0.16 mmol), tri-tert-butylphosphine (182 μL, 0.66 mmol), and NaO′Bu (0.38 g, 3.95 mmol) in anhydrous toluene (15 mL) were used to generate the crude product. The crude product was flash chromatographed on a silica gel (32-63 μm, 60 Å) column using methylene chloride as the eluent to give 3e (522 mg) of yellow solids: m.p. 112.8-114.0° C.

1-(4-N,N-Dimethylaminophenyl)-1-[4-N-ethyl-N-(N,6-dimethyl-phthalimid-3-yl)-aminophenyl]ethene (3f). The same procedure described for the synthesis of 3a was employed except that 2a (1.38 g, 4.58 mmol), 3-(N-ethylamino)-N,6-dimethylphthalimide (1.00 g, 4.58 mmol), palladium acetate (51.5 mg, 0.23 mmol), tri-tert-butylphosphine (255 μL, 0.92 mmol), and sodium tert-butoxide (0.53 g, 5.50 mmol) in anhydrous toluene (15 mL) were used to generate the crude product. The crude product was flash chromatographed on a silica gel column using 20% ethyl acetate in hexane as eluent to give 3f (580 mg, 29%) as yellow solid: ¹H NMR (DMSO-d₆) δ 01.14 (t, J=7.1 Hz, 3H), 2.61 (s, 3H), 2.91 (s, 6H), 2.97 (s, 3H), 3.82(q, J=7.0 Hz, 2H), 5.12 (d, J=1.6 Hz, 1H), 5.15 (d, J=1.5 Hz, 1H), 6.69 (d, J=8.7 Hz, 2H), 6.72 (d, J=9.0 Hz, 2H), 7.12 (d, J=8.8 Hz, 2H), 7.13 (d, J=8.7 Hz, 2H), 7.41 (d, J=8.2 Hz, 1H), 7.55 (d, J=8.6 Hz, 1H).

N-Ethyl-N-(N-methylphthalimid-4-yl)-{4-[1,5-bis(4-N,N-dimethylaminophenyl)-5-(4-N-ethyl-N-(N-methylphthalimid-4-yl)aminophenyl)-2,4-pentadienylidene]-2,5-cyclohexadien-1-ylidene}ammonium perchlorate (4a). Upon stirring a mixture of 3a (0.85 g, 2.00 mmol) and triethyl orthoformate (0.355 g, 2.40 mmol) in acetic anhydride (4 mL) under an argon atmosphere at room temperature, 70% perchloric acid (0.172 g, 1.20 mmol) was added dropwise. The blue solution was refluxed for 90 minutes and cooled to room temperature. The product 4a was obtained from the resulting mixture by filtration and washed with tetrahydrofuran twice and air-dried to give a dark blue powder (0.84 g, 0.87 mmol, 87% yield): m.p. 197.9-200.3° C. (decomposed); HRMS (FAB, M⁺, C₅₅H₅₃N₆O₄⁺) calcd. 861.4128, found 861.4163; Anal. C₅₅H₅₃N₆O₈Cl.0.29 HClO₄.0.50H₂O, calcd. C, 66.10; H, 5.48; N, 8.41, Cl 4.57, found C, 66.07; H, 5.47; N, 8.20, Cl 4.57.

N-Ethyl-N-(N-methylphthalimid-4-yl)-{4-[1,5-diphenyl-5-(4-N-ethyl-N-(N-methylphthalimid-4-yl)aminophenyl)-2,4-pentadienylidene]-2,5-cyclohexadien-1-ylidene}ammonium tetrafluoroborate (4b). Upon stirring a light yellow solution of 3b (0.54 g, 1.41 mmol) and triethyl orthoformate (283 μL, 1.72 mmol) in acetic anhydride (4 mL) at room temperature under an argon atmosphere, an ethereal solution of tetrafluoroboric acid (54 wt. %, 99 μL, 0.705 mmol) was added with a micro syringe. The solution immediately changed its color to red followed by an intense blue color after being stirred for 30 minutes. The dark blue solution was heated at 90° C. for 2 hours and allowed to cool to room temperature. Diethyl ether was added to the solution and the precipitate was collected by filtration and air-dried to give 4b as dark blue solids (0.35 g, 0.406 mmol, 57% yield): HRMS (FAB, M⁺ C₅₁H₄₃N₄O₄⁺) calcd. 775.3284, found 775.3333.

N-Ethyl-N-(N-methylphthalimid-4-yl)-{4-[1,5-bis(4-methoxyphenyl)-5-(4-N-ethyl-N-(N-methylphthalimid-4-yl)aminophenyl)-2,4-pentadienylidene]-2,5-cyclohexadien-1-ylidene}ammonium tetrafluoroborate (4c). A solution of 3c (150 mg, 0.364 mmol) in acetic anhydride (3 mL) was warmed in a water bath under an argon atmosphere until the solids had dissolved. Upon stirring and cooling the mixture to room temperature, triethyl orthoformate (90 μL, 0.55 mmol) was added followed by the addition of an ethereal solution of tetrafluoroboric acid (54 wt. %, 30 μL, 0.22 mmol) through a micro syringe. The resulting dark red solution was heated at 80° C. under an argon atmosphere for 30 minutes and allowed to cool to room temperature. Diethyl ether was added to the resulting dark blue mixture and the mixture was stood overnight to precipitate the crude product. The dark blue precipitate was collected by filtration and washed with diethyl ether. Recrystallization of the solids from methylene chloride and diethyl ether gave 4c (126 mg) as black crystal: MS (FAB, M⁺, C₅₃H₄₇N₄O₆⁺) calcd. 836, found 836.

N-Ethyl-N-(N-methylphthalimid-4-yl)-{4-[1,5-bis(4-n-butoxyphenyl)-5-(4-N-ethyl-N-(N-methylphthalimid-4-yl)aminophenyl)-2,4-pentadienylidene]-2,5-cyclohexadien-1-ylidene}ammonium tetrafluoroborate (4d). A solution of 3d (139 mg, 0.306 mmol) in acetic anhydride (3 mL) was warmed in a water bath under an argon atmosphere until the solids had dissolved. Upon stirring and cooling the mixture to room temperature, triethyl orthoformate (75 μL, 0.46 mmol) was added followed by the addition of an ethereal solution of tetrafluoroboric acid (54 wt. %, 25 μL, 0.184 mmol) through a micro syringe. The resulting dark red solution was heated at 80° C. under an argon atmosphere for 30 minutes and allowed to cool to room temperature. Diethyl ether was added to the resulting dark blue mixture and the mixture was stood overnight to precipitate the crude product. The dark blue precipitate was collected by filtration and washed with diethyl ether. Recrystallization of the solids from methylene chloride and diethyl ether gave 4d (129 mg) as black crystal: MS (FAB, M⁺, C₅₉H₅₉N₄O₆⁺) calcd. 919, found 919.

N-Ethyl-N-(N-methylphthalimid-4-yl)-{4-[1,5-bis(4-n-butylphenyl)-5-(4-N-ethyl-N-(N-methylphthalimid-4-yl)aminophenyl)-2,4-pentadienylidene]-2,5-cyclohexadien-1-ylidene}ammonium tetrafluoroborate (4e). Upon stirring a solution of 3e (80 mg, 0.18 mmol) and triethyl orthoformate (36.7 μL, 0.22 mmol) in acetic anhydride (1 mL) at room temperature under an argon atmosphere, an ethereal solution of tetrafluoroboric acid (54 wt. %, 12.8 μL, 0.09 mmol) was added with a micro syringe. The stirring was continued for 30 minutes. The solution was then heated at 110° C. for 1 hour and allowed to cool to room temperature. Diethyl ether (100 mL) was added to the solution and the precipitate was collected by filtration and air-dried to give 4e as dark blue solids (47 mg): MS (FAB, M⁺ C₅₉H₅₉N₄O₄⁺) calcd. 887, found 887.

N-Ethyl-N-(N,6-dimethylphthalimid-3-yl)-{4-[1,5-bis(4-N,N-dimethylamino-phenyl)-5-(4-N-ethyl-N-(N,6-dimethyl-phthalimid-3-yl)aminophenyl)-2,4-penta-dienylidene]-2,5-cyclohexadien-t-ylidene}ammonium perchlorate (4f). Upon stirring a mixture of 3f (142 mg, 0.323 mmol) and triethyl orthoformate (64 μL, 0.388 mmol) in acetic anhydride (2 mL) under an argon atmosphere at room temperature, 70% perchloric acid (20 μL, 0.233 mmol) was added dropwise. The resulting blue solution was stirred at 100° C. for 50 min. and cooled to room temperature. Diethyl ether was added and the precipitate was collected by filtration. Recrystallization of the solid from methylene chloride-diethyl ether mixture gave 4f (135 mg, 84.5%) as violet crystalline: MS (FAB, M⁺, C₅₇H₅₇N₆O₄⁺) calcd. 889, found 889.

N-Ethyl-N-(2,3-dihydro-1,4-phthalazinedion-6-yl)-{4-[1,5-bis(4-N,N-dimethylaminophenyl)-5-(4-N-ethyl-N-(2,3-dihydro-1,4-phthalazinedion-6-yl)aminophenyl)-2,4-pentadienylidene]-2,5-cyclohexadien-1-ylidene}ammonium perchlorate (5a). A solution of 4a (520 mg, 0.54 mmol) and potassium hydroxide (56 mg, 1.00 mmol) in methanol (120 mL) was refluxed for 1 hour and then cooled to room temperature. Hydrazine (700 μL, 22 mmol) was added and oxygen was removed from the mixture via freeze-pump-thaw cycle three times followed by reflux under Ar for 2 hours. Methanol was removed from the mixture by distillation and the yellow solids deposited were dried under vacuum. The solids were dissolved in a mixture of THF and water and the solution was adjusted to pH 2 with 70% HClO₄. The precipitate was collected by filtration, rinsed with water, and dried under vacuum to give 5a (516 mg, 0.53 mmol, 98% yield) as a blue powder: HRMS (FAB, M⁺, C₅₃H₅₁N₈O₄⁺) calcd. 863.4033, found 863.3989.

N-Ethyl-N-(2,3-dihydro-1,4-phthalazinedion-6-yl)-{4-[1,5-diphenyl-5-(4-N-ethyl-N-(2,3-dihydro-1,4-phthalazinedion-6-yl)aminophenyl)-2,4-pentadienylidene]-2,5-cyclohexadien-1-ylidene}ammonium tetrafluoroborate (5b). A solution of 4b (250 mg, 0.29 mmol) and potassium hydroxide (50 mg, 0.89 mmol) in methanol (100 mL) was refluxed for 2 hours and a gray precipitate was formed. The mixture was cooled to room temperature and hydrazine (370 μL, 11.6 mmol) was added. Oxygen was removed from the mixture via freeze-pump-thaw cycle three times and the mixture was refluxed under Ar for 2 hours. Methanol was removed from the mixture by distillation and the residue was dried under vacuum. The solids obtained were stirred in a DMF solution (5 mL) containing HBF₄ ethereal solution (54 wt. %, 100 μL) and silica gel (3 g, 32-63 μm, 60 Å) for 15 minutes. The solvent was removed at 50° C. under reduced pressure and the resulting silica gel was subjected to column chromatography using an eluent of methylene chloride:ethanol:acetic acid (90:10:1, v/v). The desired product 5b was obtained as a brown powder: MS (FAB, M⁺, C₄₉H₄₁N₆O₄⁺) calcd. 777, found 777.

N-Ethyl-N-(2,3-dihydro-1,4-phthalazinedion-6-yl)-{4-[1,5-bis(4-methoxyphenyl)-5-(4-N-ethyl-N-(2,3-dihydro-1,4-phthalazinedion-6-yl)aminophenyl)-2,4-pentadienylidene]-2,5-cyclohexadien-1-ylidene}ammonium tetrafluoroborate (5c). A solution of 4c (120 mg, 0.128 mmol) and potassium hydroxide (15 mg, 0.26 mmol) in anhydrous ethanol (30 mL) was refluxed for 1 hour and then cooled to room temperature. Hydrazine (200 μL, 6.4 mmol) was added and oxygen was removed from the mixture via freeze-pump-thaw cycle three times followed by reflux under Ar for 2 hours. Ethanol was removed from the mixture by distillation and the yellow solids deposited were dried under vacuum. The solids were dissolved in a mixture of THF and water and the solution was adjusted to pH 2 with tetrafluoroboric acid (54 wt. % in diethyl ether). The precipitate was collected by filtration, rinsed with water, and dried under vacuum. The product was stirred with anhydrous THF (20 mL) for 5 hours, filtered, and dried under vacuum to give 5c (107 mg) as blue solids: MS (FAB, M⁺, C₅₁H₄₅N₆O₆⁺) calcd. 837, found 837.

N-Ethyl-N-(2,3-dihydro-1,4-phthalazinedion-6-yl)-{4-[1,5-bis(4-n-butoxyphenyl)-5-(4-N-ethyl-N-(2,3-dihydro-1,4-phthalazinedion-6-yl)aminophenyl)-2,4-pentadienylidene]-2,5-cyclohexadien-1-ylidene}ammonium tetrafluoroborate (5d). The same procedure described for the synthesis of 5c was employed except that starting with 133 mg (0.130 mmol) of 4d to generate 74 mg of 5d as light blue solids: MS (FAB, M⁺, C₅₇H₅₇N₆O₆⁺) calcd. 921, found 921.

N-Ethyl-N-(2,3-dihydro-1,4-phthalazinedion-6-yl)-{4-[1,5-bis(4-n-butylphenyl)-5-(4-N-ethyl-N-(2,3-dihydro-1,4-phthalazinedion-6-yl)aminophenyl)-2,4-pentadienylidene]-2,5-cyclohexadien-1-ylidene}ammonium tetrafluoroborate (5e). To a solution of 4e (100 mg, 0.103 mmol) in 25 mL of anhydrous ethanol, hydrazine (131 μL, 4.1 mmol) was added and oxygen was removed from the mixture via freeze-pump-thaw cycle three times followed by reflux under argon atmosphere for 2 hours. Solvent was removed from the mixture under reduced pressure and the solids deposited was redissolved in 30 mL of anhydrous THF. Upon stirring the solution at room temperature, an ethereal solution of tetrafluoroboric acid (54 wt. %, 150 μL) was added and the stirring was continued at room temperature for 6 hours. Diethyl ether (200 mL) was added to the solution and the precipitate was collected by filtration and air-dried to give 5e as dark blue solids (70 mg): MS (FAB, M⁺ C₅₇H₅₇N₆O₄⁺) calcd. 889, found 889.

N-Ethyl-N-(8-methyl-2,3-dihydro-1,4-phthalazinedion-5-yl)-{4-[1,5-bis(4-N,N-dimethylamino-phenyl)-5-(4-N-ethyl-N-(8-methyl-2,3-dihydro-1,4-phthalazinedion-5-yl)aminophenyl)-2,4-penta-dienylidene]-2,5-cyclohexadien-1-ylidene} ammonium perchlorate (5f). To a dark blue solution of 4f (95.5 mg, 0.0965 mmol) in methylene chloride (5 mL) under stirring, potassium hydroxide in anhydrous ethanol (0.303 M, 0.520 mL, 0.158 mmol) was added dropwise until the dark blue faded to brown. The volatiles were evaporated under reduced pressure and anhydrous ethanol (30 mL) and anhydrous hydrazine (0.16 mL, 5.0 mmol) were added. The mixture was degassed via freeze-pump-thaw cycle three times and then refluxed under argon for 2 h. The volatiles were evaporated under reduced pressure and the orange residue was dissolved in THF—H₂O mixture (5:1 v/v, 6 mL). It was acidified with perchloric acid (0.1 M, 3 mL) and was evaporated under reduced pressure to remove THF. The precipitate was collected by filtration, rinsed with water, dried in vacuum. It was stirred with methylene chloride (3 mL), filtered and dried to give 5f (89 mg, 93%) as blue solid: MS (FAB, M⁺, C₅₅H₅₅N₈O₄⁺) calcd. 891, found 891.

Preparation of prodrug 6a. A mixture of 2′,3′-dideoxycytidine (19.2 mg, 0.091 mmol), sodium hydride (60% in mineral oil, 3.7 mg, 0.093 mmol), and freshly distilled DMSO (4.5 mL) were stirred under argon at room temperature for 2 hours. An aliquot (1.84 mL) of this resulting clear solution was added via a syringe to a solution containing 29.8 mg (0.0308 mmol) of 5a and 0.35 mL of freshly distilled DMSO in an argon atmosphere. The resulting mixture was stirred under the same atmosphere at room temperature for 2 hours yielding a dark green solution of 6a (0.0141 M). This solution was used for the in vitro test against HIV without further purification.

Preparation of prodrug 6b. The same procedure described for the preparation of 6a was employed to prepare a 2 mL of 0.0103 M DMSO solution of 6b. This solution was used for the in vitro test against HIV without further purification.

Preparation of prodrug 7a. A mixture of 5a (20.9 mg, 0.0217 mmol) and phosphonoformic acid trisodium salt hexahydrate (6.6 mg, 0.022 mmol) was stirred at room temperature under argon overnight in 2.0 mL of freshly distilled DMSO to give a light yellow solution of 7a (0.0109 M). This solution was used for the in vitro test against HIV without further purification.

Preparation of prodrug 8a. A mixture of acycloguanosine (40.7 mg, 0.181 mmol) and sodium hydride (60% in mineral oil, 10.5 mg, 0.263 mmol) was stirred for 3 hours at room temperature under argon in 4.5 mL of freshly distilled DMSO. An aliquot (1.126 mL) of this resulting clear solution was added via a syringe to a blue solution containing 49.1 mg (0.0510 mmol) of 5a and 0.874 mL of freshly distilled DMSO in an argon atmosphere. The resulting mixture was stirred under the same atmosphere at room temperature for 2 hours yielding a light yellow solution of 8a (0.0255 M). This solution was used for the in vitro test against HSV without further purification.

For the second type of luminides, such as MTLJ-1-Foscarnet in TABLE 5, that the chemiluminescent functionality luminols are attached to the aryl groups of a photochromic dye in TABLE 2 through molecular linkers, their corresponding carriers, such as YY99811-1, MTLJ-1 in TABLE 5, can be obtained by following Scheme II in addition to the general procedure disclosed previously [Mills, R. L. U.S. Pat. No. 5,773,592]:

A protected aminophthalhydrazide such as aminophthalimide or aminophthalic acid diester is attached through a proper molecular linker to the aryl groups of diarylketene, forming the key precursor aminophthalimide-linked diarylketene such as 12a-d for the dye such as 19a-d. Thus, as examples, the classical Friedel-Crafts acylation between benzoyl halide and 2-bromoethoxybenzene gives 4-(2-bromoethoxy)benzophenone 10. It is converted to the corresponding diarylketene 11 by reacting with methylmagnesium bromide and then dehydration with acid. Then the 1-(4-(2-bromoethoxy)phenyl-1-arylethene couples with the 4-ethylaminophthalimide to form 1-(4-(2-(phthalimide-3-amino)ethoxy)phenyl)-1-arylethene 12. Alternatively 12 can be prepared by coupling 4-(2-bromoethylamino)phthalimide 14 with sodium 4-(1-arylethenyl)phenoxide 17.

Condensation of the aminophthalimide-linked diarylketene such as 12a-d with an orthoester such as triethylorthoformate yields the aminophthalimide-linked tetraarylpolymethine dye such as 19a-d. Then the phthalimide moiety of the dye is converted to the phthalhydrazide by treating with hydrazine, forming the carrier 20a-d as follows: The cationic dyes such as 20a-d are first protected by reacting with an anion such as hydroxide, methoxide and amine, refluxed with hydrazine in a suitable solvent such as an alcoholic solvent in inert atmosphere and then treated with acid such as perchloric acid, tetrafluroboric acid to regenerate the cationic carriers.

Reacting such a carrier with one nucleophilic species of a drug such as foscarnet gives the luminide prodrug such as YY99811-1.

Alternatively the carrier such as 20a-d can also be made by condensing triethylorthoformate with aminophthalhydrazide-linked diarylketene such as 18a-d which can be made from the corresponding aminophthalimide-linked diarylketene such as 12a-d by treating with hydrazine.

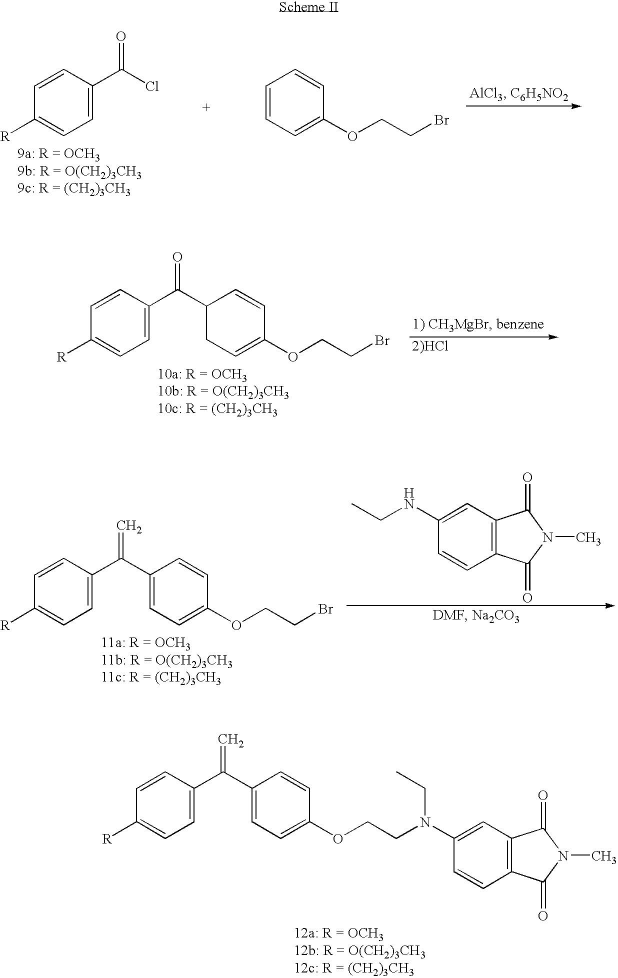

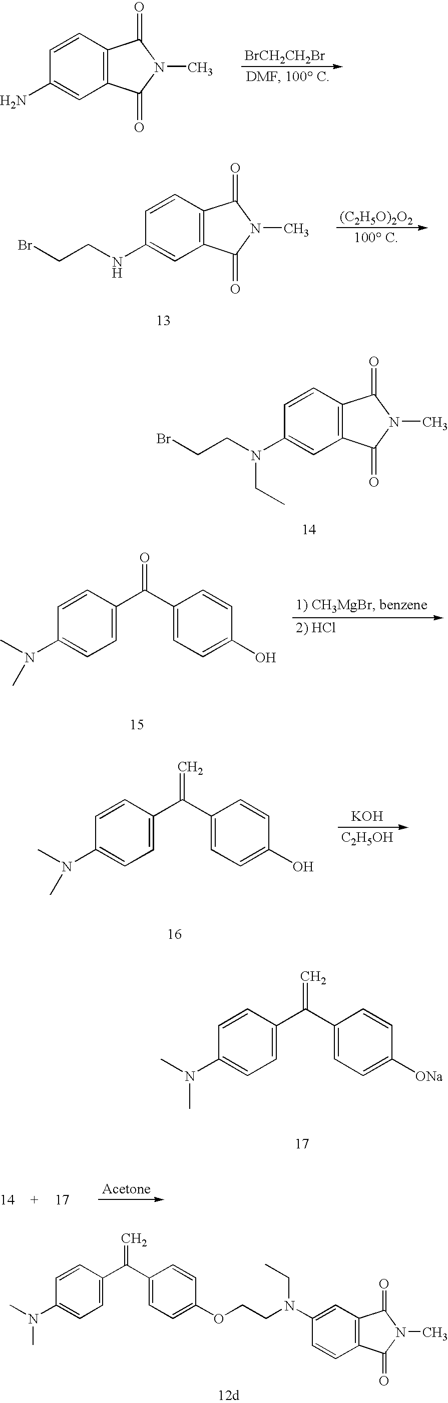

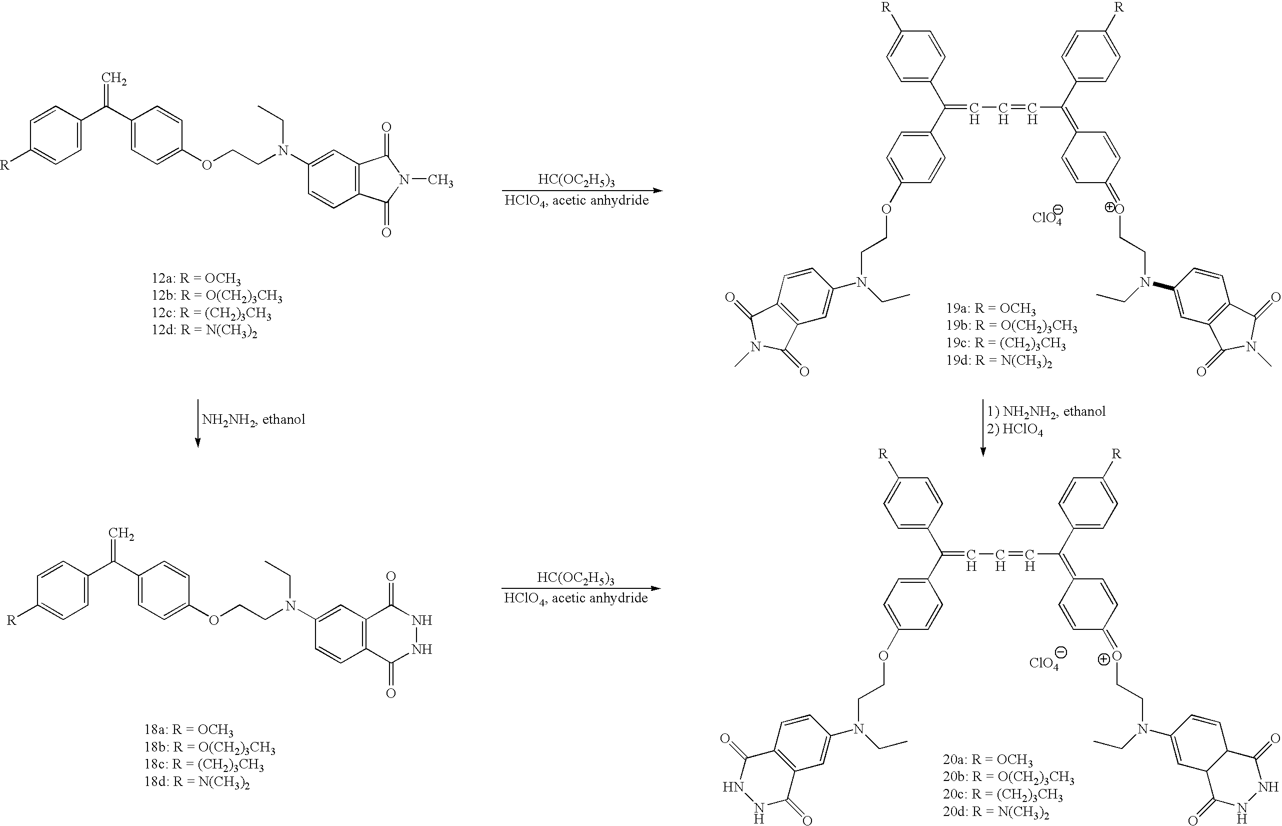

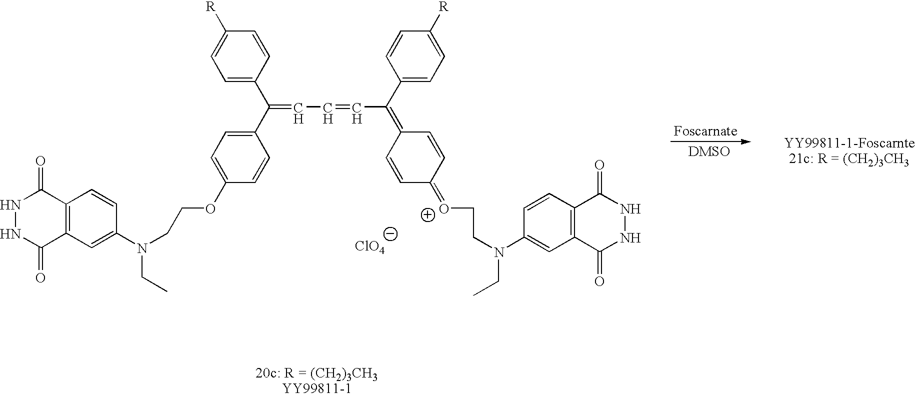

4-Methoxy-4′-(2-bromoethoxy)benzophenone (10a). Upon stirring a solution of p-anisoyl chloride (8.53 g, 50 mmol) and β-bromophenetole (10.05 g, 50 mmol) in 20 mL of anhydrous nitrobenzene at 5° C. under a nitrogen atmosphere, 7.33 g (55 mmol) of anhydrous aluminum chloride was added portionwise. The resulting mixture was stirred at room temperature for 1 hour then at a reduced pressure for 5 minutes to remove the HCl gas produced during the reaction. Stirring was continued at room temperature under the nitrogen atmosphere for another hour. Nitrobenzene was removed at 45° C./0.4 mmHg and the solids deposited were dissolved in 60 mL of chloroform. Upon stirring the solution in an ice bath, 25 mL of 2M HCl was added portionwise. The organic layer was separated and washed with 20 mL of saturated sodium hydrogencarbonate aqueous solution, dried over solid NaHCO₃, and filtered through a filter paper. Solvent was removed from the filtrate using a rotary evaporator and the crude product was purified by silica gel column chromatography (particle size 32-63) in CHCl₃. The product with R_{f}=0.26 was collected and recrystallized from chloroform/hexane to give 14.44 g (43 mmol, 86% yield) of white flakes. m.p. 112.2-113.3° C. E.A. C₁₆H₁₅BrO₃, calculated C 57.32, H 4.52, Br 23.83; found C 57.40, H 4.55, Br 23.76. ¹H-NMR (DMSO-d₆) δ 3.86 (m, 5H, —OCH₃, —OCH₂CH₂Br), 4.44 (t, 2H, J=5.40, 5.04 Hz, —OCH₂CH₂Br), 7.08 (d, 2H, J=8.64 Hz, aromatic H's), 7.11 (d, 2H, J=8.64 Hz, aromatic H's), 7.71 (d, 2H, J=2.52 Hz, aromatic H's), 7.73 (d, 2H, J=2.52 Hz, aromatic H's). ¹³C-NMR (DMSO-d₆) 31.17, 55.51, 67.97, 113.75, 114.33, 129.88, 130.44, 131.84, 161.11, 162.54, 193.12.

4-Butoxy-4′-(2-bromoethoxy)benzophenone (10b). Ketone 10b was prepared by the same proceudre as described for ketone 10a to give 7.17 g (19 mmol, 80% yield) of white flakes by using 5 g (23.5 mmol) of 4-butoxybenzoyl chloride, 4.72 g (23.5 mmol) of β-bromophenetole, 10 mL of anhydrous nitrobenzne, and 3.13 g (23.5 mmol) of anhydrous AlCl₃. m.p. 106.8-107.8° C. E.A. C₁₉H₂₁BrO₃, calculated C 60.48, H 5.62, Br 21.77; found C 60.72, H 5.71, Br 21.88. ¹H-NMR (DMSO-d₆) δ 0.94 (t, 3H, J=7.20, 7.20 Hz, —OCH₂CH₂CH₂CH₃), 1.45 (h, 2H, J=7.56, 7.20, 7.20, 7.56, 7.20 Hz, —OCH₂CH₂CH₂CH₃), 1.73 (p, 2H, J=6.84, 7.20, 7.20, 6.48 Hz, —OCH₂CH₂CH₂CH₃), 3.85 (t, 2H, J=5.40, 5.04, —OCH₂CH₂Br), 4.06 (t, 2H, J=6.48, 6.48, —OCH₂CH₂CH₂CH₃), 4.44 (t, 2H, J=5.04, 5.04, —OCH₂CH₂Br), 7.06 (d, 2H, J=8.28 Hz, aromatic H's), 7.11 (d, 2H, J=8.28 Hz, aromatic H's), 7.69 (m, 4H, aromatic H's). ¹³C-NMR (DMSO-d₆) δ 13.69, 18.72, 30.60, 31.18, 67.47, 67.93, 114.02, 114.18, 129.57, 130.32, 131.67, 160.90, 161.84, 192.82.

1-(4-Methoxyphenyl)-1-[4-(2-bromoethoxyphenyl)]ethene (11a). Upon stirring a suspension of 14.28 g (0.042 mol) of ketone (10a) in 50 mL of benzene in an ice bath under a nitrogen atmosphere, 17 mL (0.051 mol) of a 3 molar ethereal solution of methylmagnesium bromide was added via a syringe. The resulting mixture was stirred at room temperature for 17 hours under the nitrogen atmosphere. The mixture was cooled in an ice bath while 25 mL of 2M HCl solution was added portionwise. The color of the mixture changed from light gray to orange and finally to pale yellow during the addition of the HCl solution. The final solution was stirred at room temperature for 5 minutes. The organic layer was separated and the aqueous layer was extracted with 50 mL of ether. The combined extract and organic layer was washed with 50 mL of saturated NaHCO₃ aqueous solution and dried over anhydrous MgSO₄ and then filtered through a filter paper. Solvent was removed from the filtrate using a rotory evaporator and the crude yellow solids deposited contained two major products with R_{f} values of 0.61 and 0.14 by thin layer chromatography in CHCl₃. This mixture was separated by silica gel column chromatography (particle size 32-63) in CHCl₃. The first product, R_{f}=0.61, collected from the column was recrystallized from a solution of chloroform and hexanes to give 6.29 g (0.018 mol, 45% yield) of pale pink solids. m.p. 104.4-105.9° C. E.A. C₁₇H₁₇BrO₂, calculated C 61.27, H 5.15, Br 23.98; found C 61.09, H 5.11, Br 23.80. ¹H-NMR (DMSO-d₆) δ 3.77 (s, 3H, —OCH₃), 3.81 (t, 2H, J=5.76, 5.04 Hz, —OCH₂CH₂Br), 4.34 (t, 2H, J=5.40, 5.40 Hz, —OCH₂CH₂Br), 5.30 (s, 2H, ethylene H's), 6.93 (d, 2H, J=9.00 Hz, aromatic H's), 6.96 (d, 2H, J=9.00 Hz, aromatic H's), 7.21 (d, 2H, J=2.16 Hz, aromatic H's), 7.24 (d, 2H, J=1.80 Hz, aromatic H's). ¹³C-NMR (DMSO-d₆) δ 31.41, 55.09, 67.75, 111.87, 113.66, 114.37, 129.05, 129.11, 133.27, 133.88, 148.11, 157.65, 159.00. The second product, R_{f}=0.14, collected from the column was refluxed in 100 mL of benzene with the presence of catalytic amount of p-toluenesulfonic acid for 30 minutes. After cooling to room temperature, the solution was washed with 50 mL of saturated NaHCO₃ aqueous solution, dried over anhydrous MgSO₄, and filtered through a filter paper. Solvent was removed from the filtrate using a rotary evaporator and the product deposited was recrystallized from a solution of benzene and hexane to give 5.55 g (0.016 mol, 39% yield) of light green crystals. m.p. 104.4-105.9° C. Total yield of the ethylene product (11) was 11.84 g (0.035 mol, 84%).

1-(4-Butoxyphenyl)-1-[4-(2-bromoethoxyphenyl)] ethene (11b). Upon stirring a suspension of 7.13 g (0.019 mol) of ketone (10b) in 50 mL of benzene in an ice bath under a nitrogen atmosphere, 8 mL (0.024 mol) of a 3 molar ethereal solution of methylmagnesium bromide was added via a syringe. The resulting mixture was refluxed under the nitrogen atmosphere for 1 hour. The mixture was cooled in an ice bath while 30 mL of 2M HCl solution was added portionwise. The color of the mixture changed from light gray to orange and finally to colorless during the addition of the HCl solution. The final solution was stirred at room temperature for 10 minutes. The organic layer was separated and the aqueous layer was extracted with 50 mL of ether. The combined extract and organic layer was dried over anhydrous MgSO₄ overnight and then filtered through a filter paper. Solvent was removed from the filtrate using a rotory evaporator and the crude solids deposited contained one product with R_{f} values of 0.70 determined by thin layer chromatography in CHCl₃. This crude product was recrystallized from a mixture of chloroform and hexanes to give 6.60 g (0.017 mol, 93% yield) of pale pink solids. m.p. 107.8-108.7° C. E.A. C₂₀H₂₃BrO₂, calculated C 64.00, H 6.19, Br 21.29; found C 64.10, H 6.16, Br 21.33. ¹H-NMR (DMSO-d₆) δ 0.94 (t, 3H, J=7.56, 7.56 Hz, —OCH₂CH₂CH₂CH₃), 1.44 (h, 2H, J=7.56, 7.20, 7.56, 7.56, 7.56 Hz, —OCH₂CH₂CH₂CH₃), 1.70 (p, 2H, J=6.12, 7.92, 6.84, 6.48 Hz, —OCH₂CH₂CH₂CH₃), 3.81 (t, 2H, J=5.04, 6.12 Hz, —OCH₂CH₂Br), 3.97 (t, 2H, J=6.12, 6.84 Hz, —OCH₂CH₂CH₂CH₃), 4.34 (t, 2H, J=5.40, 5.04 Hz, —OCH₂CH₂Br), 5.29 (d, 2H, J=2.52 Hz, ethylene H's), 6.91 (d, 2H, J=8.64 Hz, aromatic H's), 6.95 (d, 2H, J=8.64 Hz, aromatic H's), 7.21 (t, 4H, J=8.64, 7.92 Hz, aromatic H's). ¹³C-NMR (DMSO-d₆) δ 13.75, 18.79, 30.77, 31.48, 67.09, 67.71, 111.71, 114.05, 114.26, 128.89, 129.00, 132.97, 133.76, 147.96, 157.47, 158.28.

1-(4-Butylphenyl)-1-[4-(2-bromoethoxyphenyl)]-ethene (11c). The ethene 1c was prepared from ketone 10c by the same procedure as described for ethene 11a. Anal. C₂₀H₂₃BrO, cald. C 66.85, H 6.46, Br 22.24; found C 66.91, H 6.45, Br 22.12. ¹H-NMR (DMSO-d₆) δ 0.90 (t, J=7.3 Hz, 3H), 1.31 (dt, J=7.4, 7.3 Hz, 2H), 1.56 (dd, J=7.7, 7.7 Hz, 2H), 2.58 (t, J=7.7 Hz, 2H), 3.81(t, J=5.5 Hz, 2H), 4.34 (t, J=5.4 Hz, 2H), 5.34 (s, 1H), 5.36 (s, 1H), 6.96 (d, J=8.7 Hz, 2H), 7.16-7.20 (m, 4H), 7.22 (d, J=9.0 Hz, 2H). ¹³C-NMR (DMSO-d₆) δ 67.7, 112.6, 114.4, 127.7, 128.2, 129.1, 133.7, 138.3, 142.0, 148.5, 157.6. MS (FAB) m/z 358, 360.

4-{N-Ethyl-N-[2-(4-((4-butylphenyl)ethenyl)phenoxy)-ethyl]amino}-N-methylphthalimide (12c). A mixture of 1c (1.3 g, 3.6 mmol) and 4-(N-ethylamino)-N-methylphthalimide (1.0 g, 4.9 mmol) in anhydrous DMF (10 mL) was refluxed under argon for 24 h. The solvent was evaporated under reduced pressure and the residue was dried, chromatographed on silica gel column using hexane-ether (20:1) to give the desired product 12c. ¹H-NMR (DMSO-d₆) δ 0.90 (t, J=7.3 Hz, 3H), 1.17 (t, J=7.0 Hz, 3H), 1.31 (dt, J=7.5, 7.5 Hz, 2H), 1.55 (dd, J=7.6, 7.6 Hz, 2H), 2.58 (t, J=7.7 Hz, 2H), 2.97 (s, 3H), 3.60 (q, J=7.0 Hz, 2H), 3.87 (t, J=5.4 Hz, 2H), 4.19 (t, J=5.4 Hz, 2H), 5.32 (s, 1H), □□□□□□s, 1H), 6.91 (d, J=8.7 Hz, 2H), 7.01 (dd, J=8.5, 2.4 Hz, 1H), 7.13 (d, J=2.4 Hz, 1H), 7.17 (s, 4H), □□□□□□d, J=8.7 Hz, 2H), 7.59 (d, J=8.6 Hz, 1H).

4-{N-Ethyl-N-[2-(4-((4-N,N-dimethylaminophenyl)-ethenyl)phenoxy)ethyl]amino}-N-methylphthalimide (12d). 4-(2-Bromoethyl-ethylamino)-N-methylphthalimide (14), 60 mg (0.19 mmol) and 17, 70.5 mg (0.25 mmol) were dissolved in anhydrous acetone (6 mL) and stirred at room temperature under argon for 14 h. After removing the solvent, the residue was separated on silica gel column using 15%(v/v) EtOAc in hexane to give the desired product 12 (C₂₉H₃₁N₃O₃). ¹H NMR (CDCl₃) δ 1.20 (m, 3H), 2.90 (s, 6H), 3.06 (s, 3H), 3.54 (q, 2H), 3.78 (t, 2H), 4.11 (t, 2H), 5.18 (s, 1H), 5.21 (s, 1H), 6.66 (d, 2H), 6.77 (m, 3H), 7.07 (s, 1H), 7.13-7.23 (m, 4H), 7.60 (d, 1H).

4-(2-Bromoethylamino)-N-methylphthalimide (13). 4-Amino-N-methylphthalimide (1.0 g, 5.78 mmol) and 1,2-dibromoethane (2.17 mL, 25.2 mmol) were dissolved in anhydrous DMF (15 mL). The resulting reaction mixture was heated at 100° C. under argon for 12 h. After cooling to room temperature, the mixture was treated with 5 ml of saturated sodium bicarbonate and 50 mL of deionized water. Then it was extracted with EtOAc (30 mL) twice. The organic layers were combined and washed with brine, dried over MgSO₄, concentrated, chromatographed on silica gel column using 30%(v/v) of EtOAc in hexane to give 13 (0.21 g, 13%): m.p. 212-214° C.; ¹H NMR (CDCl₃) δ 3.11 (s, 3H), 3.58 (t, 2H), 3.69 (t, 2H), 4.87 (broad, 1H), 6.75 (d, 1H), 7.00 (s, 1H), 7.60 (d, 1H). Anal. C₁₁H₁₁BrN₂O₂, calcd. C 46,67, H 3.92, N 9.89, found C 46.48, H 3.97, N 9.83.

4-(2-Bromoethyl-ethylamino)-N-methylphthalimide (14). Diethylsulfate (1 mL) and 2 (50 mg, 0.18 mmol) were heated at 110° C. uner argon for 5 h. The excess diethylsulfate was removed by vacuum evaporation. The residue was treated with saturated sodium solution carbonate, extracted with EtOAc, purified on silica gel column using EtOac-hexane (1:4) mixture and recrystalized from CHCl₃-hexane(1:10) to give desired product 14 (43 mg, 78%): m.p. 136-137° C.; Anal. C₁₃H₁₅BrN₂O₂, calcd. C, 50.18; H, 4.86; N, 9.00, found C 49.96, H 4.81, N 8.89.

1-(4-hydroxyphenyl)-1-(4-dimethylaminophenyl)ethane (16). 4-Hydroxy-4′-dimethylaminobenzophenone (2.0 g, 8.37 mmol) was dissolved in anhydrous benzene (30 mL) under argon. To the mixture methylmagnesium bromide in diethyl ether (3.0 M, 5.6 mL, 16.8 mmol) was added dropwise with a syringe. The resulting mixture was refluxed for 3 h and then cooled to 60° C. Saturated NH₄Cl aqueous solution was carefully added until the mixture was neutral (pH 7). The mixture was heated at 70° C. for 1 h and cooled to room temperature. The crude product was extracted from the mixture with EtOAc (30 mL) twice and purified on silica gel column using EtOac-hexane (1:5) to give the desired product 16 (1.15 g, 58%).

1-(4-hydroxyphenyl)-1-(4-dimethylaminophenyl) ethane sodium salt (17). Potassium hydroxide (77 mg, 1.37 mmol) was dissolved in EtOH (12 mL) under argon. To it 1-(4-hydroxyphenyl)-1-(4-dimethylaminophenyl)ethane, 16 (325 mg, 1.37 mmol) was added. The mixture was stirred at room temperature for 14 h. Evaporation of the solvent under vacuum gave the desired product 17.

4-{N-Ethyl-N-[2-(4-((4-butylphenyl)ethenyl) phenoxy)ethyl]amino}-N-methylphthalhydrazide (18c). To a solution of 12c (0.859 g, 1.8 mmol) in anhydrous ethanol (15 mL) under argon, anhydrous hydrazine (1.07 g, 34 mmol) was added through syringe. The resulting mixture was refluxed under argon for 4 h. The volatiles were evaporated under reduced pressure. The residue was chromatographed on solica gel column using diethyl ether to give desired product 18c. ¹H-NMR (DMSO-d₆) δ 0.90 (t, J=7.3 Hz, 3H), 1.18 (t, J=6.8 Hz, 3H), 1.31 (dt, J=7.5, 7.5 Hz, 2H), 1.55 (dd, J=7.6, 7.6 Hz, 2H), 2.58 (t, J=7.6 Hz, 2H), 3.59 (q, J=6.9 Hz, 2H), 3.86 (t, J=5.4 Hz, 2H), 4.20 (t, J=5.4 Hz, 2H), 5.32 (s, 1H), 5.34 (s, 1H), 6.93 (d, J=8.8 Hz; 2H), 7.17 (s, 4H), 7.19 (d, J=8.7 Hz, 2H), 7.20 (d, J=2.0 Hz, 1H), 7.28 (dd, J=9.1, 2.5 Hz, 1H), 7.87 (d, J=9.0 Hz, 1H). MS (FAB) m/z 484(MH⁺).

YY99811-1 (20c). Compound 18c (0.483 g, 1.00 mmol) was dissolved in a mixture of acetic acid (1.0 mL) and acetic anhydride (0.4 mL) by heating. The mixture was cooled and ethyl orthoformate (0.2 mL), then a solution of perchloric acid (70%) (0.0707 g) in acetic acid-acetic anhydride mixture (1.0 mL, 1:1) were added. The reaction mixture turned green immediately. It was stirred at room temperature for 48 h and precipitated with diethyl ether. The solid was collected and washed with ether again, dried to give 20c (C₆₁H₆₅N₆O₆.ClO₄). MS (FAB) m/z 977(M⁺).

YY99811-1-Foscarnate (21c). This prodrug was prepared from 20c by following the procedure for preparation of prodrug 7a.

A representative scheme of the first type of luminides synthesis wherein the dyes are the multiarylpolymethines is given in TABLE 6, Scheme Im. Other protecting forms of aminophthalhydrazide such as aminophthalic acid diester can be used instead of the aminophthalimide in the following procedures. Following the general procedure for making multiaryploymethine dyes in literature [Mills, R. L. U.S. Pat. No. 5,773,592, Appendix II, Method I], the carriers of the luminol-multiaryl-polymethine luminides can be obtained as follows: 1.) The key precursor for the dye, the aminophthalimide-substituted 1,1-diarylethene such as 3a-f is obtained as described in Scheme I. 2.) Condensing the ethene with a p-aminophenyl alkene aldehyde such as p-(dimethylamino)-cinnamaldehyde in a nonaqueous solvent such as acetic anhydride, containing an acid catalyst such as perchloric acid, tetrafluoroboric acid, to form the aminophthalimide-substituted multiarylpolymethine dye such as 22a-f. 3.) Converting the aminophthalimide moiety to the aminophthalhydrazide to obtain the carrier, such as 23a-f. The cationic dyes such as 22a-f are first protected by reacting with an anion such as hydroxide, methoxide and amine, refluxed with hydrazine in a suitable solvent such as an alcoholic solvent in inert atmosphere and then treated with acid such as perchloric acid, tetrafluroboric acid to regenerate the cationic carriers. 4.) Reacting the carrier with one nucleophilic species of a drug such as 2′,3′-dideoxycytidine, foscarnet, acycloguanosine to form the luminide prodrug, such as 24a-f.

Alternately the carriers of the luminol-multiarylpolymethine luminides, such as 23a-f, can be synthesized as follows: By starting with halo-substituted diarylketene precursor compounds such as 2a-e, proper halo-substituted multiarylpolymethine dyes, such as 1-(p-bromophenyl)-1,5-bis(p-dimethylaminophenyl)-pentadienium perchlorate, can be prepared by condensation with a p-aminophenyl alkene aldehyde such as p-(dimethylamino)cinnamaldehyde. The cationic dyes are protected by reacting with an anion such as alkoxide and then coupled with the aminophthalimide by amination of aryl halide such as the palladium-catalyzed amination of aryl halide to obtain the alkoxide-protecting aminophthalimide-substituted multiarylpolymethine dyes, such as alkoxide-protecting 22a-f. The protected dyes are refluxed with hydrazine in a suitable solvent such as an alcoholic solvent to convert the amino-phthalimide moiety to the aminophthalhydrazide moiety and then treated with acid to generate the carriers, such as 23a-f.

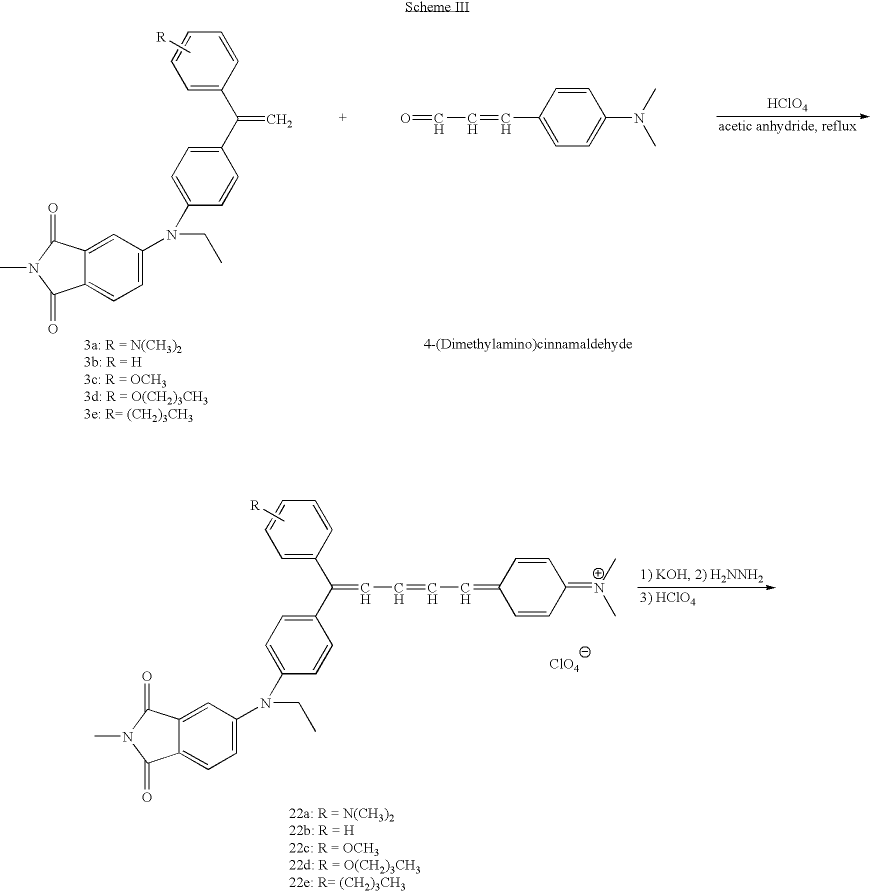

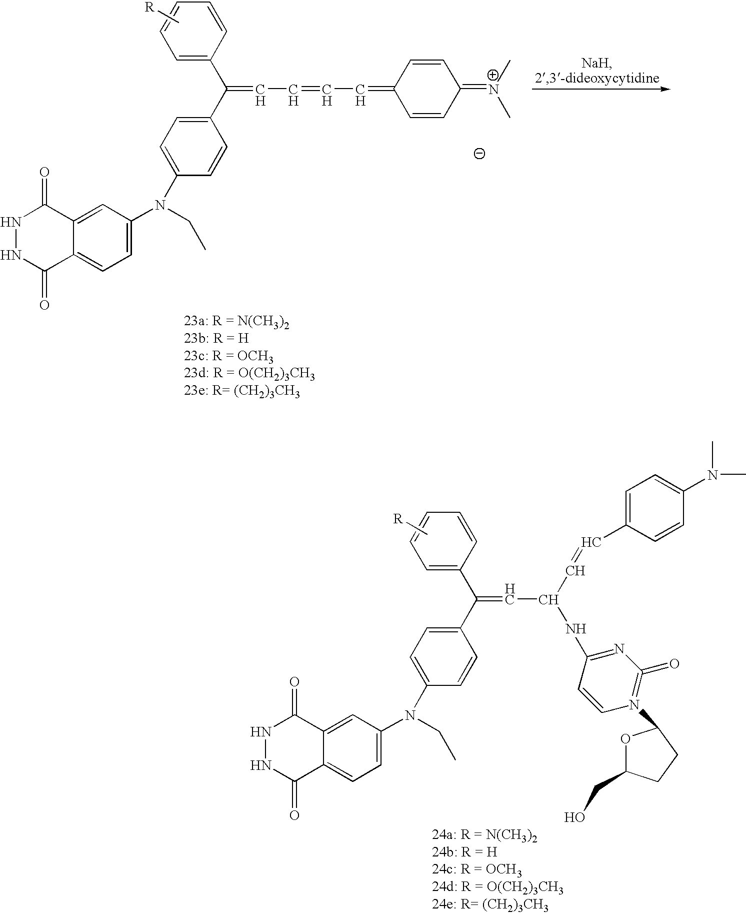

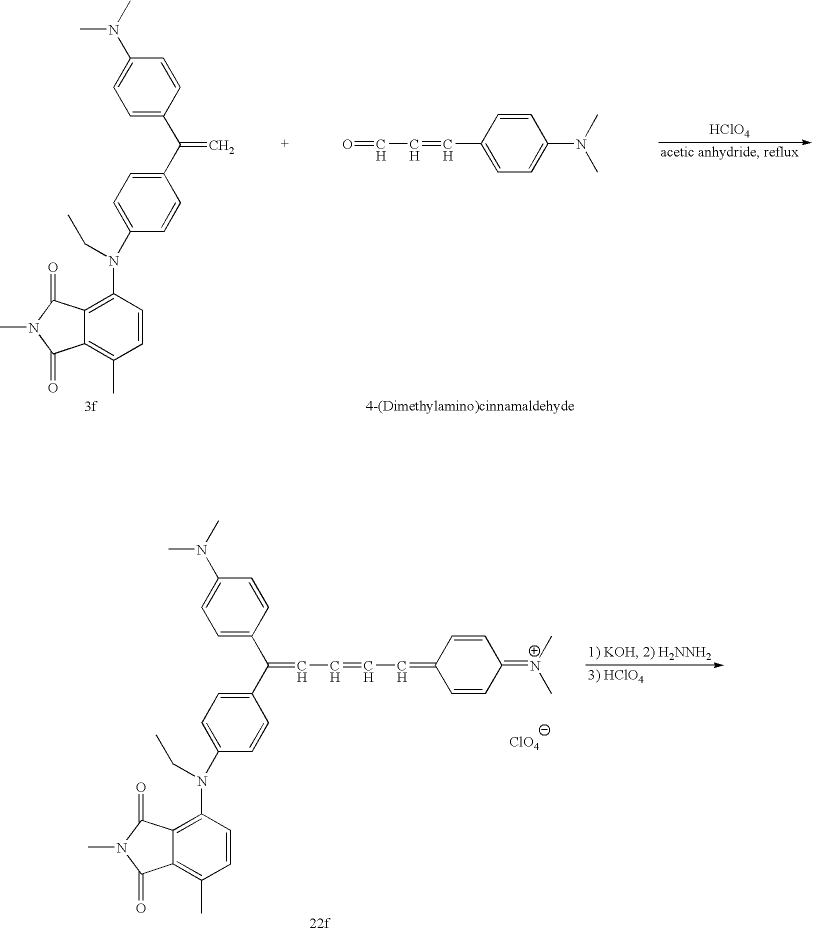

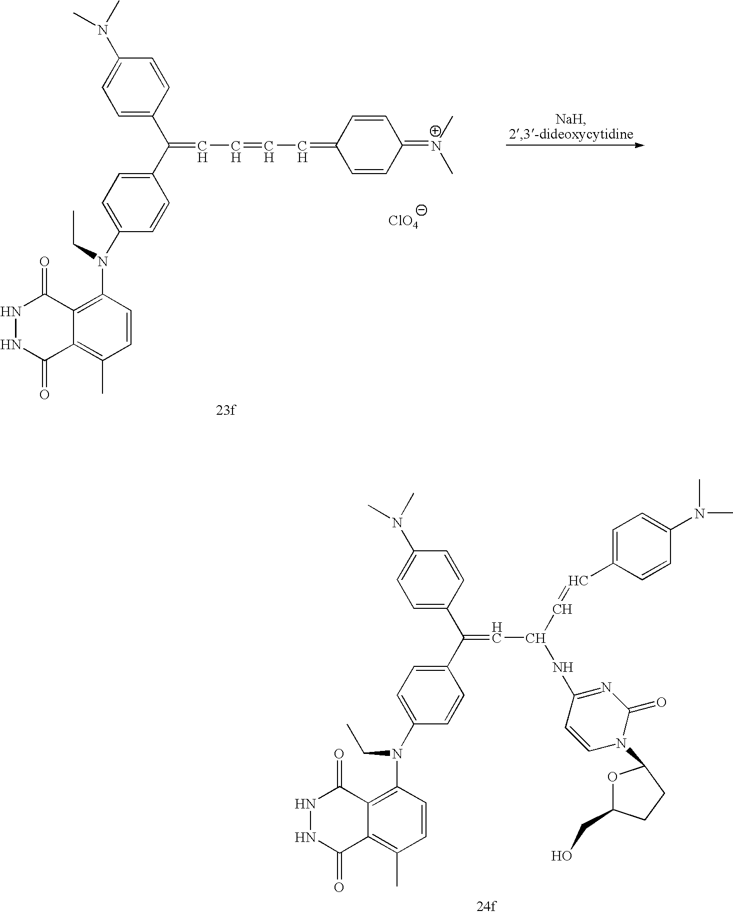

N,N-Dimethyl-{4-[5-(4-N,N-dimethylaminophenyl)-5-(4-N-ethyl-N-(N-methylphthalimid-4-yl)aminophenyl)-2,4-pentadienylidene]-2,5-cyclohexadien-1-ylidene}ammonium perchlorate (22a). 1-(4-N,N-Dimethylaminophenyl)-1-[4-N-ethyl-N-(N-methylphthalimid-4-yl)aminophenyl]ethene (3a), 500 mg (1.18 mmol), and 4-(dimethylamino)cinnamaldehyde, 206 mg (1.18 mmol), were dissolved in 12 mL of warm acetic anhydride. After cooling, 103 μL of perchloric acid (70%) in 8 mL acetic anhydride was added dropwise in 20 min. under argon. The resulting mixture was stirred at 60° C. for 30 min. It was cooled to room temperature and diethyl ether was added to precipitate the product. The precipitate was filtered and recrystalized in methylene chloride-ethyl acetate to give a black solid 22a, 479 mg (60%). UV-V is (CH₂Cl₂) λ, nm (relative F): 395 (0.206), 620 (0.200), 718 (0.412), 818 (1.00). MS (FAB, M⁺, C₃₈H₃₉N₄O₂⁺) calcd. 583, found 583.

N,N-Dimethyl-{4-[5-(4-N,N-dimethylaminophenyl)-5-(4-N-ethyl-N—(N,6-dimethylphthalimid-3-yl)aminophenyl)-2,4-pentadienylidene-2,5-cyclohexadien-1-ylidene}ammonium perchlorate (22f). 1-(4-N,N-Dimethylaminophenyl)-1-[4-N-ethyl-N—(N,6-dimethylphthalimid-3-yl)aminophenyl]ethene (3f), 200 mg (0.455 mmol), and 4-(dimethylamino)cinnamaldehyde, 96 mg (0.546 mmol), were dissolved in 2 mL of acetic anhydride under argon. To the mixture, 47 μL (0.55 mmol) of perchloric acid (70%) was added. The resulting mixture was stirred at 60° C. for 30 min. It was cooled to room temperature and diethyl ether was added to precipitate the product. The dark purple precipitate was filtered and recrystallized in methylene chloride-diethyl ether to give a dark purple solid 22f, 204 mg (64%). MS (FAB, M⁺, C₃₉H₄₁N₄O₂⁺) calcd. 597, found 597.

N,N-Dimethyl-{4-[5-(4-N,N-dimethylaminophenyl)-5-(4-N-ethyl-N-(2,3-dihydro-1,4-phthalazinedion-6-yl)aminophenyl)-2,4-pentadienylidene]-2,5-cyclohexadien-1-ylidene}ammonium perchlorate (23a). A solution of 22a (287 mg, 0.420 mmol) and potassium hydroxide (50 mg, 0.85 mmol) in methanol (80 mL) was refluxed for 1 h and then cooled to room temperature. Hydrazine (1.3 mL, 41 mmol) was added and oxygen was removed from the mixture via freeze-pump-thaw cycle three times followed by reflux under argon for 1.5 h. The volatiles were removed by evaporation under reduced pressure and the yellow solids deposited were dried under vacuum. The solids dissolved in a mixture of THF-water and the solution was adjusted to pH 1 with 20% HClO₄. The blue solution was carefully (foaming) evaporated at room temperature to remove THF. The precipitate was collected by filteration, rinsed with water, and dried under vacuum to give 10a, 213 mg (74%) as a blue powder. UV-V is (CH₂Cl₂) λ, nm (relative ε): 400 (0.381), 730 (0.823), 826 (1.00). HRMS (FAB, M⁺, C₃₇H₃₈N₅O₂⁺) calcd. 584.3026, found 584.3041.

N,N-Dimethyl-{4-[5-(4-N,N-dimethylaminophenyl)-5-(4-N-ethyl-N-(2,3-dihydro-5-methyl-1,4-phthalazinedion-8-yl)aminophenyl)-2,4-pentadienylidene]-2,5-cyclohexadien-1-ylidene}ammonium perchlorate (23f). To a solution of 22f (100 mg, 0.143 mmol) in 5 mL of methylene chloride, potassium hydroxide in ethanol (0.303 M, 0.95 mL, 0.288 mmol) was added under stirring. The resulting brown mixture was evaporated under reduced pressure to remove the volatiles. To the residue anhydrous ethanol (30 mL) and anhydrous hydrazine (0.20 mL, 6.25 mmol) were added. The mixture was degassed by three cycles of pump-filling process with argon. The reaction mixtur then was refluxed under argon for 2 h. Then it was evaporated under reduced pressure to dryness and the orange residue was dissolved in 6 mL of THF-water mixture (5:1 v/v). To the mixture, HClO₄ aqueous solution (0.1 M, 6 mL) was added slowly under stirring. The resulting blue mixture was carefully (foaming) evaporated at room temperature to remove THF. The precipitate was collected by filtration, rinsed with water, and dried under vacuum. It was stirred with methylene chloride (3 mL) and filtered to give 10f, 90 mg (90%) as a blue powder. MS (FAB, M⁺, C₃₈H₄₀N₅O₂⁺) calcd. 598, found 598.

Preparation of 23a-DDC prodrug (24a). The same procedure described for the preparation of 6a was employed to prepare the prodrug in DMSO (2 mL, 0.0115M) using 23a and DDC. This solution was used for the in vitro test against HIV without further purification.

In another embodiment, the luminide chemiluminescent functionality A comprises an active oxalate. Representative synthetic pathways are given in TABLE 7, Scheme IV, for oxamide containing carrier such as 28.

The structural characteristic of this type of carrier is that the chemiluminescent moiety N-sulfonyloxamide is directly attached to two aryl groups of tetraarylpolymethine dye, forming the cyclized carrier such as 28. The common intermediate halo-substituted diarylketene such as 2a can be aminated using methods such as the palladium-catalyzed amination of aryl halide with benzophenoneimine to give the amino diarylketene such as 25 with a good yield. The amino groups of the ketene then can be substituted as desired such as forming the corresponding sulfamide such as 26 by reacting with sulfonyl anhydride. Reacting 2 molar proportions of a N-substituted aminodiarylketene such as 26 with 1 molar oxalyl halide yields the N,N′-bisaryl oxamide such as 27. Condensing the oxamide with an orthoester such as triethylorthofomate in a nonaqueous solvent such as acetic anhydride containing acid catalyst such as tetrafluoroboric acid, results the cyclized oxamido-tetraarylpolymethine dye conjugate such as the carrier 28.

TABLE 7
Representative Schematics for the Synthesis of
Carriers and Prodrugs Comprising an
Oxalate Chemiluminescent Functionality A.

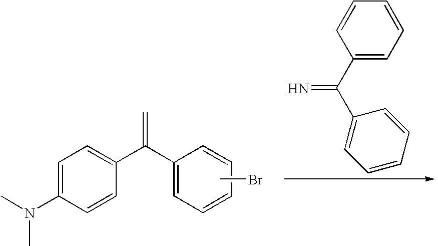

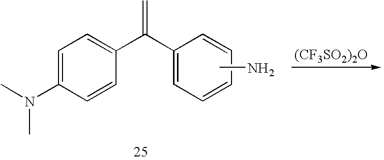

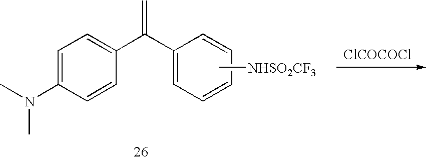

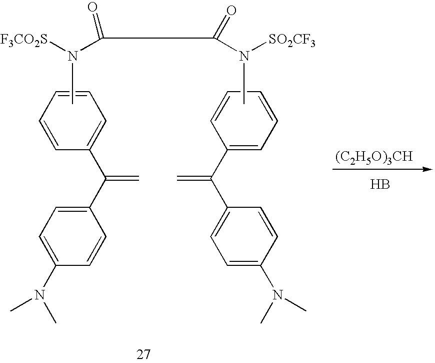

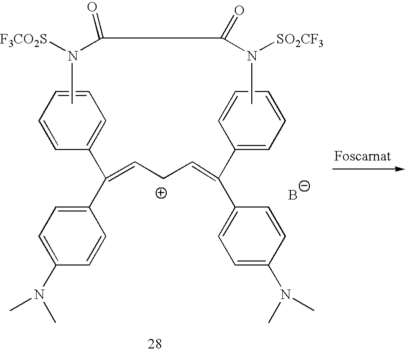

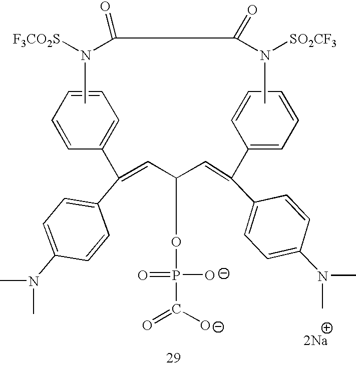

1-(4-aminophenyl)-1-(4-N,N-dimethylaminophenyl)-ethylene (25a). 1-(4-Bromophenyl)-1-(4-N,N-dimethylaminophenyl)ethylene (2a), 4.95 g (16.4 mmol), palladium(II) acetate, 113 mg (0.50 mmol), sodium tert-butoxide, 1.89 g (19.7 mmol) and toluene, 30 mL, were mixd in a flask. The mixture was flushed with argon. Then the catalyst ligand tri-tert-butylphosphine (500 μL, 2.01 mmol) and benzophenone imine, 3.02 mL (18.0 mmol) were added via syringes under argon. The resulting mixture was refluxed for 6 h and then cooled to room temperature. It was diluted with ethyl acetate (25 mL) and methanol (25 mL). Hydrochloric acid (2.0 M, 20 mL) was added dropwise and the mixture was stirred until the hydrolysis was complete (2 h). It was neutralized with 1M sodium carbonate to pH 9 and extracted with ethyl acetate (200 mL). The organic layer was separated and washed with water, brine and dried over MgSO₄, filtered and concentrated. The residue was purified on silica gel column using 30%(v/v) ethyl acetate in hexane and recrystallized from ethanol-hexane to give 25a, 3.40 g (87.1%) as a yellow crystalline: m.p. 81.4-82.4° C.; ¹H NMR (DMSO-d₆) δ 7.12 (d, J=8.9 Hz, 2H), 6.97(d, J=8.6 Hz, 2H), 6.68 (d, J=9.1 Hz, 2H), 6.52(d, J=8.6 Hz, 2H), 5.17 (s, 2H), 5.05 (d, J=1.5 Hz, 1H), 5.03 (d, J=1.8 Hz, 1H), 2.90 (s, 6H); Anal. C₁₆H₁₈N₂, calcd. C, 80.63; H, 7.61; N, 11.75, found C, 80.72; H, 7.58; N, 11.63.

1-(4-Trifluromethanesulsonamidophenyl)-1-(4-N,N-dimethylaminophenyl)-ethylene (26a). Under argon a mixture of 2.36 g (10.0 mmol) of 25a and 1.01 g (10.0 mmol) of triethylamine in dichloromethane (15 mL) was cooled to −5° C. To it trifluoromethanesulfonic anhydride, 1.68 mL (10.0 mmol) was added dropwise. The resulting blue mixture was stirred at room temperature for 4 h. The volatiles were removed by evaporation under reduced pressure. The light yellow solid collected was purified on silics gel column using 20% (v/v) ethyl acetate in hexane and recrystallized from cyclohexane to give 13a, 0.53 g (14.3%) as an off-white crystalline: m.p. 107.2-108.2° C.; ¹H NMR (DMSO-d₆) δ 7.33 (d, J=8.5 Hz, 2H), 7.25(d, J=8.6 Hz, 2H), 7.11 (d, J=8.7 Hz, 2H), 6.71(d, J=9.1 Hz, 2H), 5.36 (s, 1H), 5.23 (s, 1H), 3.38 (s, broad, 1H), 2.92 (s, 6H); Anal. C₁₇H₁₇F₃N₂O₂S, calcd. C, 55.13; H, 4.63; N, 7.56, F 15.39 found C 55.23, H 4.72, N 7.42, F 15.16.

N-4-[(4-N,N-Dimethylanilinyl)ethylenyl]phenyl-N-trifluoromethanesulfonyloxamide (27a). Under argon at 0° C., triethylamine, 100 μL (0.717 mmol) and then oxalyl chloride, 27 μL (0.310 mmol) were added dropwise to a clear solution of 208 mg (0.562 mmol) of 26a in 2-methoxyethyl ether (3 mL). The resulting reaction mixture was stirred at room temperature for 1 h and then at 60° C. for 2 h. It was cooled to room temperature, diluted with ethyl acetate (50 m]L) and washed with icy water twice. The organic layer was separated, dried over MgSO₄ and concentrated. The residue was chromatographed on silica gel column using chloroform and recrystallized from dichloromethane-hexane, yielding 123 mg (27.5%) of 27a as a white crystalline: m.p. 200° C. carbonized; MS (FAB, MH⁺) 795; ¹H NMR (DMSO-d₆) δ 7.29 (d, J=8.0 Hz, 4H), 7.21 (d, J=8.2 Hz, 4H), 7.11 (d, J=8.7 Hz, 4H), 6.71(d, J=8.9 Hz, 4H), 5.33 (s, 2H), 5.21 (s, 2H), 2.92 (s, 12H); Anal. C₃₆H₃₂F₆N₄O₆S₂, calcd. C, 54.40; H, 4.06; N, 7.05, F 14.34, found C, 54.32; H, 3.98; N, 7.09, F 14.29.

1,5-[4,4′-(N,N′-Ditrifluoromethanesulfonyloxamido)diphenyl]-1,5-bis-(dimethylanilinyl)pentadienium perchlorate (28a). Under argon to a solution of 60 mg (0.076 mmol) of 27a and 15 μL (0.090 mmol) of triethylorthoformate in 20 mL of acetic anhydride, tetrafluoroboric acid in diethyl ether (54 wt. %, 35 μL, 0.25 mmol) was added dropwise. The mixture was heated to 60° C. and stirred for 1 h. It was cooled to room temperature and diethyl ether was added to precipitate the product. The precipitate was collected by filtration and recrystallized from acetonile-diethyl ether to give 42 mg of 28a as a dark green crystalline.

Representative synthetic pathways are given in Scheme V for oxamide containing carrier such as 31 wherein the structural characteristic of this type of carrier is that the chemiluminescent moiety oxamide is attached through two molecular linkers to two aryl groups of a tetraarylpolymethine dye, forming the cyclized carrier. A convergent synthetic approach is adopted. A proper substituted amine such as N-2-bromoethylsulfamide, is reacted with oxalyl derivative such as oxalyl chloride to make the proper substituted oxamide such as N-2-bromoethyl-N-sulfonyloxamide 30. Condensing the proper substituted oxamide with a functionalized tetraarylpolymethine derivative such as a salt of the 1,5-bis(4-hydroxyphenyl)-1,5-diarylpentadiene forms the cyclized oxamido-tetraarylpolymethine carrier such as 1,5-(4,4′-(2,2′-N,N′-disulfonyloxamidodiethoxy)phenyl-1,5-diarylpentadiene cation carrier 31. Its prodrug 32 can be obtained by reacting the carrier with a nucleophilic species of a drug such as ddc.

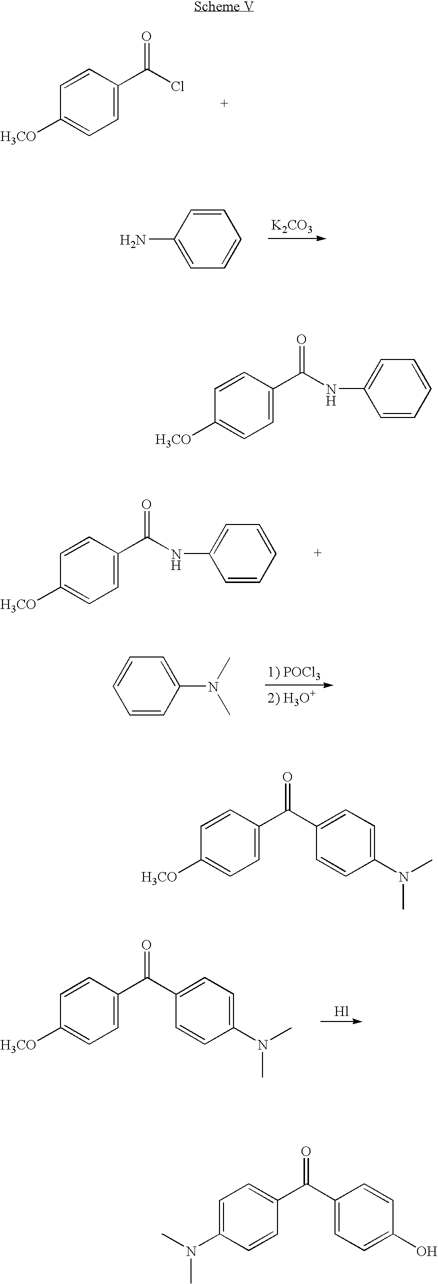

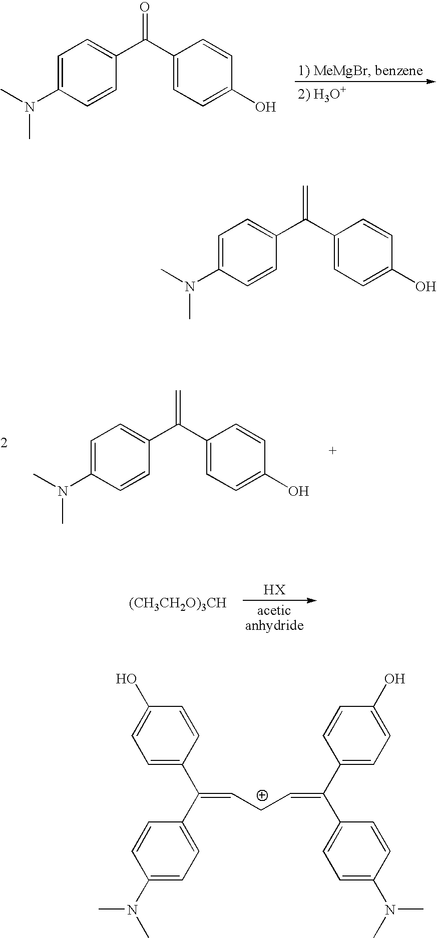

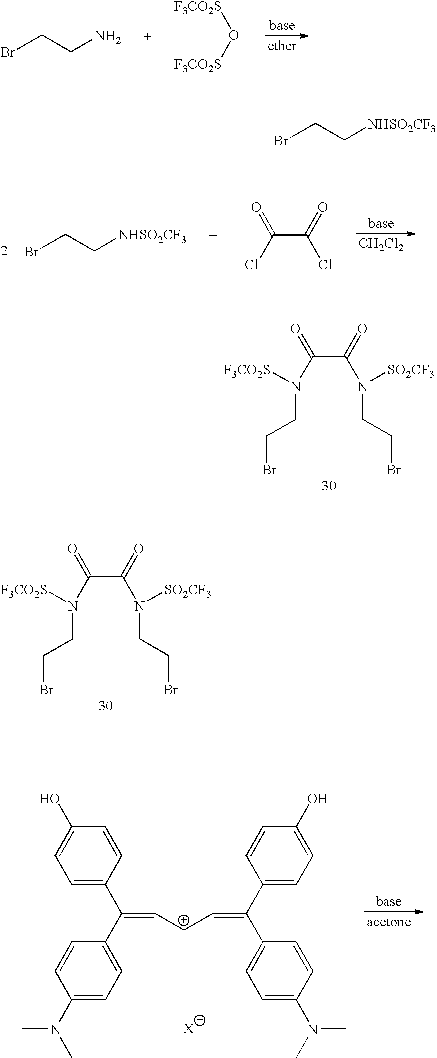

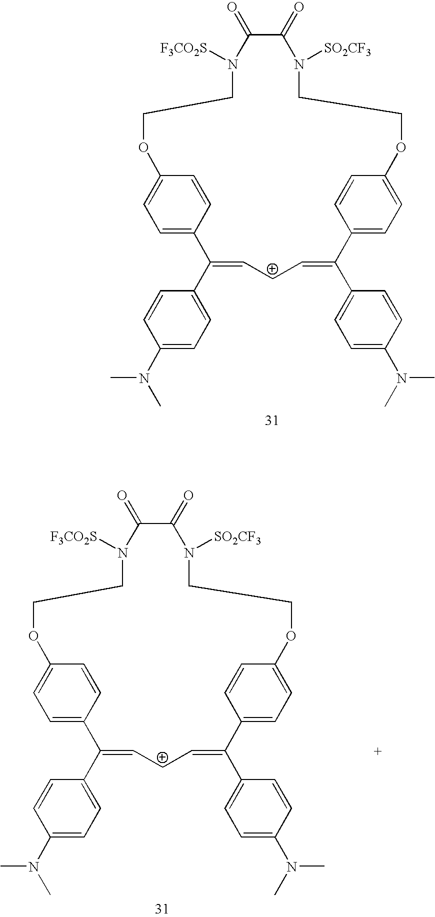

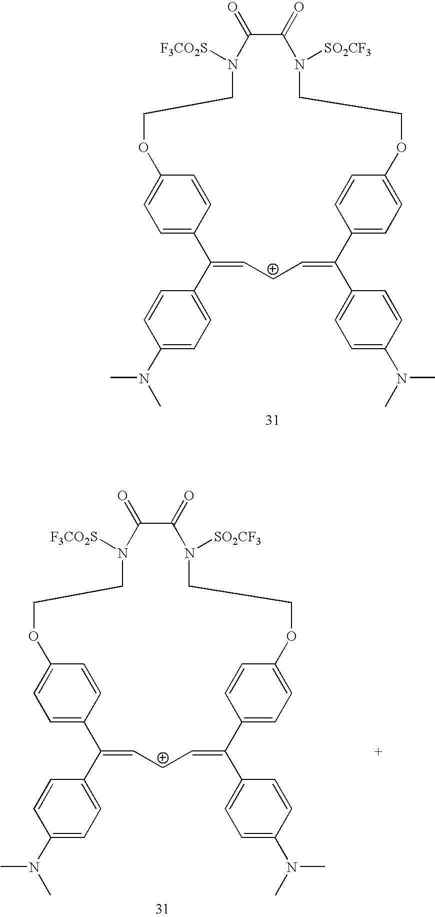

### Antiviral Tests

To facilitate intracellular delivery of hydrophilic drugs, a general lipophilic carrier molecule was designed and synthesized. The carrier comprised a chemiluminescent-photochromic conjugate that potentiates diffusion across cell membranes to enhance intracellular uptake of the drug. The designed mechanism involves activation of the chemiluminescent moiety by intracellular oxygen free radicals and intermolecular energy transfer of the excited state energy to the photochromic moiety to result in release of the drug to allow the desired pharmacological effect to occur. Prodrugs of Foscarnet and dideoxycytidine with several carriers caused suppression of a HIV infection in human cultured macrophages that was up to five times more effective than the drug alone. Successful in vivo efficacy testing of prodrug has been accomplished by demonstrating the suppression of a retroviral infection of Friends Leukemia Virus (FLV) in mice. Acute toxicity studies of the carrier indicated that it was nontoxic.

### EXPERIMENTAL

Evaluation of Prodrug Anti-HIV-1 Potency in Macrophage Assay. The anti HIV-1 evaluation of the carriers and their conjugates was performed in 6-day old monocyte/macrophages at Southern Research Institute, Frederick, Md. Briefly, peripheral blood monocytes were isolated from normal HIV-1 negative donors by plastic adherence following ficoll hypaque purification of the buffy coats. The monocytes were then cultured for 6 days to a macrophage-like phenotype. The test compounds were serially diluted and added to the cultures followed by the addition of a pretitered amount of the Ba-L strain of HIV-1 obtained from the NIAID AIDS Research and Reference Reagent Repository. Cultures were washed by media removal 24 hours post infection, fresh compound added and the cultures continued for an additional 6 days. HIV p24 antigen content to assess virus replication was measured by p24 ELISA assay. AZT and ddc were used as positive control compounds and run in parallel with each determination. Toxicity of the test materials was measured on replicate plates which did not receive virus, but were treated and setup identically to those receiving virus. At assay termination, the assay plates were stained with the tetrazolium based dye MTS to determine cell viability and quantify compound toxicity. Using a computer program at Southern Research Institute, IC₅₀ (50% inhibition of virus replication), TC₅₀ (50% cytotoxicity) and a therapeutic index (TI, T₅₀/IC₅₀) were obtained.

Evaluation Foscarnet Prodrug Anti-Retroviral Potency in a Murine Model. The effect of prodrug MTLJ-1-Foscarnet on four-week old Swiss mice infected with Friends Leukemia Virus (FLV) was tested by the Luminide Pharmaceutical Corporation. The mice were infected with FLV by an IP injection with 0.5 ml viral solution prepared by resuspending F4-6 cells (1×10⁶ cells/ml) in fresh media and harvesting that media 24 hr later and filtering through a 0.22 mm filter. The Foscarnet Group received 300 nanomoles of Foscarnet on days 5-9. The Carrier Group received 300 nanomoles of carrier on days 5-9. The MTLJ-1-Foscarnet Group received 300 nanomoles of MTLJ-1-Foscarnet on days 5-9. All mice were sacrificed on day 12. The spleens were removed and weighed. The toxicity of the carrier MTLJ-1 was also evaluated by the determination of its LD₅₀.

### Results and Discussion

Prodrugs were tested in cultured macrophages at Southern Research Institute with further testing performed under contract with NIH, and prodrugs were tested in a murine model at Luminide Pharmaceutical Corporation (LPC).

Southern Research Institute Evaluation of Prodrug Anti-HIV-1 Potency in Macrophage Assay. A negative control prodrug YY99811-1-Foscarnet versus that of 6a-Foscarnet and 6a-ddc were tested for the suppression of a HIV infection in human cultured macrophages at Southern Research Institute. The results of the tests are given in TABLES 7-9. The structures of the drug compounds, Foscarnet and ddc, and carriers, YY99811-1 versus that of 6a, are given in TABLE 5. The substitution of an oxygen for a nitrogen atom on structure YY99811-1 versus 6a results in a decreased ability for a drug to be released from the corresponding prodrug since the conjugation of the photochromic moiety is greatly diminished. The results of the tests of the negative control carrier and the corresponding Foscavir prodrug are given in TABLE 7. The data given in TABLE 7 indicates that YY99811-1 was nontoxic. The corresponding prodrug had no effect as anticipated. The Foscavir-YY99811-1 conjugate served as a negative control for prodrugs with the potential for release of the drug.

The results of ddc and Foscarnet prodrugs of carrier 6a are given in TABLES 8 and 9, respectively. In each case, the carrier was found to be nontoxic and the corresponding prodrug to be as efficacious as the free drug alone. This indicates that the prodrug was highly effective at drug release in the presence of HIV-1 infected human monocytes.

TABLE 7
Southern Research Institute test results of a negative control carrier
and the corresponding negative control Foscarnet prodrug.

| Compound | IC₅₀ (μM) | TC₅₀ (μM) | TI (TC₅₀/IC₅₀) |
|---|---|---|---|
| Foscarnet | 0.55 | >10 | >18 |
| YY99811-1 (carrier) | >10 | >10 | NA |
| YY99811-1-Foscarnet | >10 | >10 | NA |

TABLE 8
Southern Research Institute test results of the 6a carrier
and the corresponding ddc prodrug.

| Compound | IC₅₀ (μM) | TC₅₀ (μM) | TI (TC₅₀/IC₅₀) |
|---|---|---|---|
| ddc | 0.07 | >473.00 | >6757.14 |
| 6a | >100.00 | >100.00 | NA |
| (carrier) | | | |
| 6a-ddc | 0.03 | >100.00 | >3333.33 |

TABLE 9
Southern Research Institute test results of the 6a carrier
and the corresponding Foscarnet prodrug.

| Compound | IC₅₀ (μM) | TC₅₀ (μM) | TI (TC₅₀/IC₅₀) |
|---|---|---|---|
| Foscavir | 1.83 | >333.20 | >182.08 |
| 6a | 23.14 | >103.79 | >4.48 |
| (carrier) | | | |
| 6a-Foscarnet | 2.12 | 62.88 | 29.66 |

National Institute of Health (NIH) Contracted Southern Research Institute Evaluation of Prodrug Anti-HIV-1 Potency in Macrophage Assay. Southern Research Institute, under contract with NIH, advanced the tests described in the “Southern Research Institute Evaluation of Prodrug Anti-HIV-1 Potency in Macrophage Assay” section by retesting the 6a carrier as well as two additional carriers, GZW2-33-1 and GZW1-98-2. The structure of the test compounds, ddc and carriers 6a, GZW2-33-1, and GZW1-98-2 are given in TABLE 5. The results of the NIH sponsored tests are given in TABLE 10. The prodrugs caused suppression of a HIV infection in human cultured macrophages that was up to five times more effective than the very potent drug ddc alone. NIH rated the prodrugs “highly active” and determined that the prodrug was efficacious in the potentiation of ddc. The prodrugs were further demonstrated to be nontoxic.

TABLE 10
Southern Research Institute test results of ddc prodrugs of 6a,
GZW1-98-2, and GZW2-33-1 carriers performed under NIH contract.

| Compound | | | TI (TC₅₀/IC₅₀) |
|---|---|---|---|
| ddc | 0.040 | >100 | >2,500 |
| 6a | 2.62 | >100 | >38.2 |
| (carrier) | | | |
| 6a-ddc | 0.008 | >100 | >12,500 |
| GZW1-98-2 | 23.1 | >100 | >4.3 |
| (carrier) | | | |
| GZW1-98-2-ddc | 0.019 | >100 | >5,263 |
| GZW2-33-1 | 18.9 | >100 | >5.3 |
| GZW2-33-1-ddc | 0.021 | >100 | >4,762 |

LPC Evaluation of Prodrug Anti-Retroviral Potency in a Murine Model. LPC performed in vivo testing of the prodrug MTLJ-1-Foscarnet for the suppression of Friends Leukemia Virus (FLV) infection in mice as compared to Foscarnet alone. The results are given in TABLE 11. The structures of the Foscarnet, carrier MTLJ-1, and prodrug, MTLJ-1-Foscarnet are TABLE 5.

These results indicate that MTLJ-1-Foscarnet was highly effective as demonstrated by the absence of splenomegaly in the animals that were administered this compound. The spleen weights of the Virus+MTLJ-1-Foscarnet Group are the same as those of the No Virus, No Treatment Group; whereas, the spleen weights of the Virus+Foscarnet Group are the same as those of the Virus Alone Group.

The data indicate that the prodrug MTLJ-1-Foscarnet is effective and that Foscarnet is ineffective at a significance level of 0.01 as determined by the Students T test. The results of the toxicity testing of the prodrug were LD₅₀>1250 mg/kg IP which indicates that it was nontoxic. The data further indicated that MTLJ-1-Foscarnet was nontoxic by the weight gain of 2.5 grams by the Virus+MTLJ-1-Foscarnet Group as compared to the 1.5 gram weight gain by the Virus+Foscarnet Group.

TABLE 11
The effect of prodrug MTLJ-1-Foscarnet on four-week old
Swiss mice infected with Friends Leukemia Virus (FLV).

| | | | | Virus + | Virus + |
|---|---|---|---|---|---|
| | No Virus | Virus | Virus + | Carrier | MTLJ-1- |
| | No Treat. | Alone | Foscarnet | Foscarnet | Foscarnet |
| Average | 20.8 | 20.3 | 18.9 | 21.7 | 22.4 |
| Final | | | | | |
| Weight (g) | | | | | |
| Average | +1.6 | +0.1 | +1.5 | +0.4 | +2.5 |
| Weight | | | | | |
| Change (g) | | | | | |
| Average | 0.0907 | 0.1647 | 0.1727 | 0.1272 | 0.0914^{a} |
| Spleen | | | | | |
| Weight (g) | | | | | |
| Standard | 0.02 | 0.07 | 0.1 | 0.03 | 0.02 |
| Deviation | | | | | |
| of Spleen | | | | | |
| Weights (g) | | | | | |

^{a}p < 0.01 (MTLJ-1-Foscarnet)

### CONCLUSION

A chemiluminescent-photochromic conjugate carrier molecule was designed and synthesized to enhance the cellular uptake of antiHIV drugs. An increase in the activity of Foscarnet and ddc was observed in cultured macrophages infected with HIV and in mice infected with the retrovirus FLV. Since the carrier is independently of the drug, biologically active agents having the optimal structure to achieve the highest therapeutic ratio may be attached to such a carrier which is modified to achieve optimal bioavailability for a given drug. Thus, the physical-chemical properties of a prodrug which change its bioavailability, can be manipulated without altering the optimal drug structure. Thousands of existing promising drugs can be salvaged, and existing drugs can be made more potent with a higher therapeutic ratio. The discovery of a promising drug by conventional means costs the pharmaceutical industry, by one rule of thumb, approximately $10 million per application [22-23]. Because our prodrugs may be able to utilize and potentiate known biologically active compounds, tremendous potential savings in drug product development may be realized.

### REFERENCES

1. J. Gaub, et al., “The effect of Foscarnet (phosphonoformate) on human immunodeficiency virus isolation, T-cell subsets and lymphocyte function, AIDS, Gower Academic Journals Ltd, Vol. 1, No. 1, (1987), pp. 27-33.
2. F. C. Szoka, C. C. Jung, “Pharmaceutical liposomes containing phosphorous compounds for the treatment of viral infections”, WO 8905152, (1989).
3. K. Y. Hostetler, R. Kumar, “Lipid derivatives of phosphonacids for liposomal incorporation and method of use”, U.S. Pat. No. 5,463,092, (1995).
4. K. Y. Hostetler, J. W. Mellors, “Phosphonoformate lipid analogs for the treatment of drug-resistant human immunodeficiency virus infection”, WO 2001047511, (2000).
5. G. D. Kini, J. R. Beadle, H. Xie, K. A. Aldern, D. D. Richman, K. Y. Hostetler, “Alkoxy propane prodrugs of Foscarnet: effect of alkyl chain length on in vitro antiviral activity in cells infected with HIV-1, HSV-1, and HCMV”, Antiviral Res. Vol. 36, No. 1, (1997), pp. 43-53.
6. D. Herrmann, H. Opitz, H. Zilch, E. Voss, G. Zimmermann, “Covalent lipid-phosphonocarboxylic acid conjugates for use as anti-viral agents”, DE 19547022, (1997).
7. K. Y. Hostetler, G. D. Kini, J. R. Beadle, “Lipid prodrugs of pharmaceuticals with improved bioavailability”, WO 9639831, (1996).
8. J. M. Bailey, M. Lightfoote, E. Lillehoj, K. Nelson, “Synthesis and antiviral properties of sterol phosphonoformates”, Biochem, Soc. Trans. Vol. 22, No. 3, (1994), p. 353S.
9. K. Y. Hostetler, G. D. Kini, “Preparation of glycerophospholids as virucides”, WO 9615132, (1996).
10. C. E. McKenna, Z. Li, X. Liu, “Preparation and use of sulfur-containing phosphonoformate derivatives and analogs”, WO 2000002566, (2000).
11. B. Gorin, B. Pring, G. Thatcher, WO 9825938, (1998).
12. J. Maloisel, B. Pring, A. Tiden, “Preparation of novel glucosyl phosphonoformate esters as virucides”, WO 9816537, (1998).
13. J. O. Noren, E. Helgstrand, N. G. Johansson, A. Misiorny, G. Stening, “Synthesis of esters of phosphonoformic acid and their antiherpes activity”, J. Med. Chem., Vol. 26, No. 2, (1983), pp. 264-270.
14. P. Vlieghe, F. Bihel, T. Clerc, C. Pannecouque, M. Witvrouw, E. De Clercq, J. Salles, J. Chermann, J. Kraus, “New 3′-azido-3′-deoxythimidin-5′-yl O-(-hydroxyalkyl) carbonate prodrugs: synthesis and anti-HIV evaluation”, J. Med. Chem., Vol. 44, (2001), pp. 777-786.
15. R. N. MacNair, Photochemistry and Photobiology, Vol. 6, (1967), pp. 779-797.
16. J. Falt, C. Dahlgren, A. Karlsson, A. M. S. Ahmed, D. E. Minnikin, M. Ridell, “Activation of human neutrophils by mycobacterial phenolic glycolipids”, Clin. Exp. Immunol., Vol. 188, No. 2, (1999), pp. 253-260.
17. R. D. Lippman, “Site-specific chemiluminescent probes used in the analysis of pH, peroxidation and free radicals internally in metabolically active organelles”, Bioluminescence and Chemiluminescence, Academic Press, Inc. (1981), pp. 373-381.
18. F. Wilkinson, Luminescence in Chemistry, Edited by E. J. Bowen, D. Van Nostrand Co., LTD., London, (1968), Chp. 8, pp. 154-182.
19. H. Morawetz, Science, Vol. 240, (1987),172-176.
20. O. Schnepp, M. Levy, J. Am. Chem. Soc., Vol. 84, (1962), pp. 172-177.
21. D. F. Roswell, E. H. White, Methods in Enzymology, Vol. LVII (1978), Chp. 36, American Academic Press, pp. 409-445.
22. W. M. Wardell, “Scientific criteria and methods for drug assessment: requirements in the United States of America”, J. Z. Bowers and G. P. Velco, Editors, North Holland Biomedical Press, (1979), pp. 91-101.
23. C. Miller, W. Duncan, “The $120 million a month pharmaceutical race”, Today's chemist at work, April, (1997), Vol. 6, No. 4, pp. 50-57.
24. Pearson, D. E.; Buehler, C. A., Synthesis, (1972), October, pp. 533-542.
25. Yamamoto, Y.; Nishiyama, M.; Koie, Y., Tetrahedron Letters, (1998), 39, pp. 2367-2370.
26. Schroeder, H. R.; Boguslaski, R. C.; Carrico, R. J.; Buckler, R. T., “Monitoring Specific Protein-Binding Reactions with Chemiluminescence” in Methods In Enzymology, Vol. LVII, Academic Press, Inc., (1978), pp. 424-445.
27. Schuster, G. B., Schmidt, S. B., Bioluminescense and Chemiluminescence, edited by Marlene A. Deluca and William D. McElroy, (1981), pp. 23-29.
28. Berger, A. W., et al., Photochemistry and Photobiology, Vol. 4, (1965), pp. 1123-1127.
29. Philbrook, G. E., et al., Photochemistry and Photobiology, Vol. 4, (1965), pp. 1175-83.
30. White, E. H., et al., Photochemistry and Photobiology, Vol. 4, (1965), pp. 1129-55.
31. Philbrook, G. E., et al., Photochemistry and Photobiology, Vol. 4, (1965), pp. 869-76.
32. Rauhut, M. M., et al., Photochemistry and Photobiology, Vol. 4, (1965), pp. 1097-1110.
33. McCapra, F., et al., Photochemistry and Photobiology, Vol. 4, (1965), pp. 1111-1121.
34. McCapra, F., et al., Chemical Communications, No. 15, (1966), pp.522-23.
35. Gorsuch, J. D., “Studies of the Chemiluminescence of Luminol,” M.I.T. Ph.D. Thesis, 1969.
36. Legg, K. D., “The Chemiluminescence of Lucigenin,” K.I.T. Ph.d. Thesis, 1969.
37. Lytle, F. E., “Chemiluminescence and Photoluminescence of Ruthenium Chelates,” M.I.T. Ph.D. Thesis, 1968.
38. Allen, R. C., Chemical and Biological Generation of the Excited State, edited by Adam Waldeman, 1982, pp. 309-45.
39. Bowen, E. J., “Chemiluminescence in Solutions,” in Luminescence in Chemistry, Edited by E. J. Bowen, D. Van Nostrand Co. Ltd, London; (1968) pp. 183-190.
40. Tuemmler, W. B., and Widi, B. S., J Amer Chem Soc, Vol. 80, (1958), pp. 3772-75.
41. Mac Nair, R. N., Photochemistry and Photobiology, Vol. 6, (1967), pp. 779-97.
42. Chalkley, L., U.S. Pat. No. 2,885,303, (1958).
43. Chalkley, L., U.S. Pat. No. 2,936,235, (1957).
44. Chalkley, L., U.S. Pat. No. 2,829,148, (1958).
45. Chalkley, L., U.S. Pat. No. 2,839,542, (1958).
46. Chalkley, L., U.S. Pat. No. 2,839,543, (1958).
47. Brown, Glenn, Photochromism, pp. 294-375.
48. Wilkinson, F., “Intramolecular Electronic Energy Transfer Between Organic Molecules,” in Luminescence in Chemistry, Edited by E. J. Bowen, D. Van Nostrand Co. LTD., London, (1968), pp. 154-182.
49. Lippman, R. D., Bioluminescence and Chemiluminescence, Edited by Marlene A. Deluca and William D. McElroy, (1982), pp. 373-81.
50. Dreyer, J. F., “Self-Attenuating Ophthalmic Filter,” Rept. WADD-TR-60-632 February, 1961. AD 322820.
51. Dreyer, J. F., Harries, R. W., MacNair, R. N., Feldman, D., “Investigation of Materials and Systems for Protection Against Flash Blindness Effects of Nuclear Detonations,” Rept. 68-38-CM, AD 688692.
52. Polacoat, Inc., “Development of a Means to Provide Protection for Eyes Against the Dazzle Effects of Nuclear Detonations,” Quart. Rept. No. 5 on Contract DA-19-129 qm-1534, April 1961, AD 446865; also AD 447113 and 298225.
53. Van Dyke, K., Bioluminescence and Chemiluminescence, Vol. 1, CRC Press, Inc., 1985, pp. 1-42.
54. Zaklike, K. A., et. al., Photochemistry and Photobiology, Vol. 30, (1979), pp. 35-44.
55. Schaap, A. P., Burns, P. A., and Zaklika, K. A., J. Am. Chem. Soc., 99 (1977), pp. 1270-1272.
56. D'Iorio, A., Mavrides, C., Canadian Journal of pp. 1779-1784.
57. Vrang, L., Oberg, B., Antimicrobial Agents and Chemotherapy, (1986), 29, pp. 867-872.
58. Schnebli, H. P., et. al., Journal of Biological Chemistry, (1967), Vol. 242, pp. 1997-2004.
59. Roberts, E., Biochemical Pharmacology, (1974), Vol. 23, pp. 2637-2649.
60. Rando, R., Bangerter, F. W., Journal of the American Chemical Society, (1976), Vol. 98, pp. 6762-6764.
61. Tunnicliff, G., et. al., Experientia, (1977), Vol. 33, pp. 20-22.
62. Kobayashi, K., et. al., Febs Letters, (1977), Vol. 76, pp. 207-210.
63. Medicinal Chemistry A Biochemical Approach, Thomas Nogrady, (1985) Oxford University Press, N.Y., N.Y., pp. 193-203.
64. Jung, J. M., Metcalf, B. W., Biochemical and Biophysical Research Communications, (1975),
67, pp. 301-306.
65. Rando, R., Bangester, F. W., Biochemical and Biophysical Research Communications, (1977), Vol. 76, pp. 1276-1281.
66. Buu, N. T., Van Gelder, N. M. Br. J. Pharmac, (1974,), 52, pp. 401-406,
67. Enzyme Inhibitors, edited by Urs Brodbeck, (1980) pp. 85-95.
68. Enzyme Inhibitors, ed. by Urs Brodbeck, (1980), pp. 61-74.
69. Drug Action and Design: Mechanism Based Enzyme Inhibitors, ed. by Kalman, (1979), pp. 13-27.
70. Fuller, R. W., Nagarajan, R., Biochemical Pharmacology, (1978) pp. 1981-1983.
71. Barchardt, R. T., Wu, Y. S., Journal of Medicinal Chemistry, (1975), 18, pp. 300-303.
72. Enzyme Inhibitors, ed. by Urs Brodbeck, (1980), pp. 2.23-244.
73. Receptor Binding in Drug Research, ed. by Robert A. O'Brien, pp. 235-259.
74. Kandutsch, A. A., Chen, H. W., Journal of Biological Chemistry, (1973), 248, pp. 8408-8417.
75. Fimognari, G. M., Rodwell, V. W., Biochemistry, (1965) 4, pp. 2086-2090.
76. Bloxham, D. P., Biochem J., (1975), 147, pp. 531-539.
77. 3-Hydroxy-3-MethylQlutarYl Coenzvme A Reductase, ed., by John R. Sabine, CRC Series in Enzyme Biolpgy, CRC Press, Inc. Boca Raton, Fla.
78. Design of Active-Site-Directed Irreversible Enzyme Inhibitors, B. R. Baker, (1975), p. 9.
79. Inhibitors of DNA and RNA Polymerases, International Encyclopedia of Pharmacology and Therapeutics, Section 103, edited by Prem S. Sarin and Robert C. Gallo, Pergamon Press, N.Y., N.Y.
80. Mao, J. C., Robishaw, E. E., Biochemistry, (1975), 4, pp. 5475-5479.
81. Layton, D., Azzi, A., Biochemical and Biophysical Research Communications, (1974) 59, pp. 322-325.
82. Saari, W. S., et. al., J. Am. Chem. Soc., (1967), 10, pp. 1008-1014.
83. Bergstrand, H., et. al., Molecular Pharmacology, (1977), 13, pp. 38-43.
84. Fuller, R. W., et. al., Journal of Medicinal Chemistry, (1975), 18, p. 304.
85. Bricker, L. A., Levey, G. S., Journal of Biological Chemistry, (1972), 247, pp. 4914-4915.
86. Khwaja, T. A., et. al., Biochemistry, (1975), 14, pp. 4238-4244.
87. Bertelli, A., et. al., Experientia, (1976), 32, pp. 261-262.
88. Medicinal Chemistry A Biochemical Approach, Thomas Nogrady, (1985) Oxford University Press, N.Y., N.Y., pp. 274-285.
89. Prostaglandin Synthetase Inhibitors: New Clinical Applications, (1980) Alan R. Liss Inc., N.Y., N.Y., pp. 231-256.
90. The Pharmacological Basis of Therapeutics, ed. by A. G. Gilman, L. Gilman, 6th ed., (1980) MacMillan Publishing Co., N.Y., N.Y., pp 668-681.
91. Way, L., Durbin, R. P., Nature, (1969), 221, pp. 874-875.
92. Kattlove, H. E., Biochimica et. Biophysica Acta, (1974), 372, pp. 135-140.
93. Medicinal Chemistry A Biochemical Approach, Thomas Nogrady, (1985), Oxford University Press, N.Y., N.Y., pp 358-361.
94. Porter C. C., Watson, L. S., J. Am. Chem. Soc., (1967), 10, pp. 852-855.
95. Spurkes, T. L., Arch. Biochem. Biophys., (1954), 51, pp. 444-456.
96. Hartman, W. J., et. al., J. Biol. Chem., (1955), 216, pp. 507-529.
97. Collins, R. D., Stark, G. R., Journal of Biol. Chem. (1971), 246, pp. 6599-6605.
98. Lienhard, G. E., Science, (1973), 180, pp. 149-154.
99. Fall, R. R., West C. A., Journal of Biol. Chem., (1971), 246, pp. 6913-6928.
100. Coleman, J. E., Journal of Biol, Chem., (1968), 243, pp. 4574-4587.
101. Agarwal, R. P., et. al., Biochemical Pharmacology, (1975), 24, pp. 693-701.
102. Agarwal, R. P., et. al., Biochemical Pharmacology, (1977) 26, pp. 359-367.
103. Sheen, M. R., et. al., Mol. Pharmacol., (1968), 4, pp. 293-299.
104. Analysis of Insecticides and Acaricides, Gunther and Blinn, (1955), Interscience, N.Y., N.Y., pp. 401-404.
105. The PharmacoloQical Basis of Therapeutics, ed., by A. G. Gilman, L. Goodman, A. Gilman, 6th ed., (1980), MacMillan Publishing Co., N.Y., N.Y.
106. Berridge, M. J., Ann. Rev. Biochem., (1987), 56, pp. 159-93.
107. Calcium Antagonists and Cardiovascular Disease, edited by L. H. Opie, (1984), Raven Press, N.Y., pp. 165-173.
108. Johnston, G. A. R., Iversen, L. L., Journal of Neurochemistry, (1971), 18, pp. 1951-1961.
109. Chiou, C. Y., Malagodi, M. H., Br. J. Pharmac., (1975), 53, 279-285.
110. Inagaki, M., et. al., J of Biol. Chem., (1985), 260, pp. 2922-2925.
111. Inagaki, M., et. al., Biochemistry, (1984), 23, 5036.
112. Weinstock, M., Life Sciences, (1976), 19, pp. 1453-1466.
113. Hare, L. E. et. al., Journal of Medicinal Chemistry, (1974), 17, pp. 1-5.
114. Levitt, M., et. al., Biochemical Pharmacology, (1972), Vol. 16, pp. 1313-1321.
115. Nagatsu, T., et. al., Biochemical Pharmacology, (1972), Vol. 21, pp. 1945-1953.
116. Taylor, R. J., et. al., Biochemical Pharmacology, (1968), 17, pp. 1779-1788.
117. Nagatsu, T., et. al., J. Biol. Chem., (1964), 239, pp. 2910-2917.
118. Taylor, R. J., Ellenbogen, L., Life Sciences, (1967) 6, pp. 1463-1466.
119. Undenfriend, S., et. al., Biochemical
120. Weinhold, P. A., Rethy, V. B., Biochemical Pharmacology, (1969), 18, pp. 677-680.
121. Uretsky, N. J., et. al., The Journal of Pharm. and Exp. Therp., (1975), 193, pp. 73-87.
122. El Masry, M. H., et. al., Journal of Medical Chemistry., (1975), 18, pp. 16-20.
123. Counsell, R. E., et. al., Journal of Medical Chemistry, (1970), 13, pp. 1040-1042.
124. Robert, A., et. al., Life Sciences, (1974), 14, pp. 533-538.
125. Kollonitsch, J., et. al., Nature, (1978), 274, 906-908.
126. Polakoski, K. L., McRorie, R. A., Journal of Biol. Chemistry, (1973), 248, pp. 8183-8188.
127. Way, L. Durbin, R. P., Nature, (1969), 221, pp. 874-875.
128. Lippmann, w., Seethaler, K., Experientia, (1973), 29, pp. 993-995.
129. Lippmann, W., Experientia, (1973), 29, pp. 990-991.
130. Chen, F. W. K., et. al., Prostaglandins, (1977), 13, pp. 115-125.
131. Hidaka, H., Nautre, (1971), 231, pp. 54-55.
132. Hidaka, H., et al., MQI. Pharm., (1973), 9, pp. 172-177.
133. Van Der Schoot, J. B., et. al., Journal of Pharm. Exp. Therp., (1963), 141, pp. 74-78.
134. Hidaka, H., et. al., Journal of Pharm. Exp. Therp., (1974), 191, pp. 384-392.
135. Johnson, G. A., et. al., Journal of Pharm. Exp. Therp., (1969), 168, pp. 229-234.
136. Nagastu, T., et. al., Experientia, (1972), 28, pp. 779-780.
137. Lippmann, W., Lloyd, K., Biochemical Pharmacology, (1969), 18, pp. 2507-2516.
138. Diliberto, E. J., et. al., Biochemical Pharmacology, (1973), 22, pp. 2961-2972.
139. Porter, C. C., Torchiana, M. L., Biochemical Pharmacology, (1971), 20, pp. 183-191.
140. Oyama, H., et. al., Biochemical Pharmacology, (1976), 25, pp. 277-280.
141. Catignani, G. L., Neal, R. A., Life Sciences, (1975), 16, 1915-1922.
142. Hashiguchi, H., Takahashi, H., Mol. Pharm., (1977), 13, pp. 362-367.
143. Symes, A., Sourkes, T. L., Biochemical Pharmacology (1974), 23 pp. 2045-2056.
144. Tipton, K. F., Biochem. J., (1972), 128, pp. 913-919.
145. McEwen, C. M., et. al., Biochemistry, (1969), 8, pp. 3963-3972.
146. Baldessarini, R. J., Greiner, E., Biochemical Pharmacology, (1973), 22, pp. 249-256.
147. Guldberg, H. C., Marsden, C. A., Pharmacological Reviews, (1975), 27, pp. 135-206.
148. Olsen, R. W., et. al., Mol. Pharm., (1975), 11, pp. 558-565.
149. Johnson, G. A., et. al., Journal of Neurochemistry, (1976), 26, pp. 1029-1032.
150. Johnson, G. A., Iversen, L. L., Journal of Neurochemistry, (1971), 18, pp. 1939-1950.
151. Simon, J. R., Martin, D. L., Archives of Biochemistry and Biophysics, (1973), 157, pp. 348-355.
152. Springer, R. H., et. al., Journal of Med. Chem., (1967), 19, pp. 291-296.
153. Baker, B. R., Kozma, J., Journal of Med. Chemistry, (1967), 10, pp. 682-685.
154. Baker, B. R., Wood, W. F., Journal of Med. Chemistry, (1967), 10, pp. 1101-1105.
155. Spector, I., Johns, D. G., Journal of Biol. Chemistry, (1970), 245, pp. 5079-5085.
156. Baldwin, J. J., et. al., Journal of Med. Chemistry, (1975), 18, pp. 895-900.
157. Duggan, D. E., et. al., Journal of Med. Chemistry, (1975), 18, pp. 900-905.
158. Ferraccioli, G., et. al., J. Rheumatol, (1984), 11, pp. 330-332.
159. Iwata, H., et. al., Biochemical Pharm., (1969), 18, pp. 955-957.
160. Iwata, H., et. al., Biochemical Pharm., (1973), 22, pp. 1845-1854.
161. Huszti, Z., Sourkes, T. L., Journal of Pharm. Exp. Therp., (1975), 192, pp. 432-440.
162. Leinweber, F. J., Braun, G. A., Mol. Pharm., (1970), 6, pp. 146-155.
163. Lorenz, W., Werle, E., Bioch. Pharm., (1969), 17, pp. 539-549.
164. Ellenborgen, L., et. al., Biochem. Pharm., (1973), 22, pp. 939-947.
165. Ellenborgen, L., et. al., Biochem. Pharm., (1969), 18, pp. 683-685.
166. Alston, T. A., Abeles, R. H., Biochemistry, (1987),
167. Ellenbogen, L., et. al., Biochem. Pharm., (1969), 18, pp. 683-685.
168. Lukes, J. J., Neiforth, K. A., Journal of Med. Chemistry, (1975), 18, pp. 351-354.
169. Collins, D., Journal of Biol, Chemistry, (1974), 249, pp. 136-142.
170. Kandutsch, A. A., Chen, H. W., Journal of Biol. Chemistry, (1974), 249, pp. 6057-6061.
171. Kandutsch, A. A., Chen, H. W., Journal of Biol. Chemistry, (1973), 248, pp. 8408-8417.
171. Kupiecki, F. P., Marshall, N. B., Journal of Pharm. Exp. Therp., (1968), 160, pp. 166-170.
173. Kypson, J., Hait, G., Journal of Pharm. Exp. Therp., (1976), 199, pp. 565-574.
174. Pereira, J. N., Holland, G. F., Journal. Pharm. Exp. Therp., (1967), 157, pp. 381-387.
175. Dulin, W. E., et. al., Proceedings of the Society for Experimental Biology and Medicine, (1965), 118, pp. 499-501.
176. Dulin, W. E., Gerritsen, G. C., Proceedings of the Society for Experimental Biology and Medicine, (1966), 121, pp. 777-779.
177. Waldvogel, E. R. F., et. al., Mol. Pharm., (1967), 3, pp. 429-441.
178. Lowenstein, J. M., Journal of Biol. Chemistry, (1971), 246, pp. 629-632.
179. Maragoudakis, M. E., Journal of Biol. Chemistry, (1971), 246, pp. 4046-4052.
180. Maragoudakis, M. E., Hankin, H., Journal of Biol. Chemistry, (1971), 246, pp. 348-358.
181. Hashimoto, T., et. al., Eur, J., Biochem., (1971), 24, pp. 128-139.
182. Maragoudakis, M. E., Biochemistry, (1970), 9, pp. 413-417.
183. Brady, R. O., Biochem. Biophys. Acta, (1963), 70, pp. 467-468.
184. Rokujo, T., et. al., Life Sciences, (1970), 9, pp. 379-385.
185. Dalton, C., et. al., Prostaglandins, (1974), 7, pp. 319-327.
186. Windmueller, H. G., Levy, R. I., Journal of Biol. Chemistry, (1967), 9, pp. 2246-2254.
187. Roheim, P. S., et. al., Biochem. Biophys. Research. Comm., (1965), 20, pp. 416-421.
188. Smith, D. A., et. al., Geriatics, (1987), 42, pp. 55-62.
189. Falcon, M. G., Jones, B. R., Journal of Gen. Virol., (1977), 36, pp. 199-202.
190. Meek, E. S., Takahashi, M., Nature, (1968), 220, p. 882.
191. De Clercq, E., Luczak, M., Life Sciences, (1975), 17, pp. 187-194.
192. Becker, Y., Pharmac. Ther., (1980), 10, pp. 119-159.
193. Asano, T., Ochiai, Y., Mol. Pharm., (1977), 13, pp. 400-406.
194. Iwai, H., Journal of Biochem., (1974), 76, pp. 419-429.
195. Lippmann, W., Experientia, (1974), 30, pp. 237-239.
196. Nikaido, T., et. al., Planta Medica, (1981), 43, pp. 18-23.
197. Vigdahl, R. L., et. al., Biochem. Biophys. Research Comm., (1971), 42, pp. 1088-1094.
198. Tateson, J. E., Trist, D. G., Life Sciences, (1976), 18, pp. 153-162.
199. Evans, D. P., Thomson, D. S., Br. J. Pharmac., (1975), 53, pp. 409-416.
200. Simmonds, H. A., et. al., Lancet, (1978), Jan. 14, pp. 60-63.
201. Trotta, P. P., et. al., Mol. Pharm., (1978), 14, pp. 199-209.
202. Deibel, M. R., et. al., Biochemical Medicine, (1981), 25, pp. 288-297.
203. Humphrey, S. M., et. al., Journal of Surgical Research, (1987), 43, pp. 187-195.
204. Beard, N. A., et. al., Br. J. Pharmac., (1975), 54, pp. 65-74.
205. Chignard, M., Vargafting, B. B., Prostaglandins, (1977), 14, pp. 222-240.
206. Panganamala, R. V., et. al., Prostaglandins, (1977), 14, pp. 261-271.
207. Kulkami, P. S., Eakins, K. E., Prostaglandins, (1976), 12, pp. 465-469.
208. Downing, D. T., et. al., Biochem., Biophys. Research Comm., (1970), 40, pp. 218-223.
209. Gorman, R. R., et. al., Biochem. Biophys. Research Comm., (1977), 79, pp. 305-313.
210. Cushman, D. W., Cheung, H. S., Biochem. Biophys. Acta, (1976), 424, pp. 449-459.
211. Taylor, R. J., Salata, J. J., Biochemical Pharmacology, (1976), 25, pp. 2479-2484.
212. Ku, E. C., et. al., Biochemical Pharmacology, (1975), 24, pp. 641-643.
213. Burghuber, O. C., et. al., Am. Rev. Respir. Dis., (1985), 131, pp. 778-785.
214. Michelot, R. J., et. al., Mol. Pharm., (1977), 13, pp. 368-373.
215. Kessel, D., McElhinney, R. S., Biochemical Pharmacology, (1975), 24, pp. 133-137.
216. Gale, G. R., et. al., J. Med. Chem., (1970), 13, pp. 571-574.
217. The Molecular Basis of Antibiotic Action, ed. by E. F. Gale, (1981), pp. 258-401.
218. Hillcoat, B. L., et. al., J. Biol. Chem., (1967), 242, pp. 4777-4781.
219. Carlin, S., et. al., Mol. Pharm., (1974), 10, pp. 194-203.
220. Johns, D. G., et. al., Biochemical Pharmacology, (1970), 19, pp. 1528-1533.
221. Baker, B. R., et. al., J. Med. Chem., (1967), 10, pp. 1134-1138.
222. Baker, B. R., Lourens, G. J., J. Med. Chem., (1989), 1, pp. 666-672.
223. Nair, M. G., et. al., J. Med. Chem., (1974), 17, pp. 1268-1272.
224. Ferone, R., J. Biol. Chem., (1970), 245, pp. 850-854.
225. LaFon, S. W., et. al., J. Biol. Chem., (1985), 260, pp. 9660-9665.
226. Spector, T., Miller, R. L., Biochemica et. Biophysica Acta, (1976), 445, pp. 509-517.
227. Advances in Enzymology, ed. by Alton Meister, (1987) pp. 59-101.
228. Umezawa, H., Ann. Rev. Microbiol., (1982), 36, pp. 75-99.
229. Walsh, C. T., Ann. Rev. Biochem., (1984), 53, pp. 493-535.
230. Demain, A., Biochem. Soc. Symp., (1983), 48, pp. 117-132.
231. Suhadolnik, R. J., Progress in Nucleic Acid Research and Moleulor Biology, (1979), 22, pp. 193-291.
232. Free, C. A., et. al., Biochem. Pharm., (1971), 20, pp. 1421-1428.
233. Enzymes Dixon M. and Webb, F. C., Academic Press Inc., N.Y., N.Y. (1964), p. 407.
234. Kondo, T., et. al., Diabetes Care, (1982), vol. 5, Number 3, pp. 218-221.
235. Higgins, I. J., Hill, H. A. O., Plotkin, E. V., Eur. Pat. Appl. EP78,636.
236. Miller, L. L., J. Electroanal. Chem., (1981), 117, pp. 267-281.
237. Mills, R. L., U.S. patent application Ser. No. 784,243.
238. Gros, P., et. al., Cell, (1986), 47, pp. 371-380.
239. Sanchez-Pescador, R., et. al., Science, (1985), 277, pp. 484-492.
240. The Enzymes, ed. by Paul D. Boyer, (1975), 3rd edition, Vol. XII, Part B, Academic Press, N.Y., N.Y.
241. De Clercq, E., and Balzarini, J., Antiviral Research, Suppl. I, (1985), pp. 89-94.

Although the foregoing invention has been described in some detail by way of illustration and examples for clarity and understanding, it will be obvious that various modifications and changes which are within the knowledge of those skilled in the art are considered to fall within the scope of the appended claims

1. A method of synthesis of a chemical compound having the formula A-B-C
where the A is a chemiluminescent moiety,
B is an energy acceptor moiety, and
C is a biologically active moiety comprising the steps of
forming a benzophenone,
forming a diaryl ethylene, and
performing at least one of
(a) attaching a precursor to generate a phthalhydrazide such as phthalimide, aminophthalic acid diester, aminophthalic acid dihydrazide, aminophthalic anhydride and phthalhydrazide protected by a hydrolyzable group to form the precursor-ethylene conjugate, and condensing two ethylene-precursor conjugates to form a precursor-pentadiene conjugate, and
(b) condensing two diaryl ethylene to form a pentadiene, and attaching a precursor to generate a phthalhydrazide such as phthalimide, aminophthalic acid diester, aminophthalic acid dihydrazide, aminophthalic anhydride and phthalhydrazide protected by a hydrolyzable group, to form the precursor-pentadiene conjugate, and
converting the precursor to the phthalhydrazide by at least one of the corresponding reactions
phthalimide with hydrazine,
aminophthalic acid diester with hydrazine,
aminophthalic anhydride with hydrazine, and
hydrolysis of phthalhydrazide protected by a hydrolyzable group to form a carrier compound, and
reacting the carrier compound with the biologically active moiety to form a corresponding conjugate.

2. The method of synthesis of the compound of claim 1 wherein the compound serves to delivery the C moiety to a desired biological compartment.

3. The method of synthesis of the compound of claim 1 wherein the compound is a prodrug.

4. The method of synthesis of the compound of claim 3 wherein the compound serves as a prodrug for at least one of antiviral agents for the treatment of viral infections and anticancer agents for the treatment of cancers.

5. The method of synthesis of the compound of claim 4 wherein the compound serves as a prodrug for the treatment of at least one of the group of viruses comprising Human Immunodeficiency Virus (HIV), herpes viruses such as Herpes Simplex Virus, (HSV), Epstein-Barr Virus (EBV), Varicella Zoster (VZV), Cytomegalovirus (CMV), HSV-6, and HSV-8 (Kaposi's sarcoma), Human Papilloma Virus (HPV), rhinoviruses, and hepatitis-linked viruses.

6. The method of synthesis of the compound of claim 4 wherein the compound serves as a prodrug for the treatment of at least one of the group of cancers comprising colon, breast, lung, renal, retinal, and skin.

7. The method of synthesis of the compound of claim 3 wherein the prodrugs have increased bioavailability.

8. The method of synthesis of the compound of claim 2 wherein the compound is a cellular permeant prodrug.

9. The method of synthesis of the compound of claim 8 wherein intracellular drug release occurs when the prodrug reacts with cellular free radicals via a mechanism involving chemiluminescence, photochromism, and intramolecular energy transfer.

10. The method of synthesis of the compound of claim 1 wherein the C moiety is a pharmaceutical agent or drug.

11. The method of synthesis of the compound of claim 10 wherein the pharmaceutical agent is at least one of the group of antilipidemic drugs, anticholesterol drugs, contraceptive agents, anticoagulants, anti-inflamatory agents, immuno-suppressive drugs, antiarrhythmic agents, antineoplastic drugs, antihypertensive drugs, epinephrine blocking agents, cardiac inotropic drugs, antidepressant drugs, diuretics, antifungal agents, antibacterial drugs, anxiolytic agents, sedatives, muscle relaxants, anticonvulsants, agents for the treatment of ulcer disease, agents for the treatment of asthma and hypersensitivity reactions, antithroboembolic agents, agents for the treatment of muscular dystrophy, agents to effect a therapeutic abortion, agents for the treatment of anemia, agents to improve allograft survival, agents for the treatment of disorders of purine metabolism, agents for the treatment of ischemic heart disease, agents for the treatment of opiate withdrawal, agents which activate the effects of secondary messenger inositol triphosphate, agents to block spinal reflexes, and antiviral agents including a drug for the treatment of AIDS.

12. The method of synthesis of the compound of claim 1 wherein the C moiety is released by an oxidation reduction reaction with the target cell's electron carriers or by reaction with free radicals produced as a consequence of electron transport.

13. The method of synthesis of the compound of claim 12 wherein the C moiety is released into a desired compartment in active form.

14. The method of synthesis of the compound of claim 13 wherein the released C moiety has a greater therapeutic effect or therapeutic ratio relative to the free C agent alone.

15. The method of synthesis of the compound of claim 14 wherein the released C moiety has a greater therapeutic effect or therapeutic ratio relative to the free C agent alone as a consequence of at least one of altered pharmacokinetics or pharmacodynamics such as a desirable kinetics of release, a resistance to inactivation or excretion, greater solubility, enhanced absorption, a diminished toxicity, or greater access to the cellular or biological compartment which is the site of action of C.

16. The method of synthesis of the compound of claim 1 wherein A represents a functionality which undergoes at least one of
an oxidation reduction reaction where electrons are transferred directly between A and the target cell's electron carriers, and
a reaction with free radicals of oxygen which are produced as a consequence of electron transport
such that an excited state is produced in A as a consequence of its participation in one of these reactions.

17. The method of synthesis of the compound of claim 16 wherein A undergoes intramolecular energy transfer from its own excited state to the B functionality which is an energy acceptor.

18. The method of synthesis of the compound of claim 17 wherein upon receiving energy from A, B achieves an excited state which relaxes through heterolytic cleavage of the covalent bond of B with C where C is a drug moiety which is released into the environment.

19. The method of synthesis of the compound of claim 18 wherein the released drug molecule effects a therapeutic functional change by a mechanism which comprises receptor mediated mechanisms including reversible and irreversible competitive agonism or antagonism including a molecule known as a suicide substrate or a transition state analogue mechanism or a noncompetitive or uncompetitive agonism or antagonism or the action is by a nonreceptor mediated mechanism including a “counterfeit incorporation-mechanism”.

20. The method of synthesis of the compound of claim 1 wherein the chemiluminescent molecule comprises at least one of the group of
molecules undergoing reaction involving peroxides and oxygen free radicals,
molecules undergoing reaction involving oxidation or reduction, and
molecules undergoing both reaction with peroxides and oxygen free radicals followed by an oxidation or reduction reaction.

21. The method of synthesis of the compound of claim 20 wherein the chemiluminescent molecule comprises at least one of the group of luminol and its derivatives, lucigenin and its derivatives, Lophine and its derivatives, acridinium esters and acridans, tetraphenylpyrrole, phthalhydrazides, acyloins, biacridinium salts, vinylcarbonyls, vinylnitriles, tetrakis (dimethylamino) ethylene, acylperoxides, indoles, tetracarbazoles and active oxalates.

22. The method of synthesis of the compound of claim 20 wherein the chemiluminescent molecule comprises at least one of the group of ruthenium chelates 2, 6-diaminopyrene, or cation radicals and molecules which follow a Chemically Initiated Electron Exchange Luminescence mechanism such as certain dioxetans and dioxetanones.

23. The method of synthesis of the compound of claim 20 wherein the chemiluminescent molecule comprises at least one of the group of dioxene derivatives and other compounds that form a dioxetan by reaction with superoxide and then produce efficient chemiluminescence by a CIEEL mechanism.

24. The method of synthesis of the compound of claim 20 wherein the chemiluminescent molecule comprises at least one of the group of

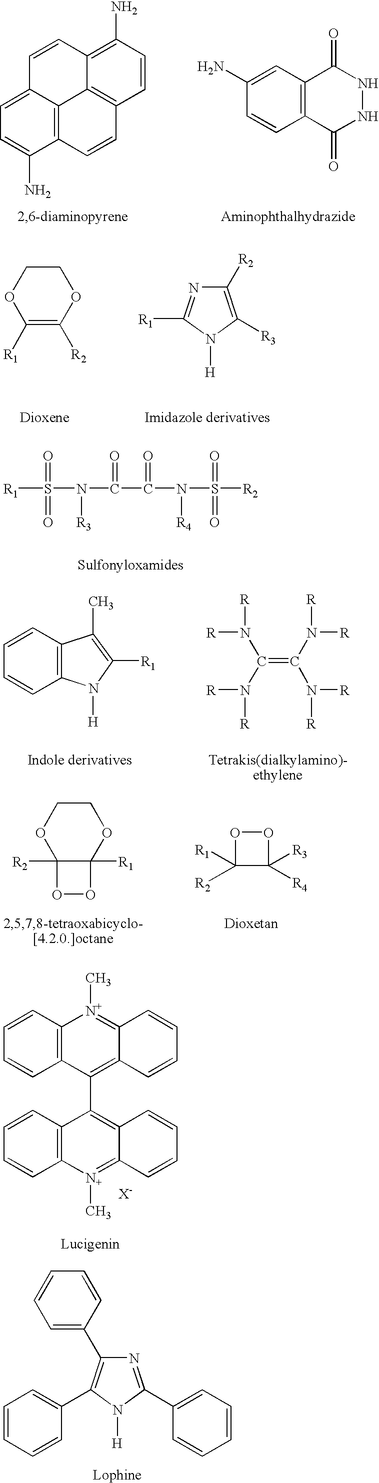

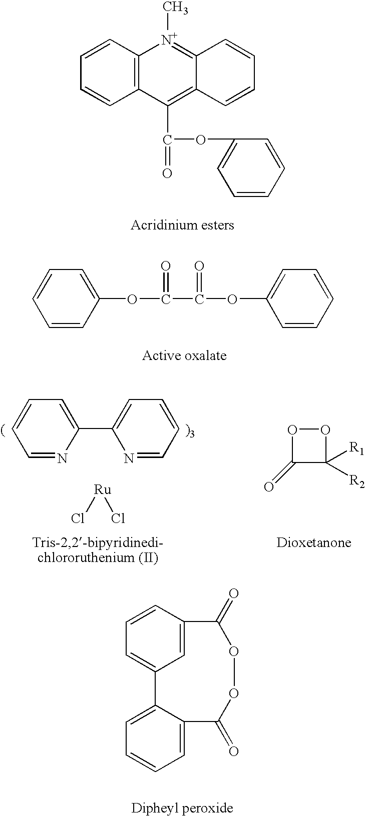

25. The method of synthesis of the compound of claim 1 wherein the B moiety is a photochromic compound.

26. The method of synthesis of the compound of claim 25 wherein the photochromic compound comprises one which demonstrate photochromic behavior with electromagnetic radiation and bleaching agents.

27. The method of synthesis of the compound of claim 26 wherein the A functionality is chemiluminescent, and the B functionality is such that the photodissociative drug release spectrum of B overlaps the chemiluminescence spectrum of A.

28. The method of synthesis of the compound of claim 25 wherein the photochromic compound comprises a cationic dye.

29. The method of synthesis of the compound of claim 28 wherein the cationic dye comprises at least one of a di and triarylmethane dyes, triarylmethane lactones and cyclic ether dyes, cationic indoles, pyronines, phthaleins, oxazines, thiazines, acridines, phenazines, and anthocyanidins, and cationic polymethine dyes and azo and diazopolymethines, styryls, cyanines, hemicyanines, dialkylaminopolyenes, and other related dyes.

30. The method of synthesis of the compound of claim 28 wherein the cationic dye comprises at least one of

| | |
|---|---|
| Malachite Green | 42000 |
| Helvetia Green | 42020 |
| Basic Blue 1 | 42025 |
| Brilliant Blue | |
| Setoglaucine | |
| Basic Green 1 | 42040 |
| Brilliant Green | |
| Acid Blue 1 | 42045 |
| Xylene Blue VS | |
| Patent Blue V | |
| Alphazurine 2G | |
| Acid Blue 3 | 42051 |
| Brilliant Blue V | |
| Patent Blue V | |
| Food Green 3 | 42053 |
| FDC Green 3 | |
| Acid Green 6 | 42075 |
| Light Green SF Bluish | |
| Acid Blue 7 | 42080 |
| Xylene Blue AS | |
| Patent Blue A | |
| Acid Green 3 | 42085 |
| Acid Blue 9 | 42090 |
| Erioglaucine | |
| Acid Green 5 | 42095 |
| Light Green SF Yellowish | |
| Acid Green 9 | 42100 |
| Erioviridene B | |
| Acid Blue 147 | 42135 |
| Xylene Cyanol FF | |
| Basic Red 9 | 42500 |
| Pararosaniline | |
| Basic Violet 14 | 42510 |
| Fuchsin | |
| Magenta | |
| Basic Fuchsin | 42510B |
| Basic Violet 2 | 42520 |
| New Magenta | |
| Hoffman Violet | 42530 |
| Iodine Violet | |
| Basic Violet 1 | 42535 |
| Methyl Violet | |
| Basic Violet 13 | 42536 |
| Methyl Violet 6B | |
| Basic Violet 3 | 42555 |
| Crystal Violet | |
| Gentian Violet | |
| Iodine Green | 42556 |
| Basic Blue 8 | 42563 |
| Victoria Blue 4R | |
| AcidBlue 13 | 42571 |
| Fast Acid Violet 10B | |
| Acid Blue 75 | 42576 |
| Eriocyanine A | |
| Methyl Green | 42585 |
| Ethyl Green | 42590 |
| Basic Violet 4 | 42600 |
| Ethyl Violet | |
| Acid Violet 49 | 42640 |
| Wool Violet 5BN | |
| Acid Blue 15 | 42645 |
| Brilliant Milling Blue B | |
| Acid Violet 17 | 42650 |
| Acid Violet 6B | |
| Wood Violet 4BN | |
| Formyl Violet | |
| Acid Violet 5BS Conc. | |
| Acid Violet 19 | 42685 |
| Acid Fuchsin | |
| Red Violet SR | 42690 |
| Acid Blue 22 | 42755 |
| Aniline Blue | |
| Soluble Blue | |
| Solvent Blue 3 | 42775 |
| Solvent Blue 3 | 42780 |
| Methyl Blue | |
| Aurin | 43800 |
| Mordant Blue 3 | 43820 |
| Eriochrome Cyanine R | |
| Acid Green 16 | 44025 |
| Naphthalene Green V | |
| Pontacyl Green NV Extra | |
| Basic Blue 11 | 44040 |
| Victoria Blue R | |
| Basic Blue 15 | 44085 |
| Night Blue | |
| Acid Green 50 | 44090 |
| Wool Green S | |
| Kiton Green S. Conc. | |
| Basic Green 3 | |
| Sevron Green B | |
| Brilliant Blue F & R Extra | |
| Brilliant Green Sulfonate | |
| Hexakis (hydroxyethyl) | |

Pararosaniline 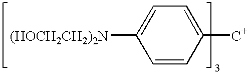
New Green 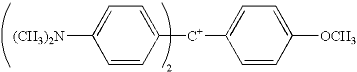
Phenolphthalein 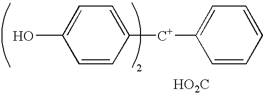
Malachite Green Ethiodide 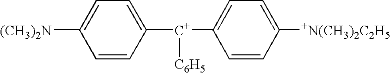
Hydroxyalkylated Pararosanilines 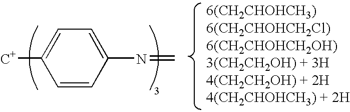
Hydroxyalkylated New Fuchsins 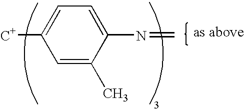
New Yellow 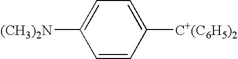
Dochner's Violet 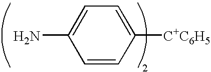
New Red 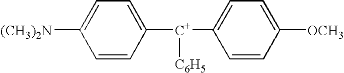
Bis(hydroxyethyl) Doebner's Violet 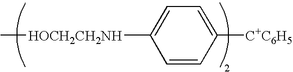
“New Magenta” 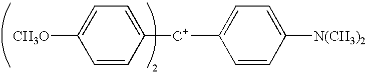
Tetrakis(hydroxyethyl) Doebeer's Violet 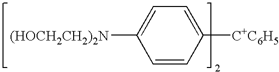
Trichloro Crystal Violet 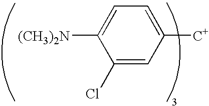
Slow Red 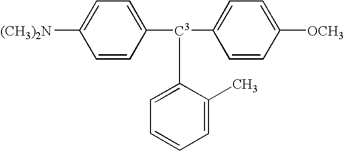 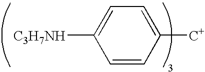 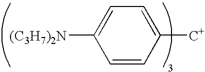 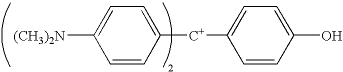 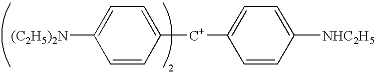 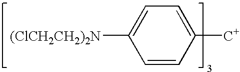 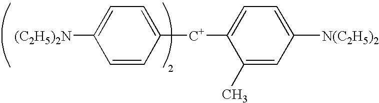 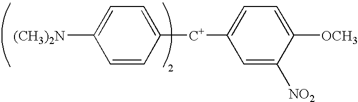 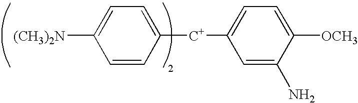 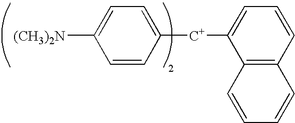 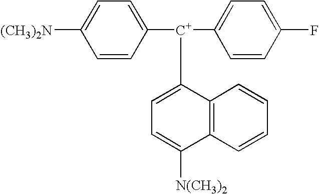 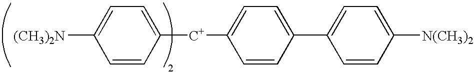 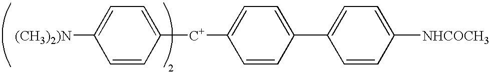 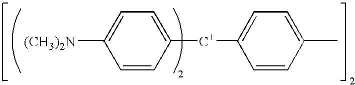
^{a}Only the cyanide, bisulfite, and hydroxide ions are considered, regardless of the other anions present
in the solution.
^{b}More detailed descriptions of the compositions of photochromic materials tested are given in
Macnair's review [255; tables 1A-4].
^{c}Ethanol.
^{d}Diethyl ether.
^{e}1,2-Dichloroethane.
^{f}1,1-Dichloroethane, cyclohexane-1,1-dichloroethane, or cyclohexane-1,2-dichloroethane mixtures.
^{g}Benzene.
^{h}Dimethylsulfoxide, neat and aqueous.
ⁱAcetone.
~^{j}ACETIC ACID.
^{k}Ethyl acetate.
^{l}Ethyl bromide.
^{m}2-Methoxyethanol
ⁿChloroform.
^{o}Ethanol with KCN.
^{p}Ethanol wiih KOH.
^{q}Carboxylic acids-acetic to stearic; hydrocinnamic acid; ethyl and butyl acid phthalates.
^{r}Octadecylnitrile, tributyl phosphate, aniline, 2-(p-tert-butylpheno xy)ethanol, tetraethyleneglycol
dimethyl ether, or poly(ethylene glycols).
^{s}Amides-formamide to stearamide; methylformamide or methylacetamide; dimethyl- or diethyl-
formamide or acetamide.
^{t}Three-to-one solutions of cellulose acetate with any of the following five-to-one plasticizer mixtures:
butyl stearate, Polyethylene Glycol 600-butyl acetoxystearate, butyl stearate, or Dowanol EP-butyl
acetoxystearate.
^{u}Water containing SO₂.
^{v}Water containing bisulfite and papain.
^{w}Poly(vinyl alcohol) with dimethylsulfoxide (5:1).
^{x}Films, containing residual solvent, cast from the following solutions: ethanol-acetone solutions of
vinyl acetate-vinyl alcohol copolymer; aqueous poly(vinyl alcohol); aqueous poly(vinyl pyrrolidone);
or aqueous methyl vinylether-maleic acid copolymer.
^{y}Methanol-dioxane with aqueous NH₄ HSO₃.
^{z}Paper impregnated with a toluene solution of poly(methyl methacrylate), stearic acid, and 2-(p-tert-
butylphenoxy)ethanol, then dried.
^{aa}Intramicellar impregnation of cellulose with the following swelling agents: n-propylamine, n-
butylamine, n-hexylamine, 2-aminoethanol, dimethylformamide, acetic acid, dimethylsulfoxide,
methylacetamide, dimethylacetamide, or formamide.
^{bb}Films cast from an approximately 4:3 mixture of a 20% solution and cellulose acetate butyrate in
toluene-ethyl acetate (1:1) and triallycyanurate in dioxane.
^{CC}FILMS CAST FROM A 2:1 MIXTURE OF A 25% SOLUTION OF CELLULOSE ACETATE
BUTYRATE IN TOLUENE-ETHYL ACETATE (1:1) AND THE TITANIUM ESTERS OF N,N,N′,N′-
TETRAKIS(2-HYDROXYPROPYL) ETHYLENEDIAMINE.
^{dd}Pure water.
^{ee}Films cast from aqueous gelatin or other hydrocolloids.
^{ff}Dimethylsulfoxide with methanolic KCN.
^{gg}2-Methoxyethanol with methanolic KCN.
^{hh}Water or aqueous methanol containing bisulfite.
ⁱⁱPaper impregnated with m-dimethoxybenzene, acetonitrile, acetic acid, or phenyl methyl carbinol.
^{jj}Ethanol-benzene.
^{kk}Aqueous ethanol, methanol, aqueous methanol, aqueous acetone, benzene-methanol, carbon
tetrachloride-methanol, cyclohexane-methanol, or chloroform-methanol.
^{ll}Films cast from 3:1 solutions of cellulose acetate and either Polyethylene Glycol 600 .RTM. or
ethylene glycol phenyl ether as plasticizer.
^{mm}Films, containing residual solvent, cast from solutions of either cellulose acetate in 2-
methoxyethanol or poly(vinyl alcohol) in aqueous ethanol.
ⁿⁿFilms, containing residual solvent, cast from solutions of either cellulose acetate butyrate in 2-
methoxyethanol or poly(vinyl acetate) in methanol.
^{oo}Ethanol containing ammonia.
^{pp}Aqueous methanol containing NH₄ HSO₃ and urease.
^{qq}Aqueous methanol containing NH₄ HSO₃, with or without sodium dithionite.
^{rr}Aqueous acid at pH 1.
^{ss}Aqueous ammonia containing KCN.
^{tt}Paper impregnated with aqueous solutions with or without hydrocolloids.
^{uu}2-Methoxyethanol containing HCl.
^{vv}Aqueous methanol containing NH₄ HSO₃, and glucose oxidase.
^{ww}9:1 Methanol-water.
^{xx}Aqueous NaOH. 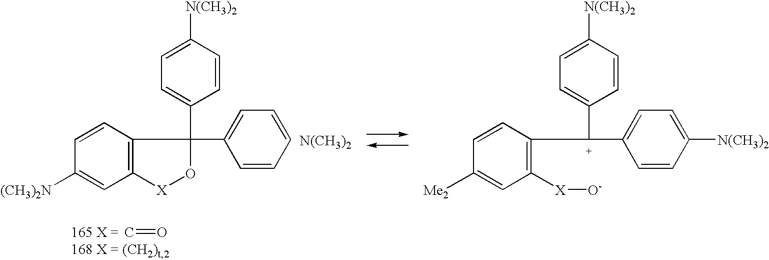 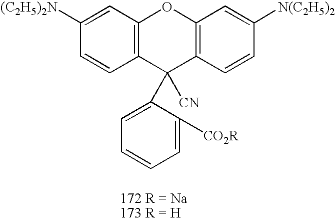 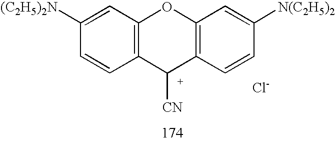 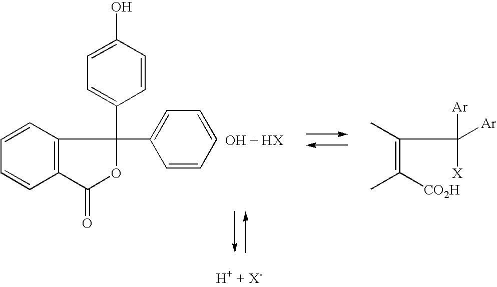 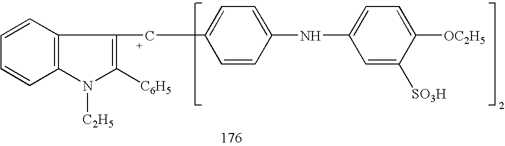 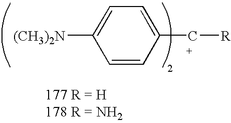 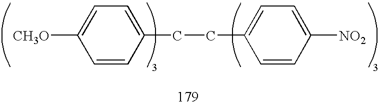 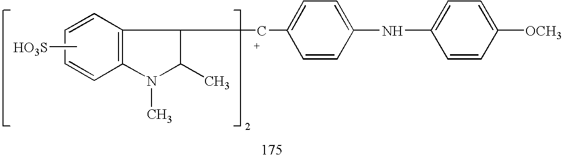
Photochromic Polymethine Dyes

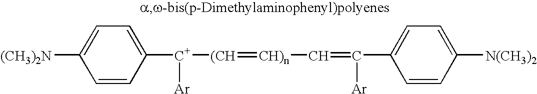

| Ar | n |
|---|---|
| C₆H₅ | 0, 1, 2 |
| 4-(CH₃)₂NC₆H₄ | 0, 1, 2 |
| 4-(CH₃)₂CHC₆H₄ | 0, 1, 2, 3, 4 |
| 4-CH₃OC₆H₄ | 0, 1, 2 |
| 4-C₄H₉OC₆H₄ | 0, 1, 2 |
| 3-CH₃C₆H₄ | 1, 2 |
| 4-t-C₄H₉C₆H₄ | 1, 2 |
| 4-C₂H₅OC₆H₄ | 1, 2 |
| 4-C₅H₁₁C₆H₄ | 1, 2 |
| 4-FC₆H₄ | 1 |
| 4-Fsub₃CC₆H₄ | 1 |
| 2-(C₆H₅)2NC6H4 | 1 |
| 3,4-H₂N(OCH₃)C₆H₃ | 1 |
| 2-Naphthyl | 1, 2 |
| 4-ClC₆H₄ | 2 |
| 2,4-Cl₂C₆H₃ | 2 |
| 1 -Naphthyl | 2 |

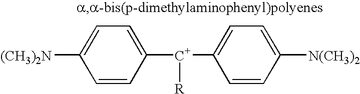
| R | R |
|---|---|
| 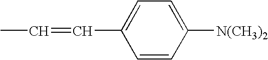 | 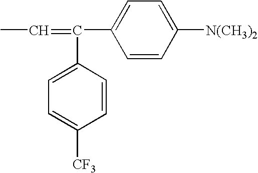 |
| 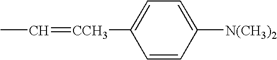 | |
| 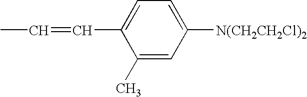 | 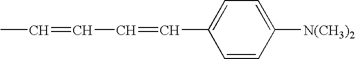 |
| 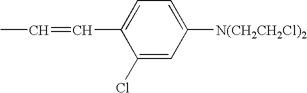 | 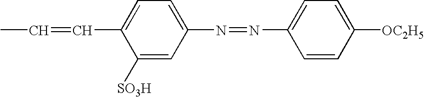 |
| 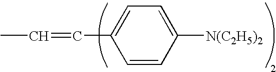 | —CH═N—N(C₆H₅)₂ |
| 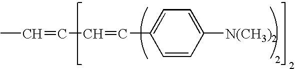 | 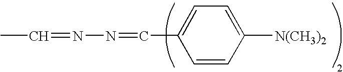 |
| | 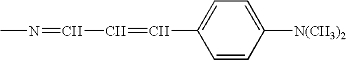 |
| 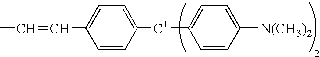 | 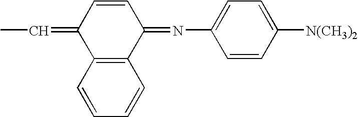 |
| 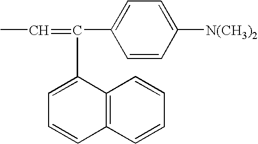 | 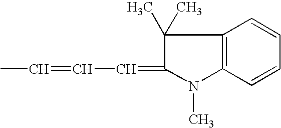 |

Miscellaneous polyenes 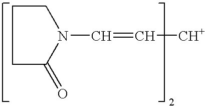 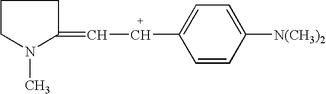 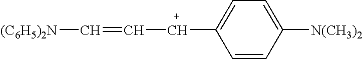 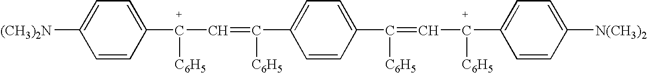 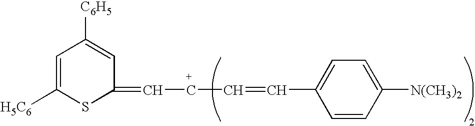 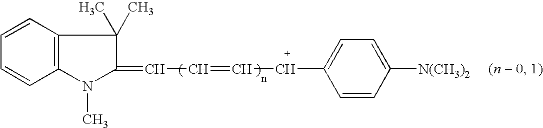 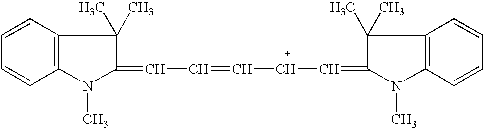
Basic Red 13 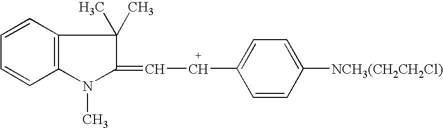
Basic Violet 7 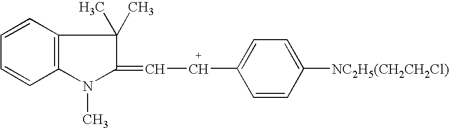
Basic Red 14
Basic Red 15
Basic Violet 15 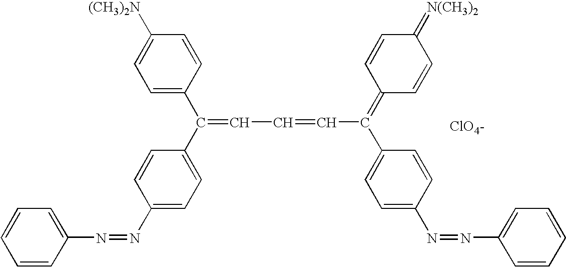 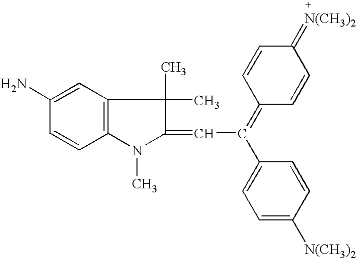 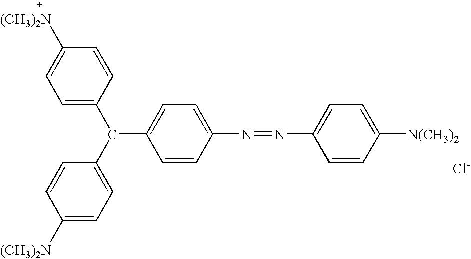 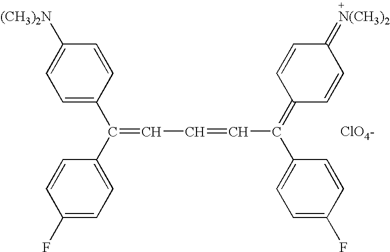 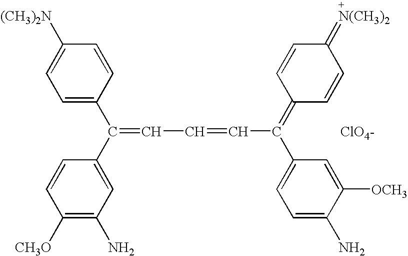 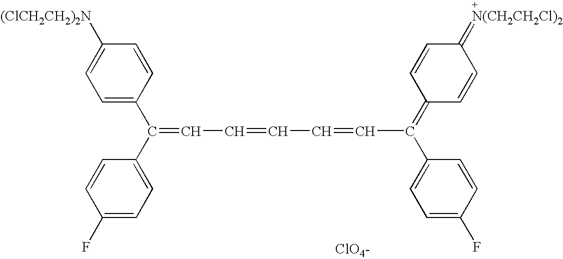 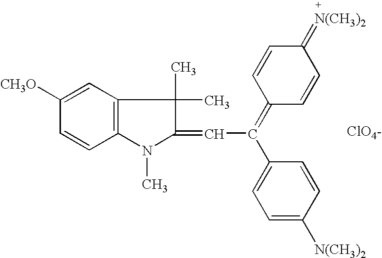 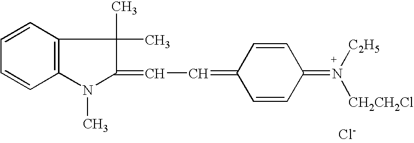 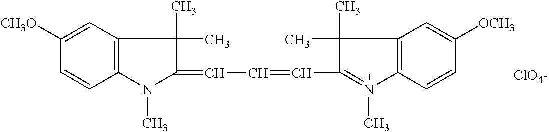 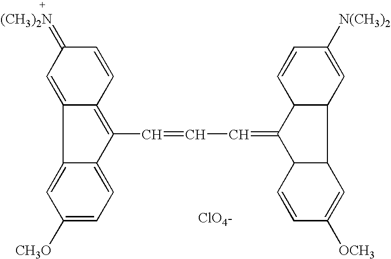 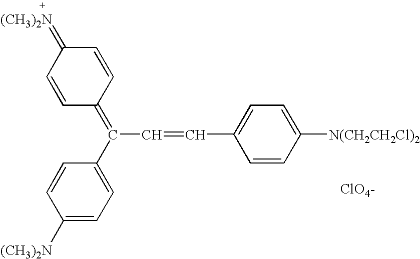 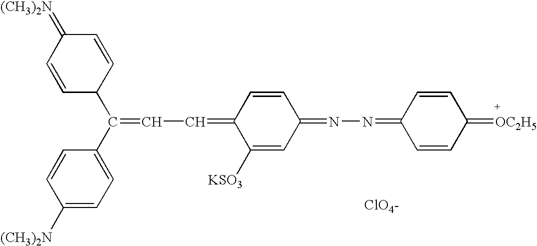 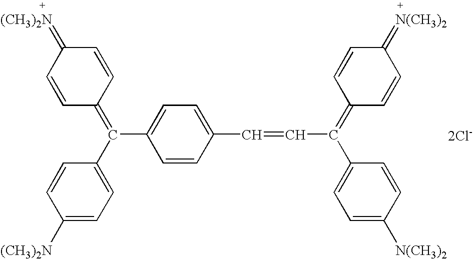 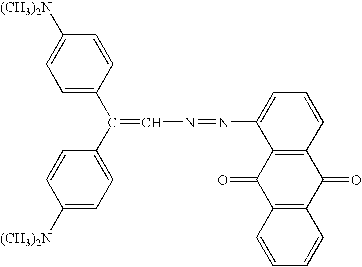 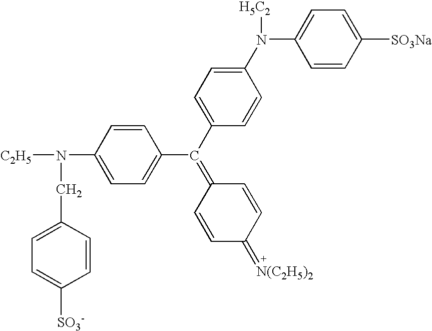 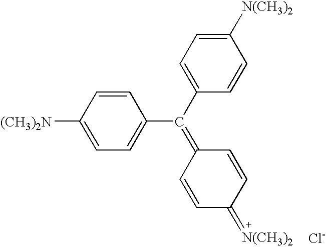 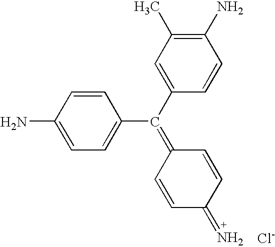 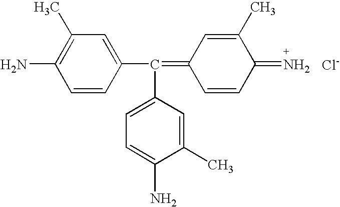 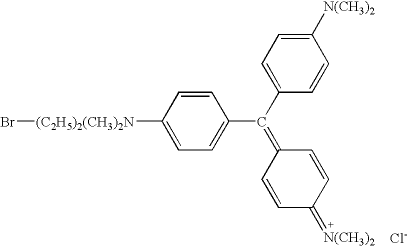 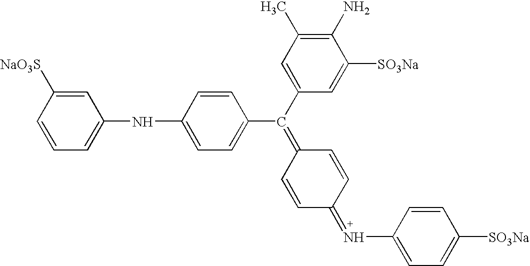 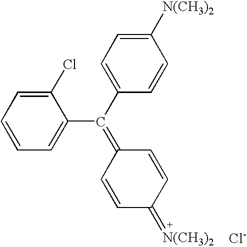 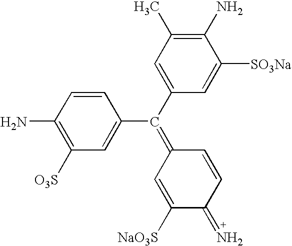 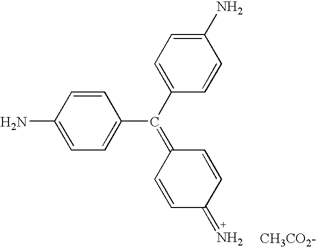 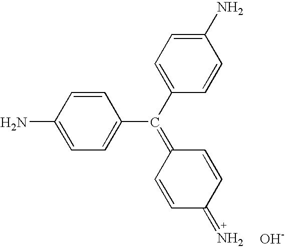 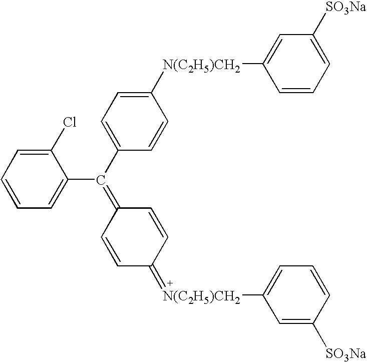 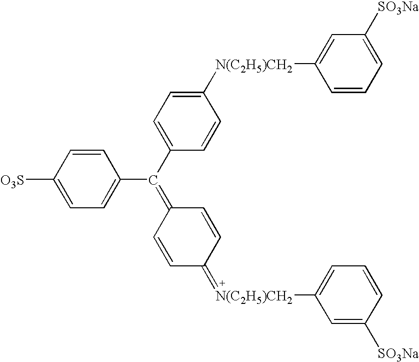 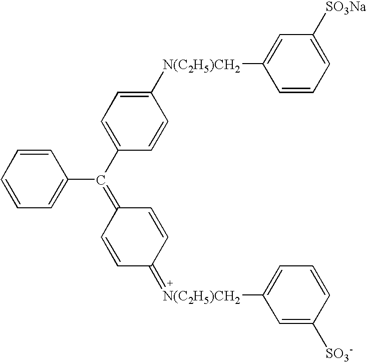 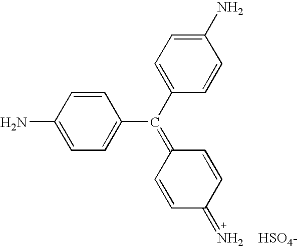
Salt-isomerism type phototropic dyes

| | |
|---|---|
| Night Blue | 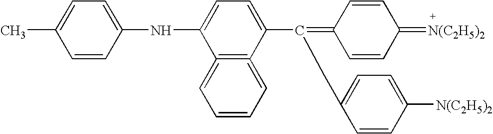 |
| Victoria Blue R | 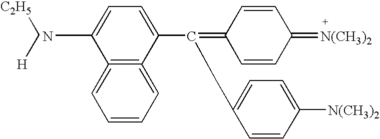 |
| Brilliant Milling Blue B Brilliant Blue F & R Ex. Eriocyanine A | 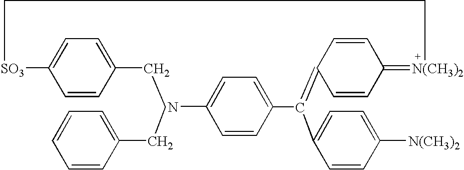 |
| Methyl Blue | 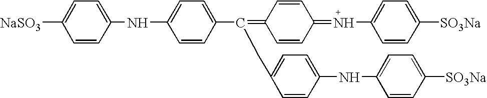 |
| Aniline Blue | 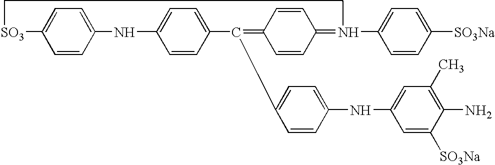 |
| Eriochrome Cyanine R | 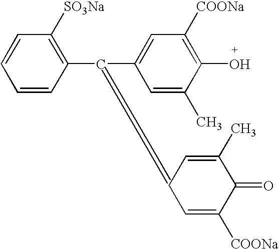 |
| Methyl Violet 6B | 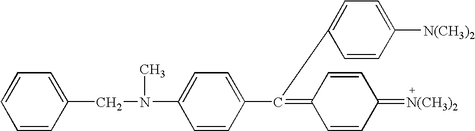 |
| Iodine Green | 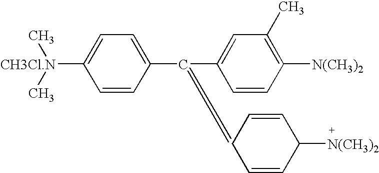 |
| Aniline Blue | 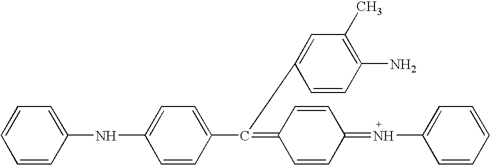 |
| Wool Violet 5 BN | 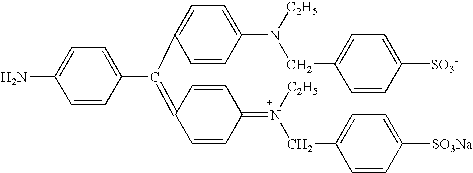 |
| Wool Violet 4 EM | 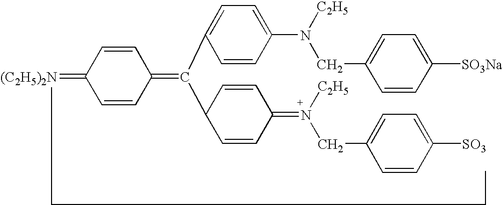 |
| Light Green SF Yellowish | 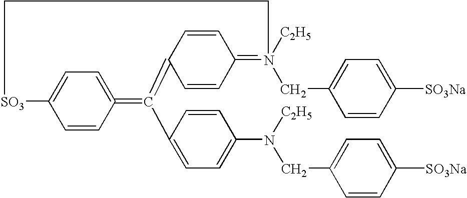 |
| Iodine Violet | 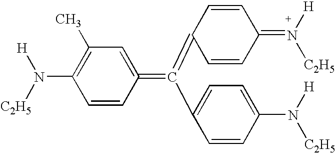 |
| Methyl Violet | 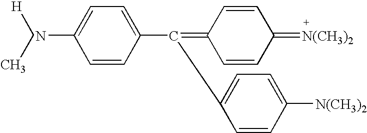 |
| Crystal Violet | 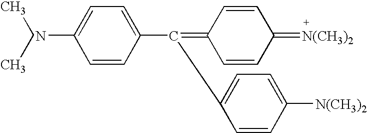 |
| Ethyl Violet | 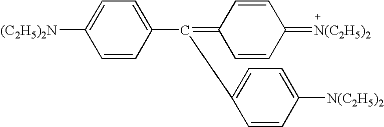 |
| Acid Green L Extra | 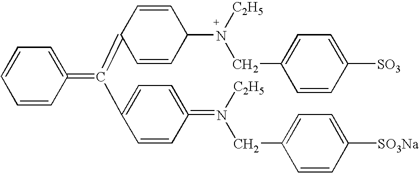 |
| Erioviridene B | 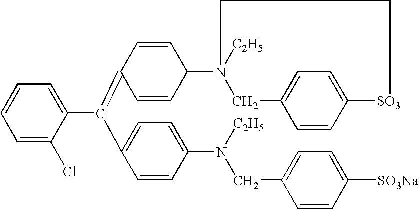 |
| Light Green SF | 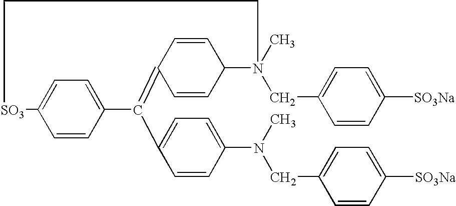 |
| Victoria Green (Malachite Green) | 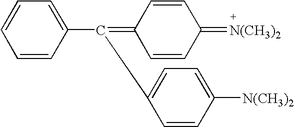 |
| Red-Violet SR | 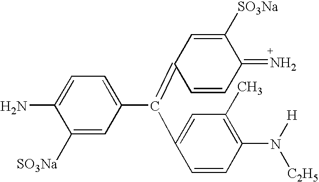 |
| Brilliant Green “B” | 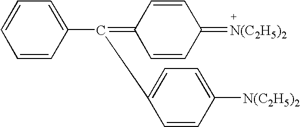 |
| Di-[4(N,N-diethylamime)phenyl]-[4- (N,N-diethyl-amine-2- methyl) phenyl] methyl carbonium | 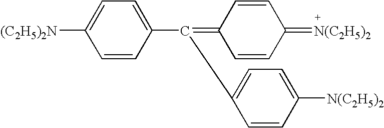 |
| Tri-[4(N,N-dipropylamino)phenyl]methyl carbonium | 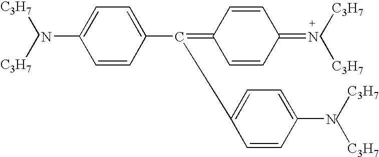 |
| Di-[4(N,N-diethylamino)phenyl]- [4(ethylamino)- phenyl] methyl carbonium | 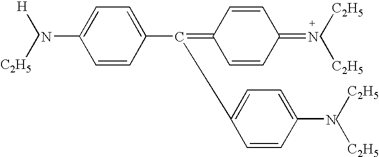 |
| Di-[4(N,N-diethylamino)phenyl]- [4(N,N-diethyl- amino)naphthyl]methyl carbonium | 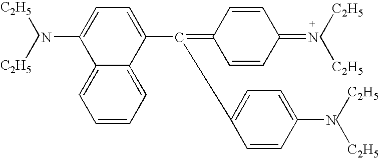 |
| Di-[4(N,N-dimethylamino)phenyl9 - [4(hydroxy)phenyl]methyl carbonium | 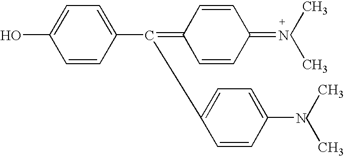 |
| Tri-[4(N-propylamina)phenyl]methyl carbonium | 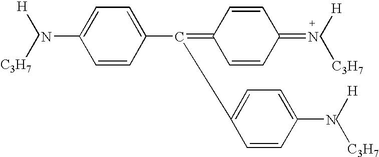 |
| Hectalene Blue DS-1398 Hectolene Blue DS-1823 Sevron Brilliant Red 4G Di-[4(N,N-dimethylamino)phenyl]- [4(hydroxy)phenyl]methyl carbonium | 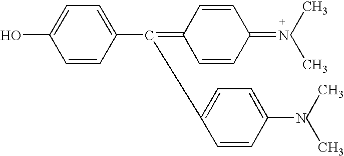 |
| Tri-[4(N-propylamino)phenyl]methyl carbonium | 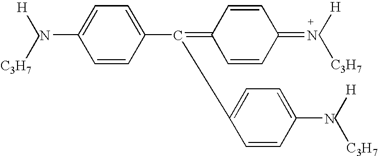 |
| Hectolene Blue DS-1398 Hectolene Blue DS-1823 Sevron Brilliant Red 4G Genacryl Red 6B Genacryl Pink G Sevrun Brilliant - Red B Sevron Brilliant - Red 3B 1,5-bis-[4(N,N-dimethylamiao)phenyl]- 1,5-bis-(phenyl)divinyl carbonium trifluoroacetate | 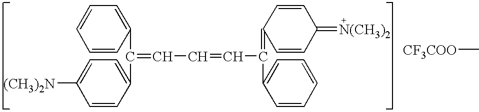 |
| 1,1,3,3-tetrakis[4(N,N- dimethylamino)phenyl]vinyl carbonium perchlorate | 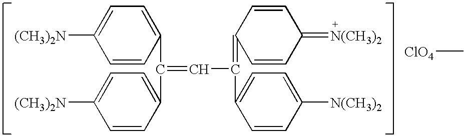 |
| 1,5-bis-[4(N,N-dimethylamino)phenyl]- 1,5-bis-(phenyl) divinyl carbonium p-toluenesulfonate | 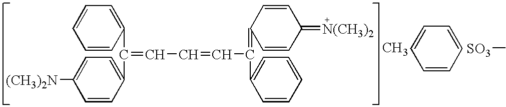 |
| 1,7-bis[4(N,N-dimethylamino)phenyl]- 1,7-bis-(2,4- dichlorophenyl) trivinyl carbonium perchlorate | 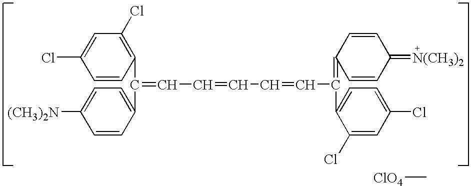 |
| Di-[4(N,N-dimethylamino)phenyl vinyl]-[2,4-di-phenyl-6- methane thiopyran]methyl carbonium perchlorate | 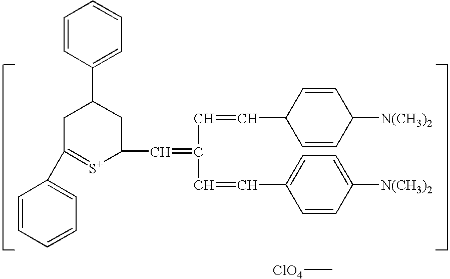 |
| 1,7-bis-[4(N,N-dimethylamino)phenyl]- 1,7-bis-(4-chlorophenyl) trivinyl carbonium trifluoroacetate | 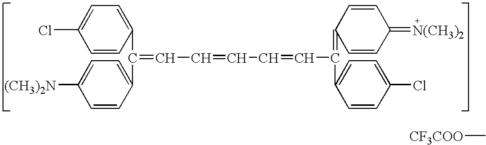 |
| 1,1,3-tris-[4-(N,N-dimethylamino) phenyl]]divinyl carbonium perchlorate | 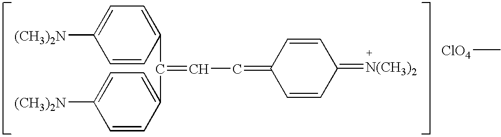 |
| 1,1,7,7-tetrakis-[4-(N,N- dimethylamino)phenyl]trivinyl carbonium perchlorate | 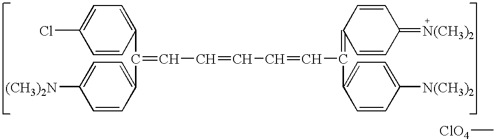 |
| 1,3-bis-[4-(N,N-dimethylamino) phenyl]-1,3-bis- (phenyl) vinyl carbonium perchlorate | 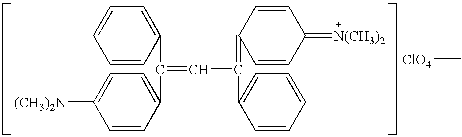 |
| 1,1,5,5-tetrakis-[4-(N,N- dimethylamino)phenyl]divinyl carbonium perchlorate | 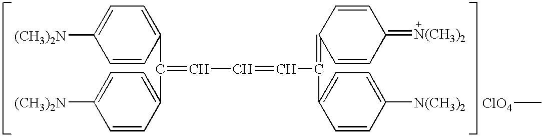 |
| 1,5-bis-[4-(N,N-dimethylamino) phenyl]-1,5-bis-(phenyl) divinyl carbonium perchlorate | 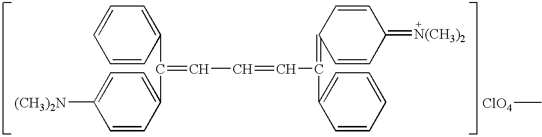 |
| 1,7-bis-[4-(N,N-dimethylamino) phenyl]-1,7-bis-(phenyl) trivinyl carbonium trifluoroacetate | 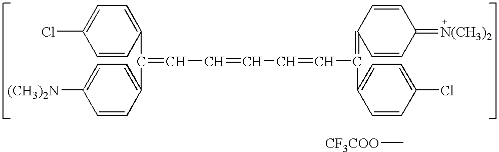 |
| 1(1,3,3-trimethyl indoline)-2-[4- (N,N-dimethyl-amino) phenyl] ethylene carbonium perchlorate | 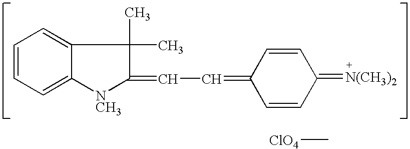 |
| 1(1,3,3-trimethyl indoline)-4-[4- (N,N-dimethyl-amino) phenyl] butylene carbonium perchlorate | 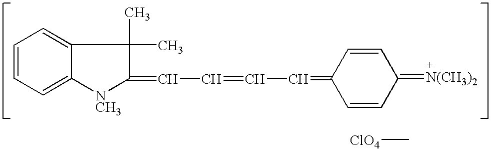 |
| 1,1,3,3-tetrakis-[4(N,N- diethylamino)phenyl]vinyl carbonium perchlorate | 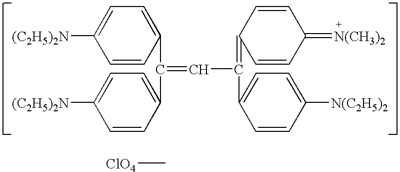 |
| 1,1-bis-[4-(N,N-diethylamino) phenyl]-3,3-bis- [4-(N,N-dimethylamino)phenyl]vinyl carbonium perchlorate | 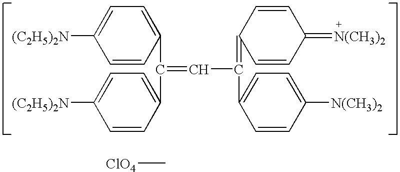 |
| 1,1,5,5-tetrakis-[4-(N,N- diethylamino)phenyl]divinyl carbomum perchlorate | 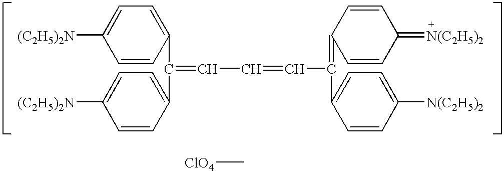 |
| 1,1-bis-[4-(N,N- dimethylamino)phenyl]-3- [4-(amino)phenyl]-3- methylvinyl carbonium perchlorate | 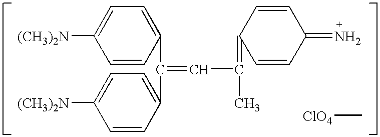 |
| Tris-[1,1-bis-[4(N,N- dimethylamino)phenyl]ethylene] methyl carbonium perchlorate | 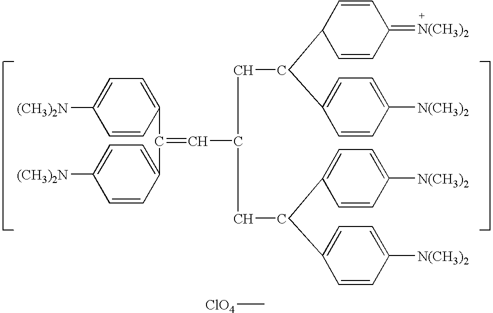 |
| Tris-[1,1-bis-[4-(N,N- diethylamino)phenyl]ethylene] methyl carbonium perchlorate | 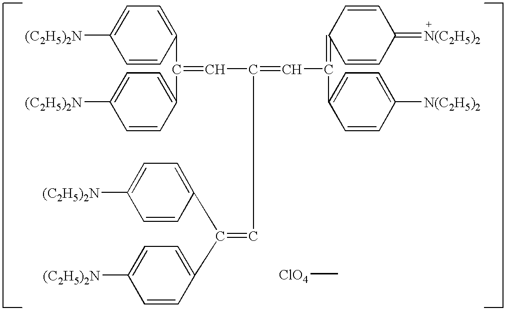 |
| 1,1,5-Tris-[4-(N,N- dimethylamino)phenyl]divinyl carbonium perchlorate | 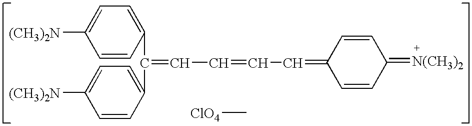 |
| N[4-(N,N-dimethylamino) cinnaniylidene] auramine | 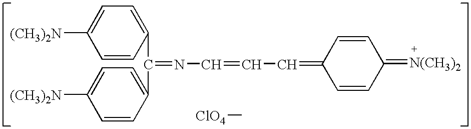 |
| 1,1-bis-[4-(N,N- dimethylamino)phenyl-3,4-bis- (phenyl)]-3,4-diazo butene carbanium | 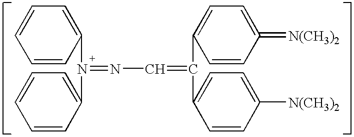 |
| 1,1,5,5-tetrakis-[4-(N,N- dimethylamino)phenyl]- 2,3-diazo pentene carbonium | 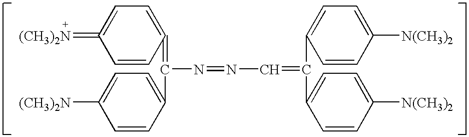 |
| N-(N′,N′-dimethylamino cinnamylidene)-N,N- diphenyl ammonium | 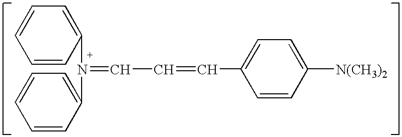 |
| Azo Polymethines Dyes of the general structural type | 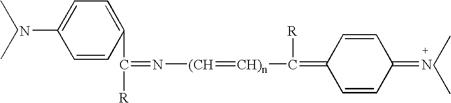 |

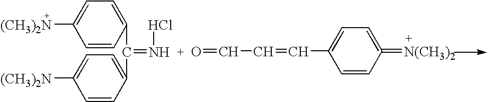

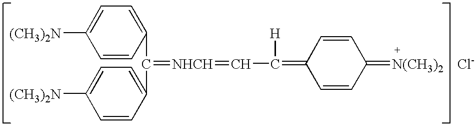
| | |
|---|---|
| Photochronic diazopolymethines | 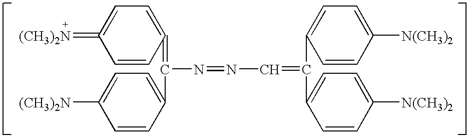 |
| 1,1,5,5-tetrakis-[4-(N,Np- dimethylamino)phenyl]- 2,3-diazo pentene carbonium | 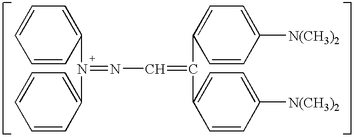 |
| 1,1-bis-[4-(N,N- | |
| dimethylamino)phenyl-3,4-bis- | |
| (phenyl)]-3,4-diazo | |
| butene carbonium | |

31. The method of synthesis of the compound of claim 10 wherein the C moiety is any molecule which exhibits bleaching behavior with the B moiety and has an increased therapeutic effect or therapeutic ratio as a consequence of its delivery as part of a prodrug.

32. The method of synthesis of the compound of claim 29 wherein the C moiety has a nucleophilic group that bonds to the B moiety.

33. The method of synthesis of the compound of claim 32 wherein the C moiety is derivatized to have a nucleophilic group that bonds to the B moiety.

34. The method of synthesis of the compound of claim 33 wherein the C moiety is derivatized by at least one of the nucleophilic groups comprising cinnamate, sulfite, phosphate, carboxylate, thiol, amide, alkoxide, or amine.

35. The method of synthesis of the compound of claim 10 wherein the C moiety is at least one of the group of

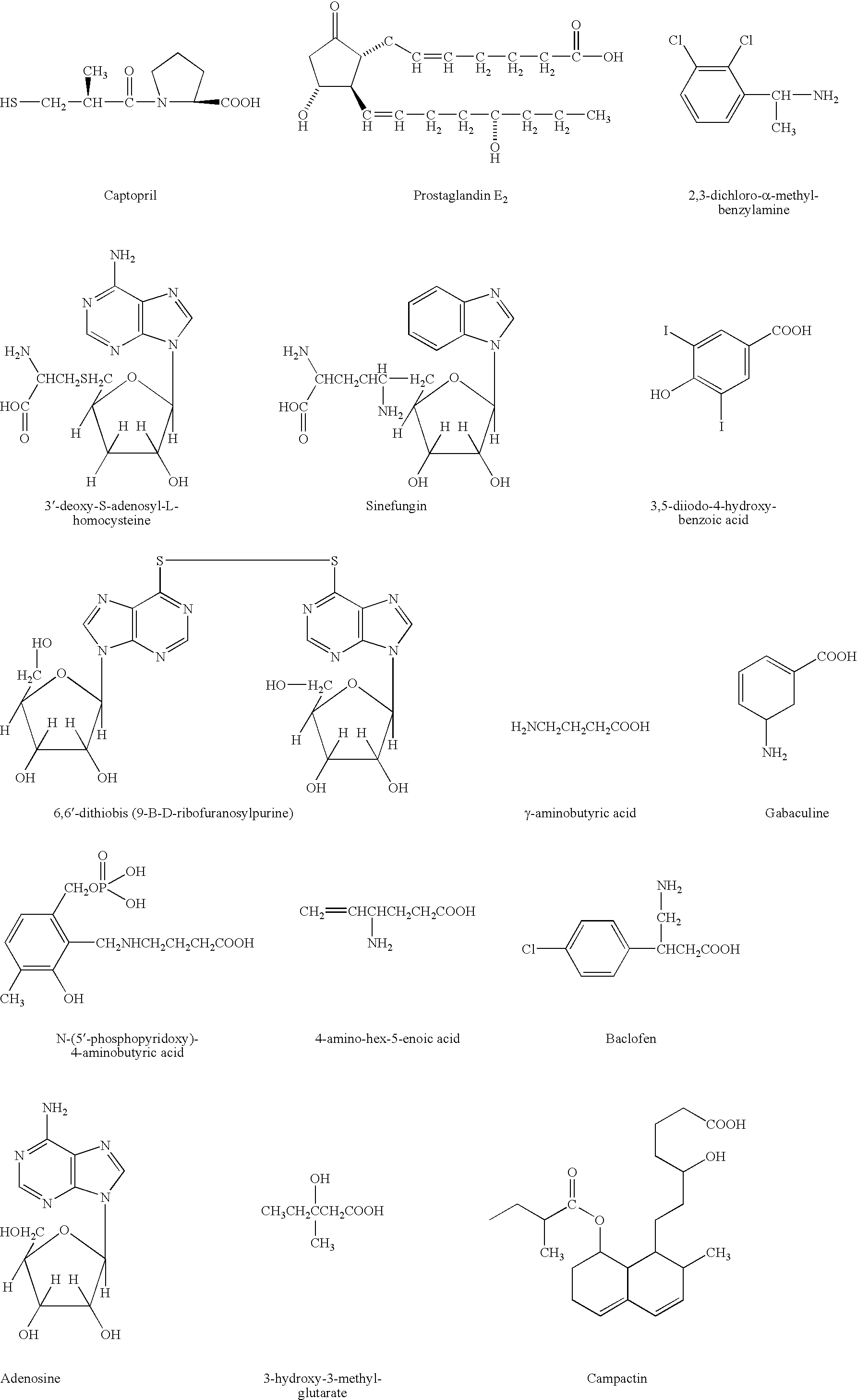

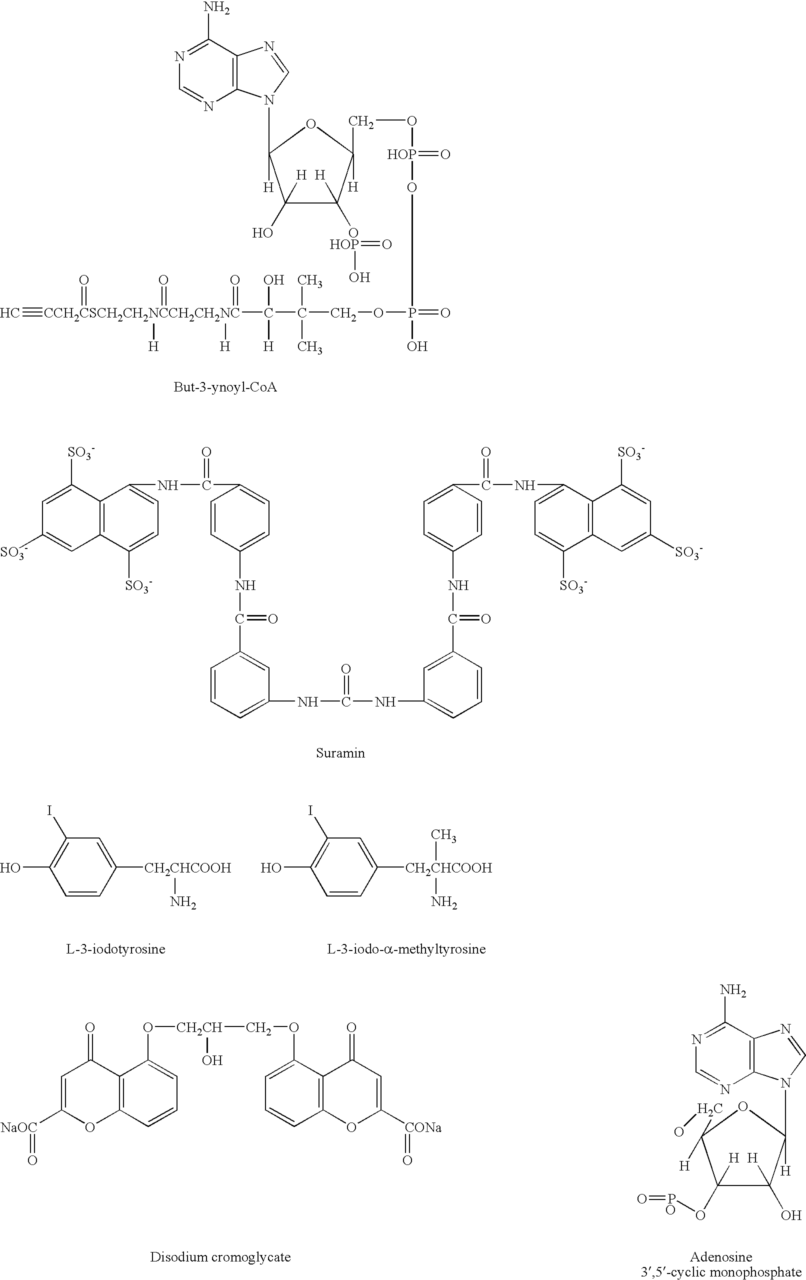

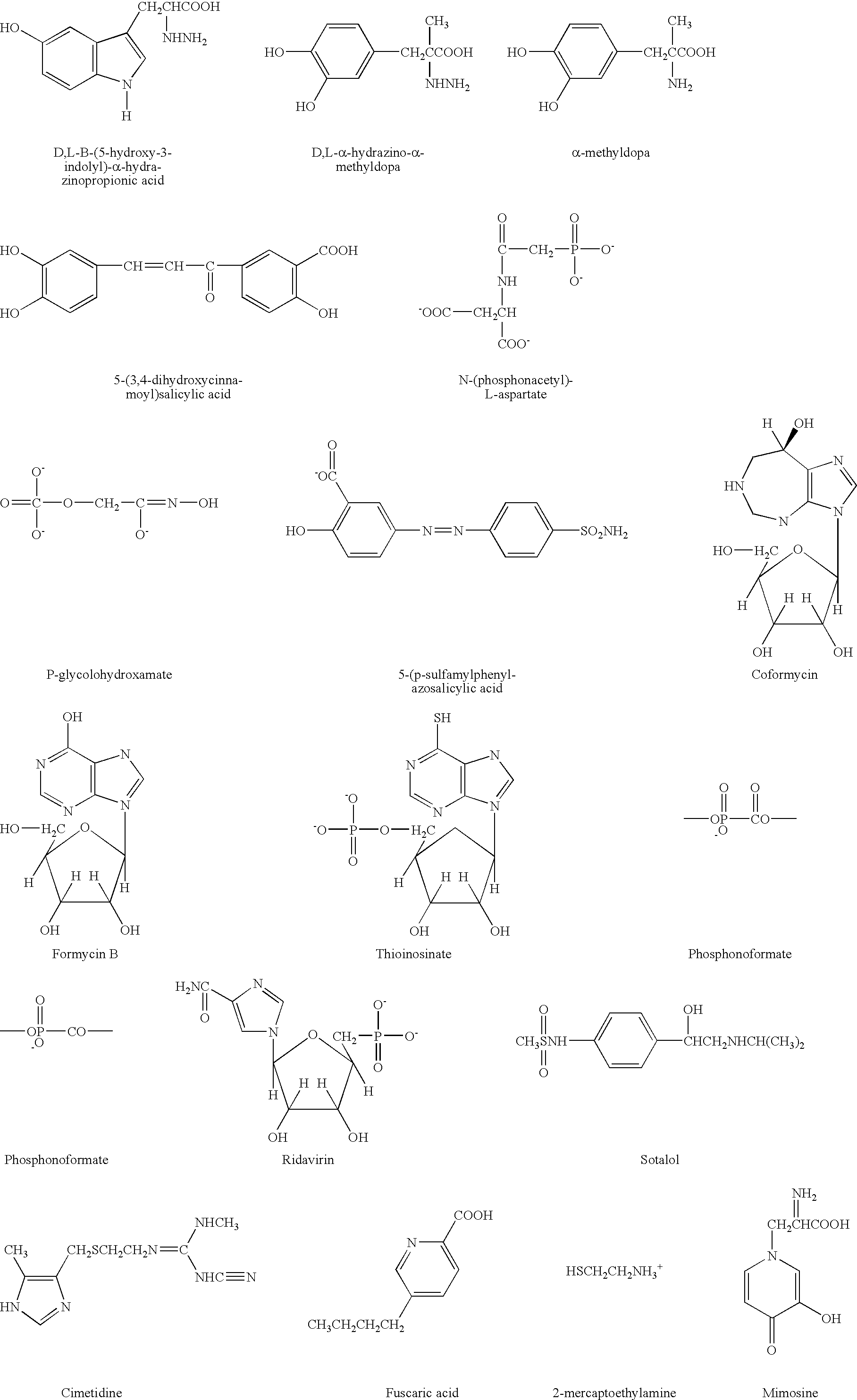

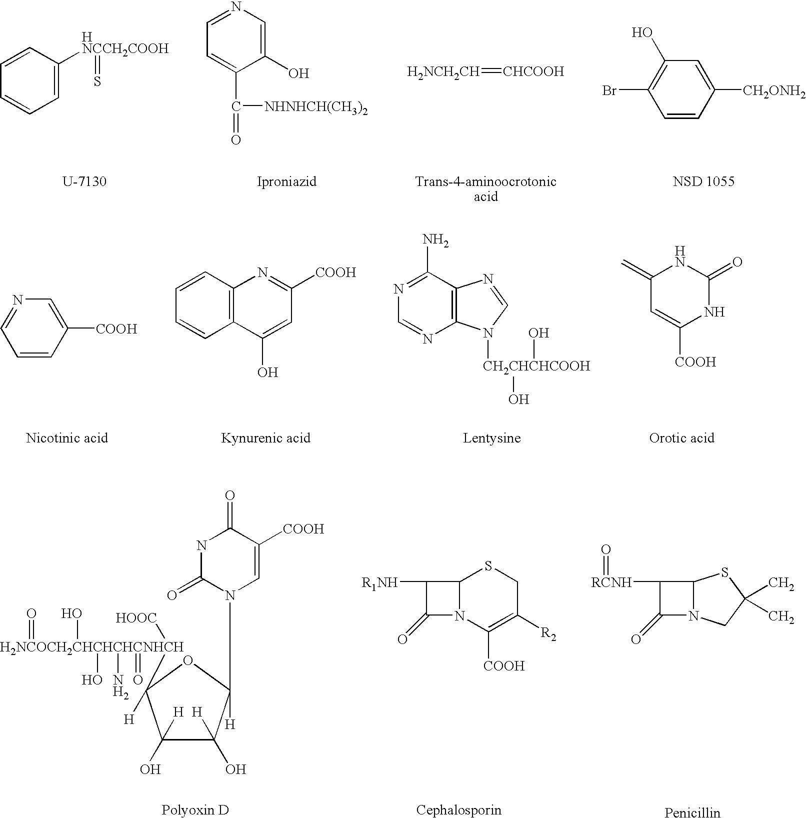

36. The method of synthesis of the compound of claim 10 wherein the C moiety is at least one or a derivative or analog of one of the group of
prostaglandins
prostaglandin A.sub.1 A.sub.2 B.sub.1 E.sub.1, E.sub.2 or an analog which possesses a vasodilatory effect on coronary arteries and other human vascular beds
prostaglandin E, F, A or an analog which possesses a positive cardiac inotropic effect
prostaglandin A, E, or an analogue prostaglandin which possesses natriuretic and diuretic activity
prostaglandin A, G, E.sub.1, E.sub.2 or an analogue such as 15(S)-15-methyl PGE 2 methylester, 16,16-dimethyl PGE.sub.2, . . . AY-22,093, AY . . . 0.22,469, AY-22,443, or 15(R)-15-methyl PGE.sub.2 which inhibits gastric acid secretion
prostaglandin D.sub.2, E.sub.1 or an analogue which inhibits platelet aggregation
prostaglandin E.sub.1, E.sub.2 or an analogue which causes bronchial dilatation
prostaglandin F2 or an analogue which causes abortion by luteolysis
prostaglandin A.sub.2, E.sub.1, E.sub.2, or an analogue which induces erythropoiesis
prostaglandin E or an analogue which modulates T lymphocytes to decrease their ability to reject an allogenic graft
2′-isopropyl-4′-(trimethylammonium chloride)-5′-methylphenyl piperidine-1-carboxylate (Amo 1618) or an analog which inhibits the cyclization of trans-geranyl-geranyl-PP to copalyl-PP during Kaurene synthesis
adenosine cyclic 3′,5′-monophosphate or an analogue which inhibits the release and formation of phlogistic mediators such as histamine and kinins
4′-sulfamylphenyl
2-azo-7-acetamid-1-hydroxynaphthalene-3,6-disulfonate (Neoprontosil), 4′-sulfamyl-2, 4-diaminoazobenzene (Prontosil), or 5-(p-sulfamylphenylazo) salicylic acid (Lutazol) or analog which possess potent carbonic acid anhydrase inhibition
analogue of S-adenosyl homocysteine or sinefungin
phosphoglycolohydroxamate which inhibits Class II aldolases present in bacterial and fungi and is noninhibitory of Class I aldolases present in animals,
inosine analogue such as formycin B which inhibits nucleotide phosphorylase during nucleotide metabolism
phosphonoformate (Foscarnet) or an analog which inhibits the HIV reverse transcriptase enzyme
gamma.-amino-butyric acid (GABA) or an analog which is the major inhibitory neurotransmitter in the mannalian central nervous system
gabaculine, N-(5′-phosphopyridoxyl)-4-aminobutyric acid, ethanolamine-o-sulfate, .gamma.-vinyl GABA, or .gamma.-acetylenic GABA or an analog that is an inhibitor of the GABA-degrading enzyme, GABA: 2-oxoglutarate aminotransferase
Baclofen or a compound that inhibits GABA release an oligonucleotide which binds to RNA or DNA and blocks transcription or translation of HIV or P-glycoprotein gene products adenosine which binds to brain purinergic receptors to suppress opiate withdrawal
adensoine whihc causes coronary vasodilatation
3-hydroxy-3-methylglutarate, 3-hydroxybutyrate, 3-hydroxy-3-methylpentanoate, 4-bromocrotonyl-CoA, but-3-ynoyl-CoA, pent-3-ynoyl-CoA, dec-3-ynoyl-CoA, ML-236A, ML-236B (compactin), ML-236C, mevinolin, mevinolinic acid, or a mevalonic acid analogue which is an inhibitor of 3-hydroxy-3-methylglutaryl-CoA reductase which catalyzes the rate-limiting and irreversible step of cholesterol synthesis where inhibition at this step does not lead to the accumulation of nonmetabolizable precursors
thioinosinate which suppresses T lymphocytes
Suramin, which is a powerful inhibitor of energy driven calcium uptake by the sarcoplasmic reticulum and is an intracellular inhibitor of Na.sup.+-K.sup.+ATPase where both activities increase intracellular calcium concentrations with a concomitant inotropic effect
norepinephrine N-methyltransferase inhibitor such as 2,3-dichloro-.alpha.-methylbenzylamine, 2,3-dichlorobenzylamine, 2,3-dichlorobenzamidine, or 3,4-dichlorophenylacetamidine
adenosine cyclic 3′,5′-monophosphate or a cAMP analogue which blocks the synthesis of fatty acids and cholesterol in the liver is an antilipidemic agent,
an inhibitor of dihydroxyphenylalanine decarboxylase during the synthesis of epinephrine and norepinephrine such as psitectorigenin, genistein, 3′, 4′,5,7-tetrahydroxy-8-methylisoflavone, orbol, 8-hydroxygenistein, 3′,5,7-trihydroxy-4′,6-dimethylisoflavone, 3′,5,7-trihydroxy-4′,8-dimethoxyisoflavone, D,L-B-(5-hydroxy-3-indolyl)-.alpha.-hydrazinopropionic acid, D,L-.alpha.-hydrazino-.alpha.-methyldopa, D,L-B-(3-indolyl), -.alpha.-hydrazinopropionic acid, a derivative of phenylalanine such as N-methyl-3,4-dopa, .alpha.-acetamido-3,4-dimethyoxycinnamic acid, DL-.alpha.-methyl-3,4-dopa, .alpha.-methyl-B-(3-hydroxy-4-methoxyphenyl)alanine, alpha.-methyl-3,4-dimethoxyphenylalanine, or d-catechin; D,L-B-(3-indolyl)-.alpha.-methyl-.alpha.-hydrazinopropionic acid (R)-303,4-dihydroxyphenyl!-1-fluoropropylamine, (S)-.alpha.-fluoromethyldopa, (S)-.alpha.-fluoromethyltyrosine, 5-(3,4-dihydroxycinnamoyl) salicylic acid, 3-hydroxycinnamic acid, caffeic acid, 3-mercaptocinnamic acid, .alpha.-methyl-3-hydroxycinnamic acid, .alpha.-ethyl-3-hydroxycinnamic acid, 3-hydroxy-w-nitrostyrene, 3,4-dihydroxyhydrocinnamic acid, 3-hydroxybenzalacetone, 3-hydroxychalone, 3-hydroxybenzal furanyl ketone, 3-hydroxybenzal thiophenyl ketone, 3′,4′-dihydroxyflavone, 8-O-glucoseflavone, flavone, 3-hydroxyphenyl pyruvic acid, 3,4-dihydroxyphenylpyruvic acid phenylthiopyruvic acid, 4-hydroxyphenylpyruvic acid, dithiosalicyclic acid, 1-hydroxy-2-naphthoic acid, 3-hydroxy-7-sulfo-2-naphtholic acid, 3,5-dihydroxy-2-naphtholic acid, 4-chlorocinnamic acid, 2-chlorocinnamic acid, 2,4-dichlorocinnamic acid, 3-nitrocinnamic acid, 3,5-dibromo-2-hydroxycinnamic acid, 2,4,6-triiodo-3-hydroxycinnamic acid, 2-hydroxy-4′-cyanochalone, 4-(4-hydroxycinnamoyl) benzylnitrile, 2-(4-hydroxycinnamoyl)-1,4-dihydroxybenzene, quercetin-6′-sulfonic acid, 5-(2-hydroxy-3,5-dibromocinnamoyl) salicylic acid or 5-(3-hydroxycinnamoyl) salicylic acid
an inhibitor of acrosin, a proteolytic enzyme located in the acrosome of sperm, such as tosyl lysine chloromethyl ketone, N-.alpha.-tosyl-L-arginine chloromethyl ketone, or ethyl p-guanidinobenzoate,
adenosine cyclic 3′,5′-monophosphate (cAMP), N.sup.6, O.sup.2-dibutyryladenosine cyclic 3′,5′-monophosphate or an analogue which produces an inotropic response,
adenosine kinase enzyme inhibitor such as 6,6′-dithiobis (9-B-D-ribofuranosylpurine),
inhibitor of monoamine oxidase such as phenylhydrazine, phenylethylidenehydrazine, isopropylhydrazine, or iproniazid,
an inhibitor of catechol-o-methyltrasferase such as 3,5-diiodo-4-hydroxybenzoic acid, S-3′-deoxyadenosylL-homocysteine, pyrogallol, R04-4602, gallic acid, 3,5-dihydroxy-4-methylbenzoic acid, 1,3-dihydroxy-2-methoxybenzene, 1-hydroxy-2,3-dimethoxybenzene, 2-hydroxy-1,3-dimethoxybenzene, 1,3-dihydroxy-4-methoxybenzene, catechol, 3,4-dihydroxybenzoic acid, caffeic acid, 5,6-dihydroxyindole, noradnamine, dopacetamide, H 22/54, quercetin, nordihydroguaiaretic acid, U-0521, arterenone, methylspinazarin, MK 486, dopa, papaveroline, isoprenaline, 7,8-dihydroxy-chlorpromazine, 3-hydroxy-4-pyridone, tetrahydroisoquinoline pyridoxal 5′-phosphate, iodoacetic acid, 3-mercaptotyramine, dehydrodicaffeic acid dilactone, methylspinazorin, 3′,5,7-trihydroxy-4′,6-dimeth-oxyisoflavone, 3′,5,7-trihydroxy-4′,8-dimeth-oxyisoflavone, 6,7-dihydromethylspinazarin, S-adenosylhomocysteine, S-tubercidinylhomocysteine, 3′,8-dihydroxy-4′,6,7-trimethoxyisoflavone, 7-O-methylspi nochrome B, 6-(3-hydroxybutyl)-7-O-methylspinachrome B, 3,5-diiodosalicyclic acid, or pyridoxal-5′-phosphate,
an inhibitor of adenosine deaminase which blocks the metabolism of adenosine such as coformycin, arabinosyl-6-thiopurine, 6-methylthioinosine, 6-thioinosine, 6-thioguanosine, N.sup.1-methyladenosine, N.sup.6-methyladenosine, 2-fluorodeoxyadenosine, 2-fluoroadenosine, inosine, 2′-deoxyinosine, deoxycoformycin, 1,6-dihydro-6-hydroxymethyl purine ribonucleoside, erythro-9-(2-hydroxy-3-nonyl)adenine, or 9-B-D-arabinofuiranosyl-6-hydroxylaminopurine,
an inhibitor of adenylate kinase, 5′-nucleotidase, and adenosine translocase such as p.sup.1 p.sup.5-diadenosine pentaphosphate, .alpha.,.beta.-methylene adenosine diphosphate, and nitrobenzyl-6-thioinosine, respectively,
an inhibitor of .GAMMA.-aminobutyric acid uptake such as D,L-2,4-diaminobutyric acid, D,L-B-hydroxy GABA, (−)-nipecotic acid, trans-4-aminocrotonic acid, cis-3-aminocyclopentane-1-carboxylic acid, trans-3-aminocyclopentane-1-carboxylic acid, B-guanidinopropionic acid, homohypotaurine, 4-aminopentanoic acid, homotaurine, B-alanine, imidazoleacetic acid, 6-aminohexanoic acid, D,L-carnitine, D,L-2,6-diaminopimetic acid, D,L-2-fluoro GABA, guanidino acetic acid, 2-hydrazinopropionic acid, taurine, D,L-omithine, or sulphanilamine which potentiates the inhibitory action of GABA,
inositol 1,4,5-triphosphate,
guanosine 5′ cyclic monophosphate or 8-bromo guanosine 5′ cyclic monophosphate which relaxes smooth muscle,
an inhibitor of the uptake system for glycine, the inhibitory synaptic transmitter of the spinal cord, such as hydrazinoacetic acid,
isoquinoline-sulfonamide inhibitor of protein kinase C, cAMP-dependant protein kinase, or cGMP-dependent protein kinase such as N-(2-aminoethyl)-5-isoquino-linesulfonamide,
Ribavirin which is active against HSV-1 and 2, hepatitis, and influenza viruses, or phosphonoacetic acid which is a highly specific inhibitor of Herpes Simplex virus induced polymerase and is active against HSV-1 and HSV-2, or adenine arabinoside (ara-A), cytosine arabinoside (Ara-C), ara-A 5′-monophosphate (ara-AMP), or hypoxanthine arabinoside (ara-Hx) which is active against HSV or phagicin which is active against vaccinia and HSV, or 4-fluoroimidazole, 4-fluoroimidazole-5-carboxylic acid, 4-fluoroimidazole-5-carboxamide, 5-fluoro-1-B-D-ribofurano-sylimidazole-4-carboxamide, 5-amino-1-B-D-ribofuranosyl-imidazole-4-carboxamide, poly (I).multidot.poly (C), sinefungin, iododeoxyuridine, 9-(2-hydroxy-ethoxymethyl) guanine, gliotoxin, distamycin A, netropsin, congocidine, cordycepin, 1-B-D-arabinofuranosylthymine, 5,6-di-hydroxy-5-azathymidine, pyrazofurin, toyocamycin, or tunicamycin,
an inhibitor of fungal chitin synthetase such as polyoxin D, nikko-mycin Z, or nikkomycin X,
an impermeant antifungal agent such as ezomycin A.sub.1, A.sub.2, B.sub.1, B.sub.2, C.sub.1, C.sub.2, D.sub.1, or D.sub.2 or platenocidin, septacidin, sinefumgin, A9145A, A9145C, or thraustomycin,
an inhibitor of central nervous system carbonic anhydrase such as methazolamide, or 2-benzoylimino-3-methyl-.DELTA.sup.4-1,3,4-thiadiazoline-5-sulfonamide subsgituted at the benzolyl group with 3,4,5-trimethoxy, 2,4,6-trimethoxy, 2,4,5-trimethoxy, 4-chloro, 4-bromo, 4-iodo, or hydrogen,
an inhibitor of dopamine-B-hydroxylase during the synthesis of norepinephrine and epinephrine such as fuscaric acid, 5-(3′,4′-dibromobutyl)picolinic acid, 5-(3′-bromobutyl) picolinic acid, 5-(3′,4′-dichlorobutylpicolinic acid, YP-279, benxyloxyamine, p-hydroxybenzyloxyamine, U-21,179, U-7231, U-6324, U-0228, U-5227, U-10,631, U-10,157, U-1238, U-19,963, U-19,461, U-6628, U-20,757, U-19,440, U-15,957, U-7130, U-14,624, U-22,996, U-15,030, U-19,571, U-18,305, U-17,086, U-7726, dimethyldithiocarbamate, diethyldithiocarbamate, ethyldithiocarbamate, 2-mercaptoethylguanidine, thiophenol, 2-mercaptoethylamine, 3-mercaptopropylguanidine, 3-mercap-toprbpyl-N-methylguanidine, 2-mercaptoethanol, 2-mercaptoethyl-N-methylguanidine, 2-mercaptoethyl-N,N′-dimethylguanidine, 4,4,6-trimethyl-3,4-dihydropyrimidine-2-thiol, N-phenyl-N′-3-(4H-1,2,4-trizolyl)thiourea, methylspinazarin, 6,7-dimethylspinazarin, 7-O-methy-spinochrome B, 6-(3-hydroxybutyl)-7-O-methylspinachrome B, aquayamycin, chrothiomycin, frenoclicin, N-n-butyl-N′-3-(4H-1,2,4-trazolyl) thiourea, propylthiouracil, mimosine, mimosinamine, or mimosinic acid,
an inhibitor of histidine decarboxylation during the synthesis of histamine such as .sup.2-hydroxy-5-carbomethoxybenzyloxyamine, 4-toluene-sulfonic acid hydrazide, 3-hydroxy benzyloxyamine, hydroxylamine, aminooxyacetic acid, 4bromo-3-hydroxybenzyloxyamine (NSD-1055), rhodanine substituted in the 3 position with p-chlorophenethyl, p-chlorobenzyl, p-methylthiobenzyl, p-methylbenzyl, p-fluorobenzyl, amino, 3,4-dichlorobenzyl, p-bromobenzyl, p-methoxybenzyl, p-bromoanilino, p-iodoanilino, p-chloroanilino, p-toluidino, anilino, 2,5-dichloroanilino, dimethylamino, or p-methoxyphenyl; 2-mercaptobenzimidazole-1,3-dimethylol, 4-bromo-3-hydroxy-benzoic acid, 4-bromo-3-hydroxybenzyl alcohol, 4-bromo-3-hydroxyhippuric acid, (R,S)-.alpha.-fluoromethyl-histidine, (S)-.alpha.-fluoromethylester, L-histidine ethyl ester, L-histidinamide, D,L-3-amino-4-(4-imidazolyl)-2-butanone, 2-bromo-3-hydroxybenzyloxyamine, 5-bromo-3-hydroxybenzyloxyamine, 4,6-dibromo-3-hydroxybenzyloxyamine, aminooxypropionic acid, benzyloxyamine, 4-bromo-3-benzenesulfonyloxybenzyloxyamine, 3′,5,7-trihydroxy-4′,6-dimethoxyisoflavone, lecanoric acid, N-(2,4-dihydroxybenzoyl)-4-aminosalicylic acid, or 3′,5,7-trihydroxy-4′,8-dimethoxyisoflavone,
an pharamaceutical aget of drug that appear in Physicians Desk Reference, Edward R. Barnhart, 41th ed., 1987, Medical Economics Company Inc., N.J.; USAN and the Dictionary of Drug Names, ed. by Mary C. Griffiths, The United States Pharmacopedial Convention, (1986); and The Pharmacological Basis of Therapeutics, ed. by A. G. Gilman, L. Goodman, A. Gilman, 7th ed., (1985), MacMillan Publishing Co., N.Y., N.Y.,
a centrally acting converting enzyme inhibitor such as captopril,
an antibacterial agent such as penicillin, cephalosporin, or cephamycin, with B-lactamase resistance,
an agent which blocks bacterial synthesis of tetrahydrofolate such as a sulfonamide (an analogue of p-aminobenzoic acid) including sulfanilamide, sulfadiazine, sulfamethoxazole, sulfisoxazole, or sulfacetamide
an inhibitor of dihydrofolate reductace including pyrimethamine, cycloguanil, trimethoprin, isoaminopterin, 9-oxofolic acid, or isofolic acid,
a bactericidal agent such as nalidixic acid or oxolinic acid,
an inhibitor of bacterial protein synthesis such as vancomycin, an aminogylcoside, erythromycin, tetracyclin, or chloramphenicol,
an inhibitor of viral DNA polymerase such as vidarabine,
tuberculostatic or tuberculocidal agent such as isoniazid or aminosalicyclic acid,
an anthelmintic agent such as oxamniquine, piperazine, metronidazole, diethylcarbamazine, paromomycin, niclosamide, bithionol, metrifonate, hycanthone, dichlorophen, or niclosamide,
an H.sub.2-blocking agent such as cimetidine or ranitidine,
an agent which blocks release of norepinephrine such as sotalol, guanethidine, pindolol, pronethalol, KO 592, practolol, oxprenolol, or pronethalol,
a xanthine oxidase inhibitor such as allopurinol, thioinosinate, 5,7-dihydroxypyrazolo õ1,5-a! pyrimidine substituted at the 3 position with hydrogen, nitro, bromo, chloro, phenyl, 3-pyridyl, p-bromophenyl, p-chlorophenyl, p-acetylanilino, p-tolulyl, m-tolulyl, naphthyl, or 3,4-methylenedioxyphenyl; 8-(m-bromoacetamidobenzylthio)hypoxanthine, 8-(m-bromoacetamidobenzylthio)hypoxanthine, guanine substituted at the 9 position with phenyl, 4-chlorophenyl, 3-chlorophenyl, 3,4-dichlorophenyl, 4-methoxyphenyl, 3,4-dimethoxyphenyl, 4-dimethylaminophenyl, 4-aminophenyl, 3-aminophenyl, 3-trifluormethylphenyl, 4-benzamido, 4-carboxylphenyl, 4-methylpheyl, 4-ethylphenyl, 3-methylphenyl, B-naphthyl, or 4-ethoxyphenyl; 4,6-dihydroxypyrazolo õ3,4-d! pyrimidine, 4-trifluoromethylimidazoles substituted at the 2 position with phenyl, p-chlorophenyl, p-methoxyphenyl, p-acetylanilino, p-nitrophenyl, p-dimethylaminophenyl, p-cyanophenyl, p-fluorophenyl, p-carboxyphenyl, m-chlorophenyl, 3,4-dichlorophenyl, 4-pyridyl, 3-pyridyl, 2-quinolyl, 6-quinolyl, 4-quinolyl, 7-quinolyl, 2-pyrazinyl, or 1-(2-pyridyl-4-trifluoromethyl-5-bromoimidazolyl; 5-(4-pyridyl)-1,2,4-triazoles substituted at the 5 position with 4-pyridyl, 3-pyridyl, 2-pyridyl, phenyl, p-chlorophenyl, m-chlorophenyl, p-sulfonamidophenyl, 3,5-dichlorophenyl, 3,5-dicarboxyphenyl, 6-quinolyl, 2-furyl, 4-pyridazinyl, 2-thienyl, 2-pyrimidinyl, 4-pyrimidinyl, or 4-pyrazinyl; difunisal, 4(or 5)-(2-aminoethylthio-azo)imidazole-5(or 4)-carboxamide, 4 (or 5)-diazoimidazole-5(or 4)-carboxamide, or S-õ5(or 4)-carbamoyl-4(or 5)-imidazolyl azo! cysteine,
an agent which inhibits DNA synthesis such as a bis-thiosemicarbazone, 3,5-diisopropylsalicyl-hydroxamic acid, 4-hydroxybenzoylhydroxamic acid, 3-methylsalicylhydroxamic acid 2,5-dihydroxybenzoylhydroxamic acid, or 2-hydroxy-3,4,5-trimethoxybenzoylhydroxamic acid; or which inhibits nucleotide synthesis such as N-(phosphoacetyl)-L-aspartate which inhibits asparatate transcarbamylase during pyrimidine synthesis, or azaserine or 6-diazo-5-oxo-L-norleucine which inhibits purine synthesis at the phosphoribosyl-formyl-glycineamidine synthetase step; or which is an antifolate such as methotrexate, 2,4-diamino-5-benxyl-6-(4-phenylbutyl) pyrimidine, 2,4-diamino-5-phenyl-6-(4-phenylbutyl) pyrimidine, 2,4-diamino-5-phenyl-6-(3-anilinopropyl) pyrimidine, 2-amino-4-hydroxy-5-phenyl-6-(3-p-aminobenzoylglutamic acid propyl) pyrimidine, N-(p-oo(2,4-diamino-6-quinazolinyl)methyl-methylamino-benzoyl-L-glutamic acid, N-õp-õ2,4-diamino-5-methylquinazolinyl)methylamino!benzoyl-L-aspartic acid, N-op-õõ(2-amino-4-hydroxy-6-quinazolinyl) methyl-!methylamino!benzoyl!-L-glutamic acid, 2,4-diaminoquinazolines:
CCNSC 105952, CCNSC 112846, CCNSC 121346, CCNSC 122761, CCNSC 122870, CCNSC 529859, CCNSC 529860, or CCNSC 529861; 8-aza GMP, 7-deaza-8-aza GMP, 2′-dGMP, B-D-arabinosyl GMP, pentopyranine A-G, B-ribofuranosyl-1,3-oxazine-2,4-dione, pyrazofurin, 6-(p-chloroacetylanilinomethyl)-5-cetylvinylanilinomethyl)-5-(p-chlorophen yl)-2,4-diaminopyridine, 6-(p-chloroacetyl-ethylanilino-methyl)-5-(p-chlorophenyl)-2,4-diamino pyridine, 6-(p-chlorophenylbutylanilinomethyl)-5-(p-chlorophenyl)-2,4-diamino pyridine, p-(2,6-diamino-1,2-dihydro-2,2-dimethyl-S-triazin-1-yl) phenylpropionyl sulfanilylfluoride or variants of the propionamide bridge of acrylamido, N-ethylsulfonamido, N-ethylcaboxamido, oxyacetamido, or oxythyloxy; or which inhibits purine or pyrimidine synthesis such as xylosyladenine, 6-azauridine, 5-aminouridine, 5-azaorotic acid; or which inhibits nucleotide interconversion such as hadacidin, 6-mercaptopurine, azathioprine, nitro-dUMP, psicofuranine, decoyinine, 5-fluorouracil, 5-fluorodeoxyuridine, shadowmycin; or which inhibits nucleotide utilization such as cytosine arabinoside, arabinosyladenine; or which becomes incorporated into polynucleotides such as 8-azaguanine, tubercidine, toyocamycin, sangivamycin, formycin, 7-deazainosine, 8-azainosine, or 7-thia-7,9-dideazainosine; or which is a glyoxalase inhibitor such as Glyo-I, or Glyo-II,
an agent which blocks synthesis of prostaglandin A.sub.2 which effects platelett aggregation such as salicylic acid, pyrogallol, 5,8,11,14-eicosatetraynoic acid, .alpha.-naphthol, guaiacol, propylgallate, nordihydroguiaretic acid, N-0164, benzydamine, 9,11-azoprosta-5, 13-dienoic acid, 2-isopropyl-3-nicotinylindole,
an agent which blocks prostaglandin synthetase such as indomethacin, sulindac, tolmetin, mefenamic acid, ibuprofen, naprozen, fenoprofen, fluribiprofen, ketoprofen, meclofenamic acid, flufenamic acid, niflumic acid, benzydamine, oxyphenbutazone, asprin, acetaminophen, salicylamide, O-carboxydiphenylamine, tolectin, diclofenac, 2,7-dihydroxynaphthalene, 5-(4-chlorobenzoyl)-1-methylpyrrole-2-acetic acid, 5-(4-methylbenzoyl)-1,4-dimethylpyrrole-2-acetic acid, 5-(4-chlorobenzoyl)-1,4-dimethylpyrrole-2-acetic acid, 5-(4-fluorobenzoyl)-1,4-dimethylpyrrole-2-acetic acid, 5-(4-chlorobenzoyl)-1,4-dimethylpyrrole-2-(2-propionic acid), 5,6-dehydroarachidonate, 11,12-dehydroarachidonate, or 5,8,11,14-eicosatetraynoate; or of an agent which blocks lipoxygenase or blocks leukotriene action such as BW755C, FPL 55712, or U-60,257
an antiarrhythmic agent such as procainamide or quinidine,
an inhibitor of hepatic synthesis of Vitamin K dependent clotti-ng factors such as warfarin sodium, dicumarol, 4-hydroxycoumarin, phenprocoumon, or acenocoumarol,
an agent which relaxes vascular smooth muscle such as hydralazine, minoxidil, or isoxsuprine,
a Na.sup.+-K.sup.+-ATPase inhibitor such as digtoxigenin, digoxigenin, cymarol, periplogenin, or strophanthidiol, or ouabain glycosides, cardenolides, or basic esters, or ICI-63,632, ICI-63,605, ICI-62-655, ICI-62,838, ICI-69,654, ICI-58,622, ICI-61,374, ICI-57,267, ICI-61,424, ICI-61,411, ICI-65,199, ICI-70,898, ICI-70,899, ICI-70,900, ICI-70,901, ICI-62,966, ICI-65,210, ICI-63,116, ICI-62,936, ICI-65,551, ICI-63,978, ICI-62,276, ICI-63,056, ICI-67,135, ICI-67,167, ICI-67,134, ICI-67,875, ICI-67,880, or ICI-61,558,
a calcium channel blocker such as prenylamine, verapamil, fendiline, gallopamil, cinnarizine, tiapamil, diltiazem, bencyclan, or nifedipine; or an agent which stabalizes calcium binding to cellular calcium stores and thereby inhibits the release of this calcium by contractile stimuli such as 8-(N,N-diethylamino)-octyl 3,4,5-trimethoxybenzoate (TMB-8),
a monoamine oxidase inhibitor such as tranylcypromine, phenylethylamine, trans-cinnamic acid, phenelzine, or isocarboxazid,
a benzodiazepine compound such as clorazepate,
valproic acid,
an agent which causes repression of the synthesis of HMG-COA reductase such as 20-.alpha.-hydroxycholesterol, 22-ketocholesterol, 22-.alpha.-hydroxycholesterol, 25-hydroxycholesterol, 22-B-hydroxycholesterol, 7-.alpha.-hydroxycholesterol, 7-B-hydroxycholesterol, 7-ketocholesterol, or kryptogenin; or of an agent which inhibits HMG-COA reductase such as, lorelco; or of an agent which inhibits lipolysis such as 5-methylpyrazole-3-carboxylic acid (U-19425), nicotinic acid, uridine, inosine, 3,5-dimethylisoxazole (U-21221), 3,5-dimethypyrazole, prostaglandin E.sub.2, eritadenine, or eritadenine isoamyl ester; or of an agent which inhibits lipogenesis such as ascofuranone, (−)-hydroxycitrate, or tetrolyl-CoA; or of an agent which is hypocholesterolemic such as lentysine; or of an agent which lowers triglycerides such as lopid; or of an agent which is an inhibitor of acetyl-CoA carboxylase during lipogenesis such as 2-methyl-2-õp-(1,2,3,4-tetrahydro-1-naphthyl)-phenoxy!-propionat e (SU13437), .sup.2-(p-chlorophenoxy)-2-methylpropionate, kynurenate, xanthurenate, kynurenine, 3-hydroxyanthranilate, or 2-methyl-2-õp-(p-chlorophenyl)phenoxy! propionate; or of an agent which is an inhibitor of hepatic B-lipoprotein production such as orotic acid,
a vasodilater such as WS-1228A, or WS-1228B; or of an anti-inflammatory agent such as amicomacin A,
a protease inhibitor such as leupeptin; or which is an inhibitor of pepsin such as a pepstatin, a pepstanone, or a hydroxypepstatin,
an inhibitor of cell surface enzymes such as bestatin, amastatin, forphenicine, ebelactone, or forphenicin,
a phosphodiesterase inhibitor such as theophyllineacetic acid, theophylline, dyphylline, disodium cromoglycate, 6-n-butyl-2,8-dicarboxy-4,10-dioxo-1,4,7,10-tetrahydro-1,7-phenanthrolin, 2-chloroadenosine, dipyridamole, EG 626, AY-17,605, AY-17,611, AY-22,252, AY-22,241, cis-hinokiresinol, oxy-cis-hinokiresinol, tetrahydro-cis-hinokiresinol, trans-hinokiresinol, dehydrodicaffeic acid, 2,6,4′-trihydroxy-4-methoxybenzophenone, p-hydroxyphenyl crotonic acid, papaverine, 3-(5-tetrazolyl)-thioxanthone-10,10-dioxide, 3-carboxythioxanthone-10,10-dioxide, W-7, HA-558, MY-5445, OPC-3689, OPC-13135, or OPC-13013, reticulol, PDE-I, or PDE-II,
an inhibitor of tyrosine hydroxylase, the enzyme catalyzing the rate-limiting reaction in the biosynthesis of norepinephrine, such as azadopamine, isopropylazadopamine, dimethylazadopamine; triphenolic compounds such as n-propylgallate; diphenolic benzoic acid derivatives such as 3,4-dihydroxybenzoic acid; phenylcarbonyl derivatives such as 3,4-dihydroxybenzaldehyde, arterenone, or adrenalone H 22/54, 3-iodo-L-tyrosine, D,L-.alpha.-methyl-p-tyrosine, L-3-iodo-.alpha.-methyltyrosine, 3-bromo-.alpha.-methyltyrosine, gentistic acid, 3-chloro-.alpha.-methyltyrosine, phenylalanine derivatives, 3,5-diiodo-L-tyrosine, 3,5-dibromo-L-tyrosine, 3-bromo-.alpha.-methyl-L-tyrosine, 3-fluro-.alpha.-methyl-L-tyrosine, catechol analogues, 3,4-dihydroxyphenylethylacetamide, 3,4-dihydroxyphenyliso-proplyacetamide, 3,4-dihydroxyphenylbutylacetamide, 3,4-di-hydroxyphenylisobutylacetamide, D,L-.alpha.-methylphenylalanine, D,L-3-iodophenylalanine, D,L-4-iodophenylalanine, D,L-.alpha.-methyl-3-iodophenylalanine, D,L-a-methyl-3-bromophenylalanine, D,L-.alpha.-methyl-3-chlorophenylalanine, D,L-.alpha.-methyl-3-fluorophenylalanine, mimosine, mimosinamine, mimosinic acid, 7-O-methylspinochrome B, 6-(3-hydroxybutyl)-7-O-methylspinachrome B, aquayamycin, chrothiomycin, frenolicin, fuscaric acid, pentylpicolinic acid, dopstatin, methylspinazarin, 6,7-dihydroxymethylspinazarin, 3-ethyl-.alpha.-methyltyrosine, 3-methyl-.alpha.-methyltyrosine, 3-isopropyl-x-methyltyrosine, 3-allyl-.alpha.-methyltyrosine, 3-õ4-hydroxy-3-(2-methylallyl)-phenyl !-2-methylalanine, 3-õ3-(2,3-epoxypropyl)-4-hydroxyphenyl!-2-methylalanine, 3-isobutyl-.alpha.-methyltyrosine, 3-methylvinyl-.alpha.-methyltyrosine, 5-methyl-6,7-diphenyltetrahydropterin, 3-(2,3-dihydro-2,2-dimethyl-5-benzofuranyl!-2-methylalanine, 3-õ2,3-dihydro-2,2-dimethyl-5-benzofuranyl!-2-methylalan ine, alpha.-methyldopa, or ethyl-3-amino-4H-pyrrolo õ3,4c! isoxazole carboxylate, and
proteins including enzymes and hormones such as insulin, erythropoietin, interleuken 2, interferon, growth hormone, atrial natriuretic factor, tissue plasminogen activator.

37. The method of synthesis of the compound of claim 1 wherein the C moiety comprises at least one of the group of herbicides, fungicides, miticides, nematocides, fumigants, growth regulators, repellants, defoliants, rodenticides, molluscicides, algicides, desicants, antehelmintics, and bactericides.

38. The method of synthesis of the compound of claim 37 wherein the C moiety is one from the those given in Chemical Week Pesticides Register, Robert P. Ovellette and John A. King, 1977, McGraw-Hill Book Company.

39. A method of synthesis of a chemical compound having the formula (A-B-C)ₓ-P-E_{y}
where the A is a chemiluminescent moiety,
B is an energy acceptor moiety, and
C is a biologically active moiety, and
P is a substrate
E is an enzyme and x and y are integers
comprising the steps of
forming a benzophenone,
forming a diaryl ethylene,
attaching a phthalimide moiety to at least one of the aryl groups of the ethylene to form a phthalimide-ethylene conjugate,
condensing two ethylene-phthalimide conjugates to form a phthalimide-pentadiene conjugate,
converting the phthalimide to the phthalhydrazide by reaction with hydrazine to form a carrier compound, and
reacting the carrier compound with a biologically active moiety to form a corresponding conjugate,
reacting A-B-C with a polymer to form (A-B-C)ₓ-P, and
reacting E with (A-B-C)ₓ-P to form (A-B-C)ₓ-P-E_{y}.

40. The method of synthesis of the compound of claim 39 wherein the compound provides controlled extra cellular release of the C moiety.

41. The method of synthesis of the compound of claim 39 wherein the C moiety comprises at least one of drugs and proteins including enzymes and hormones.

42. The method of synthesis of the compound of claim 41 wherein the C moiety comprises at least one insulin, erythropoietin, interleuken 2, interferon, growth hormone, atrial natriuretic factor, tissue plasminogen activator, an anti-inflammatory drug, an antihypertensive drug, an inotropic drug, and a contraceptive drug.

43. The method of synthesis of the compound of claim 40 wherein extraacellular drug release occurs when the prodrug reacts with cellular free radicals via a mechanism involving chemiluminescence, photochromism, and intramolecular energy transfer.

44. The method of synthesis of the compound of claim 41 wherein the pharmaceutical agent is at least one of the group of antilipidemic drugs, anticholesterol drugs, contraceptive agents, anticoagulants, anti-inflamatory agents, immuno-suppressive drugs, antiarrhythmic agents, antineoplastic drugs, antihypertensive drugs, epinephrine blocking agents, cardiac inotropic drugs, antidepressant drugs, diuretics, antifungal agents, antibacterial drugs, anxiolytic agents, sedatives, muscle relaxants, anticonvulsants, agents for the treatment of ulcer disease, agents for the treatment of asthma and hypersensitivity reactions, antithroboembolic agents, agents for the treatment of muscular dystrophy, agents to effect a therapeutic abortion, agents for the treatment of anemia, agents to improve allograft survival, agents for the treatment of disorders of purine metabolism, agents for the treatment of ischemic heart disease, agents for the treatment of opiate withdrawal, agents which activate the effects of secondary messenger inositol triphosphate, agents to block spinal reflexes, and antiviral agents including a drug for the treatment of AIDS.

45. The method of synthesis of the compound of claim 43 wherein the C moiety is released by an oxidation reduction reaction with the target cell's electron carriers or by reaction with free radicals produced as a consequence of electron transport.

46. The method of synthesis of the compound of claim 43 wherein A represents a functionality which undergoes at least one of
an oxidation reduction reaction where electrons are transferred directly between A and the target cell's electron carriers, and
a reaction with free radicals of oxygen which are produced as a consequence of electron transport
such that an excited state is produced in A as a consequence of its participation in one of these reactions.

47. The method of synthesis of the compound of claim 46 wherein A undergoes intramolecular energy transfer from its own excited state to the B functionality which is an energy acceptor.

48. The method of synthesis of the compound of claim 47 wherein upon receiving energy from A, B achieves an excited state which relaxes through heterolytic cleavage of the covalent bond of B with C where C is a drug moiety which is released into the environment.

49. The method of synthesis of the compound of claim 39 wherein the chemiluminescent molecule comprises at least one of the group of
molecules undergoing reaction involving peroxides and oxygen free radicals,
molecules undergoing reaction involving oxidation or reduction, and
molecules undergoing both reaction with peroxides and oxygen free radicals followed by an oxidation or reduction reaction.

50. The method of synthesis of the compound of claim 49 wherein the chemiluminescent molecule comprises at least one of the group of luminol and its derivatives, lucigenin and its derivatives, Lophine and its derivatives, acridinium esters and acridans, tetraphenylpyrrole, phthalhydrazides, acyloins, biacridinium salts, vinylcarbonyls, vinylnitriles, tetrakis (dimethylamino) ethylene, acylperoxides, indoles, tetracarbazoles and active oxalates.

51. The method of synthesis of the compound of claim 49 wherein the chemiluminescent molecule comprises at least one of the group of ruthenium chelates 2, 6-diaminopyrene, or cation radicals and molecules which follow a Chemically Initiated Electron Exchange Luminescence mechanism such as certain dioxetans and dioxetanones.

52. The method of synthesis of the compound of claim 49 wherein the chemiluminescent molecule comprises at least one of the group of dioxene derivatives and other compounds that form a dioxetan by reaction with superoxide and then produce efficient chemiluminescence by a CIEEL mechanism.

53. The method of synthesis of the compound of claim 49 wherein the chemiluminescent molecule comprises at least one of the group of
TABLE 1 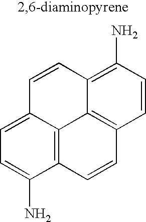 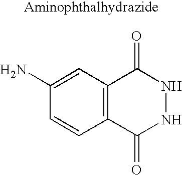
Representative Chemiluminescent Molecules

| Name | Structure |
|---|---|
| Dioxene | 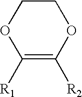 |
| Imidazole derivatives | 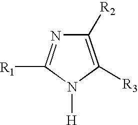 |
| Sulfonyloxamides | 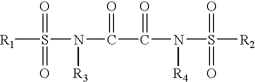 |
| Indole derivatives | 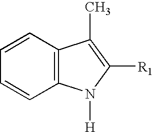 |
| Tetrakis(dialkyl- amino)-ethylene | 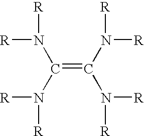 |
| 2,5,7,8-tetraoxa- bicyclo-[4.2.0]octane | 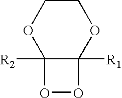 |
| Dioxetan | 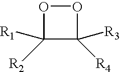 |
| Lucigenin | 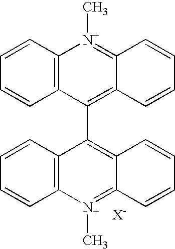 |
| Lophine | 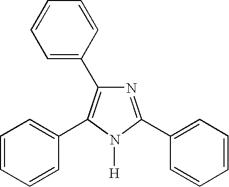 |
| Acridinium esters | 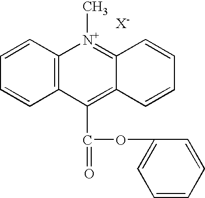 |
| Active oxalate | 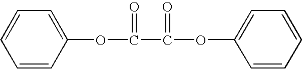 |
| Tris-2,2′-bipyridine- dichlororuthenium (II) | 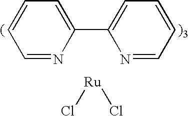 |
| Dioxetanone | 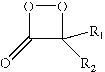 |
| Dipheyl peroxide | 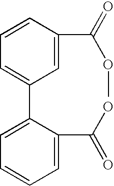 |

54. The method of synthesis of the compound of claim 39 wherein the B moiety is a photochromic compound.

55. The method of synthesis of the compound of claim 54 wherein the photochromic compound comprises one which demonstrate photochromic behavior with electromagnetic radiation and bleaching agents.

56. The method of synthesis of the compound of claim 55 wherein the A functionality is chemiluminescent, and the B functionality is such that the photodissociative drug release spectrum of B overlaps the chemiluminescence spectrum of A.

57. The method of synthesis of the compound of claim 54 wherein the photochromic compound comprises a cationic dye.

58. The method of synthesis of the compound of claim 57 wherein the cationic dye comprises at least one of a di and triarylmethane dyes, triarylmethane lactones and cyclic ether dyes, cationic indoles, pyronines, phthaleins, oxazines, thiazines, acridines, phenazines, and anthocyanidins, and cationic polymethine dyes and azo and diazopolymethines, styryls, cyanines, hemicyanines, dialkylaminopolyenes, and other related dyes.

59. The method of synthesis of the compound of claim 57 wherein the cationic dye comprises at least one of

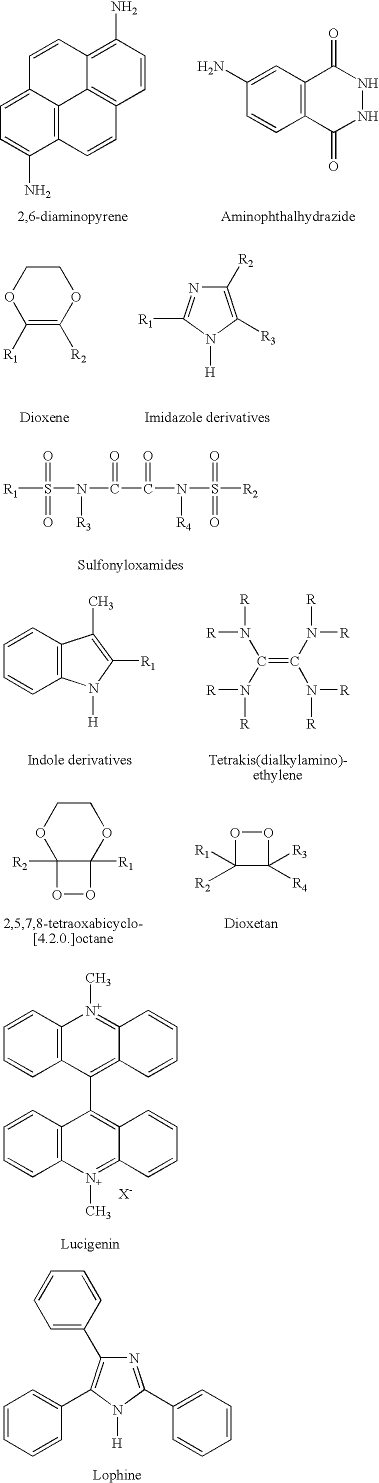

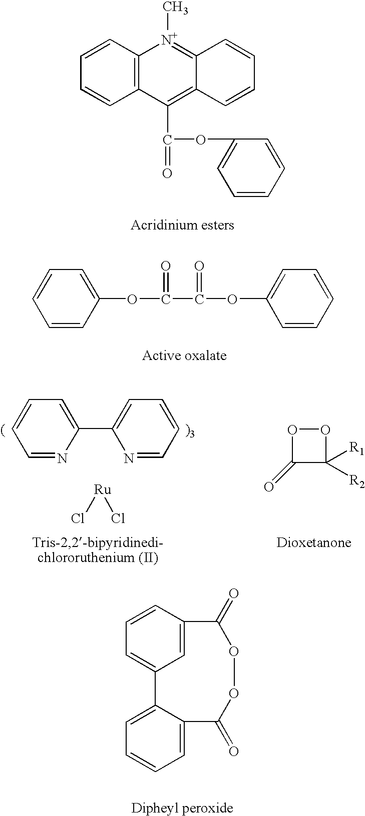

60. The method of synthesis of the compound of claim 39 wherein the C moiety is any molecule which exhibits bleaching behavior with the B moiety and has an increased therapeutic effect or therapeutic ratio as a consequence of its delivery as part of a prodrug.

61. The method of synthesis of the compound of claim 39 wherein the C moiety has a nucleophilic group that bonds to the B moiety.

62. The method of synthesis of the compound of claim 61 wherein the C moiety is derivatized to have a nucleophilic group that bonds to the B moiety.

63. The method of synthesis of the compound of claim 62 wherein the C moiety is derivatized by at least one of the nucleophilic groups comprising cinnamate, sulfite, phosphate, carboxylate, thiol, amide, alkoxide, or amine.

64. The method of synthesis of the compound of claim 39 wherein the C moiety is at least one of the group of

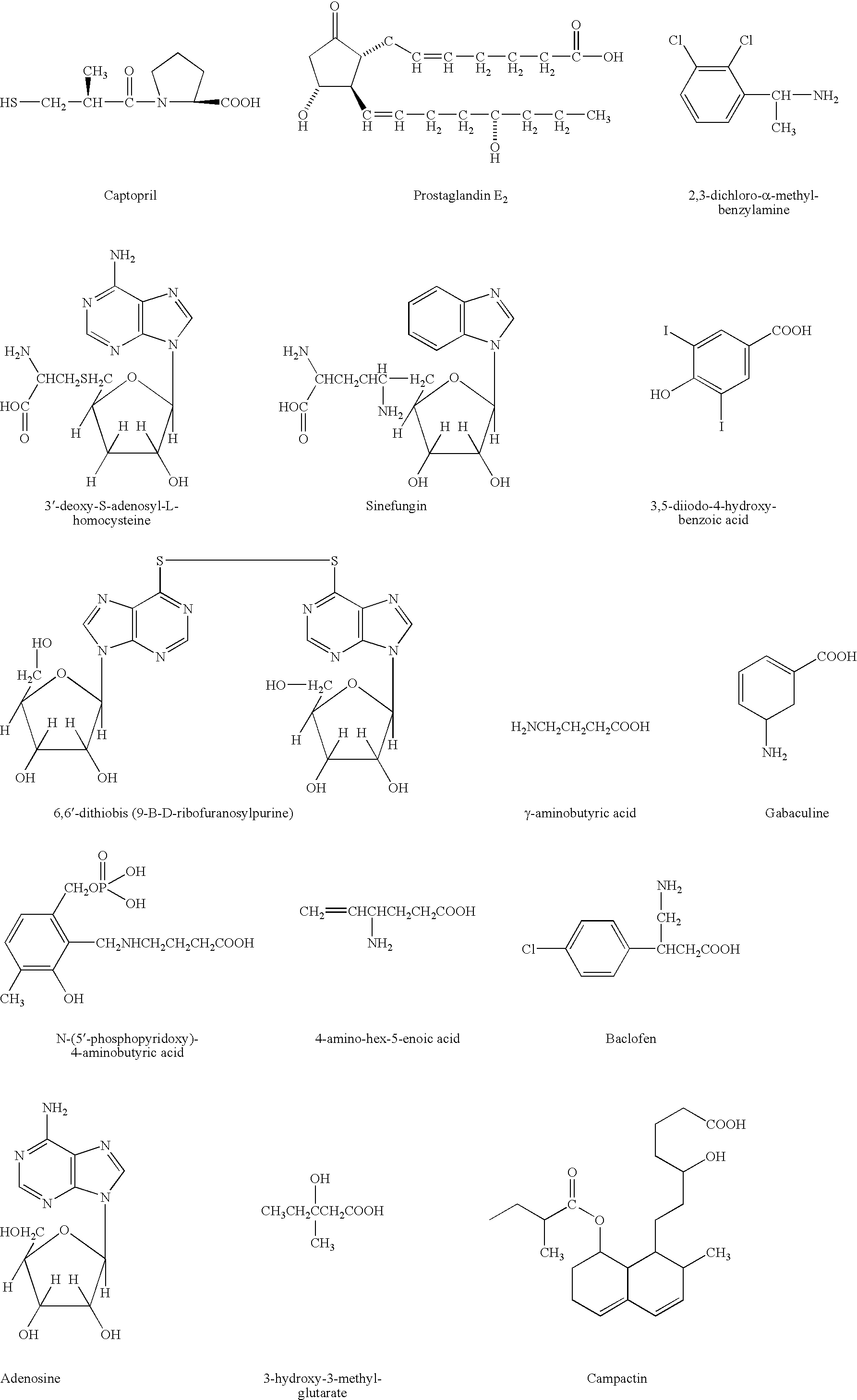

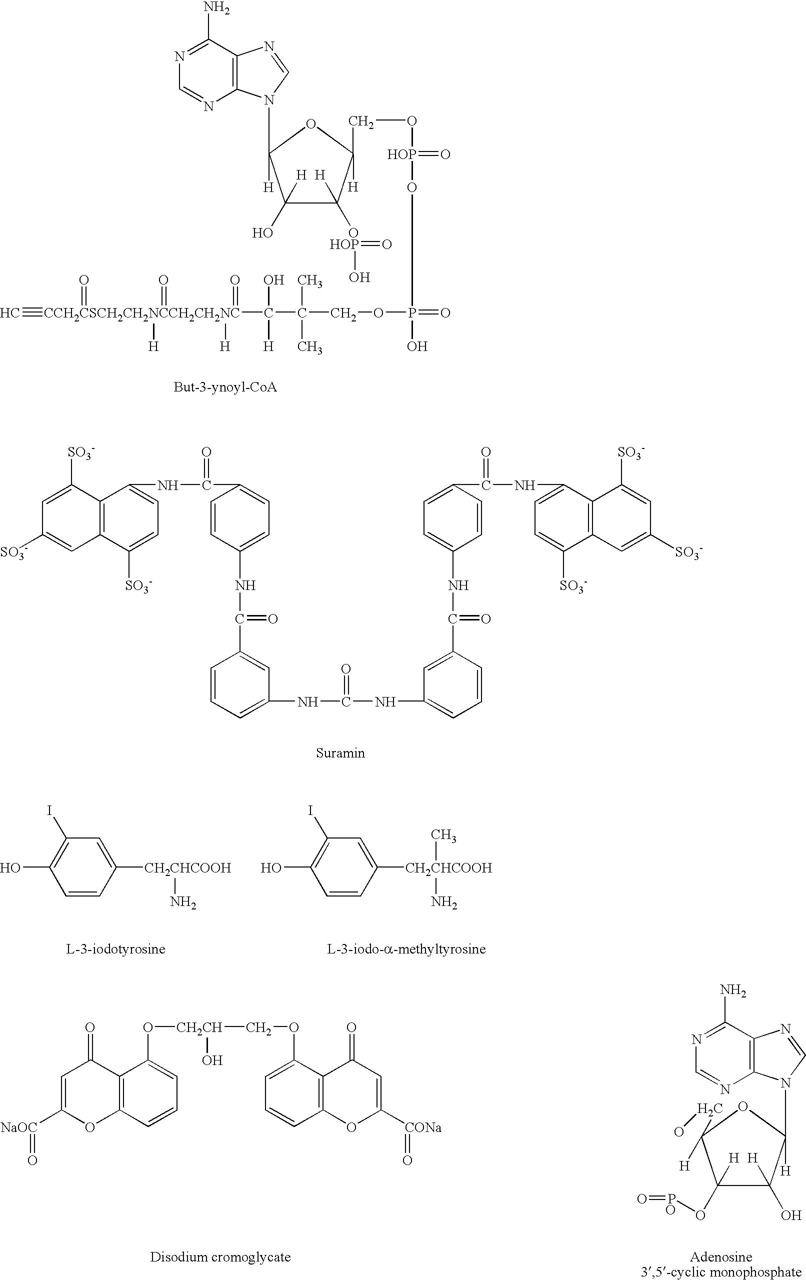

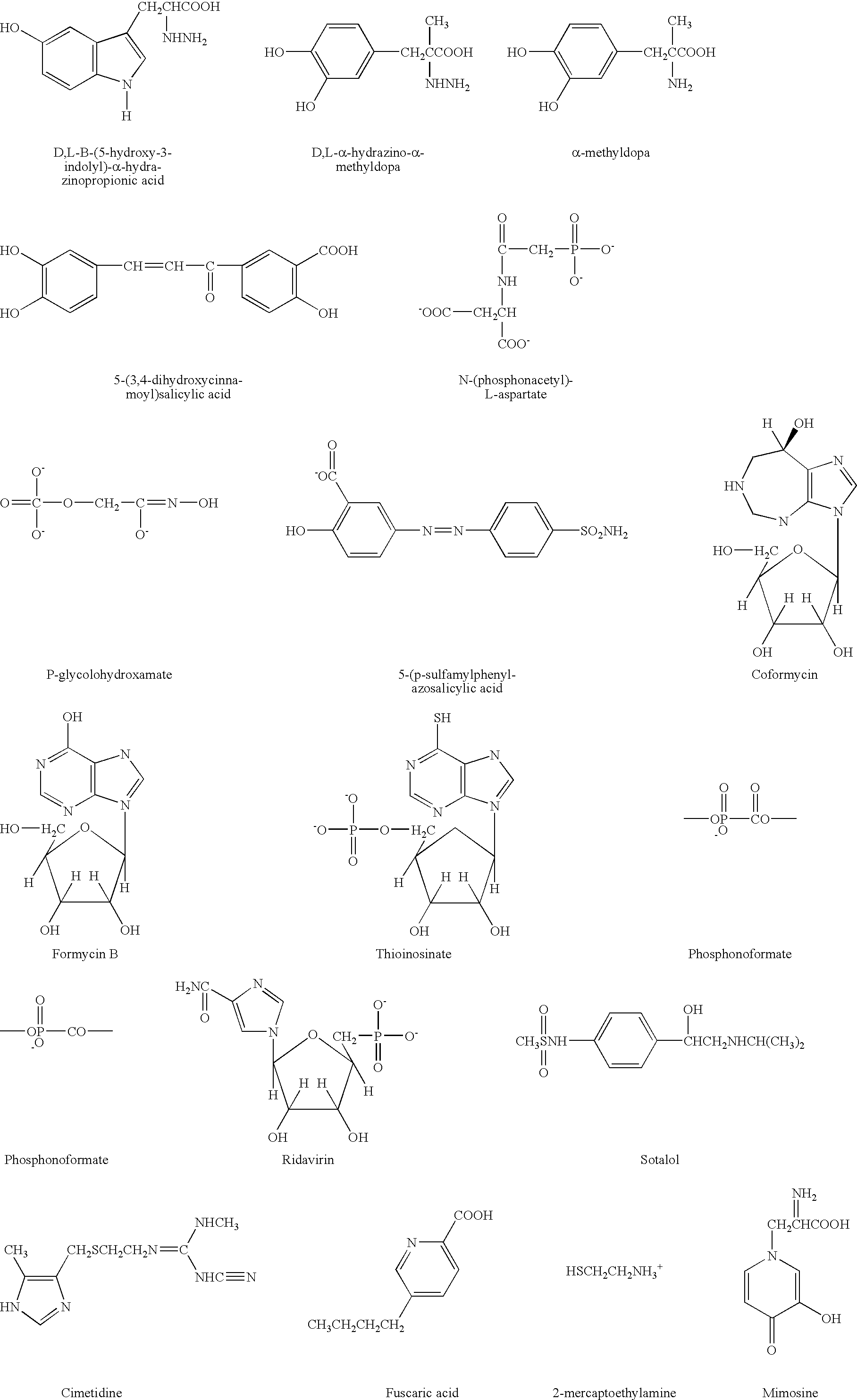

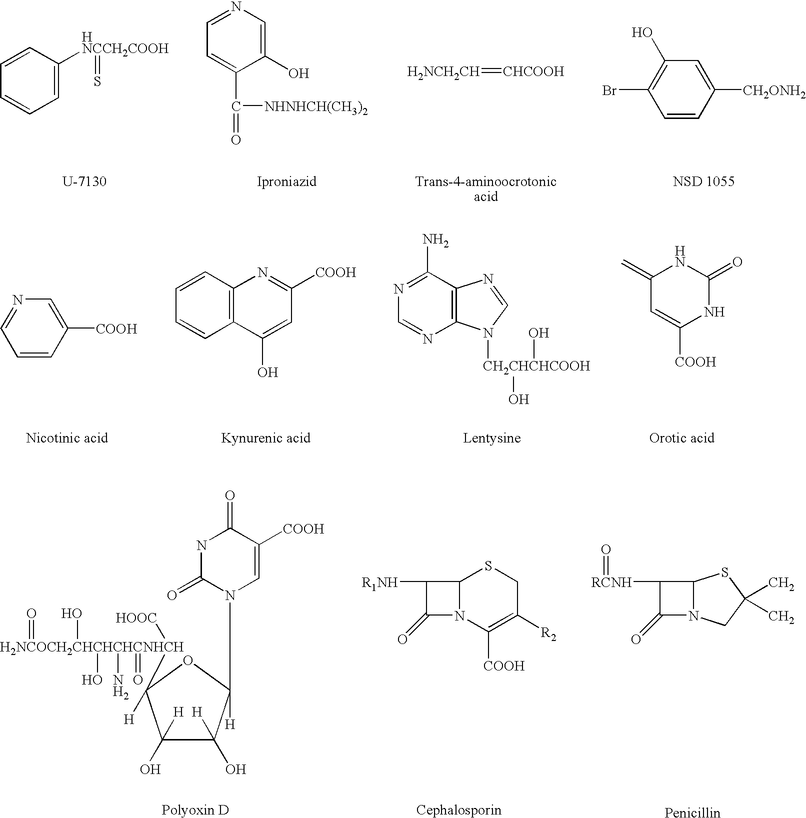

65. The method of synthesis of the compound of claim 39 wherein the A-B-C moieties are attached to P by a bond between P and at least one of A and B.

66. The method of synthesis of the compound of claim 39 wherein the E moieties are attached to (A-B-C)ₓ-P by a bond between E and at least one of A, B, and P.

67. The method of synthesis of the compound of claim 39 wherein the E moieties are enzymes that react with a desired substrate and form substances that cause the release of C from A-B-C.

68. The method of synthesis of the compound of claim 39 wherein the E moieties are enzymes that react with a desired substrate and form peroxide or free radicals that cause the release of C from A-B-C.

69. The method of synthesis of the compound of claim 67 wherein the E moiety, substrate, and C moiety are at least one of the group of
glucose oxidase, glucose, and insulin, and
xanthine oxidase, xanthine, and tissue plasminogen activator (TPA).

70. A method of synthesis of a chemical compound having the formula (A-B-C)ₓ-P
where the A is a chemiluminescent moiety,
B is an energy acceptor moiety, and
C is a biologically active moiety, and
P is a substrate and x is an integer
comprising the steps of
forming a benzophenone,
forming a diaryl ethylene,
attaching a phthalimide moiety to at least one of the aryl groups of the ethylene to form a phthalimide-ethylene conjugate,
condensing two ethylene-phthalimide conjugates to form a phthalimide-pentadiene conjugate,
converting the phthalimide to the phthalhydrazide by reaction with hydrazine to form a carrier compound, and
reacting the carrier compound with a strong base such as an alkali hydride and the biologically active moiety to form a corresponding conjugate,
reacting A-B-C with a polymer to form (A-B-C)ₓ-P.

71. The method of synthesis of the compound of claim 1 wherein one or more of the moieties can be modified to further candidate components by addition of functional groups.

72. The method of synthesis of the compound of claim 71 wherein the groups comprise at least on of alkyl, cycloalkyl, alkoxycarbonyl, cyano, carbamoyl, heterocyclic rings containing C, O, N, S, sulfo, sulfamoyl, alkoxysulfonyl, phosphono, hydroxyl, halogen, alkoxy, alkylthiol, acyloxy, aryl, alkenyl, aliphatic, acyl, carboxyl, amino, cyanoalkoxy, diazonium, carboxyalkylcarboxamido, alkenylthio, cyanoalkoxycarbonyl, carbamoylalkoxycarbonyl, alkoxy carbonylamino, cyanoalkylamino, alkoxycarbonylalkylamino, sulfoalkylamino, alkylsulfamoylaklylamino, oxido, hydroxy alkyl, carboxy alkylcarbonyloxy, cyanoalkyl, carboxyalkylthio, arylamino, heteroarylamino, alkoxycarbonyl, alkylcarbonyloxy, cyanoalkoxy, alkoxycarbonylalkoxy, carbamoylalkoxy, carbamoylalkyl carbonyloxy, sulfoalkoxy, nitro, alkoxyaryl, halogenaryl, amino aryl, alkylaminoaryl, tolyl, alkenylaryl, allylaryl, alkenyloxyaryl, allyloxyaryl, cyanoaryl, carbamoylaryl, carboxyaryl, alkoxycarbonylaryl, alkylcarbonyoxyaryl, sulfoaryl, alkoxysulfoaryl, sulfamoylaryl, and nitroaryl.

73. The method of synthesis of the compound of claim 1 wherein the compound has the structure of general formula

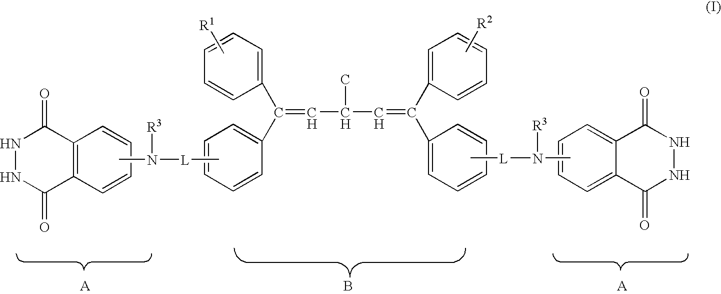

74. The method of synthesis of the compound of claim 73 wherein the functionality A is at least one of aminophthalhydrazide derivatives, sulfonyloxamides and active oxalates,
the functionality B is at least one of 1,1,5,5-tetrakisarylpentadiene and 1,1,5-trisarylpentadiene derivatives,
the functionality C is a drug molecule such as Foscarnate, or ddc; and
R is a functional group, and
L is a linker such as an aliphatic chain between A and B.

75. The method of synthesis of the compound of claim 74 wherein the L functionality is between one 20 carbon atoms.

76. The method of synthesis of the compound of claim 1 wherein B is a 1,1,5-trisarylpentadiene derivative and the compound has the formula

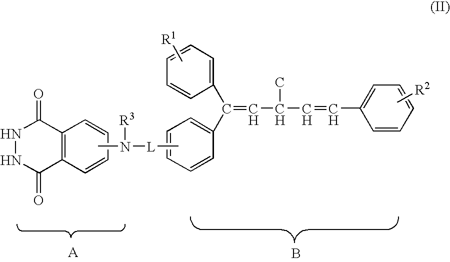

77. The method of synthesis of the compound of claim 1 wherein A is a sulfonyloxamide or active oxalate and the compound has the formula

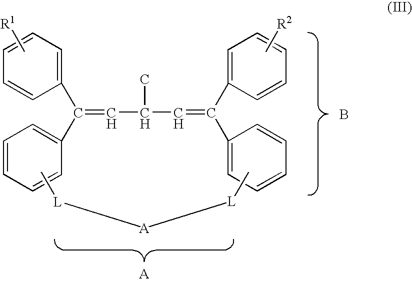

78. The method of synthesis of the compound of claim 1 wherein a luminol derivative is directly attached through one or more amino groups to the aryl groups of a photochromic dye.

79. The method of synthesis of the compound of claim 78 wherein
C comprises the formula of at least one of

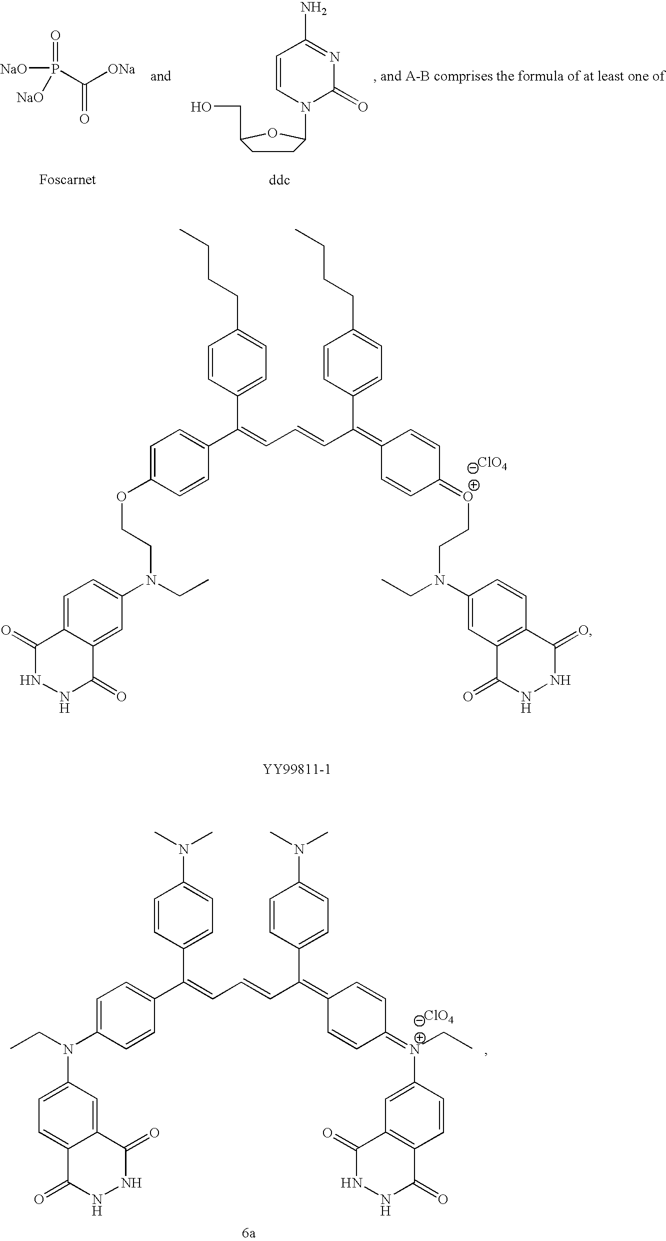

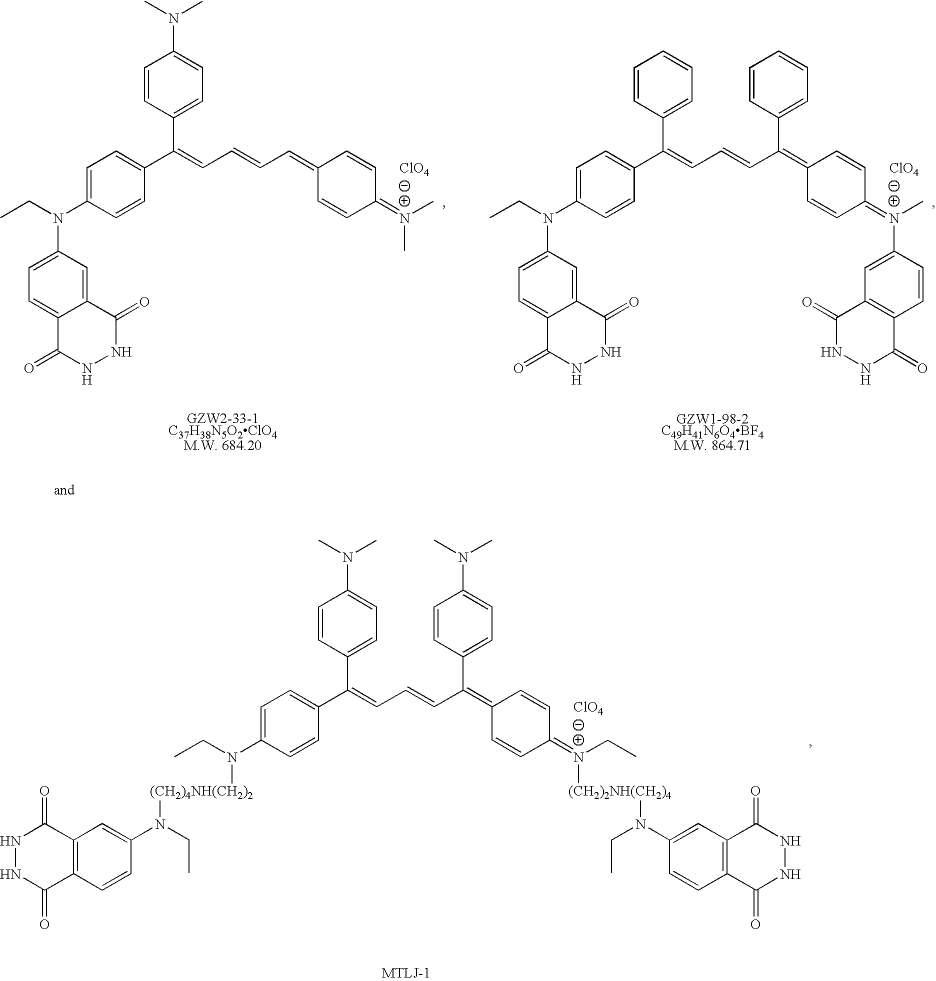

80. The method of synthesis of the compound of claim 78 wherein the compound comprises the formula

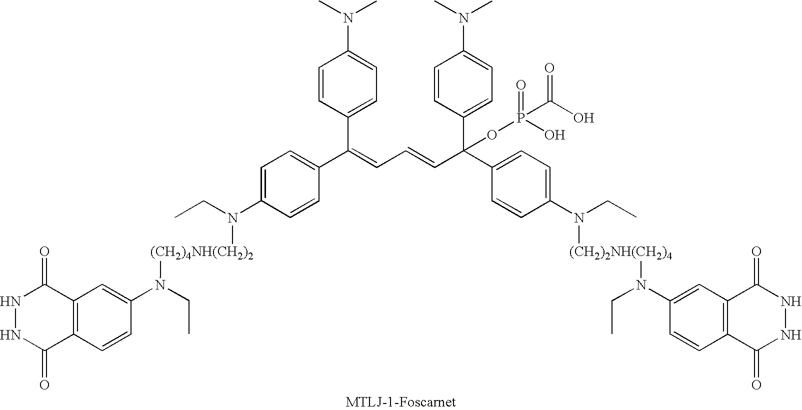

81. The method of synthesis of the compound of claim 1 wherein the hydrolyzable group that protects phthalhydrazide is at least one of acetyl and t-butyloxycarbonyl.

82. The method of synthesis of the compound of claim 1 wherein the aminophthalimide-substituted precursors for the dye are prepared through amination of an aryl halide such as palladium-catalyzed amination of aryl halides.

83. The method of synthesis of the compound of claim 1 wherein halo-substituted aryl groups of a starting B moiety or an intermediate are coupled with the aminophthalimide by methods such as the aryl amination under palladium catalysis to form the aminophthalimide-substituted precursors for the dye.

84. The method of synthesis of the compound of claim 1 wherein halo-substituted aryl groups of a starting phthalimide or an intermediate are coupled with the amino-substituted dye by methods such as the aryl amination under palladium catalysis to form the aminophthalimide-substituted precursors for the dye.

85. The method of synthesis of the compound of claims 83 and 84 wherein amino-substituted aryl groups are obtained by the amination of the halo-substituted compounds with an imine such as benzophenoneimine.

86. The method of synthesis of the compound of claim 1 wherein the aminophthalimide-attached dye is formed by the condensation of two aminophthalimide-attached ethylene molecules by reaction with triethyl orthoformate and a strong acid such as perchloric acid in acetic anhydride or acetic acid.

87. The method of synthesis of the compound of claim 1 wherein during the step of converting the phthalimide moiety to the aminophthalhydrazide to obtain A-B, the B moiety is protected from reaction with hydrazine by reacting with base such as sodium hydroxide, sodium methoxide and amines.

88. The method of synthesis of the compound of claim 87 wherein the phthalimide-B conjugate with a protected B moiety is refluxed with hydrazine in a suitable solvent such as an alcoholic solvent in inert atmosphere and then treated with acid such as perchloric acid, tetrafluroboric acid to regenerate a corresponding unaltered B moiety of the A-B conjugate.

89. The method of synthesis of the compound of claim 88 wherein A-B is reacted with one nucleophilic species of C to form A-B-C.

90. The method of synthesis of the compound of claim 1 wherein A-B is formed by starting with B comprising halo-substituted dyes, such as 1,5-bis(p-bromophenyl)-1,5-bis(p-dimethylaminophenyl)-pentadienium perchlorate.

91. The method of synthesis of the compound of claim 90 wherein cationic dyes are protected by reacting with base such as alkoxide and then coupled with the aminophthalimide by amination of aryl halide such as the palladium-catalyzed amination of aryl halide to obtain the alkoxide-protecting aminophthalimide-substituted dyes.

92. The method of synthesis of the compound of claim 91 wherein the aminophthalimide-B conjugate with a protected B moiety is refluxed with hydrazine in a suitable solvent such as an alcoholic solvent to convert the amino-phthalimide moiety to the aminophthalhydrazide moiety and then treated with acid to generate A-B.

93. The method of synthesis of the compound of claim 1 wherein the B comprises a tetraarylpolymethine, the aminophthalhydrazide precursor is an aminophthalic acid diester and the conjugate to form A-B is amino-phthalimideluminol-tetraaryl-polymethine.

94. The method of synthesis of the compound of claim 1 wherein halo-substituted diarylketone are formed by at least one of direct acylation of arene with halo-substituted benzoyl halide under ferric chloride catalysis according to the following representative scheme 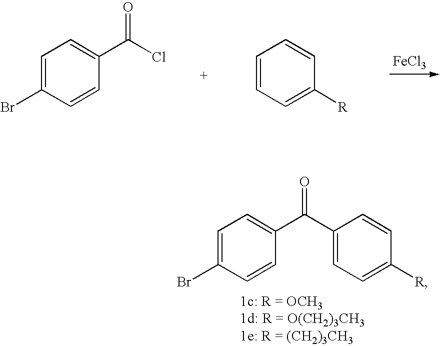
and or indirect acylation according to the following representative scheme

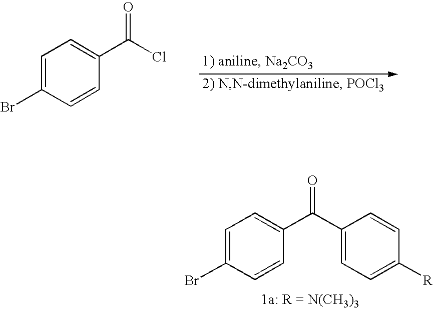

95. The method of synthesis of the compound of claim 1 wherein a halo-substituted diarylketone is converted to the corresponding halo-substituted diarylketene such as halo-substituted 1,1-diarylethene.

96. The method of synthesis of the compound of claim 95 wherein the halo-substituted diarylketene is coupled with a precursor of amino-phthalhydrazide such as aminophthalimide, aminophthalic acid diester, by aryl amination such as the palladium-catalyzed amination of aryl halides to form the aminophthalimide-substituted 1,1-diarylethene.

97. The method of synthesis of the compound of claim 96 wherein the ethene is condensed with an orthoester such as triethylorthoformate in a nonaqueous solvent such as acetic anhdydide, containing an acid catalyst such as perchloric acid, tetrafluoroboric acid, to form the aminophthalimide-substituted tetraarylpolymethine dye.

98. The method of synthesis of the compound of claim 97 wherein the aminophthalimide moiety is converted to the aminophthalhydrazide to obtain A-B.

99. The method of synthesis of the compound of claim 98 wherein the B moiety is a cationic dye that is first protected by reacting with an anion such as hydroxide, methoxide and amine and the phthalimide-B conjugate with a protected B moiety is refluxed with hydrazine in a suitable solvent such as an alcoholic solvent in inert atmosphere and then treated with acid such as perchloric acid, tetrafluroboric acid to regenerate a corresponding unaltered B moiety of the A-B conjugate.

100. The method of synthesis of the compound of claim 99 wherein A-B is reacted with one nucleophilic species of a C such as a drug 2′,3′-dideoxycytidine, Foscarnet, acycloguanosine to form A-B-C comprising a prodrug.

101. The method of synthesis of the compound of claim 95 wherein two halo-substituted diarylketene precursor compounds are condensed with an orthoester such as triethylorthoformate in a nonaqueous solvent such as acetic anhydride containing acid catalyst such as perchloric acid, tetrafluoroboric acid to form the halo-substituted tetraarylpolymethine dyes such as 1,5-bis(p-bromophenyl)-1,5-bis(p-dimethylaminophenyl)-pentadienium perchlorate.

102. The method of synthesis of the compound of claim 101 wherein the B moiety is a cationic dye that is protected by reacting with an anion such as alkoxide and then coupled with the aminophthalimide by amination of aryl halide such as the palladium-catalyzed amination of aryl halide to obtain the alkoxide-protected aminophthalimide-substituted tetraarylpolymethine dye.

103. The method of synthesis of the compound of claim 103 wherein the alkoxide-protected aminophthalimide-substituted tetraarylpolymethine dye is refluxed with hydrazine in a suitable solvent such as an alcoholic solvent to convert the amino-phthalimide moiety to the aminophthalhydrazide moiety and then treated with acid to generate A-B comprising a luminol-tetraarylpolymethine compound.

104. The method of synthesis of the compound of claim 1 comprising the general steps given by following representative formula

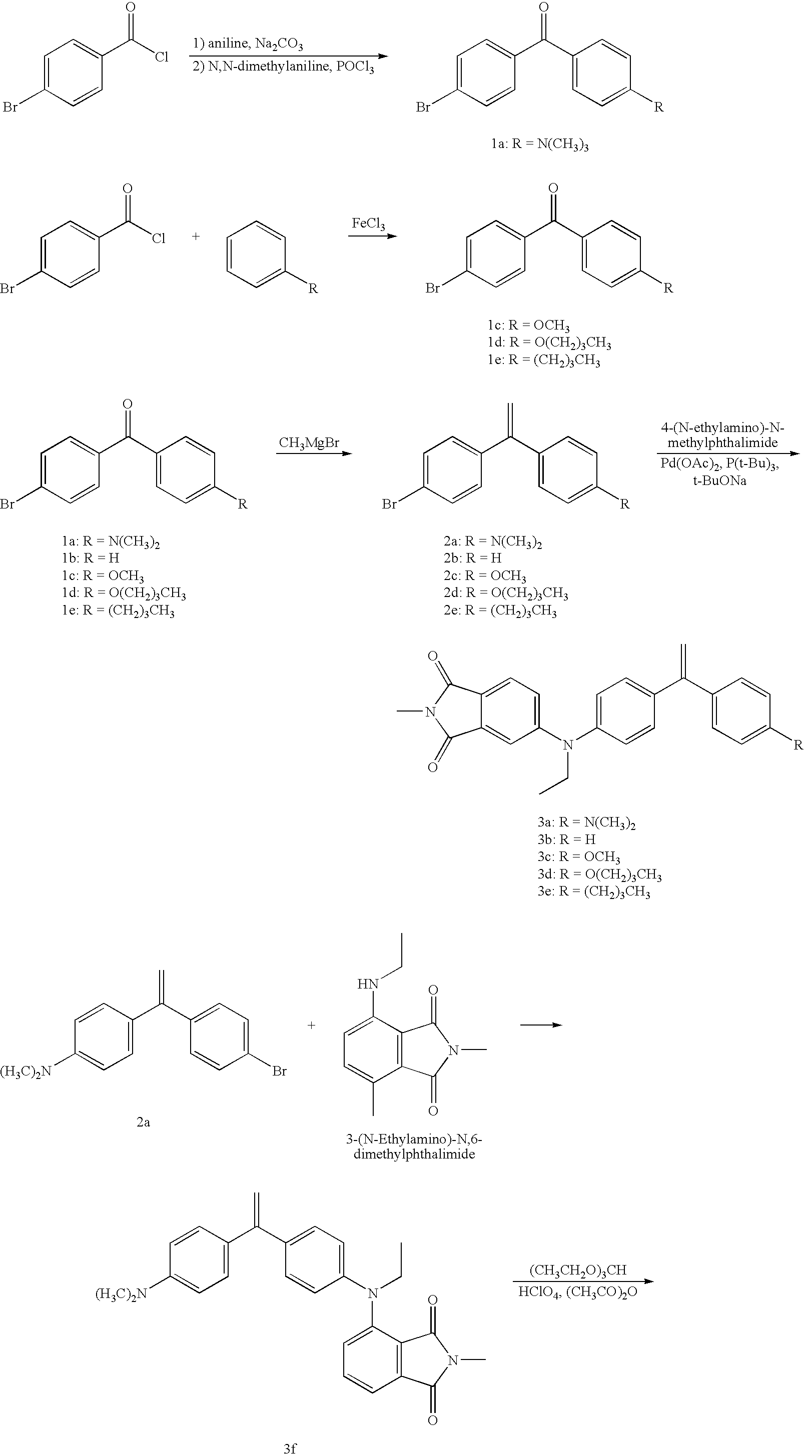

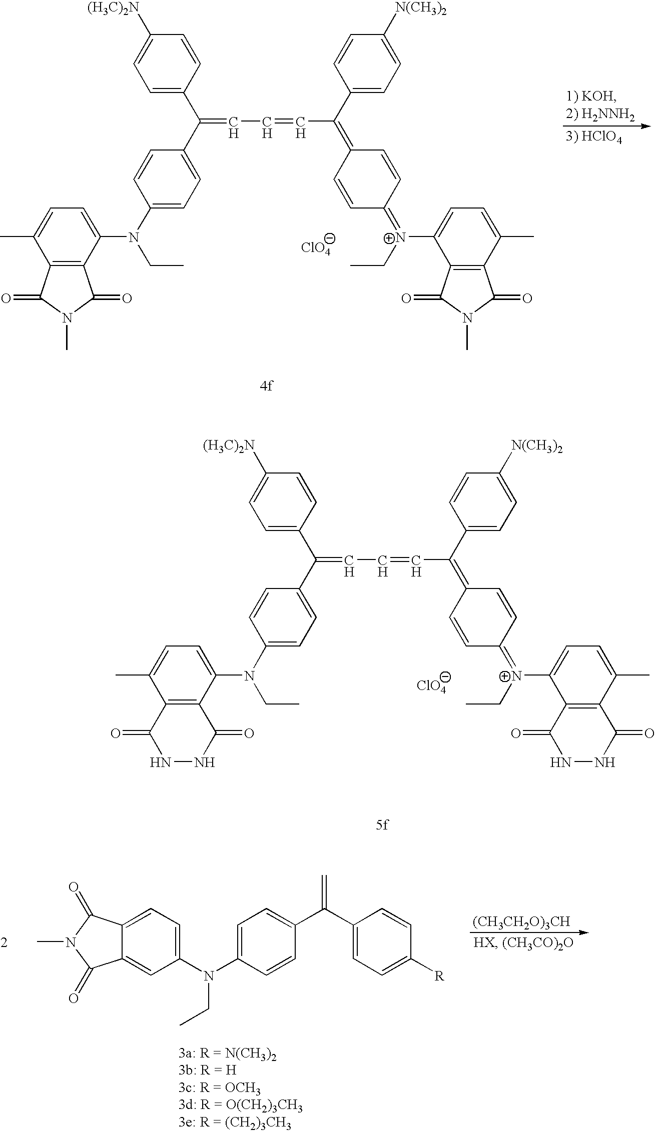

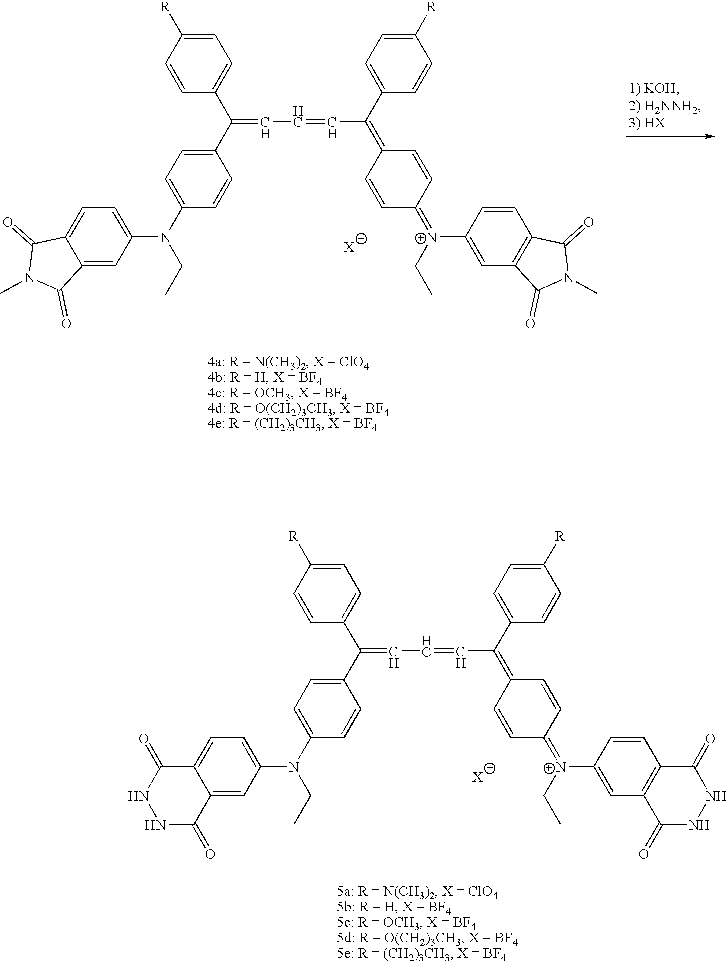

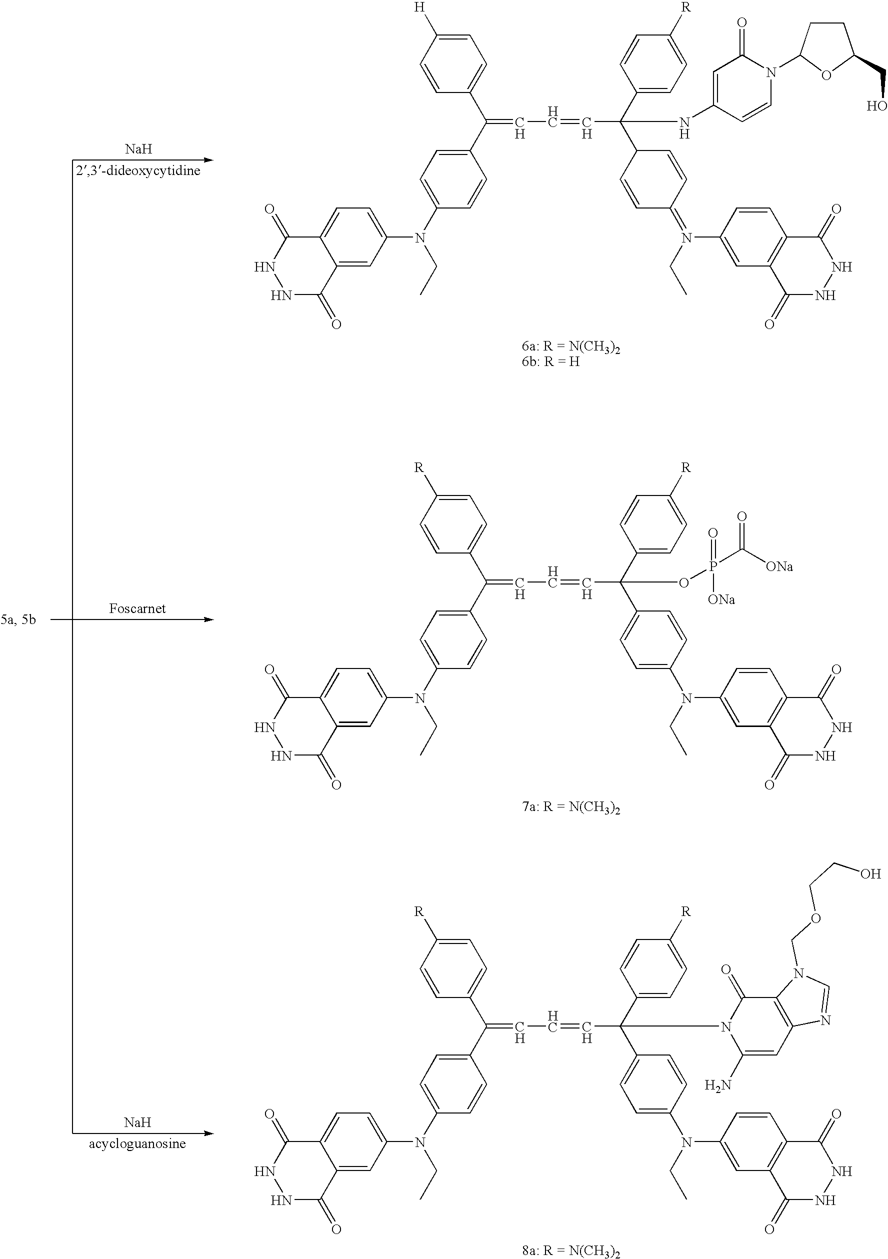

105. The method of synthesis of the compound of claim 1 wherein the A functionality comprises a phthalhydrazide such as a luminol derivative and the B functionality comprises a photochromic dye wherein A is attached to aryl groups of B comprising the steps of
forming a diaryl ketone,
forming a diaryl ketene from the diaryl ketone,
forming a protected aminophthalhydrazide such as aminophthalimide or aminophthalic acid diester,
adding a hydrocarbon linker to the protected aminophthalhydrazide, and
attaching the protected aminophthalhydrazide through the molecular linker to the aryl groups of diarylketene to form the precursor aminophthalimide-linked diarylketene, and reacting according to at least one of
(a) forming the A functionality from the precursor, and condensing two molecules of B precursor linked to A to form A-B, and
(b) condensing two precursor aminophthalimide-linked diarylketene molecules to form A precursor linked to B, and
forming the A functionality from the A precursor to form A-B.

106. The method of synthesis of the compound of claim 105 wherein the diaryl ketone is formed by a classical Friedel-Crafts acylation between a benzoyl halide and aryl compound with a hydrocarbon linker having a leaving group.

107. The method of synthesis of the compound of claim 106 wherein the aryl compound with a hydrocarbon linker having a leaving group comprises at least one of a halogenated-alkyl-aryl ether and a halogenated-aklyl-aryl amine wherein the halogen is the leaving group.

108. The method of synthesis of the compound of claim 107 wherein the halogenated-alkyl-aryl ether comprises 2-bromoethoxybenzene to give an aryl ketone such as 4-(2-bromoethoxy)benzophenone.

109. The method of synthesis of the compound of claim 107 wherein the halogenated-aklyl-aryl amine comprises 2-bromoethyl aminobenzene to give an aryl ketone such as 4-(2-bromoethyl amino)benzophenone.

110. The method of synthesis of the compound of claim 105 wherein the diaryl ketone is converted to the corresponding diarylketene by reacting with a methylating reagent such as a methyl Grignard reagent, methyl lithium reagent, lithium dimethylcopper reagent and then dehydration with acid.

111. The method of synthesis of the compound of claim 110 wherein the diaryl ketone is converted to the corresponding diarylketene by reacting with methylmagnesium bromide and then dehydration with acid.

112. The method of synthesis of the compound of claim 105 wherein the diaryl ketone is converted to the corresponding diarylketene by a Wittig reaction.

113. The method of synthesis of the compound of claim 105 wherein a linker is attached to the protected aminophthalhydrazide by a reaction of a nucleophilic group of the linker or protected aminophthalhydrazide with a leaving group of the linker or protected aminophthalhydrazide.

114. The method of synthesis of the compound of claim 105 wherein a linker is attached to the protected aminophthalhydrazide by reaction to form a bond between at least one of a nitrogen, oxygen, or carbon atom of the linker and at least one of a nitrogen, oxygen, or carbon atom of group of the protected aminophthalhydrazide by an addition or a substitution reaction of a leaving group.

115. The method of synthesis of the compound of claim 114 wherein a linker is attached to the protected aminophthalhydrazide by a substitution reaction of at least one of a halogen, tosylate group, ester group with a nitrogen, oxygen, or carbon atom.

116. The method of synthesis of the compound of claim 105 wherein attaching the protected aminophthalhydrazide through the molecular linker to one of the aryl groups of diarylketene to form the precursor aminophthalimide-linked diarylketene is by a reaction of a nucleophilic group of the linker or aryl group of diarylketene with a leaving group of the linker or aryl group of diarylketene.

117. The method of synthesis of the compound of claim 116 wherein a linker is attached to the aryl group of diarylketene by reaction to form a bond between at least one of a nitrogen, oxygen, or carbon atom of the linker and at least one of a nitrogen, oxygen, or carbon atom of group of the protected aminophthalhydrazide by an addition or a substitution reaction of a leaving group.

118. The method of synthesis of the compound of claim 117 wherein a linker is attached to the aryl group of diarylketene by a substitution reaction of at least one of a halogen, tosylate group, ester group with a nitrogen, oxygen, or carbon atom.

119. The method of synthesis of the compound of claim 105 wherein the precursor aminophthalimide-linked diarylketene is further reacted by condensation of two aminophthalimide-linked diarylketenes with an orthoester to form B linked to the A precursor.

120. The method of synthesis of the compound of claim 119 wherein condensing reagent is triethylorthoformate.

121. The method of synthesis of the compound of claim 119 wherein the precursor aminophthalimide-linked diarylketene comprises at least one of the formula 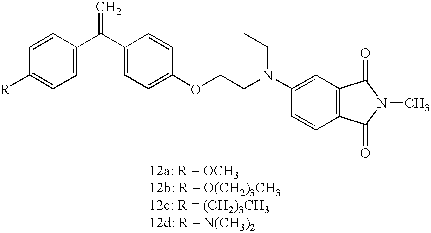
and the precursor of A-B comprises at least one of the formula

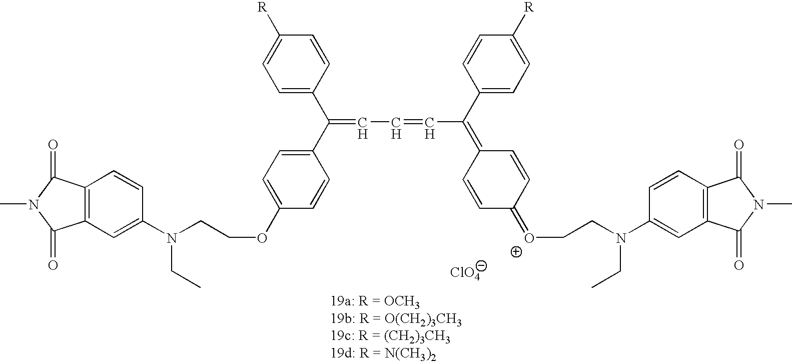

122. The method of synthesis of the compound of claim 119 wherein the phthalimide moiety of the A precursor is converted to the phthalhydrazide A functionality by treating with hydrazine, forming A-B.

123. The method of synthesis of the compound of claims 122 wherein the B functionality is protected by reacting with an anion such as hydroxide, methoxide and amine, the A-B precursor is refluxed with hydrazine in a suitable solvent such as an alcoholic solvent in inert atmosphere and then treated with acid such as perchloric acid, tetrafluroboric acid to form A-B.

124. The method of synthesis of the compound of claim 105 wherein the phthalimide moiety of the A precursor of the precursor aminophthalimide-linked diarylketene is converted to the phthalhydrazide A functionality by treating with hydrazine, forming A attached to a B precursor.

125. The method of synthesis of the compound of claim 124 wherein the A-linked diarylketene is further reacted by condensation of two A-linked diarylketenes with an orthoester to form A-B.

126. The method of synthesis of the compound of claim 125 wherein condensing reagent is triethylorthoformate.

127. The method of synthesis of the compound of claim 126 wherein the A-linked diarylketene comprises at least one of the formula 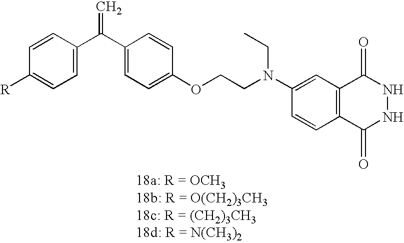
and A-B comprises at least one of the formula

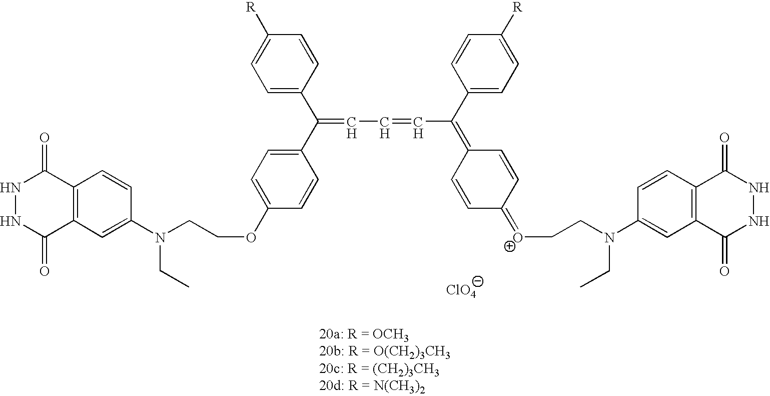

128. The method of synthesis of the compound of claims 123 and 125 further comprising the step of reacting the B functionality with one nucleophilic species of a C functionality such as Foscarnet to form A-B-C.

129. The method of synthesis of the compound of claim 105 comprising the general steps given by following representative formula

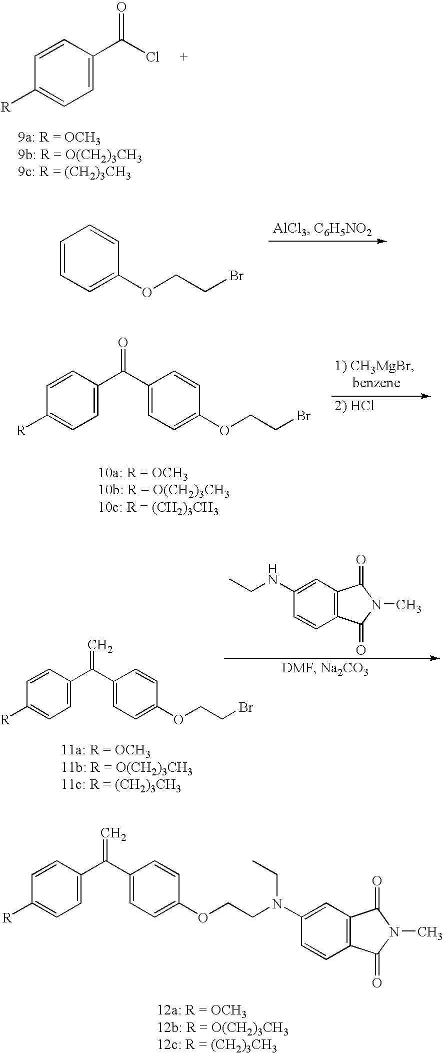

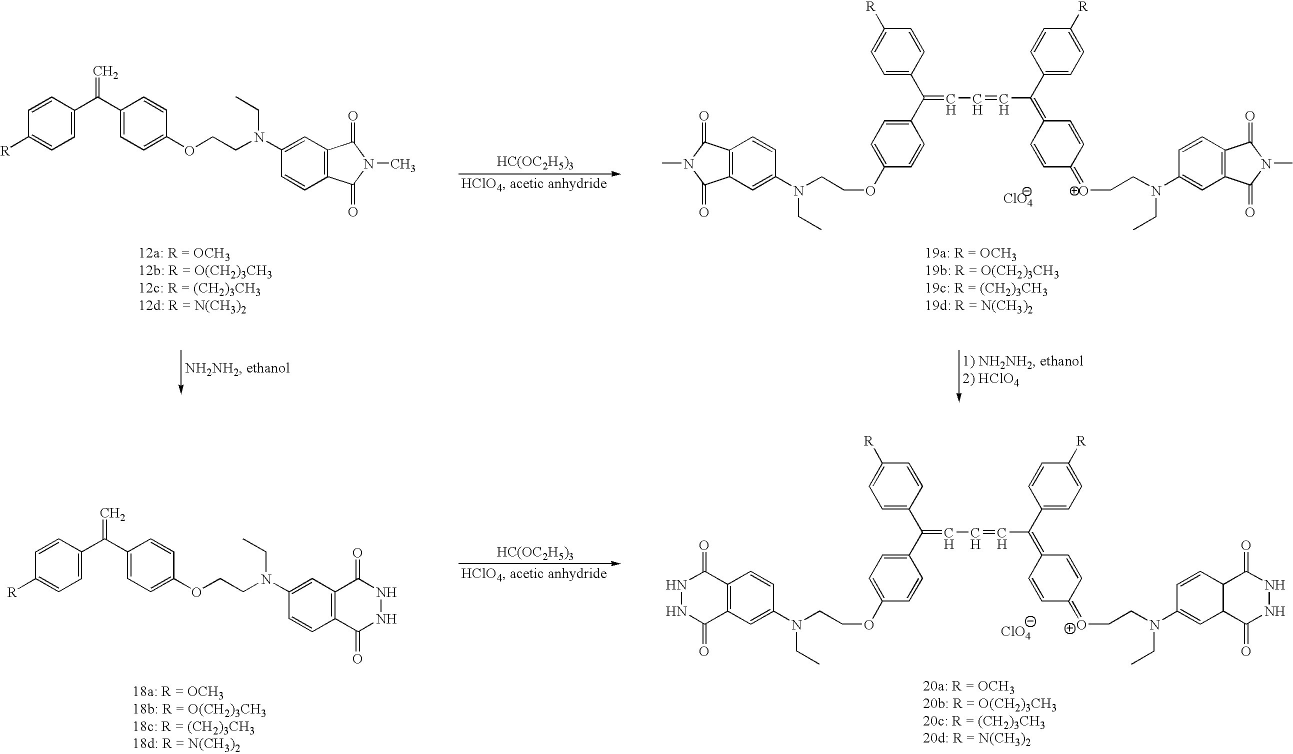

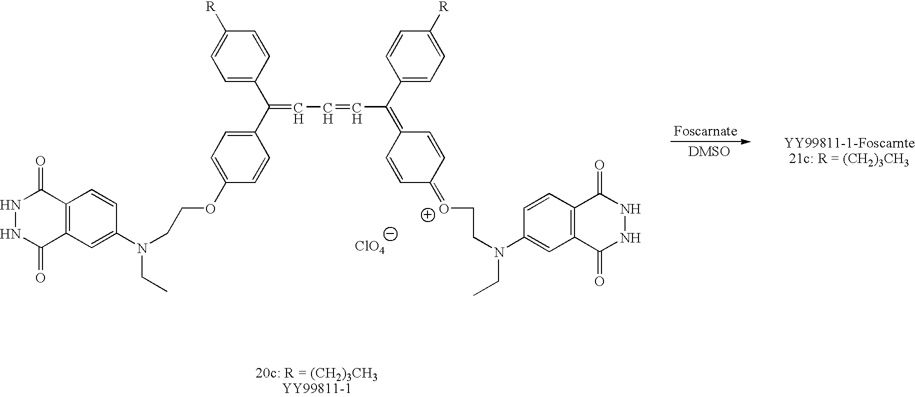

130. The method of synthesis of the compound of claim 105 comprising the general steps given by following representative formula

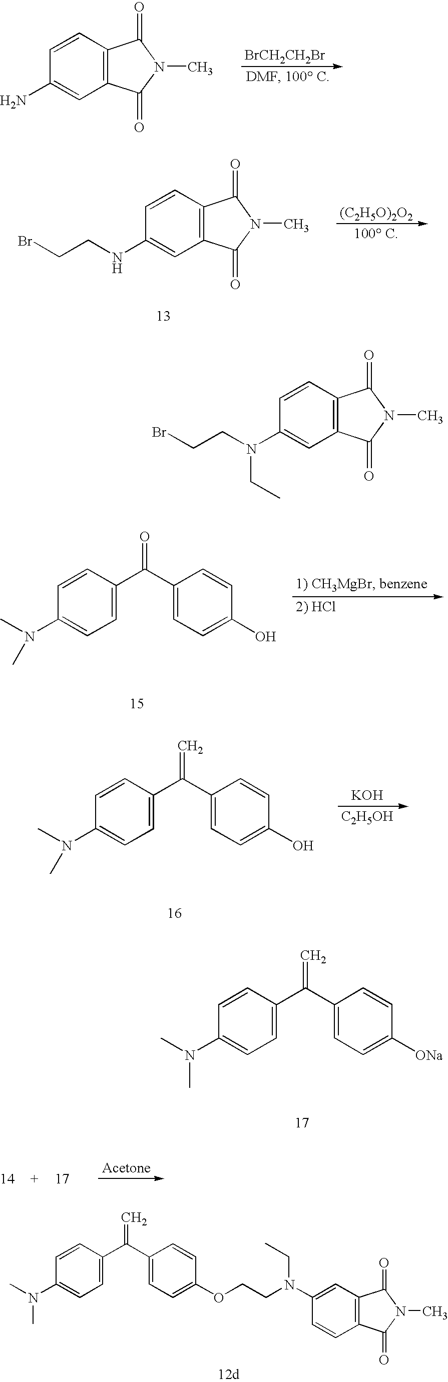

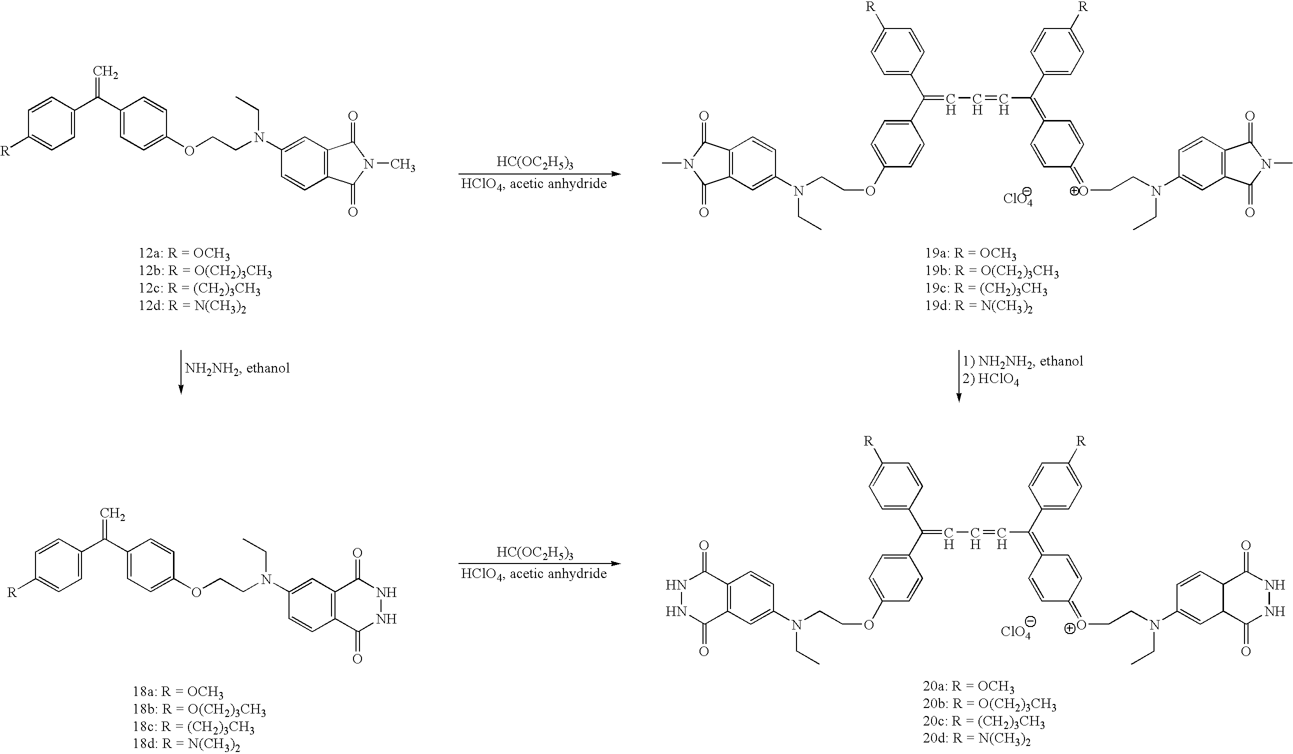

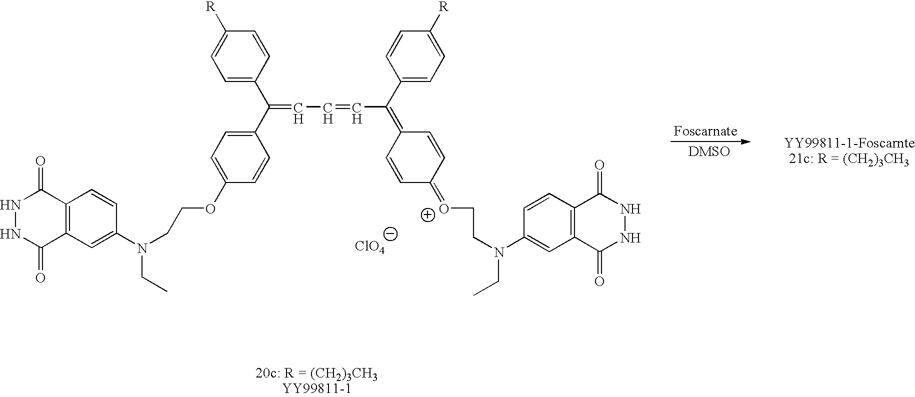

131. The method of synthesis of the compound of claim 1 wherein the A functionality comprises a phthalhydrazide such as a luminol derivative and the B functionality comprises a triarylpolymethine photochromic dye wherein A is attached to aryl groups of B comprising the steps of
forming a diaryl ketone,
forming a diaryl ketene from the diaryl ketone,
forming a protected aminophthalhydrazide such as aminophthalimide or aminophthalic acid diester,
adding a hydrocarbon linker to the protected aminophthalhydrazide, and
attaching the protected aminophthalhydrazide through the molecular linker to the aryl groups of diarylketene to form the precursor aminophthalimide-linked diarylketene, and reacting according to at least one of
(a) forming the A functionality from the precursor, and condensing the A-linked diarylketene with an aryl alkene aldehyde to form A-B, and
(b) condensing the precursor aminophthalimide-linked diarylketene with an aryl alkene aldehyde to form A precursor linked to B, and
forming the A functionality from the A precursor to form A-B.

132. The method of synthesis of the compound of claim 131 wherein the diaryl ketone is formed by a classical Friedel-Crafts acylation between a benzoyl halide and aryl compound with a hydrocarbon linker having a leaving group.

133. The method of synthesis of the compound of claim 132 wherein the aryl compound with a hydrocarbon linker having a leaving group comprises at least one of a halogenated-alkyl-aryl ether and a halogenated-aklyl-aryl amine wherein the halogen is the leaving group.

134. The method of synthesis of the compound of claim 133 wherein the halogenated-alkyl-aryl ether comprises 2-bromoethoxybenzene to give an aryl ketone such as 4-(2-bromoethoxy)benzophenone.

135. The method of synthesis of the compound of claim 133 wherein the halogenated-aklyl-aryl amine comprises 2-bromoethyl aminobenzene to give an aryl ketone such as 4-(2-bromoethyl amino)benzophenone.

136. The method of synthesis of the compound of claim 131 wherein the diaryl ketone is converted to the corresponding diarylketene by reacting with a methylating reagent such as a methyl Grignard reagent, methyl lithium reagent, lithium dimethylcopper reagent and then dehydration with acid.

137. The method of synthesis of the compound of claim 136 wherein the diaryl ketone is converted to the corresponding diarylketene by reacting with methylmagnesium bromide and then dehydration with acid.

138. The method of synthesis of the compound of claim 131 wherein the diaryl ketone is converted to the corresponding diarylketene by a Wittig reaction.

139. The method of synthesis of the compound of claim 131 wherein a linker is attached to the protected aminophthalhydrazide by a reaction of a nucleophilic group of the linker or protected aminophthalhydrazide with a leaving group of the linker or protected aminophthalhydrazide.

140. The method of synthesis of the compound of claim 131 wherein a linker is attached to the protected aminophthalhydrazide by reaction to form a bond between at least one of a nitrogen, oxygen, or carbon atom of the linker and at least one of a nitrogen, oxygen, or carbon atom of group of the protected aminophthalhydrazide by an addition or a substitution reaction of a leaving group.

141. The method of synthesis of the compound of claim 140 wherein a linker is attached to the protected aminophthalhydrazide by a substitution reaction of at least one of a halogen, tosylate group, ester group with a nitrogen, oxygen, or carbon atom.

142. The method of synthesis of the compound of claim 131 wherein attaching the protected aminophthalhydrazide through the molecular linker to one of the aryl groups of diarylketene to form the precursor aminophthalimide-linked diarylketene is by a reaction of a nucleophilic group of the linker or aryl group of diarylketene with a leaving group of the linker or aryl group of diarylketene.

143. The method of synthesis of the compound of claim 142 wherein a linker is attached to the aryl group of diarylketene by reaction to form a bond between at least one of a nitrogen, oxygen, or carbon atom of the linker and at least one of a nitrogen, oxygen, or carbon atom of group of the protected aminophthalhydrazide by an addition or a substitution reaction of a leaving group.

144. The method of synthesis of the compound of claim 143 wherein a linker is attached to the aryl group of diarylketene by a substitution reaction of at least one of a halogen, tosylate group, ester group with a nitrogen, oxygen, or carbon atom.

145. The method of synthesis of the compound of claim 131 wherein the precursor aminophthalimide-linked diarylketene is further reacted by condensation with an aryl alkene aldehyde in a nonaqueous solvent, containing an acid catalyst to form B linked to the A precursor.

146. The method of synthesis of the compound of claim 145 wherein the precursor aminophthalimide-linked diarylketene is an aminophthalimide-substituted 1,1-diarylethene,
the aryl alkene aldehyde is a p-aminophenyl alkene aldehyde such as p-(dimethylamino)cinnamaldehyde,
the nonaqueous solvent is acetic anhydride,
the acid catalyst is at least one of perchloric acid and tetrafluoroboric acid, and
the B linked to the A precursor comprises a aminophthalimide-substituted multiarylpolymethine dye.

147. The method of synthesis of the compound of claim 145 wherein the precursor aminophthalimide-linked diarylketene comprises at least one of the formula 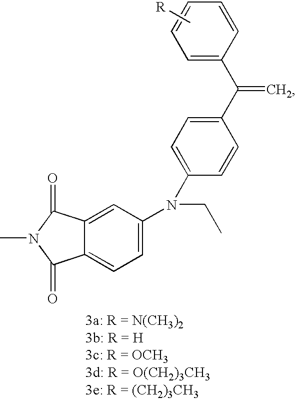
the aryl alkene aldehyde has the formula 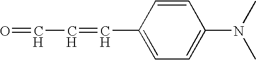
4-(Dimethylamino)cinnamaldehyde, and
the precursor of A-B comprises at least one of the formula

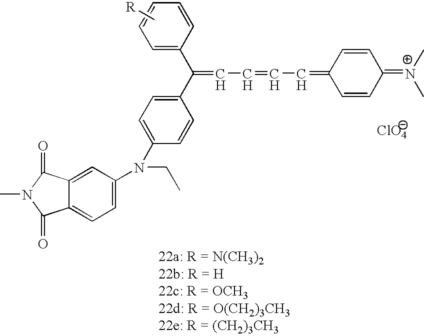

148. The method of synthesis of the compound of claim 145 wherein the phthalimide moiety of the A precursor is converted to the phthalhydrazide A functionality by treating with hydrazine, forming A-B.

149. The method of synthesis of the compound of claims 148 wherein the B functionality is protected by reacting with an anion such as hydroxide, methoxide and amine, the A-B precursor is refluxed with hydrazine in a suitable solvent such as an alcoholic solvent in inert atmosphere and then treated with acid such as perchloric acid, tetrafluroboric acid to form A-B.

150. The method of synthesis of the compound of claim 131 wherein the phthalimide moiety of the A precursor of the precursor aminophthalimide-linked diarylketene is converted to the phthalhydrazide A functionality by treating with hydrazine, forming A attached to a B precursor.

151. The method of synthesis of the compound of claim 150 wherein the A-linked diarylketene is further reacted by condensation with an aryl alkene aldehyde in a nonaqueous solvent, containing an acid catalyst to form A-B.

152. The method of synthesis of the compound of claim 151 wherein the A-linked diarylketene is an aminophthalhydrazide-substituted 1,1-diarylethene,
the aryl alkene aldehyde is a p-aminophenyl alkene aldehyde such as p-(dimethylamino)cinnamaldehyde,
the nonaqueous solvent is acetic anhydride,
the acid catalyst is at least one of perchloric acid and tetrafluoroboric acid, and
A-B comprises a aminophthalhydrazide-substituted multiarylpolymethine dye.

153. The method of synthesis of the compound of claim 152 wherein the A-linked diarylketene comprises at least one of the formula 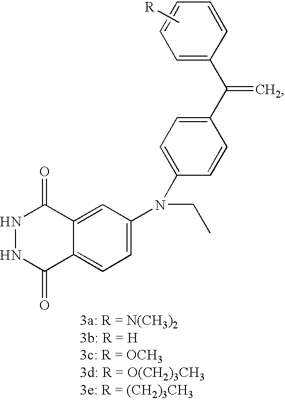
the aryl alkene aldehyde has the formula 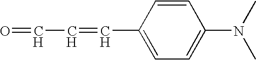
4-(Dimethylamino)cinnamaldehyde, and
A-B comprises at least one of the formula

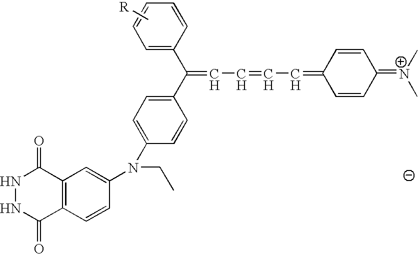

154. The method of synthesis of the compound of claims 149 and 151 further comprising the step of reacting the B functionality with one nucleophilic species of a C functionality such as Foscarnet to form A-B-C.

155. The method of synthesis of the compound of claim 131 comprising the general steps given by following representative formula

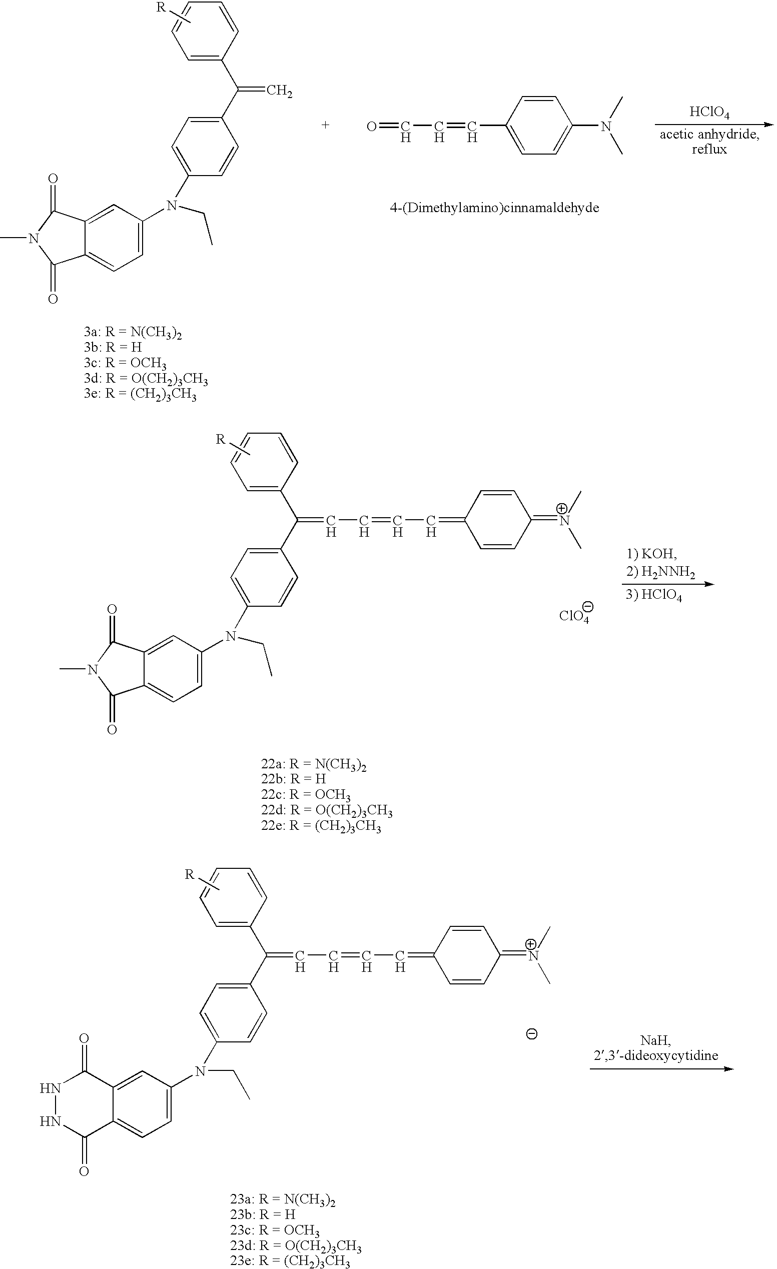

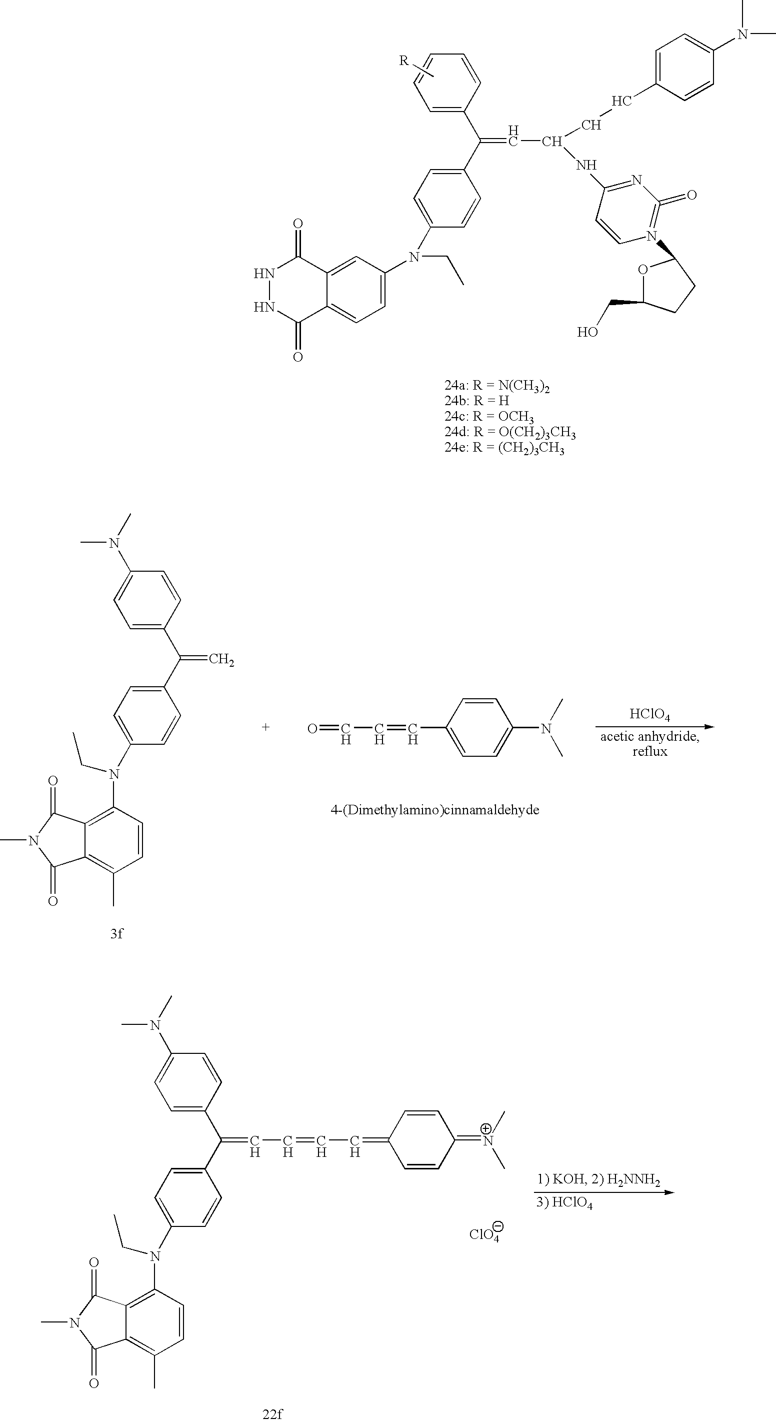

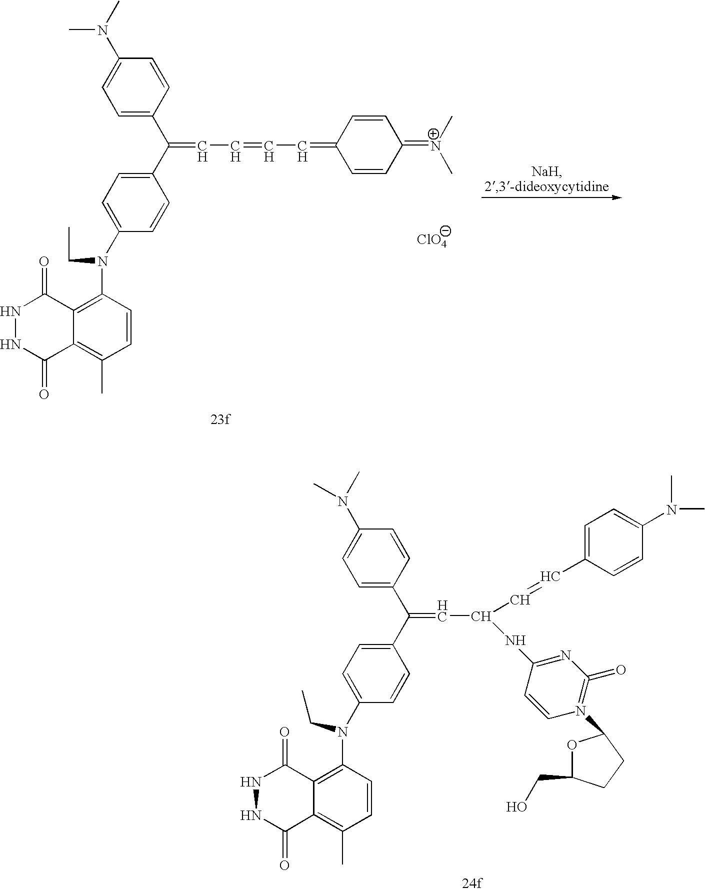

156. The method of synthesis of the compound of claim 131 comprising the general steps given by following representative formula

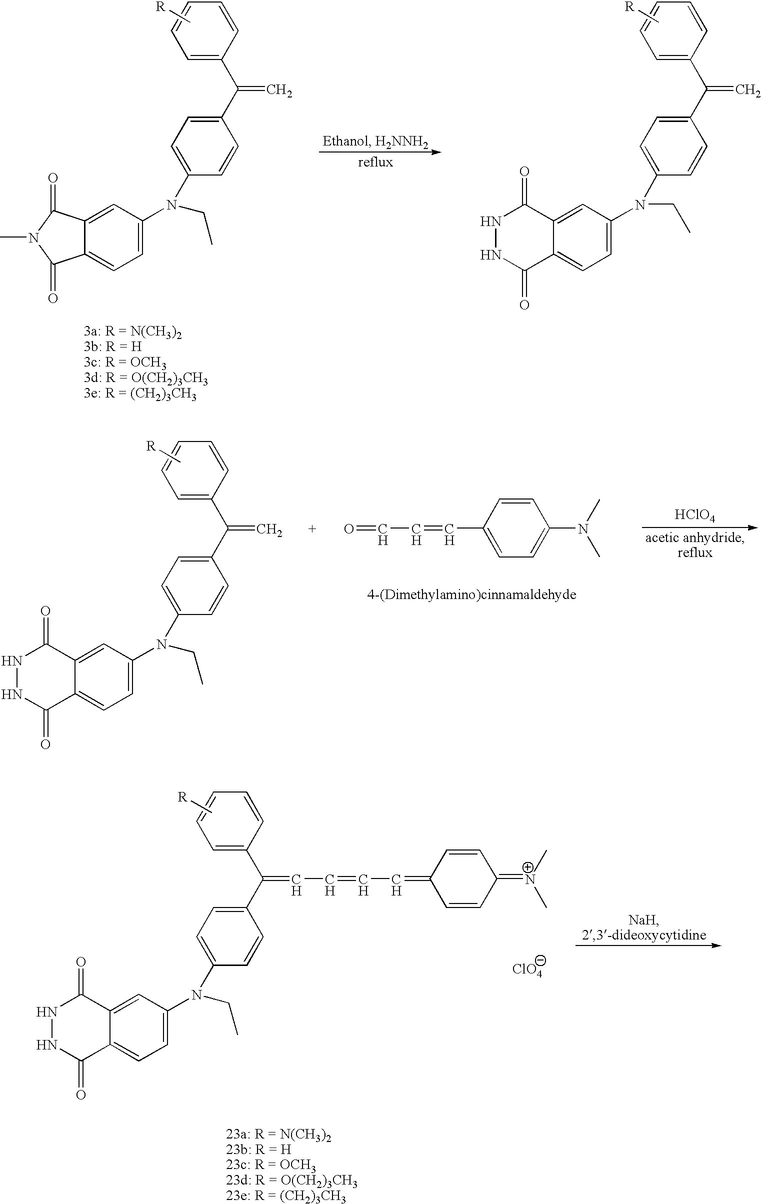

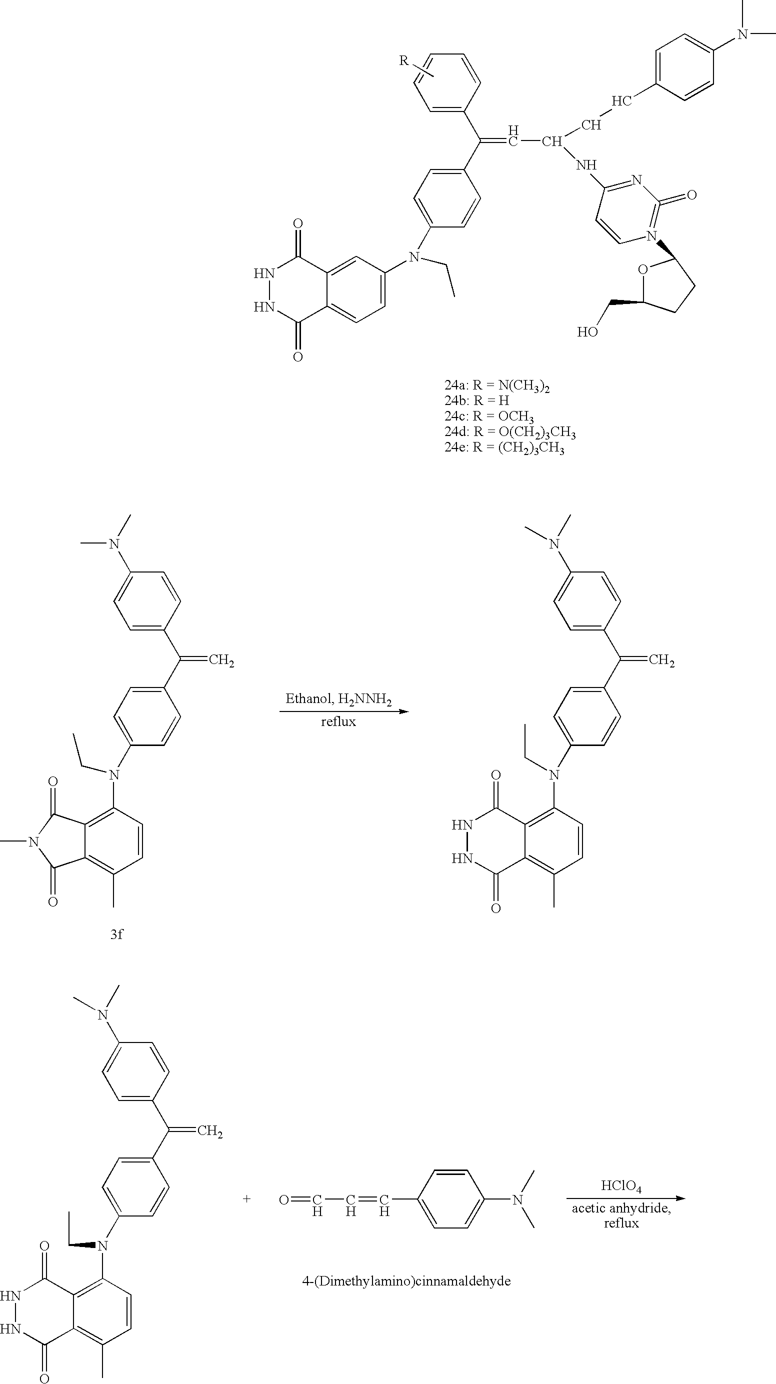

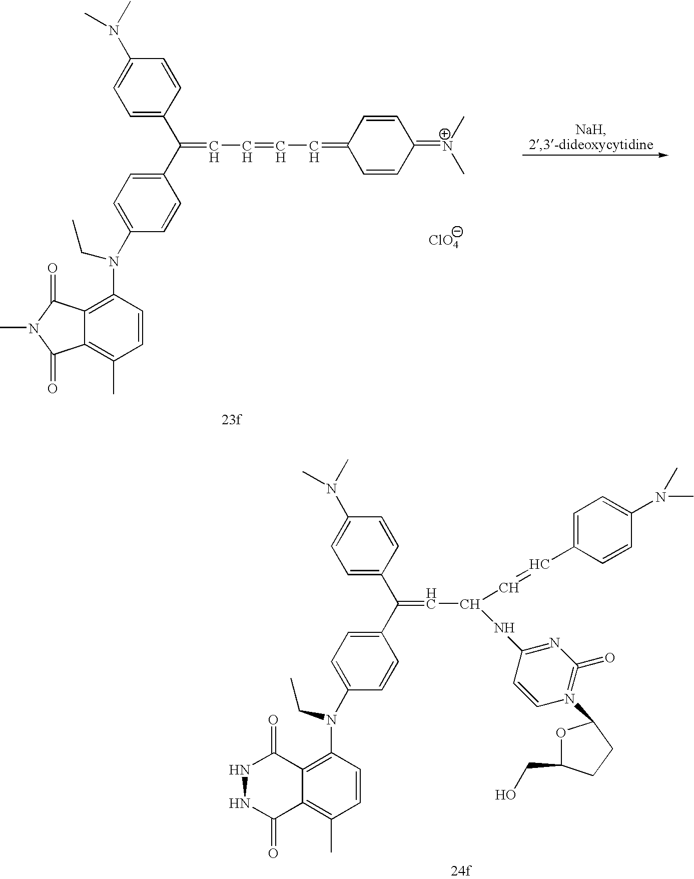

157. The method of synthesis of the compound of claim 1 wherein the A functionality comprises a phthalhydrazide such as a luminol derivative and the B functionality comprises a triarylpolymethine photochromic dye wherein A is attached to aryl groups of B comprising the steps of
forming a diaryl ketone,
forming a diaryl ketene from the diaryl ketone,
condensing the diarylketene with an aryl alkene aldehyde to form B
forming a protected aminophthalhydrazide such as aminophthalimide or aminophthalic acid diester,
adding a hydrocarbon linker to the protected aminophthalhydrazide, and
attaching the protected aminophthalhydrazide through the molecular linker to the aryl groups of B to form the precursor aminophthalimide-linked B, and
forming the A functionality from the precursor to form A-B.

158. The method of synthesis of the compound of claim 157 wherein at least one of the diaryl ketone and diarylketene is halo-substituted and the protected aminophthalhydrazide is attached through the linker by an amination reaction.

159. The method of synthesis of the compound of claim 158 wherein the halo-substituted diarylketene precursor compounds comprises the formula of at least one of 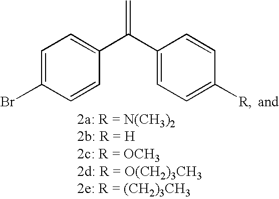
and the halo-substituted multiarylpolymethine dyes, such as 1-(p-bromophenyl)-1,5-bis(p-dimethylaminophenyl)-pentadienium perchlorate, are be prepared by condensation with a p-aminophenyl alkene aldehyde such as p-(dimethylamino)cinnamaldehyde.

160. The method of synthesis of the compound of claim 158 wherein B is protected by reacting with an anion such as alkoxide and then coupled with A by amination of aryl halide such as the palladium-catalyzed animation of aryl halide to obtain the alkoxide-protected aminophthalimide-substituted multiarylpolymethine dye.

161. The method of synthesis of the compound of claim 160 wherein the protected aminophthalhydrazide-linked to B from the alkoxide-protected aminophthalimide-substituted multiarylpolymethine dye comprises at least one of the formula

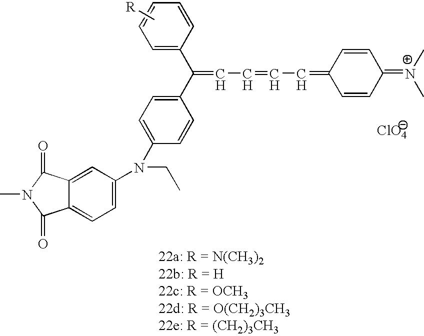

162. The method of synthesis of the compound of claim 160 wherein the alkoxide-protected aminophthalimide-substituted multiarylpolymethine dye is refluxed with hydrazine in a suitable solvent such as an alcoholic solvent to convert the amino-phthalimide moiety to the aminophthalhydrazide moiety and then treated with acid to generate A-B.

163. The method of synthesis of the compound of claim 162 wherein A-B comprises at least one of the formula

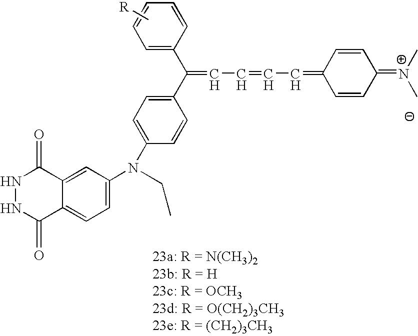

164. The method of synthesis of the compound of claim 162 further comprising the step of reacting the B functionality with one nucleophilic species of a C functionality such as Foscarnet to form A-B-C.

165. The method of synthesis of the compound of claim 157 wherein at least one of the diaryl ketone and diarylketene is halo-substituted and an aminophthalhydrazide is attached through the linker by an amination reaction.

166. The method of synthesis of the compound of claim 1 wherein the A functionality comprises an active oxalate and the B functionality comprises a multiarylpolymethine photochromic dye wherein A is attached to aryl groups of B comprising the steps of
forming a halo-substituted diaryl ketone,
forming a halo-substituted diaryl ketene from the diaryl ketone,
amination of the halo-substituted diaryl ketene to give amino diarylketene,
substitution at the amino group of the ketene to forming the corresponding sulfonamide,
condensing the sulfonamide with a catalyst, and
react with oxalyl halide to form A-B.

167. The method of synthesis of the compound of claim 1 wherein the A functionality comprises an cyclized active oxalate and the B functionality comprises a multiarylpolymethine photochromic dye wherein A is attached to aryl groups of B comprising the steps of
forming a halo-substituted diaryl ketone,
forming a halo-substituted diaryl ketene from the diaryl ketone,
amination of the halo-substituted diaryl ketene to give amino diarylketene,
substitution at the amino group of the ketene to forming the corresponding sulfonamide,
reacting 2 molar proportions of a N-substituted aminodiarylketene with 1 molar oxalyl halide to yield the N,N′-bisaryl oxamide,
condensing the oxamide with a catalyst to form A-B.

168. The method of synthesis of the compound of claim 167 wherein the halo-substituted diaryl ketene is aminated using methods such as the palladium-catalyzed amination of aryl halide with benzophenoneimine to give the amino diarylketene.

169. The method of synthesis of the compound of claim 167 wherein the amino groups of the ketene are substituted forming the corresponding sulfonamide by reacting with sulfonyl anhydride.

170. The method of synthesis of the compound of claim 167 wherein the oxamide is condensed with an orthoester such as triethylorthofomate in a nonaqueous solvent such as acetic anhydride containing acid catalyst such as tetrafluoroboric acid, to form the cyclized oxamido-tetraarylpolymethine dye comprising A-B.

171. The method of synthesis of the compound of claim 166 further comprising the step of reacting the B functionality with one nucleophilic species of a C functionality such as Foscarnet to form A-B-C.

172. The method of synthesis of the compound of claim 167 wherein the general steps are given by following representative formula

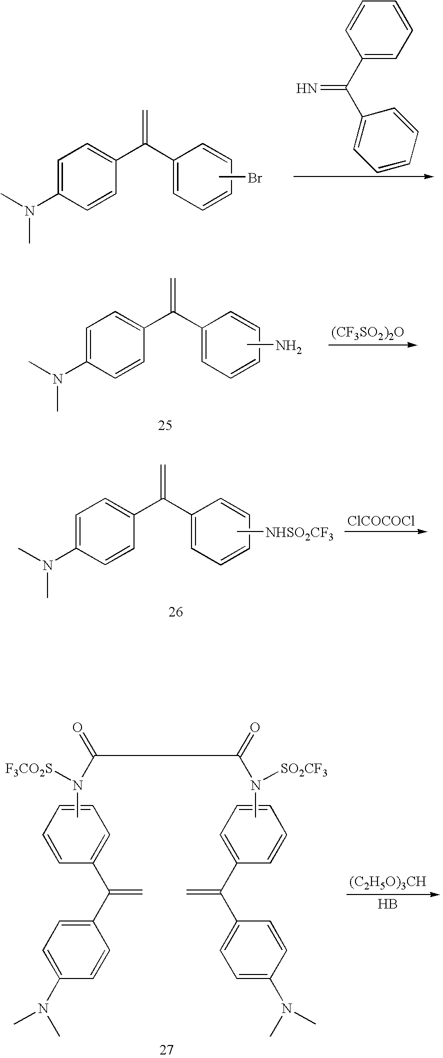

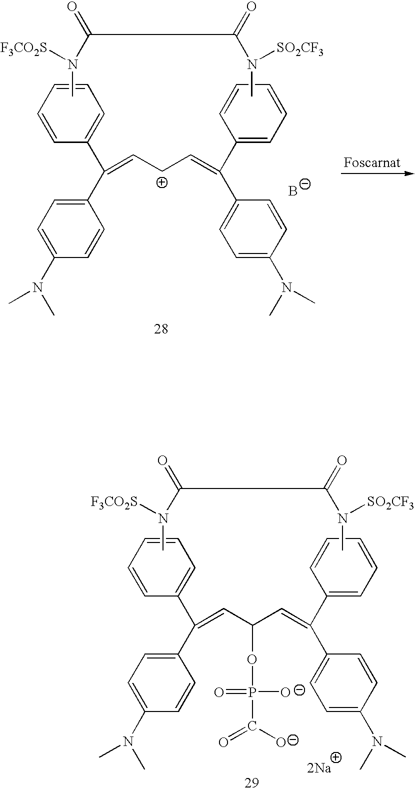

173. The method of synthesis of the compound of claim 1 wherein the A functionality comprises an active oxalate and the B functionality comprises a multiarylpolymethine photochromic dye wherein A is attached to aryl groups of B through a molecular linker comprising the steps of
forming B comprising a functionalized tetraarylpolymethine dye,
reacting a substituted amine with a sulfonyl anhydride to form a substituted alkyl sulfonamide,
reacting the substituted alkyl sulfonamide with an oxalyl derivative to form a substituted oxamide,
reacting the substituted oxamide with the functionalized tetraarylpolymethine dye to form A-B comprising a cyclized oxamido-tetraarylpolymethine.

174. The method of synthesis of the compound of claim 173 wherein the substituted amine is N-2-bromoethylsulfamide.

175. The method of synthesis of the compound of claim 173 wherein the oxalyl derivative is oxalyl chloride.

176. The method of synthesis of the compound of claim 173 wherein the oxamide is a N-2-bromoethyl-N-sulfonyloxamide derivative.

177. The method of synthesis of the compound of claim 173 wherein the oxalyl derivative is oxalyl chloride.

178. The method of synthesis of the compound of claim 173 wherein the functionalized tetraarylpolymethine derivative is a salt of a 1,5-bis(4-hydroxyphenyl)-1,5-diarylpentadiene derivative.

179. The method of synthesis of the compound of claim 173 wherein the cyclized oxamido-tetraarylpolymethine A-B compound is a 1,5-(4,4′-(2,2′-N,N′-disulfonyloxamidodiethoxy)phenyl-1,5-diarylpentadiene cation derivative.

180. The method of synthesis of the compound of claim 173 further comprising the step of reacting the B functionality with one nucleophilic species of a C functionality such as Foscarnet to form A-B-C.

181. The method of synthesis of the compound of claim 173 comprising the general steps given by following representative formula

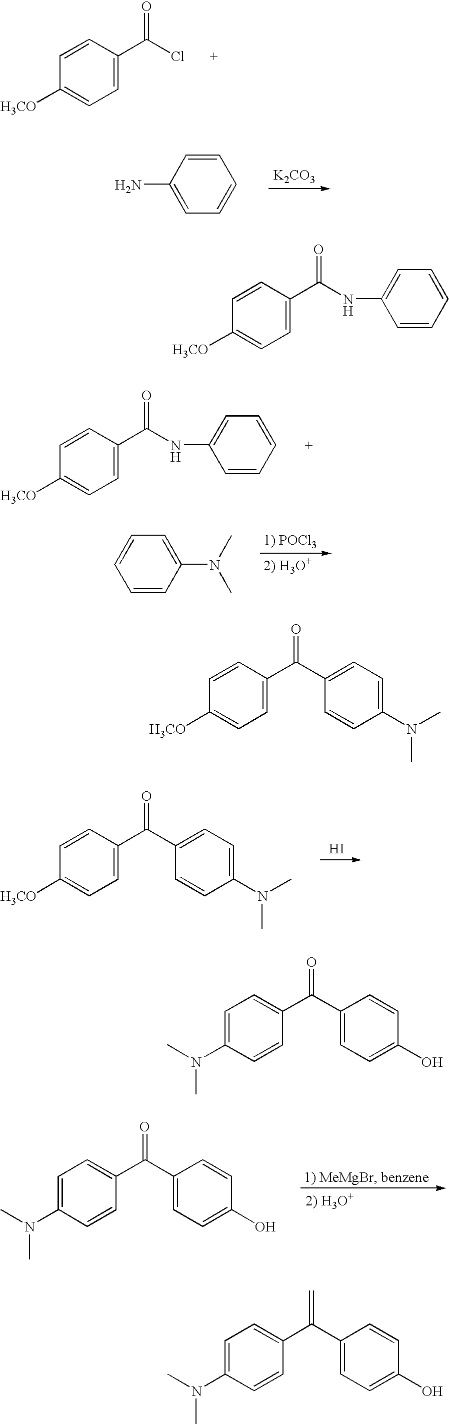

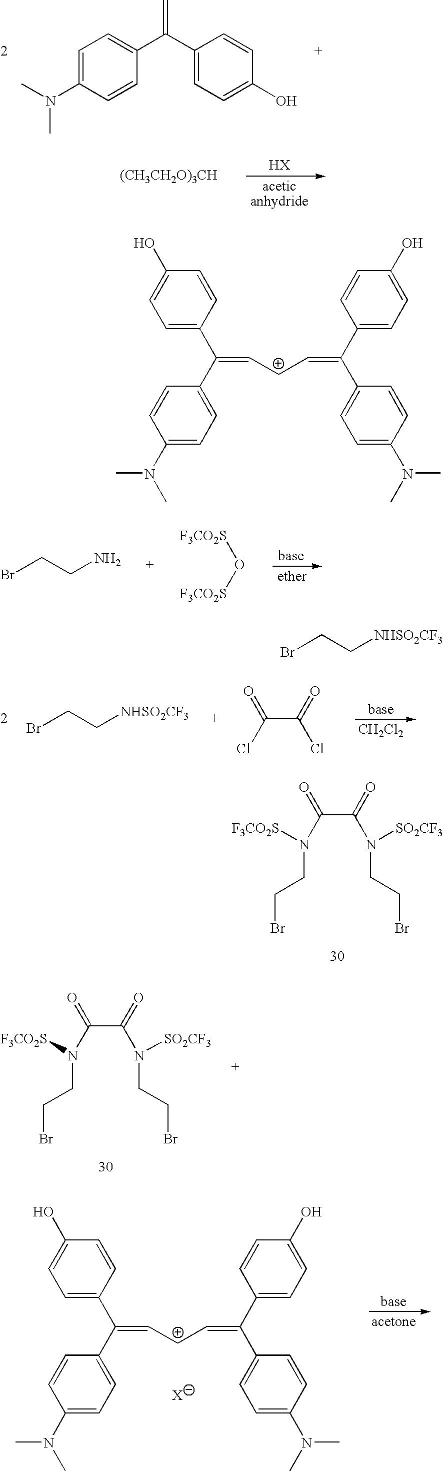

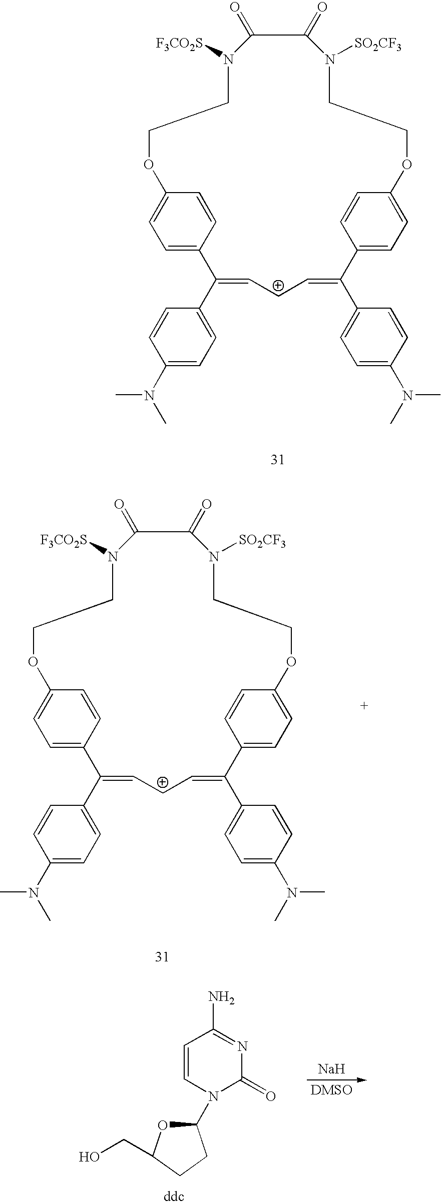

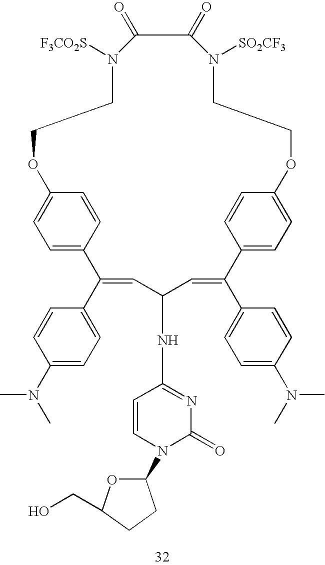

182-227 (Cancelled)
